SEQUENCE LISTING (79 sequences)

SEQ ID NO: 1 - protein, 30 aa, Artificial Sequence
  Recombinant sequence
AQLEKELQALEKENAQLEWELQALEKELAQ

SEQ ID NO: 2 - protein, 30 aa, Artificial Sequence
  Recombinant sequence
AQLKKKLQALKKKNAQLKWKLQALKKKLAQ

SEQ ID NO: 3 - protein, 33 aa, Artificial Sequence
  Recombinant sequence
AQLEKELQALEKENAQLEWELQALEKELAQGGC

SEQ ID NO: 4 - protein, 33 aa, Artificial Sequence
  Recombinant sequence
AQLKKKLQALKKKNAQLKWKLQALKKKLAQGGC

SEQ ID NO: 5 - DNA, 141 bp, Artificial Sequence
  Recombinant sequence
cccccgaattcggcgcggcgcagctggaaaaagaactgcaggcgctggaaaaagaaaacgcgcagctggaatgggaactgcaggcgctggaaaaagaactggcgcagggcggctgcccggcgggcgcgggggatccccccc

SEQ ID NO: 6 - protein, 39 aa, Artificial Sequence
  Recombinant sequence
GAAQLEKELQALEKENAQLEWELQALEKELAQGGCPAGA

SEQ ID NO: 7 - DNA, 226 bp, Artificial Sequence
  Recombinant sequence
gcatgctttatcaaaaagagtgttgacttgtgagcggataacaatgatacttagattcatcgagagggacacggcgaaaaataaggaggaaaaaaaaatggcgaataacgatctctttcaggcatcacgtcggcgttttctggcacaactcggcggcttaaccgtcgccgggatgctggggccctcattgttaacgccgcgacgtgcgactgcgagatctgcatgc

SEQ ID NO: 8 - protein, 15 aa, Artificial Sequence
  Recombinant sequence
MSIYVYALPLKMLNI

SEQ ID NO: 9 - protein, 15 aa, Artificial Sequence
  Recombinant sequence
ALPLKMLNIPSINVH

SEQ ID NO: 10 - protein, 13 aa, Artificial Sequence
  Recombinant sequence
PKYVKQNTLKLAT

SEQ ID NO: 11 - protein, 15 aa, Artificial Sequence
  Recombinant sequence
INLMKLPLAYVYISM

SEQ ID NO: 12 - protein, 6 aa, Artificial Sequence
  Recombinant sequence
SSGSSG

SEQ ID NO: 13 - protein, 13 aa, Artificial Sequence
  Recombinant sequence
TSLYNLRRGTALA

SEQ ID NO: 14 - DNA, 141 bp, Artificial Sequence
  Recombinant sequence
cccccgcatgctttatcaaaaagagtgttgacttgtgagcggataacaatgatacttagattcatcgagagggacacggcgaaaaataaggaggaaaaaaccatggagatctaagcttctcgagaggcctgcatgcccccc

SEQ ID NO: 15 - protein, 307 aa, Artificial Sequence
  Recombinant sequence
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIP

SEQ ID NO: 16 - DNA, 927 bp, Artificial Sequence
  Recombinant sequence
atggcggtgatggcgccgcgcaccctgctgctgctgctgagcggcgcgctggcgctgacccagacctgggcgggcagccatagcatgcgctatttttttaccagcgtgagccgcccgggccgcggcgaaccgcgctttattgcggtgggctatgtggatgatacccagtttgtgcgctttgatagcgatgcggcgagccagaaaatggaaccgcgcgcgccgtggattgaacaggaaggcccggaatattgggatcaggaaacccgcaacatgaaagcgcatagccagaccgatcgcgcgaacctgggcaccctgcgcggctattataaccagagcgaagatggcagccataccattcagattatgtatggctgcgatgtgggcccggatggccgctttctgcgcggctatcgccaggatgcgtatgatggcaaagattatattgcgctgaacgaagatctgcgcagctggaccgcggcggatatggcggcgcagattaccaaacgcaaatgggaagcggtgcatgcggcggaacagcgccgcgtgtatctggaaggccgctgcgtggatggcctgcgccgctatctggaaaacggcaaagaaaccctgcagcgcaccgatccgccgaaaacccatatgacccatcatccgattagcgatcatgaagcgaccctgcgctgctgggcgctgggcttttatccggcggaaattaccctgacctggcagcgcgatggcgaagatcagacccaggataccgaactggtggaaacccgcccggcgggcgatggcacctttcagaaatgggcggcggtggtggtgccgagcggcgaagaacagcgctatacctgccatgtgcagcatgaaggcctgccgaaaccgctgaccctgcgctgggaactgagcagccagccgaccattccgtaataa

SEQ ID NO: 17 - protein, 39 aa, Artificial Sequence
  Recombinant sequence
GAAQLKKKLQALKKKNAQLKWKLQALKKKLAQGGCPAGA

SEQ ID NO: 18 - DNA, 117 bp, Artificial Sequence
  Recombinant sequence
ggcgcggcgcagctgaaaaaaaaactgcaggcgctgaaaaaaaaaaacgcgcagctgaaatggaaactgcaggcgctgaaaaaaaaactggcgcagggcggctgcccggcgggcgcg

SEQ ID NO: 19 - DNA, 18 bp, Artificial Sequence
  Recombinant sequence
ggcagcagcggcagcagc

SEQ ID NO: 20 - protein, 99 aa, Artificial Sequence [sequence omitted]

SEQ ID NO: 21 - DNA, 297 bp, Artificial Sequence
  Recombinant sequence
attcagcgcaccccgaaaattcaggtgtatagccgccatccggcggaaaacggcaaaagcaactttctgaactgctatgtgagcggctttcatccgagcgatattgaagtggatctgctgaaaaacggcgaacgcattgaaaaagtggaacatagcgatctgagctttagcaaagattggagcttttatctgctgtattataccgaatttaccccgaccgaaaaagatgaatatgcgtgccgcgtgaaccatgtgaccctgagccagccgaaaattgtgaaatgggatcgcgatatg

SEQ ID NO: 22 - DNA, 1418 bp, Artificial Sequence
  Recombinant sequence
cccccgatccggaggaaaaaaaaatgggcgcggcgcagctgaaaaaaaaactgcaggcgctgaaaaaaaaaaacgcgcagctgaaatggaaactgcaggcgctgaaaaaaaaactggcgcagggcggctgcccggcgggcgcgggcagcagcggcagcagcatggcggtgatggcgccgcgcaccctgctgctgctgctgagcggcgcgctggcgctgacccagacctgggcgggcagccatagcatgcgctatttttttaccagcgtgagccgcccgggccgcggcgaaccgcgctttattgcggtgggctatgtggatgatacccagtttgtgcgctttgatagcgatgcggcgagccagaaaatggaaccgcgcgcgccgtggattgaacaggaaggcccggaatattgggatcaggaaacccgcaacatgaaagcgcatagccagaccgatcgcgcgaacctgggcaccctgcgcggctattataaccagagcgaagatggcagccataccattcagattatgtatggctgcgatgtgggcccggatggccgctttctgcgcggctatcgccaggatgcgtatgatggcaaagattatattgcgctgaacgaagatctgcgcagctggaccgcggcggatatggcggcgcagattaccaaacgcaaatgggaagcggtgcatgcggcggaacagcgccgcgtgtatctggaaggccgctgcgtggatggcctgcgccgctatctggaaaacggcaaagaaaccctgcagcgcaccgatccgccgaaaacccatatgacccatcatccgattagcgatcatgaagcgaccctgcgctgctgggcgctgggcttttatccggcggaaattaccctgacctggcagcgcgatggcgaagatcagacccaggataccgaactggtggaaacccgcccggcgggcgatggcacctttcagaaatgggcggcggtggtggtgccgagcggcgaagaacagcgctatacctgccatgtgcagcatgaaggcctgccgaaaccgctgaccctgcgctgggaactgagcagccagccgaccattccgtaataaggaggaaaaaaaaatgattcagcgcaccccgaaaattcaggtgtatagccgccatccggcggaaaacggcaaaagcaactttctgaactgctatgtgagcggctttcatccgagcgatattgaagtggatctgctgaaaaacggcgaacgcattgaaaaagtggaacatagcgatctgagctttagcaaagattggagcttttatctgctgtattataccgaatttaccccgaccgaaaaagatgaatatgcgtgccgcgtgaaccatgtgaccctgagccagccgaaaattgtgaaatgggatcgcgatatgtaataagaattcccccc

SEQ ID NO: 23 - protein, 11 aa, Artificial Sequence
  Recombinant sequence
FVEENLQHWSY

SEQ ID NO: 24 - DNA, 33 bp, Artificial Sequence
  Recombinant sequence
tttgtggaagaaaacctgcagcattggagctat

SEQ ID NO: 25 - protein, 9 aa, Artificial Sequence
  Recombinant sequence
VSEVVKDAY

SEQ ID NO: 26 - DNA, 27 bp, Artificial Sequence
  Recombinant sequence
gtgagcgaagtggtgaaagatgcgtat

SEQ ID NO: 27 - protein, 9 aa, Artificial Sequence
  Recombinant sequence
LTDDDLLRY

SEQ ID NO: 28 - DNA, 27 bp, Artificial Sequence
  Recombinant sequence
ctgaccgatgatgatctgctgcgctat

SEQ ID NO: 29 - protein, 11 aa, Artificial Sequence
  Recombinant sequence
YSWHQLNEEVF

SEQ ID NO: 30 - DNA, 33 bp, Artificial Sequence
  Recombinant sequence
tatagctggcatcagctgaacgaagaagtgttt

SEQ ID NO: 31 - DNA, 77 bp, Artificial Sequence
  Recombinant sequence
cccccagatctggaggaaaaaaaaatgtttgtggaagaaaacctgcagcattggagctattaataactcgagccccc

SEQ ID NO: 32 - DNA, 71 bp, Artificial Sequence
  Recombinant sequence
cccccagatctggaggaaaaaaaaatggtgagcgaagtggtgaaagatgcgtattaataactcgagccccc

SEQ ID NO: 33 - DNA, 71 bp, Artificial Sequence
  Recombinant sequence
cccccagatctggaggaaaaaaaaatgctgaccgatgatgatctgctgcgctattaataactcgagccccc

SEQ ID NO: 34 - DNA, 77 bp, Artificial Sequence
  Recombinant sequence
cccccagatctggaggaaaaaaaaatgtatagctggcatcagctgaacgaagaagtgttttaataactcgagccccc

SEQ ID NO: 35 - protein, 10 aa, Artificial Sequence
  Recombinant sequence
SSGSSGSSPG

SEQ ID NO: 36 - protein, 10 aa, Artificial Sequence
  Recombinant sequence
SSGSSGSPSG

SEQ ID NO: 37 - protein, 10 aa, Artificial Sequence
  Recombinant sequence
SSGSSGPSSG

SEQ ID NO: 38 - protein, 10 aa, Artificial Sequence
  Recombinant sequence
SSGSSPGSSG

SEQ ID NO: 39 - protein, 10 aa, Artificial Sequence
  Recombinant sequence
SSGSPSGSSG

SEQ ID NO: 40 - protein, 10 aa, Artificial Sequence
  Recombinant sequence
SSGPSSGSSG

SEQ ID NO: 41 - protein, 306 aa, Artificial Sequence
  Recombinant sequence
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLWQRDGEDQTQDTELVETRPAGDGTFQKWAAVVVPSGEEQRYTCHVQHEGLPKPLTLRWELSSQPTIP

SEQ ID NO: 42 - DNA, 927 bp, Artificial Sequence; identical to SEQ ID NO: 16
  Recombinant sequence

SEQ ID NO: 43 - DNA, 117 bp, Artificial Sequence; identical to SEQ ID NO: 18
  Recombinant sequence

SEQ ID NO: 44 - protein, 6 aa, Artificial Sequence
  Recombinant sequence
GSSGSS

SEQ ID NO: 45 - DNA, 18 bp, Artificial Sequence
  Recombinant sequence
ggcagcagcggcagcagc

SEQ ID NO: 46 - protein, 12 aa, Artificial Sequence
  Recombinant sequence
SSGPSSGSSGPS

SEQ ID NO: 47 - protein, 108 aa, Artificial Sequence
  Recombinant sequence
MATRTPKLVKHTLLTRFKDEITREQIDNYINDYTNLLDLIPSMKSFNWGTDLGMESAELNRGYTHAFESTFESKSGLQEYLDSAALAAFAEGFLPTLSQRLVIDYFLY

SEQ ID NO: 48 - protein, 307 aa, Artificial Sequence; identical to SEQ ID NO: 15
  Recombinant sequence

SEQ ID NO: 49 - DNA, 42 bp, Artificial Sequence
  Recombinant sequence
  misc_feature (7)..(8): n is a, c, g, or t
  misc_feature (10)..(11): n is a, c, g, or t
  misc_feature (13)..(14): n is a, c, g, or t
  misc_feature (19)..(20): n is a, c, g, or t
  misc_feature (22)..(23): n is a, c, g, or t
  misc_feature (28)..(29): n is a, c, g, or t
  misc_feature (31)..(32): n is a, c, g, or t
  misc_feature (34)..(35): n is a, c, g, or t
gctctgnnknnknnsgytnnknnsctgnnknnknnsatgcat

SEQ ID NO: 50 - DNA, 33 bp, Artificial Sequence
  Recombinant sequence
cagccagttgctctcgagggacagagcatgcat

SEQ ID NO: 51 - DNA, 28 bp, Artificial Sequence
  Recombinant sequence
ggttcacaggcttggtctgcaggctctg

SEQ ID NO: 52 - DNA, 22 bp, Artificial Sequence
  Recombinant sequence
ggttcacaggcttggtctgcag

SEQ ID NO: 53 - DNA, 21 bp, Artificial Sequence
  Recombinant sequence
cagccagttgctctcgaggga

SEQ ID NO: 54 - DNA, 29 bp, Artificial Sequence
  Recombinant sequence
agcggctggatatgcggatggtcgcgtag

SEQ ID NO: 55 - DNA, 33 bp, Artificial Sequence
  Recombinant sequence
gcgcagttgcatcggagtcgatgcatgcctacg

SEQ ID NO: 56 - DNA, 20 bp, Artificial Sequence
  Recombinant sequence
atggcagtagtcgaaagtac

SEQ ID NO: 57 - DNA, 20 bp, Artificial Sequence
  Recombinant sequence
gtacattcgactactagtag

SEQ ID NO: 58 - DNA, 20 bp, Artificial Sequence
  Recombinant sequence
gtgtgagtcgtgcgttctcg

SEQ ID NO: 59 - DNA, 20 bp, Artificial Sequence
  Recombinant sequence
ccgtgacgcacgacttagcg

SEQ ID NO: 60 - DNA, 20 bp, Artificial Sequence
  Recombinant sequence
ccaatgtgaccgtagcgtag

SEQ ID NO: 61 - DNA, 20 bp, Artificial Sequence
  Recombinant sequence
ctgagctacggtcactaagc

SEQ ID NO: 62 - DNA, 20 bp, Artificial Sequence
  Recombinant sequence
cacaggctgagctgaggaca

SEQ ID NO: 63 - DNA, 20 bp, Artificial Sequence
  Recombinant sequence
agacctcagctcagctgcac

SEQ ID NO: 64 - DNA, 117 bp, Artificial Sequence
  Recombinant sequence
ggcgcggcgcagctggaaaaagaactgcaggcgctggaaaaagaaaacgcgcagctggaatgggaactgcaggcgctggaaaaagaactggcgcagggcggctgcccggcgggcgcg

SEQ ID NO: 65 - DNA, 117 bp, Artificial Sequence
  Recombinant sequence
ggcgcggctcagctggaaaaagaactgcaggctctggaaaaagagaacgcgcagctggaatgggagctgcaggcgctggagaaagaactggctcagggcggctgcccagcgggcgcg

SEQ ID NO: 66 - DNA, 117 bp, Artificial Sequence
  Recombinant sequence
ggcgctgcgcagctggaaaaagagctgcaggcgctggagaaagaaaacgcgcaactggaatgggaactgcaagcgctggaaaaagaactggcgcaaggcggctgcccggcgggcgcc

SEQ ID NO: 67 - DNA, 117 bp, Artificial Sequence
  Recombinant sequence
ggcgcggcgcaactggaaaaagaactgcaagcgctggaaaaagaaaacgcccagctggaatgggagctgcaggcgctggagaaagaactggctcagggcggctgcccggctggcgcg

SEQ ID NO: 68 - DNA, 117 bp, Artificial Sequence
  Recombinant sequence
ggcgcggcccagctggaaaaagagctgcaggccctggaaaaagaaaacgcgcaactggaatgggaactgcaagcgctggaaaaagaactggcacagggcggctgcccggcgggcgcc

SEQ ID NO: 69 - DNA, 117 bp, Artificial Sequence
  Recombinant sequence
ggcgcggcgcaactggaaaaagaactgcaagcgctggaaaaagaaaacgcccagctggaatgggagctgcaggcgctggagaaagaactggcgcaaggcggctgcccagcgggcgcg

SEQ ID NO: 70 - DNA, 30 bp, Artificial Sequence
  Recombinant sequence
agcagcggcagcagcggcagcagcccgggc

SEQ ID NO: 71 - DNA, 30 bp, Artificial Sequence
  Recombinant sequence
agtagcggtagcagcggcagcccgagcggc

SEQ ID NO: 72 - DNA, 30 bp, Artificial Sequence
  Recombinant sequence
agcagcggcagcagtggcccgagcagcggc

SEQ ID NO: 73 - DNA, 30 bp, Artificial Sequence
  Recombinant sequence
agcagtggtagcagcccgggcagcagcggc

SEQ ID NO: 74 - DNA, 30 bp, Artificial Sequence
  Recombinant sequence
agtagcggcagcccgagcggcagcagcggc

SEQ ID NO: 75 - DNA, 30 bp, Artificial Sequence
  Recombinant sequence
agcagcggcccgagcagcggcagcagcggc

SEQ ID NO: 76 - DNA, 903 bp, Artificial Sequence
  Recombinant sequence
ggcccagccggccggcgcggcgcagctggaaaaagaactgcaggcgctggaaaaagaaaacgcgcagctggaatgggaactgcaggcgctggaaaaagaactggcgcagggcggctgcccggcgggcgcgagcagcggcagcagcggcagcagcccgggcggcgcggctcagctggaaaaagaactgcaggctctggaaaaagagaacgcgcagctggaatgggagctgcaggcgctggagaaagaactggctcagggcggctgcccagcgggcgcgagtagcggtagcagcggcagcccgagcggcggcgctgcgcagctggaaaaagagctgcaggcgctggagaaagaaaacgcgcaactggaatgggaactgcaagcgctggaaaaagaactggcgcaaggcggctgcccggcgggcgccagcagcggcagcagtggcccgagcagcggcggcgcggcgcaactggaaaaagaactgcaagcgctggaaaaagaaaacgcccagctggaatgggagctgcaggcgctggagaaagaactggctcagggcggctgcccggctggcgcgagcagtggtagcagcccgggcagcagcggcggcgcggcccagctggaaaaagagctgcaggccctggaaaaagaaaacgcgcaactggaatgggaactgcaagcgctggaaaaagaactggcacagggcggctgcccggcgggcgccagtagcggcagcccgagcggcagcagcggcggcgcggcgcaactggaaaaagaactgcaagcgctggaaaaagaaaacgcccagctggaatgggagctgcaggcgctggagaaagaactggcgcaaggcggctgcccagcgggcgcgagcagcggcccgagcagcggcagcagcggcgcggccgc

SEQ ID NO: 77 - DNA, 93 bp, Artificial Sequence
  Recombinant sequence
gggccctcattgttaacgccgcgacgtgcgactgcgatgagcatctatgtgtatgcgctgccgctgaaaatgctgaacattuaauaaagatct

SEQ ID NO: 78 - DNA, 152 bp, Artificial Sequence
  Recombinant sequence
gacgtctttatcaaaaagagtgttgacttgtgagcggataacaatgatacttagattcatcgagagggacacgagatctggtaccgtcgacatctacattactgagctaataacaggcctgctggtaatcgcaggccuuuuuatttgacgtc

SEQ ID NO: 79 - DNA, 236 bp, Artificial Sequence
  Recombinant sequence
cggcatgctttatcaaaaagagtgttgacttgtgagcggataacaatgatacttagattcatcgagagggacacggcgaaaaataaggaggaaaaaaaaatggcgaataacgatctctttcaggcatcacgtcggcgttttctggcacaactcggcggcttaaccgtcgccgggatgctggggccctcattgttaacgccgcgacgtgcgactgcgagatctgcatgcataatgtg

(19) United States
(12) Patent Application Publication
(10) Pub. No.: US 2024/0271120 A1
(43) Pub. Date: Aug. 15, 2024

(54) PMHC MULTIPLEXERS FOR DETECTION OF ANTIGEN-SPECIFIC CELLS

(71) Applicant: Gigavax ApS, Holte (DK)

(72) Inventors: Henrik Pedersen, Bagsværd (DK); Hans-Henrik Kristensen, Holte (DK)

(21) Appl. No.: 18/263,039

(22) PCT Filed: Jan. 28, 2022
(86) PCT No.: PCT/EP2022/052119

§ 371 Date: Jul. 26, 2023

(30) Foreign Application Priority Data
Jan. 28, 2021 (DK) PA 2021 00087
Jan. 28, 2021 (DK) PA 2021 00091
Jan. 28, 2021 (DK) PA 2021 00092
Jan. 28, 2021 (DK) PA 2021 00093
Jan. 28, 2021 (DK) PA 2021 00094
Jan. 28, 2021 (DK) PA 2021 00095
Jan. 28, 2021 (DK) PA 2021 00096
Jan. 28, 2021 (DK) PA 2021 00097
Jan. 28, 2021 (DK) PA 2021 00098
Jan. 28, 2021 (DK) PA 2021 00099
Jan. 28, 2021 (DK) PA 2021 00100
Jan. 28, 2021 (DK) PA 2021 00101
Jan. 28, 2021 (DK) PA 2021 00102

(51) Int. Cl. C12N 15/10; C07K 14/74; C12N 7/00

(52) U.S. Cl. CPC C12N 15/1037; C07K 14/70539; C12N 7/00; C12N 2795/14151

(57) ABSTRACT

This invention describes the production and properties of a pMHC Multiplexer. The pMHC Multiplexer is a spatially limited composition of two different molecules, an encoding molecule (i.e. an RNA or DNA molecule), and an encoded peptide, where said encoded peptide is encoded by said encoding molecule. Furthermore, the peptide is complexed to a MHC complex and thus is part of a pMHC complex. A preferred embodiment of the invention describes the production and properties of an example pMHC Multiplexer that is a phage particle carrying on its surface a number of identical pMHC complexes, where the peptide of the pMHC complexes is encoded by the DNA contained within the phage particle, and where a covalent or non-covalent bond links a phage coat protein with a pMHC complex and/or a pMHC Multimer. Another preferred embodiment of the invention describes the production and properties of an example pMHC Multiplexer that is a eukaryotic cell carrying on its surface a number of identical pMHC complexes, where the peptide of the pMHC complexes is encoded by the DNA contained within the cell. Yet another preferred embodiment of the invention describes the production and properties of an example pMHC Multiplexer where the encoding molecule is a DNA or RNA, and where the binding of pMHC Multiplexer to T cell receptor (TCR) can be detected by PCR-based analysis. Yet another preferred embodiment of the invention describes the production and properties of an example pMHC Multiplexer that comprises one or more identical pMHC complexes, where the encoding molecule is directly linked to at least one peptide (p) of one of the pMHC complexes, and thus, the peptide (p) of the pMHC complex(es) is encoded by said encoding molecule directly linked to it.

### BACKGROUND

The adaptive immune system is operating by way of specific interactions between immune cells such as CD8+ or CD4+ T cells and antigen-presenting cells such as dendritic cells, virus-infected cells, and cancer cells. T-lymphocytes (T cells) specifically recognize and bind target cells by interaction of their T cell receptors with Major Histocompatibility Complexes (MHC) on the target cells. MHC complexes are typically bound to a peptide, and the complexes may then be termed pMHC complexes.

MHC complexes are found in three variations: MHC class 1 (MHC1), MHC class 2 (MHC2), and MHC-like complexes and proteins.

Fluorescent-labelled MHC Multimers, consisting of multiple copies of a unique pMHC complex and carrying a fluorochrome compatible with flow cytometry, are typically used for detection of antigen-specific T cells by flow cytometry.

The human population represents an enormous diversity of antigen-specific T cells, and it is a significant limitation to the development of e.g., personalized cancer vaccines and virus- and bacteria-targeting vaccines that only a few different antigen-specific T cell specificities can be tested in parallel, as is often the situation.

### DESCRIPTION

The present invention aims at achieving a higher-throughput screening of antigen-specific T cells.

### GENERIC PRINCIPLE

This invention describes the formation and use of collections of pMHC Multiplexers. A pMHC Multiplexer is a spatially confined composition of at least two molecules, an encoding molecule and a peptide (and/or protein) that is encoded by said encoding molecule. Said at least two molecules are functionally linked, and the properties of the functional link determines the boundaries of the spatial confinement.

The functional link may be chemical (i.e. covalent or non-covalent) bond(s) between the molecules of the composition, or it may be mechanical bond(s) between the molecules of the composition, or any other type of functional link that keeps the encoding molecule and the encoded peptide and/or protein within a certain maximum distance of each other.

A pMHC Multiplexer is shown in FIG. 1. The outer circle symbolizes the confined space of the composition containing the encoding molecule and the encoded peptide and/or protein; the straight line symbolizes the encoding molecule; the wavy line symbolizes the encoded peptide of the pMHC complex; the Y-shape symbolizes the (possibly encoded) MHC complex of the pMHC complex; the wavy line and the Y-shape together symbolizes the pMHC complex; and the dashed line symbolizes the functional link between the encoding and encoded molecules.

A pMHC Multiplexer may contain one or more pMHC complexes, such as 1-2, 3-4, 5-6, 7-8, 9-10, 11-15, 16-25, 26-40, 41-100, or more than 100 pMHC complexes.

Collections of pMHC Multiplexers may be used in screening or selection processes that identify or quantitate or purify or isolate antigen-specific T cells. For screening purposes, the pMHC Multiplexer may carry a label such as a fluorophore. T cells that are recognized and bound by the pMHC Multiplexer will become fluorescent and can be detected e.g., in a flow cytometry screening process. For selection purposes, one may take advantage of the fact that pMHC complexes, especially when operating in concert with several other pMHC complexes, can stimulate growth and proliferation of T cells upon binding to these. Thus, after exposure to specific pMHC complexes, T cells may proliferate and eventually become a dominant clone in a collection of T cells. This process thus serves to preferentially amplify certain specific T cells, in effect enriching (selecting) for these.

An antigen-specific T cell may be identified by its ability to recognize a specific pMHC complex and/or a specific pMHC Multiplexer. If this is combined with sequencing of the DNA of the T cell, identification of specific pMHC complex-T cell receptor pairs may be achieved. Such information can be used in the design of antigen-specific T cell-based vaccines.

### GUIDELINES FOR USING THE PRESENT INVENTION

When using the present invention to generate vaccine candidates, the desired characteristics of the identified pool of T cells, pMHC specificities or T cell/pMHC pairs must first be defined.

As an example, it must be decided whether the goal to identify as many candidates as possible or rather a few representing strong interactions between the T cells and the pMHC complexes. In this case e.g. the screening conditions may be set up in a way so as to generate a large output (e.g. by having relatively non-stringent screening conditions), or so as to generate a small output representing epitopes of high affinity of epitopes (e.g. by having relatively stringent screening conditions).

Similarly, it must be decided which display system to use (e.g. cell display or phage display), and whether both MHC1 and MHC2 peptide specificities are desired, or only one of these.

If the candidate epitopes are to be selected from a relatively small pool of potential epitopes (e.g. to be selected from e.g 1-10,000 MHC1 epitopes that can encompass 100 mutations of a cancer patient), an embodiment of the present invention that involves the placement of individual candidate epitopes in separate microtiterplate wells may be preferred, whereas in other cases where there is a much larger pool of potential epitopes, such as in the search for virus epitopes encoded by a 1,000,000 bp-genome, it will be more appropriate to use an embodiment of the present invention that does not require the placement of individual epitopes in separate wells.

In a simple embodiment of the invention, a collection of pMHC Multiplexers carrying cancer-specific potential peptide epitopes is prepared, and the collection of pMHC Multiplexers are employed in a screening process to identify cancer-specific T cells from e.g. the blood or tumor of a cancer patient.

The identified epitopes, capable of recognizing cancer-specific T cells when displayed in a pMHC context, can e.g. be used in vaccine- or immunotherapeutic applications including proliferation of transplant bone marrow immune cells.

In another simple embodiment of the invention, a collection of pMHC Multiplexers carrying Influenza-specific potential peptide epitopes is prepared, and the collection of pMHC Multiplexers are employed in a screening process whereby Influenza-specific T cells of the blood of a confirmed Influenza-infected individual are identified.

The identified epitopes may now be used in a general vaccine formulation, to give protection against Influenza infection and disease development.

When using the present invention it must be decided what the purpose is:
Purpose 1-Detection: The purpose is to detect and/or identify a certain type of cell or cells, e.g. identify (i) one or more antigen-specific T cells, (ii) one or more antigen-presenting cells, or (iii) one or more “antigen-specific T cell/antigen-presenting cell pairs”, i.e. to identify one or more antigen-specific T cells and their cognate antigen-presenting cells.
Purpose 2-Modification: The purpose is to activate, stimulate, proliferate, kill or in other ways modify a certain type of cell or cells, being e.g. (i) one or more antigen-specific T cells, (ii) one or more antigen-presenting cells, or (iii) one or more “antigen-specific T cell/antigen-presenting cell pairs”.
Purpose 3-Isolation and/or enrichment: The purpose is to isolate, enrich or diminish or in other ways change the relative numbers of specific cells, being e.g. (i) antigen-specific T cells, (ii) antigen-presenting cells, and/or (iii) “antigen-specific T cell/antigen-presenting cell pairs”.

In a preferred embodiment of the invention, the purpose is to detect antigen-specific T cells of a particular specificity (e.g. cancer-specific T cells), and the invention may be divided into two parts, A and B, as follows.
Part A—preparation of pMHC Multiplexers: a collection of n different pMHC Multiplexers (e.g comprising n different cancer-specific epitopes) are prepared.
Before preparing a collection of pMHC Multiplexers, the type and specific version of each component may be decided. Thus, a choice may be made on the following components: display system (e.g. phage display, virus display, cell display), type of MHC (e.g. MHC class 1 and/or MHC class II), type of peptide epitope (e.g. length, origin, modifications, natural or unnatural amino acids), type of multimer scaffold (e.g. tetramer or dextramer), dimerization or multimerization components (e.g. acid-base peptides, fos-jun peptides, streptavidin), and label (e.g. DNA oligonucleotide, fluorophore, rare element).
Part B—use of the pMHC Multiplexers: The collection of pMHC Multiplexers is then screened in order to detect antigen-specific T cells of a particular specificity (e.g. cancer-specific T cells).
This screening performed in Part B can be done in many different ways. As a first step, the pMHC Multiplexers prepared in Part A may be mixed and incubated with a sample comprising T cells (e.g. blood, synovial fluid, bone marrow). pMHC Multiplexers capable of recognizing specific T cells will now have bound to these T cells. If the pMHC Multiplexers are all labelled with the same fluorophore FI but each type of pMHC Multiplexer is labelled with a specific DNA tag (e.g. a collection of 1000 different pMHC Multiplexers all comprise the A0201 heavy chain of the MHC complex, but 1000 different peptides; and each of the 1000 different pMHC Multiplexers carry a unique peptide as well as a unique DNA-tag, and the identity of each DNA-tag and the peptide it is linked to is known), then the T cells that have bound to a pMHC Multiplexer will now be labelled with the fluorophore FI. These T cells that have become labelled with fluorophore FI can now be isolated by the use of flow sorting whereby e.g. fluorophore FI-labelled cells are directed to Flask 1 and the non-labelled cells are directed to Flask 2. Therefore, Flask 1 will be enriched for the FI-labelled cells, i.e. cells capable of binding to pMHC Multiplexers.
Sequencing of the tags carried by the pMHC Multiplexers of Flask 1 will now reveal the identity of the peptides of the pMHC complexes of the pMHC Multiplexers that were able to bind to T cells of the sample comprising T cells.
In the example above, flow sorting and labelling of pMHC Multiplexers with fluorophore and DNA-tag allow the screening process to be performed by flow sorting.

Any of the collections of pMHC Multiplexers prepared in Part A can be combined with any of the screening processes of Part B, and lead to the identification of antigen-specific T cells, antigen-presenting cells, or pairs of antigen-specific T cells and their cognate antigen-presenting cells.

In a preferred embodiment of the invention the purpose of the efforts (analysis) is to identify one or more specificities of antigen-presenting cells (i.e. identify the peptides and/or MHC complex of antigen-presenting cells) (Purpose 1,ii) that allow binding to antigen-specific T cells, where the peptide antigen is characteristic of a certain disease state, e.g. infection by virus, bacterium, fungi, or other microbe, or cancer. Once the disease state has been defined, the peptide identities (e.g. peptide sequences) of the pMHC Multiplexers (to be prepared in Part A) can be defined. And once the peptide identities have been defined, the synthetic route for preparing the peptides or for preparing the genes encoding the peptides can be decided.
For example, if the disease state is a Covid-19 infection, the peptides of the pMHC Multiplexers may be all the peptides encoded by the Covid-19 Corona virus. The synthetic route for preparing the peptides may then be e.g. cloning of cDNA corresponding to the Covid-19 genome, as dsDNA fragments of between 20 and 50 bp, by first isolating Covid-19 genome DNA, cleaving it with e.g. DNAse, and then cloning into e.g. a M13 phagemid. Alternatively, DNA oligos of 20 nt in length with sequences corresponding to all possible 20 nt-fragments of the Covid-19 genome may be prepared by standard DNA oligonucleotide synthesis, and then cloned into e.g. M13 phage DNA by pool cloning.
The collection of pMHC Multiplexers prepared in this way will thus each display multiple identical pMHC complexes where the peptide component is encoded by the Covid-19 genome.
For example, if the disease state involves a cancer tumor, the peptides of interest may be the mutant peptides encoded by the tumor cells. As an example, by sequencing the DNA of a patient's tumor cells, 100 mutations are identified. For each of these mutations, 10 oligos of each 30 nt in length and comprising the mutation, and all of which overlap with at least one of the other oligos, are synthesized. Then these 100×10=1000 oligos are cloned into e.g. a phagemid, and phage-based pMHC Multiplexers each comprising multiple copies of one of the 1000 peptides encoded by one of the 1000 oligos are prepared. Upon screening e.g. the patient's blood or a blended cell suspension of the patient's tumor, pMHC multiplexers capable of binding antigen-specific T cells may be identified. The DNA-tag of each of these pMHC multiplexers will reveal the identity of the peptides of the pMHC complexes capable of binding the T cells. These peptide sequences may be used in the design of cancer immunotherapeutics or cancer vaccines for that patient.

### COMPONENTS OF THE INVENTION

### Display Systems.

A number of display systems are applicable to the present invention, including i) phage display, virus display, and cell display, all of which consists of a physical boundary within which the encoding molecule (RNA or DNA) is confined, and an encoded peptide that is chemically linked to the surface of the cell, virus or phage; ii) polymeric molecule displays carrying more than two pMHC complexes and a DNA molecule that encodes the peptide of the pMHC complexes, e.g., PROfusion display systems where the encoding molecule is the mRNA that served as the messenger RNA for the peptide that becomes attached to it, or DNA-tagged MHC Dextramers where the polymeric molecule is a dextran and the encoding molecule is a DNA oligo attached to the backbone dextran, or Ribosome display where the ribosomes serve as a chemical link between the encoding molecule (mRNA) and the encoded molecule (peptide or protein); and iii) small organic molecule display, where both a DNA tag and the encoded molecule is attached to the same molecule scaffold. Cell display includes prokaryotic cell display, e.g., proteins and peptides displayed on the surface of E. coli cells, Bacillus cells and Salmonella cells, and eukaryotic cell display, e.g., yeast cell display or human cell display such as dendritic cell display. In the latter case, the gene encoding the unique peptide of the pMHC complex may be encoded by a vector such as a virus, a MRNA, or a DNA, and may be introduced into the cell by transformation, infection, transduction, using vesicles, lipid-nanoparticles, or any other way of introducing the encoding molecule into the cell. In this case the display of the pMHC complex will be mediated through the cell's natural systems for pMHC1 or pMHC2 display.

The functional link between the encoding molecule and the encoded molecule can be a chemical link and/or a mechanical link. There are two types of chemical link, namely covalent and non-covalent link. A mechanical link, on the other hand, is neither covalent nor non-covalent.

A mechanical link involves a molecular structure that keeps two or more molecules in the vicinity of each other by physical rather than chemical means. Thus, it is a physical boundary that keep the molecules from separating, rather than covalent or non-covalent bonds between the molecules. Example physical boundaries and the functionally linked molecules are: i) two molecules, e.g., a DNA and a peptide, are both kept within the boundary of a micelle. The separation of the DNA from the peptide to a distance larger than the micelle diameter would require breakage of the micelle wall; ii) a DNA kept within the boundaries of a cell, and a peptide chemically linked (covalently or non-covalently) to the surface of said cell. The DNA and peptide are not attached to each other, but nevertheless a separation of the DNA from the peptide to a distance larger than the cell diameter would require breakage of the cell membrane/wall, or a breakage of the chemical link that keeps the peptide associated with the cell surface; iii) a DNA kept within the boundaries of a phage or virus particle, and a peptide chemically linked (covalently or non-covalently) to the surface of said phage or virus particle.

The filamentous phage M13 display system has a mechanical link between the encoding molecule (phage DNA kept inside the phage particle by the boundaries of the phage coat) and the encoded molecule (e.g., a peptide attached to one of the phage coat proteins). The phage coat proteins include the pIII coat protein, present in approximately 4-5 copies at the tip of the phage, and pVIII covering the majority of the surface of the M13 phage, and present in about 3000 copies. There are several other coat proteins that may be used to display peptides and proteins as well, but mostly the pIII and pVIII coat proteins are used in display systems.

The pIII coat protein is usually used for low valency display. The encoded peptide is typically displayed as a protein fusion with pIII, and as a result the phage will display the peptide in a few copies on the surface of the phage particle while keeping in its interior the DNA that encodes the displayed peptide.

The pVIII coat protein is usually used for high valency display. The encoded peptide is typically displayed as a protein fusion with pVIII, and as a result the phage will display the peptide in many copies on the surface of the phage particle while keeping in its interior the DNA that encodes the displayed peptide.

By expressing both the wildtype coat protein (e.g., pIII or pVIII) and the display peptide-coat protein fusion on the phage genome, the phage coat will consist of both wildtype coat protein and peptide-coat protein fusion protein. This therefore serves as a means to adjust the average valency of display by the phages from near zero to about 5 (pIII display) and from near zero to about 3000 (pVIII display).

In a preferred embodiment of the invention, one of the phages listed below is employed.

Fd-tet
F8-1
PM48
PM54
PM52
f88-4
fd88-4:
fth1
pA2
PC89

pMHC

The term “MHC complexes” includes MHC1 protein (also called empty MHC1), MHC2 protein (also called empty MHC2), pMHC1 complex and pMHC2 complex. The term “MHC-like complexes” shall include CD1a, CD1b, CD1c, CD1D, and other MHC-like proteins.

Unless indicated specifically, the terms “MHC”, “MHC complex”, “pMHC”, or “pMHC complex” shall herein this invention denote the extracellular domains of the proteins of the MHC complexes, and thus shall correspond to the usual part of the MHC and MHC-like proteins (e.g., HC, beta2M, alpha, beta) that are employed in MHC Multimers such as MHC Tetramers (from Beckman Coulter) and MHC Dextramers (from Immudex).

For attachment of the pMHC1 complex to a Multimer scaffold or other structure or molecule, the N-terminus of the Heavy Chain (HC) may be modified chemically, e.g., it may be fused to the Acceptor Peptide (AP), capable of being biotinylated in vitro or in vivo by biotin ligase (BirA), or it may be fused to the Acid Peptide or Base Peptide, in order to dimerize with the Base Peptide or Acid Peptide, respectively. In another preferred embodiment, the C-terminus of HC or the N- or C-terminus of beta2M may be used to attach the pMHC1 complex to a Multimer scaffold or other structure or molecule.

Similarly, for attachment of the pMHC2 complex, the N- or C-terminus of the alpha or beta subunits may be modified chemically or may be fused to e.g., the AP-, Acid- or Base Peptide.

### Peptide-Receptive MHC1 Complexes

Peptide-receptive MHC1 complexes are MHC class I molecules stabilized—in some instances—by a disulfide bond to link the α1 and α2 helices close to the F pocket and are described in Saini et al., Sci. Immunol. 4, eaau9039 (2019). These disulfide-stabilized MHC class I molecules can be loaded with peptide in the multimerized form allowing for easy binding and display of different peptide epitopes and corresponding formation of functional pMHC1 complexes.

A specific disulfide mutant variant of the human MHC-I protein HLA-A*02:01 has been constructed by introducing two cysteines at positions 84 and 139 (replacing tyrosine and alanine, respectively. This variant had restricted conformational flexibility and a consequently increased stability of the peptide-free state compared with wild-type. Introducing the disulfide bond between the α1 and α2 helices at positions 84 and 139 eliminated the tendency of the empty binding groove to collapse and keeping the capacity to bind exogenous peptide.

A similar peptide-receptive MHC1 complex has been constructed with human MHC-I molecule HLA-A*24:02.

Peptide-receptive MHC1 complexes can be constructed using other isotypes including but not limited to HLA-A (HLA-A), HLA-B (HLA-B), HLA-C(HLA-C), and some less polymorphic such as HLA-E (HLA-E), HLA-F (HLA-F), HLA-G (HLA-G).

### Peptide

The peptide of a pMHC complex may comprise 2-1000 amino acid residues, such as 2-4, 5-6, 7-8, 9-10, 11-12, 13-15, 16-20, 21-30, or 30-50 amino acid residues, or more.

The peptide of a pMHC complex is also called the epitope, neoepitope, peptide epitope, or peptide neoepitope.

The sequence of the peptide of the pMHC complex may be nonsense, i.e. not originate from any known peptide sequence in Nature, or may be identical to a sequence in the human genome, a virus genome, a bacterial genome, a parasite genome, or a mutant sequence identified in a patient, e.g., in a biopsy from a cancer patient.
In a preferred embodiment where the pMHC complex is a pMHC1 complex, the peptide of the pMHC1 complex is a peptide of 7-11 alpha amino acid residues.
In a preferred embodiment the peptide of the pMHC1 complex is a peptide of 7-11 alpha amino acid residues identical to a peptide sequence encoded by the human genome.
In a preferred embodiment the peptide of the pMHC1 complex is a peptide neoepitope of 7-11 alpha amino acid residues encoded by the DNA of cells of the tumor of a human cancer patient.
In a preferred embodiment the peptide of the pMHC1 complex is a peptide of 7-11 alpha amino acid residues encoded by the DNA of a virus, bacteria or fungus capable of infecting humans or other mammals.
In a preferred embodiment where the pMHC complex is a pMHC2 complex, the peptide of the pMHC2 complex is a peptide of 7-30 alpha amino acid residues.
In a preferred embodiment the peptide of the pMHC2 complex is a peptide of 7-30 alpha amino acid residues identical to a peptide sequence encoded by the human genome.
In a preferred embodiment the peptide of the pMHC2 complex is a peptide neoepitope of 7-30 alpha amino acid residues encoded by the DNA of cells of the tumor of a human cancer patient.
In a preferred embodiment the peptide of the pMHC2 complex is a peptide of 7-50 alpha amino acid residues encoded by the DNA of a virus, bacteria or fungus capable of infecting humans or other mammals.

### MHC Multimers

Multimer scaffolds are molecular structures or particles to which can be attached two or more MHC or pMHC complexes. See also definition of multimer scaffolds.

### Dimerization- and Multimerization Components

In principle, all of the multimer scaffolds used as a backbone in MHC multimers may be used as homo- or heteromultimerization components as well.

The Acid Peptide (sequence AQLEKELQALEKENAQLEWELQALEKELAQ) and the Base Peptide (sequence AQLKKKLQALKKKNAQLKWKLOALKKKLAQ) may form the heterodimer Acid Peptide-Base Peptide. If both the Acid Peptide and the Base Peptide are each attached to a molecule or structure, the heterodimerization of Acid Peptide and Base Peptide will attach the two molecules or structures to each other. The linkage will be non-covalent.

Variants of the Acid- and Base Peptide may be used that carry each a cysteine; these are called Acid-cys Peptide (AQLEKELQALEKENAQLEWELQALEKELAQGGC) and Base-cys Peptide (sequence AQLKKKLQALKKKNAQLKWKLQALKKKLAQGGC). Upon heterodimer-formation the two cysteines may form a disulfide bond between the Acid- and Base Peptide. This linkage will thus be covalent.

In this invention, the Acid Peptide and Base Peptide dimerization domains are used for exemplification; any other homo- or heterodimerization domains may be used in place of Acid Peptide and Base Peptide. Streptavidin (SA) is a tetramerization domain. By mixing biotinylated pMHC complexes and streptavidin one may form pMHC Tetramers.

The Pentamer from ProImmune is a homo-pentameric structure comprising five pMHC complexes, held together by a coil-coil structure.

Any homo- or heterodimerization domains can be used in this invention, in the same way that Acid-Base Peptide dimerization domains are used. Likewise, any dimerization domain and any dimer may be used to attach any two molecules.

Streptavidin (SA) is a tetramerization domain. By mixing biotinylated pMHC complexes and streptavidin one may form pMHC Tetramers.

The Pentamer from ProImmune is a homo-pentameric structure comprising five pMHC complexes, held together by a coil-coil structure.

All of the multimer scaffolds used as a backbone in MHC multimers may be used as homo- or heteromultimerization components as well.

### Label

The pMHC Multiplexers and pMHC Multimers of this invention can be labelled with any label including DNA oligonucleotides, fluorochromes (e.g., FITC, PE (phycoerythrin), PerCP, APC, GFP, etc.), Lanthanides such as e.g., Lanthanum, Cerium, Praseodymium, Neodymium, Promethium, Samarium, Europium, Gadolinium, Terbium, Dysprosium, Holmium, Erbium, Thulium, Ytterbium, Lutetium, or other chromophores.

The label may be attached to the pMHC Multiplexer by covalent or non-covalent bond, or by a mechanical bond. For example, filamentous phage M13 may be labelled with anti-M13 phage antibodies carrying a fluorochrome or a chromophore, where the antibodies bind to the coat of the phage. Alternatively, anti-beta2M antibodies or anti-HC antibodies, recognizing beta2M and HC, respectively, carrying fluorochromes or fluorophores may be used.

Applications of pMHC Multiplexers.

pMHC Multiplexers or collections (“libraries”) of pMHC Multiplexers of the present invention, or combinations thereof, may be used for e.g., the screening for e.g., i) disease-specific T cells, ii) cells of a certain haplotype or general ability to bind a certain peptide epitope or T cell receptor or T cell, iii) pairs of T cell receptors and pMHC complexes, iv) or interrecognizing pairs of T cell and antigen-presenting cells; or may be used for e.g., the modification (e.g., activation, stimulation, proliferation, killing or other types of modification) of cells such as antigen-specific T cells or antigen-presenting cells such as dendritic cells; or may be used for the sorting and/or enrichment of certain types of cells such as e.g., (i) antigen-specific T cells, (ii) antigen-presenting cells, and/or (iii) “antigen-specific T cell/antigen-presenting cell pairs”. Procedures for their use in these applications are described below, in “Applications 1-9”.

In the applications below, and in other parts of the description of this invention, the detection, sorting or modification of T cells in various kinds of samples are described. Such samples comprising T cells include the following samples: Blood, Serum, Plasma, Synovial fluid, Interstitial fluid, Lymphatic fluid, Cerebrospinal fluid, Peritoneal fluid, Pleural fluid, Amniotic fluid, Bone marrow, Tumor Biopsy, Liquid biopsy, Fluid in joint, Saliva, Semen, Vaginal fluids, Mucus, Urine, Connective tissue, Epithelial tissue, Muscle tissue, Nervous tissue.

The present invention describes the preparation of a number of pMHC Multiplexer libraries. These libraries can all be used in the following “Applications 1-9”.

“Application 1”: Screening a Cancer Patient's Blood Using pMHC Multiplexers, by i) Centrifugation, ii) Fluorescence-Based Flow Sorting, or iii) Immobilization on Anti-CD8 Antibody-Coated Beads.

In one embodiment of the present invention, the invention is used to rapidly produce a large number (N) of different pMHC Multiplexers, where N may be any number between 2 and 10¹⁵. Following their production they are pooled into one solution (if they are not already in one solution), to generate a composition (also called a library) of a large number of pMHC Multiplexers. This solution may be added to a sample containing T cells, such as e.g., a blood sample, a biopsy from a tumor of a human cancer patient, or a bone marrow sample, allowing for e.g., parallel analysis and possible detection or isolation of one or more antigen-specific T cells.

Once the cell sample has been incubated with the pMHC Multiplexers, the T cells that become bound by the pMHC Multiplexers may be identified, quantified or further manipulated, as follows.
Selection by centrifugation. The incubation mixture of cells and pMHC Multiplexers from above may be centrifuged, to form a cell pellet. After the supernatant has been removed, the cell pellet may optionally be resuspended in physiological buffer, and centrifuged again, and the supernatant removed. Optionally, the washing process can be repeated one or more times. After one or more washes, most of the pMHC Multiplexers isolated with the cell pellet will be pMHC Multiplexers that bind antigen-specific T cells of the cell sample. Sequencing the encoding molecule component (e.g., the DNA) of a recovered pMHC Multiplexer will reveal the identity of the peptide (p) of the pMHC component carried by that pMHC Multiplexer, in turn identifying the binding specificity of the T cell of the original cell sample that bound to this pMHC Multiplexer.
Isolation and identification of pMHC complex-T cell receptor pairs by flow cytometry. The pMHC Multiplexers of the incubation mixture of cells and pMHC Multiplexers from above may be labelled with fluorescent (e.g., PE-labelled) anti-bodies against part of the pMHC Multiplexer, e.g., by addition of anti-HC antibody if the pMHC Multiplexers carry MHC1 complexes. Then the cells are sorted by flow sorting using a flow cytometer; all cells carrying the pMHC Multiplexer-specific label (e.g., PE) (and optionally other markers of the target cells) can then be isolated. By sequencing the encoding molecule (e.g., DNA) of the pMHC Multiplexer the identity of the peptide component of the pMHC Multiplexer is revealed, in turn revealing the binding specificity of the T cells that were isolated. By single cell sequencing of the isolated T cells along with sequencing of the encoding molecule of the pMHC Multiplexer that bound to it, it is possible to identify the pMHC complex and the corresponding T cell (and specific T cell receptor) that bound it.

In a preferred embodiment of the invention, the pMHC Multiplexers are used in a screening of patient samples, as follows.

Using a library of e.g., 960 unique pMHC Multiplexers, a patient sample such as blood or tumor material, can be screened for e.g., cancer-specific epitopes by the use of flow sorting, as follows:
Step a: The blood is treated using standard procedures, and the library comprising 960 DNA-tagged pMHC Multiplexers is added under standard conditions, and incubation proceeds for approximately 30 minutes.
Step b: Flow sorting is used to enrich (collect) those T cells that bind a significant number of pMHC Multiplexers.
Step c: The encoding molecule of the pMHC Multiplexers are sequenced—e.g., if the encoding molecule is a DNA the DNA molecules of the pMHC Multiplexers attached to the collected cells are amplified by PCR, after addition of external primers, using standard techniques, and the amplified DNA-tags are then sequenced. From the knowledge of which DNA-tags were attached to which peptides in the pMHC Multiplexer library, the identity of the peptides in the pMHC Multiplexers that bound the T cells and therefore were collected, can now be deduced from the sequence of the recovered and amplified DNA molecules. These peptides represent potential patient- and disease-specific epitopes.

The screening thus provides information about potential disease-specific epitopes of the patient from which the blood- or tumor sample was taken. This information can be used as a diagnostics tool, as well as a means for designing disease- and patient-specific vaccines and therapeutic treatments.

In a preferred embodiment the pMHC Multiplexer is a filamentous phage particle displaying pMHC complexes on its surface and comprising within its interior the DNA encoding the peptide component of said pMHC complexes. A library (collection) of such pMHC Multiplexers may be screened, and potential peptide epitopes identified, by any of the following approaches:
Approach 1: Detection by flow cytometry. A cell sample comprising T cells is mixed with the library of pMHC Multiplexers and incubated. Fluorescently labelled anti-CD8 antibodies and fluorescently labelled anti-pVIII antibodies are added, and incubation continued. Then flow sorting is used to isolate cytotoxic T cells (binds CD8 antibodies) that recognize and bind specific pMHC Multiplexers (binds anti-pVIII antibodies). Following the isolation of CD8+, pMHC Multiplexer+ T cells, the DNA of the pMHC Multiplexers is sequenced. This will reveal the identity of the peptides of the pMHC complexes (of the pMHC Multiplexers) that were recognized by the T cell receptors of the T cells. These peptides are (potential) peptide epitopes.
Approach 2: Detection by immobilization on anti-CD8 antibody-coated beads. A cell sample comprising T cells is mixed with the library of pMHC Multiplexers, and incubated. Magnetic beads are coated with anti-CD8 antibodies. The mixture of cells and pMHC Multiplexers from above are added to the anti-CD8-coated beads, and is incubated. Then the CD8+ T cells, bound to the anti-CD8-coated beads, are removed from the solution, and transferred to another tube, and washed a few times in physiological buffer while on the magnetic beads. The pMHC Multiplexers that bind the CD8+ T cells after several washes will be those pMHC Multiplexers that carry pMHC complexes able to bind T cell receptors of the immobilized T cells. Sequencing of the DNA comprised within the bound pMHC Multiplexers will reveal the identity of the peptides of these pMHC complexes. These peptides are (potential) peptide epitopes.
Approach 3: Cell precipitation by centrifugation. A cell sample comprising T cells is mixed with the library of pMHC Multiplexers and is incubated. Then the solution is centrifuged. The cell precipitate will contain the pMHC Multiplexers that were bound to T cells. The cells may be resuspended and re-centrifuged a few times, to wash the cells. The pMHC Multiplexers that bind the T cells after several washes will be those pMHC Multiplexers that carry pMHC complexes able to bind T cell receptors of the T cells. Sequencing of the DNA comprised within the pMHC Multiplexers will reveal the identity of the peptides of these pMHC complexes. These peptides are (potential) peptide epitopes.

In a preferred embodiment, individual wells of a microtiter-plate each comprise multiple copies of a unique pMHC Multiplexer, where the pMHC Multiplexer is a filamentous phage particle displaying pMHC complexes on its surface and comprising within its interior the DNA encoding the peptide component of said pMHC complexes. The pMHC Multiplexer of each of the wells may be screened, and potential peptide epitopes identified, by any of the following approaches:
Approach 1: Detection by flow cytometry. A cell sample comprising T cells is added to each of a number of wells comprising a unique pMHC Multiplexer in multiple copies, and is incubated. Fluorescently labelled anti-CD8 antibodies and fluorescently labelled anti-pVIII antibodies are added, and incubation continued. Then flow sorting applied to the contents of each well, one at a time, is used to identify cytotoxic T cells (binds anti-CD8 antibodies) that recognize and bind specific pMHC Multiplexers (binds anti-pVIII antibodies). In case T cells are identified by the flow cytometry analysis, the DNA of the pMHC Multiplexer in the corresponding well is sequenced. This will reveal the identity of the peptide of the pMHC complex (of the pMHC Multiplexer) that was recognized by the T cell receptors of the T cells. This peptide is a (potential) peptide epitope.
Approach 2: Differential proliferation of antigen-specific T cells. To each of the wells comprising each a unique pMHC Multiplexer, a T cell sample (e.g., blood sample) is added. Appropriate cytokines/interleukins and other effector molecules are added, in order to allow stimulation and proliferation of T cells that are activated by encounter with a pMHC complex (of a pMHC Multiplexer) that is capable of binding a T cell receptor and thereby stimulating the corresponding T cell. After a few days of proliferation, the wells that contain pMHC Multiplexers that recognize T cells will have more growth of T cells than those that do not. The DNA of the pMHC Multiplexers in those wells can be sequenced, to reveal the identity of the peptides of the pMHC complexes (of the pMHC Multiplexers) that were recognized by the T cell receptors of the T cells. These peptides are (potential) peptide epitopes.
   “Application 2”: Screening for Antigen-Specific T Cells Using pMHC2 Multiplexers and Including a Pre-Enrichment Step that Improves the Relative Proportion of pMHC2 Multiplexers with Relevant Haplotypes for the Patient T Cell Sample in Question.

In one embodiment of the present invention, the invention is used to rapidly produce a large number (N) of different pMHC Multiplexers, where N may be any number between 2 and 10¹⁵. Following their production they are pooled into one solution (if they are not already in one solution), to generate a composition (also called a library) of a large number of pMHC Multiplexers. This solution may be added to a sample containing T cells, such as e.g., a blood sample, a biopsy from a tumor of a human cancer patient, or a bone marrow sample, allowing for e.g., parallel analysis and possible detection or isolation of one or more antigen-specific T cells.

Once the cell sample has been incubated with the pMHC Multiplexers, the T cells that become bound by the pMHC Multiplexers may be identified, quantified or further manipulated, as follows.
Selection by centrifugation. The incubation mixture of cells and pMHC Multiplexers from above may be centrifuged, to form a cell pellet. After the supernatant has been removed, the cell pellet may optionally be resuspended in physiological buffer, and centrifuged again, and the supernatant removed. Optionally, the washing process can be repeated one or more times. After one or more washes, most of the pMHC Multiplexers isolated with the cell pellet will be pMHC Multiplexers that bind antigen-specific T cells of the cell sample. Sequencing the encoding molecule component (e.g., the DNA) of a recovered pMHC Multiplexer will reveal the identity of the peptide (p) of the pMHC component carried by that pMHC Multiplexer, in turn identifying the binding specificity of the T cell of the cell sample that bound to this pMHC Multiplexer.
Isolation and identification of pMHC complex-T cell receptor pairs by flow cytometry. The pMHC Multiplexers of the incubation mixture of cells and pMHC Multiplexers from above may be labelled with fluorescent (e.g., PE-labelled) antibodies against part of the pMHC Multiplexer, e.g., by addition of anti-alpha antibody or anti-beta antibody (where beta and alpha are the two proteins of the MHC2 complex). Then the cells are sorted by flow sorting using a flow cytometer; all cells carrying the pMHC Multiplexer-specific label (e.g., PE) (and optionally other markers of the target cells) can then be isolated. By sequencing the encoding molecule (e.g., DNA) of the pMHC Multiplexer the identity of the peptide component of the pMHC Multiplexer is revealed, in turn revealing the binding specificity of the T cells that were isolated. By single cell sequencing of the isolated T cells along with sequencing of the encoding molecule of the pMHC Multiplexer that bound to it, it is possible to identify both the pMHC complex and the corresponding T cell receptor that bound it.

In a preferred embodiment of the invention, the pMHC Multiplexers are used in a screening of patient samples, as follows.

Using a library of e.g., 960 unique pMHC Multiplexers, a patient sample such as blood or tumor material, can be screened for e.g., cancer-specific epitopes by the use of flow sorting, as follows:
Step a: The blood is treated using standard procedures, and the library comprising 960 DNA-tagged pMHC Multiplexers is added under standard conditions, and incubation proceeds for approximately 30 minutes.
Step b: Flow sorting is used to enrich (collect) those T cells that bind a significant number of pMHC Multiplexers.
Step c: The encoding molecule of the pMHC Multiplexers are sequenced—e.g., if the encoding molecule is a DNA the DNA molecules of the pMHC Multiplexers attached to the collected cells are amplified by PCR, after addition of external primers, using standard techniques, and the amplified DNA-tags are then sequenced. The identity of the peptides in the pMHC Multiplexers that bound the T cells and therefore were collected, can now be deduced from the sequence of the recovered and amplified DNA molecules. These peptides represent potential patient- and disease-specific epitopes.

The screening thus provides information about potential disease-specific epitopes of the patient from which the blood- or tumor sample was taken. This information can be used as a diagnostics tool, as well as a means for designing disease- and patient-specific vaccines and therapeutic treatments.

In a preferred embodiment the pMHC Multiplexer is a M13 filamentous phage particle displaying pMHC complexes on its surface and comprising within its interior the DNA encoding the peptide component of said pMHC complexes. A library (collection) of such pMHC Multiplexers may be screened, and potential peptide epitopes identified, by any of the following approaches:
Approach 1: Detection by flow cytometry. A cell sample comprising T cells is mixed with the library of pMHC Multiplexers, and incubated. Fluorescently labelled anti-CD8 antibodies and fluorescently labelled anti-pVIII antibodies are added, and incubation continued. Then flow sorting is used to isolate cytotoxic T cells (binds anti-CD8 antibodies) that recognize and bind specific pMHC Multiplexers (binds anti-pVIII antibodies). Following the isolation of CD8+, pMHC Multiplexer+ T cells, the DNA of the pMHC Multiplexers is sequenced. This will reveal the identity of the peptides of the pMHC complexes (of the pMHC Multiplexers) that were recognized by the T cell receptors of the T cells. These peptides are (potential) peptide epitopes.
Approach 2: Detection by immobilization on anti-CD8 antibody-coated beads. A cell sample comprising T cells is mixed with the library of pMHC Multiplexers, and incubated. Magnetic beads are coated with anti-CD8 antibodies. The mixture of cells and pMHC Multiplexers from above are added to the anti-CD8-coated beads, and is incubated. Then the CD8+ T cells, bound to the anti-CD8-coated beads, are removed from the solution, transferred to another tube, and washed a few times in physiological buffer while on the magnetic beads. The pMHC Multiplexers that bind the CD8+ T cells after several washes will be those pMHC Multiplexers that carry pMHC complexes able to bind T cell receptors of the immobilized T cells. Sequencing of the DNA comprised within the bound pMHC Multiplexers will reveal the identity of the peptides of these pMHC complexes. These peptides are (potential) peptide epitopes.
Approach 3: Cell precipitation by centrifugation. A cell sample comprising T cells is mixed with the library of pMHC Multiplexers and is incubated. Then the solution is centrifuged. The cell precipitate will contain the pMHC Multiplexers that were bound to T cells. The cells may be resuspended and re-centrifuged a few times, to wash the cells. The pMHC Multiplexers that bind the T cells after several washes will be those pMHC Multiplexers that carry pMHC complexes able to bind T cell receptors of the T cells. Sequencing of the DNA comprised within the pMHC Multiplexers will reveal the identity of the peptides of these pMHC complexes. These peptides are (potential) peptide epitopes.

Using the same type of screening set-ups, other filamentous phage display systems may be used, and other coat proteins may be employed for the display of pMHC2 complexes. Likewise, other phage types and viruses and cells may be used with appropriate modification of the experimental details.

As described in Example 4, it may improve the screening process to include a pre-enrichment step where phage particles that have not been exposed to empty MHC2 complexes are screened for the ability to complex with empty MHC2, and where those that cannot bind the empty MHC2 are removed before the screening process is initiated. Optionally, the pre-enriched pMHC Multiplexers may be amplified (i.e. each of the copies turned into multiple copies), by re-infection in E. coli and preparation of pMHC Multiplexer from the amplified phage particles.

“Application 3”: Isolation and Identification of pMHC Complex-T Cell Receptor Pairs by Flow Cytometry, Using Fluorescent-Labelled Anti-Alpha Antibody or Anti-Beta Antibody, and Isolation and Identification by Immobilization on Anti-CD4 Antibody-Coated Beads.

In one embodiment of the present invention, the invention is used to rapidly produce a large number (N) of different pMHC Multiplexers, where N may be any number between 2 and 10¹⁵. Following their production they are pooled into one solution (if they are not already in one solution), to generate a composition (also called a library) of a large number of pMHC Multiplexers. This solution may be added to a sample containing T cells, such as e.g., a blood sample, a biopsy from a tumor of a human cancer patient, or a bone marrow sample, allowing for e.g., parallel analysis and possible detection or isolation of one or more antigen-specific T cells.

Once the cell sample has been incubated with the pMHC Multiplexers, the T cells that become bound by the pMHC Multiplexers may be identified, quantified or further manipulated, as follows.
Selection by centrifugation. The incubation mixture of cells and pMHC Multiplexers from above may be centrifuged, to form a cell pellet. After the supernatant has been removed, the cell pellet may optionally be resuspended in physiological buffer, and centrifuged again, and the supernatant removed. Optionally, the washing process can be repeated one or more times. After one or more washes, most of the pMHC Multiplexers isolated with the cell pellet will be pMHC Multiplexers that bind antigen-specific T cells of the cell sample. Sequencing the encoding molecule component (e.g., the DNA) of a recovered pMHC Multiplexer will reveal the identity of the peptide (p) of the pMHC component carried by that pMHC Multiplexer, in turn identifying the binding specificity of the T cell of the cell sample that bound to this pMHC Multiplexer.
Isolation and identification of pMHC complex-T cell receptor pairs by flow cytometry. The pMHC Multiplexers of the incubation mixture of cells and pMHC Multiplexers from above may be labelled with fluorescent (e.g., PE-labelled) antibodies against part of the pMHC Multiplexer, e.g., by addition of anti-alpha antibody or anti-beta antibody (where beta and alpha are the two proteins of the MHC2 complex). Then the cells are sorted by flow sorting using a flow cytometer; all cells carrying the pMHC Multiplexer-specific label (e.g., PE) (and optionally other markers of the target cells) can then be isolated. By sequencing the encoding molecule (e.g., DNA) of the pMHC Multiplexer the identity of the peptide component of the pMHC Multiplexer is revealed, in turn revealing the binding specificity of the T cells that were isolated. By single cell sequencing of the isolated T cells along with sequencing of the encoding molecule of the pMHC Multiplexer that bound to it, it is possible to identify both the pMHC complex and the corresponding T cell receptor that bound it.

In a preferred embodiment of the invention, the pMHC Multiplexers are used in a screening of patient samples, as follows.

Using a library of e.g., 960 unique pMHC Multiplexers, a patient sample such as blood or tumor material, can be screened for e.g., cancer-specific epitopes by the use of flow sorting, as follows:
Step a: The blood is treated using standard procedures, and the library comprising 960 DNA-tagged pMHC Multiplexers is added under standard conditions, and incubation proceeds for approximately 30 minutes.
Step b: Flow sorting is used to enrich (collect) those T cells that bind a significant number of pMHC Multiplexers.
Step c: The encoding molecule of the pMHC Multiplexers are sequenced—e.g., if the encoding molecule is a DNA the DNA molecules of the pMHC Multiplexers attached to the collected cells are amplified by PCR, after addition of external primers, using standard techniques, and the amplified DNA-tags are then sequenced. The identity of the peptides in the pMHC Multiplexers that bound the T cells and therefore were collected, can now be deduced from the sequence of the recovered and amplified DNA molecules. These peptides represent potential patient- and disease-specific epitopes.

The screening thus provides information about potential disease-specific epitopes of the patient from which the blood- or tumor sample was taken. This information can be used as a diagnostics tool, as well as a means for designing disease- and patient-specific vaccines and therapeutic treatments.

In a preferred embodiment the pMHC Multiplexer is a M13 filamentous phage particle displaying pMHC complexes on its surface and comprising within its interior the DNA encoding the peptide component of said pMHC complexes. A library (collection) of such pMHC Multiplexers may be screened, and potential peptide epitopes identified, by any of the following approaches:
Approach 1: Detection by flow cytometry. A cell sample comprising T cells is mixed with the library of pMHC Multiplexers, and incubated. Fluorescently labelled anti-CD4 antibodies and fluorescently labelled anti-pVIII antibodies are added, and incubation continued. Then flow sorting is used to isolate Helper T cells (bind anti-CD4 antibodies) that recognize and bind specific pMHC Multiplexers (bind anti-pVIII antibodies). Following the isolation of CD4+, pMHC Multiplexer+ T cells, the DNA of the pMHC Multiplexers is sequenced. This will reveal the identity of the peptides of the pMHC complexes (of the pMHC Multiplexers) that were recognized by the T cell receptors of the T cells. These peptides are (potential) peptide epitopes.
Approach 2: Detection by immobilization on anti-CD4 antibody-coated beads. A cell sample comprising T cells is mixed with the library of pMHC Multiplexers, and incubated. Magnetic beads are coated with anti-CD4 antibodies. The mixture of cells and pMHC Multiplexers from above are added to the anti-CD4-coated beads, and is incubated. Then the CD4+ T cells, bound to the anti-CD4-coated beads, are removed from the solution, transferred to another tube, and washed a few times in physiological buffer while on the magnetic beads. The pMHC Multiplexers that bind the CD4+ T cells after several washes will be those pMHC Multiplexers that carry pMHC complexes able to bind T cell receptors of the immobilized T cells. Sequencing of the DNA comprised within the bound pMHC Multiplexers will reveal the identity of the peptides of these pMHC complexes. These peptides are (potential) peptide epitopes.
Approach 3: Cell precipitation by centrifugation. A cell sample comprising T cells is mixed with the library of pMHC Multiplexers and is incubated. Then the solution is centrifuged. The cell precipitate will contain the pMHC Multiplexers that were bound to T cells. The cells may be resuspended and re-centrifuged a few times, to wash the cells. The pMHC Multiplexers that bind the T cells after several washes will be those pMHC Multiplexers that carry pMHC complexes able to bind T cell receptors of the T cells. Sequencing of the DNA comprised within the pMHC Multiplexers will reveal the identity of the peptides of these pMHC complexes. These peptides are (potential) peptide epitopes.

Using the same type of screening set-ups, other filamentous phage display systems may be used, and other coat proteins may be employed for the display of pMHC2 complexes. Likewise, other phage types and viruses and cells may be used with appropriate modification of the experimental details.

As described in Example 4, it may improve the screening process to include a pre-enrichment step where phage particles that have not been exposed to empty MHC2 complexes are screened for the ability to complex with empty MHC2, and where those that cannot bind the empty MHC2 are removed before the screening process is initiated. Optionally, the pre-enriched pMHC Multiplexers may be amplified (i.e. each of the copies turned into multiple copies), by re-infection in E. coli and preparation of pMHC Multiplexer from the amplified phage particles, following e.g., the procedure described above.

“Application 4”: Induced Proliferation of Antigen-Specific T Cells by pMHC Multiplexers, where the pMHC Multiplexers are Filamentous Phage Particles Displaying pMHC Complexes on their Surface.

In one embodiment of the present invention, the invention is used to rapidly produce a large number (N) of different pMHC Multiplexers, where N may be any number between 2 and 10¹⁵. Following their production they are pooled into one solution (if they are not already in one solution), to generate a composition (also called a library) of a large number of pMHC Multiplexers. This solution is added to a sample containing T cells, such as e.g., a blood sample, a biopsy from a tumor of a human cancer patient, or a bone marrow sample, allowing for e.g., parallel analysis and possible detection or isolation of one or more antigen-specific T cells. Once the cell sample has been incubated with the pMHC Multiplexers, the T cells that become bound by the pMHC Multiplexers may be identified, quantified or further manipulated, as follows.
Selection by centrifugation. The incubation mixture of cells and pMHC Multiplexers from above may be centrifuged, to form a cell pellet. After the supernatant has been removed, the cell pellet may optionally be resuspended in physiological buffer, and centrifuged again, and the supernatant removed. Optionally, the washing process can be repeated one or more times. After one or more washes, most of the pMHC Multiplexers isolated with the cell pellet will be pMHC Multiplexers that bind antigen-specific T cells of the cell sample. Sequencing the encoding molecule component (e.g., the DNA) of a recovered pMHC Multiplexer will reveal the identity of the peptide (p) of the pMHC component carried by that pMHC Multiplexer, in turn identifying the binding specificity of the T cell of the original cell sample that bound to this pMHC Multiplexer.
Isolation and identification of pMHC complex-T cell receptor pairs by flow cytometry. The pMHC Multiplexers of the incubation mixture of cells and pMHC Multiplexers from above may be labelled with fluorescent (e.g., PE-labelled) anti-bodies against part of the pMHC Multiplexer, e.g., by addition of anti-HC antibody if the pMHC Multiplexers carry MHC1 complexes. Then the cells are sorted by flow sorting using a flow cytometer; all cells carrying the pMHC Multiplexer-specific label (e.g., PE) (and optionally other markers of the target cells) can then be isolated. By sequencing the encoding molecule (e.g., DNA) of the pMHC Multiplexer the identity of the peptide component of the pMHC Multiplexer is revealed, in turn revealing the binding specificity of the T cells that were isolated. By single cell sequencing of the isolated T cells along with sequencing of the encoding molecule of the pMHC Multiplexer that bound to it, it is possible to identify both the pMHC complex and the corresponding T cell receptor that bound it.

In a preferred embodiment of the invention, the pMHC Multiplexers are used in a screening of patient samples, as follows.

Using a library of e.g., 960 unique pMHC Multiplexers, a patient sample such as blood or tumor material, can be screened for e.g., cancer-specific epitopes by the use of flow sorting, as follows:
Step a: The blood is treated using standard procedures, and the library comprising 960 DNA-tagged pMHC Multiplexers is added under standard conditions, and incubation proceeds for approximately 30 minutes.
Step b: Flow sorting is used to enrich (collect) those T cells that bind a significant number of pMHC Multiplexers.
Step c: The encoding molecule of the pMHC Multiplexers are sequenced—e.g., if the encoding molecule is a DNA the DNA molecules of the pMHC Multiplexers attached to the collected cells are amplified by PCR, after addition of external primers, using standard techniques, and the amplified DNA-tags are then sequenced. From the knowledge of which DNA-tags were attached to which peptides in the pMHC Multiplexer library, the identity of the peptides in the pMHC Multiplexers that bound the T cells and therefore were collected, can now be deduced from the sequence of the recovered and amplified DNA molecules. These peptides represent potential patient- and disease-specific epitopes.

The screening thus provides information about potential disease-specific epitopes of the patient from which the blood- or tumor sample was taken. This information can be used as a diagnostics tool, as well as a means for designing disease- and patient-specific vaccines and therapeutic treatments.

In a preferred embodiment the pMHC Multiplexer is a filamentous phage particle displaying pMHC complexes on its surface and comprising within its interior the DNA encoding the peptide component of said pMHC complexes. A library (collection) of such pMHC Multiplexers may be screened, and potential peptide epitopes identified, by any of the following approaches:
Approach 1: Detection by flow cytometry. A cell sample comprising T cells is mixed with the library of pMHC Multiplexers, and incubated. Fluorescently labelled anti-CD8 antibodies and fluorescently labelled anti-pVIII antibodies are added, and incubation continued. Then flow sorting is used to isolate cytotoxic T cells (binds anti-CD8 antibodies) that recognize and bind specific pMHC Multiplexers (binds anti-pVIII antibodies). Following the isolation of CD8+, pMHC Multiplexer+ T cells, the DNA of the pMHC Multiplexers is sequenced. This will reveal the identity of the peptides of the pMHC complexes (of the pMHC Multiplexers) that were recognized by the T cell receptors of the T cells. These peptides are (potential) peptide epitopes.
Approach 2: Detection by immobilization on anti-CD8 antibody-coated beads. A cell sample comprising T cells is mixed with the library of pMHC Multiplexers, and incubated. Magnetic beads are coated with anti-CD8 antibodies. The mixture of cells and pMHC Multiplexers from above are added to the anti-CD8-coated beads, and is incubated. Then the CD8+ T cells, bound to the anti-CD8-coated beads, are removed from the solution, transferred to another tube, and washed a few times in physiological buffer while on the magnetic beads. The pMHC Multiplexers that bind the CD8+ T cells after several washes will be those pMHC Multiplexers that carry pMHC complexes able to bind T cell receptors of the immobilized T cells. Sequencing of the DNA comprised within the bound pMHC Multiplexers will reveal the identity of the peptides of these pMHC complexes. These peptides are (potential) peptide epitopes.
Approach 3: Cell precipitation by centrifugation. A cell sample comprising T cells is mixed with the library of pMHC Multiplexers and is incubated. Then the solution is centrifuged. The cell precipitate will contain the pMHC Multiplexers that were bound to T cells. The cells may be resuspended and re-centrifuged a few times, to wash the cells. The pMHC Multiplexers that bind the T cells after several washes will be those pMHC Multiplexers that carry pMHC complexes able to bind T cell receptors of the T cells. Sequencing of the DNA comprised within the pMHC Multiplexers will reveal the identity of the peptides of these pMHC complexes. These peptides are (potential) peptide epitopes.

In a preferred embodiment, individual wells of a microtiter-plate each comprise multiple copies of a unique pMHC Multiplexer, where the pMHC Multiplexer is a filamentous phage particle displaying pMHC complexes on its surface and comprising within its interior the DNA encoding the peptide component of said pMHC complexes. The pMHC Multiplexer of each of the wells may be screened, and potential peptide epitopes identified, by any of the following approaches:
Approach 1: Detection by flow cytometry. A cell sample comprising T cells is added to each of the wells comprising a unique pMHC Multiplexer in multiple copies and is incubated. Fluorescently labelled anti-CD8 antibodies and fluorescently labelled anti-pIII antibodies are added, and incubation continued. Then flow sorting applied to the contents of each well, one at a time, is used to identify cytotoxic T cells (binds anti-CD8 antibodies) that recognize and bind specific pMHC Multiplexers (binds anti-pIII antibodies). In case T cells are identified by the flow cytometry analysis, the DNA of the pMHC Multiplexer in the corresponding well is sequenced. This will reveal the identity of the peptide of the pMHC complex (of the pMHC Multiplexer) that was recognized by the T cell receptors of the T cells. This peptide is a (potential) peptide epitope.
Approach 2: Differential proliferation of antigen-specific T cells. To each of the wells comprising each a unique pMHC Multiplexer, a T cell sample (e.g., blood sample) is added. Appropriate cytokines/interleukins and other effector molecules are added, in order to allow stimulation and proliferation of T cells that are activated by encounter with a pMHC complex (of a pMHC Multiplexer) that is capable of binding a T cell receptor and thereby stimulating the corresponding T cell. After a few days of proliferation, the wells that contain pMHC Multiplexers that recognize T cells will have more growth of T cells than those that do not. The DNA of the pMHC Multiplexers in those wells can be sequenced, to reveal the identity of the peptides of the pMHC complexes (of the pMHC Multiplexers) that were recognized by the T cell receptors of the T cells. These peptides are (potential) peptide epitopes.
   “Application 5”: The Use of pMHC1 Multiplexers in the Screening, Flow Sorting, and Induced Proliferation of T Cells.

In one embodiment of the present invention, the invention is used to rapidly produce a large number (N) of different pMHC Multiplexers, where N may be any number between 2 and 10¹⁵. Following their production they are pooled into one solution (if they are not already in one solution), to generate a composition (also called a library) of a large number of pMHC Multiplexers. This solution may be added to a sample containing T cells, such as e.g., a blood sample, a biopsy from a tumor of a human cancer patient, or a bone marrow sample, allowing for e.g., parallel analysis and possible detection or isolation of one or more antigen-specific T cells.

Once the cell sample has been incubated with the pMHC Multiplexers, the T cells that become bound by the pMHC Multiplexers may be identified, quantified or further manipulated, as follows.
Selection by centrifugation. The incubation mixture of cells and pMHC Multiplexers from above may be centrifuged, to form a cell pellet. After the supernatant has been removed, the cell pellet may optionally be resuspended in physiological buffer, and centrifuged again, and the supernatant removed. Optionally, the washing process can be repeated one or more times. After one or more washes, most of the pMHC Multiplexers isolated with the cell pellet will be pMHC Multiplexers that bind antigen-specific T cells of the cell sample. Sequencing the encoding molecule component (e.g., the DNA) of a recovered pMHC Multiplexer will reveal the identity of the peptide (p) of the pMHC component carried by that pMHC Multiplexer, in turn identifying the binding specificity of the T cell of the original cell sample that bound to this pMHC Multiplexer.
Isolation and identification of pMHC complex-T cell receptor pairs by flow cytometry. The pMHC Multiplexers of the incubation mixture of cells and pMHC Multiplexers from above may be labelled with fluorescent (e.g., PE-labelled) anti-bodies against part of the pMHC Multiplexer, e.g., by addition of anti-HC antibody if the pMHC Multiplexers carry MHC1 complexes. Then the cells are sorted by flow sorting using a flow cytometer; all cells carrying the pMHC Multiplexer-specific label (e.g., PE) (and optionally other markers of the target cells) can then be isolated. By sequencing the encoding molecule (e.g., DNA) of the pMHC Multiplexer the identity of the peptide component of the pMHC Multiplexer is revealed, in turn revealing the binding specificity of the T cells that were isolated. By single cell sequencing of the isolated T cells along with sequencing of the encoding molecule of the pMHC Multiplexer that bound to it, it is possible to identify both the pMHC complex and the corresponding T cell receptor that bound it.

In a preferred embodiment of the invention, the pMHC Multiplexers are used in a screening of patient samples, as follows.

Using a library of e.g., 960 unique pMHC Multiplexers, a patient sample such as blood or tumor material, can be screened for e.g., cancer-specific epitopes by the use of flow sorting, as follows:
Step a: The blood is treated using standard procedures, and the library comprising 960 DNA-tagged pMHC Multiplexers is added under standard conditions, and incubation proceeds for approximately 30 minutes.
Step b: Flow sorting is used to enrich (collect) those T cells that bind a significant number of pMHC Multiplexers.
Step c: The encoding molecule of the pMHC Multiplexers are sequenced—e.g., if the encoding molecule is a DNA the DNA molecules of the pMHC Multiplexers attached to the collected cells are amplified by PCR, after addition of external primers, using standard techniques, and the amplified DNA-tags are then sequenced. The identity of the peptides in the pMHC Multiplexers that bound the T cells and therefore were collected, can now be deduced from the sequence of the recovered and amplified DNA molecules. These peptides represent potential patient- and disease-specific epitopes.

The screening thus provides information about potential disease-specific epitopes of the patient from which the blood- or tumor sample was taken. This information can be used as a diagnostics tool, as well as a means for designing disease- and patient-specific vaccines and therapeutic treatments.

In a preferred embodiment the pMHC Multiplexer is a filamentous phage particle displaying pMHC complexes on its surface and comprising within its interior the DNA encoding the peptide (p) component of said pMHC complexes. A library (collection) of such pMHC Multiplexers may be screened, and potential peptide epitopes identified, by any of the following approaches:
Approach 1: Detection by flow cytometry. A cell sample comprising T cells is mixed with the library of pMHC Multiplexers, and incubated. Fluorescently labelled anti-CD8 antibodies and fluorescently labelled anti-pVIII antibodies are added, and incubation continued. Then flow sorting is used to isolate cytotoxic T cells (bind anti-CD8 antibodies) that recognize and bind specific pMHC Multiplexers (bind anti-pVIII antibodies). Following the isolation of CD8+, pMHC Multiplexer+ T cells, the DNA of the pMHC Multiplexers is sequenced. This will reveal the identity of the peptides of the pMHC complexes (of the pMHC Multiplexers) that were recognized by the T cell receptors of the T cells. These peptides are (potential) peptide epitopes.
Approach 2: Detection by immobilization on anti-CD8 antibody-coated beads. A cell sample comprising T cells is mixed with the library of pMHC Multiplexers, and incubated. Magnetic beads are coated with anti-CD8 antibodies. The mixture of cells and pMHC Multiplexers from above are added to the anti-CD8-coated beads, and is incubated. Then the CD8+ T cells, bound to the anti-CD8-coated beads, are removed from the solution, transferred to another tube, and washed a few times in physiological buffer while on the magnetic beads. The pMHC Multiplexers that bind the CD8+ T cells after several washes will be those pMHC Multiplexers that carry pMHC complexes able to bind T cell receptors of the immobilized T cells. Sequencing of the DNA comprised within the bound pMHC Multiplexers will reveal the identity of the peptides of these pMHC complexes. These peptides are (potential) peptide epitopes.
Approach 3: Cell precipitation by centrifugation. A cell sample comprising T cells is mixed with the library of pMHC Multiplexers and is incubated. Then the solution is centrifuged. The cell precipitate will contain the pMHC Multiplexers that were bound to T cells. The cells may be resuspended and re-centrifuged a few times, to wash the cells. The pMHC Multiplexers that bind the T cells after several washes will be those pMHC Multiplexers that carry pMHC complexes able to bind T cell receptors of the T cells. Sequencing of the DNA comprised within the pMHC Multiplexers will reveal the identity of the peptides of these pMHC complexes. These peptides are (potential) peptide epitopes.

In a preferred embodiment, individual wells of a microtiter-plate each comprise multiple copies of a unique pMHC Multiplexer, where the pMHC Multiplexer is a filamentous phage particle displaying pMHC complexes on its surface and comprising within its interior the DNA encoding the peptide component of said pMHC complexes. The pMHC Multiplexer of each of the wells may be screened, and potential peptide epitopes identified, by any of the following approaches:
Approach 1: Detection by flow cytometry. A cell sample comprising T cells is added to each of the wells comprising a unique pMHC Multiplexer in multiple copies, and is incubated. Fluorescently labelled anti-CD8 antibodies and fluorescently labelled anti-pIII antibodies are added, and incubation continued. Then flow sorting applied to the contents of each well, one at a time, is used to identify cytotoxic T cells (binds anti-CD8 antibodies) that recognize and bind specific pMHC Multiplexers (binds anti-pIII antibodies). In case T cells are identified by the flow cytometry analysis, the DNA of the pMHC Multiplexer in the corresponding well is sequenced. This will reveal the identity of the peptide of the pMHC complex (of the pMHC Multiplexer) that was recognized by the T cell receptors of the T cells. This peptide is a (potential) peptide epitope.
Approach 2: Differential proliferation of antigen-specific T cells. To each of the wells comprising each a unique pMHC Multiplexer, a T cell sample (e.g., blood sample) is added. Appropriate cytokines/interleukins and other effector molecules are added, in order to allow stimulation and proliferation of T cells that are activated by encounter with a pMHC complex (of a pMHC Multiplexer) that is capable of binding a T cell receptor and thereby stimulating the corresponding T cell. After a few days of proliferation, the wells that contain pMHC Multiplexers that recognize T cells will have more growth of T cells than those that do not. The DNA of the pMHC Multiplexers in those wells can be sequenced, to reveal the identity of the peptides of the pMHC complexes (of the pMHC Multiplexers) that were recognized by the T cell receptors of the T cells. These peptides are (potential) peptide epitopes.
   “Application 6”: Detection of Antigen-Specific T Cells Using pMHC Multiplexers Comprising Dendritic Cells Displaying pMHC Complexes on their Surface, where the Screening Process Involves Immobilization of T Cells on Anti-CD8 Antibody-Coated Magnetic Beads.

In one embodiment of the present invention, the invention is used to rapidly produce a large number (N) of different pMHC Multiplexers, where N may be any number between 2 and 10¹⁰. Following their production they are pooled into one solution (if they are not already in one solution), to generate a composition (also called a library) of a large number of pMHC Multiplexers. This solution may be added to a sample containing T cells, such as e.g., a blood sample, a biopsy from a tumor of a human cancer patient, or a bone marrow sample, allowing for e.g., parallel analysis and possible detection or isolation of one or more antigen-specific T cells.

Once the cell sample has been incubated with the pMHC Multiplexers, the T cells that become bound by the pMHC Multiplexers may be identified, quantified or further manipulated, as follows.
Isolation and identification of pMHC complex-T cell receptor pairs by flow cytometry. The pMHC Multiplexers of the incubation mixture of cells and pMHC Multiplexers from above may be labelled with fluorescent (e.g., PE-labelled) anti-bodies against part of the pMHC Multiplexer, e.g., by addition of anti-HC antibody if the pMHC Multiplexers carry MHC1 complexes. Then the cells are sorted by flow sorting using a flow cytometer; all cells carrying the pMHC Multiplexer-specific label (e.g., PE) (and optionally other markers, e.g., markers of the target cells, in this case CD8+ T cells) can then be isolated. By identifying the encoding molecule (e.g., DNA) of the pMHC Multiplexer the identity of the peptide component of the pMHC Multiplexer is revealed, in turn revealing the binding specificity of the T cells that were isolated. By single cell sequencing of the isolated T cells along with sequencing of the encoding molecule of the pMHC Multiplexer that bound to it, it is possible to identify both the pMHC complex and the corresponding T cell receptor that bound it.

In a preferred embodiment of the invention, the pMHC Multiplexers are used in a screening of patient samples, as follows.

Using a library of e.g., 960 unique pMHC Multiplexers, a patient sample such as blood or tumor material, can be screened for e.g., cancer-specific epitopes by the use of flow sorting, as follows:
Step a: The blood is treated using standard procedures, and the library comprising 960 DNA-tagged pMHC Multiplexers is added under standard conditions, and incubation proceeds for approximately 30 minutes.
Step b: Flow sorting is used to enrich (collect) those T cells that bind a significant number of pMHC Multiplexers.
Step c: The encoding molecule of the pMHC Multiplexers are sequenced—e.g., if the encoding molecule is a DNA the DNA molecules of the pMHC Multiplexers attached to the collected cells are amplified by PCR, after addition of external primers, using standard techniques, and the amplified DNA-tags are then sequenced. From the knowledge of which DNA-tags were attached to which peptides in the pMHC Multiplexer library, the identity of the peptides in the pMHC Multiplexers that bound the T cells and therefore were collected, can now be deduced from the sequence of the recovered and amplified DNA molecules. These peptides represent potential patient- and disease-specific epitopes.

The screening thus provides information about potential disease-specific epitopes of the patient from which the blood- or tumor sample was taken. This information can be used as a diagnostics tool, as well as a means for designing disease- and patient-specific vaccines and therapeutic treatments.

In a preferred embodiment the pMHC Multiplexer comprises a dendritic cell displaying pMHC complexes on its surface and comprising within its interior the DNA encoding the peptide component of said pMHC complexes. A library (collection) of such pMHC Multiplexers may be screened, and potential peptide epitopes identified, by any of the following approaches:
Approach 1: Detection by flow cytometry. A cell sample comprising T cells is mixed with the library of pMHC Multiplexers, and incubated. Fluorescently labelled anti-CD8 antibodies and fluorescently labelled antibodies that recognize a specific molecule on the surface of dendritic cells are added, and incubation continued. Then flow sorting is used to isolate cytotoxic T cells (binds anti-CD8 antibodies) that recognize and bind specific pMHC Multiplexers (binds anti-dendritic cell antibodies). Following the isolation of CD8+, pMHC Multiplexer+ T cells, the DNAs of the pMHC Multiplexers are sequenced. This will reveal the identity of the peptides of the pMHC complexes (of the pMHC Multiplexers) that were recognized by the T cell receptors of the T cells. These peptides are (potential) peptide epitopes.
Approach 2: Detection by immobilization on anti-CD8 antibody-coated beads. A cell sample comprising T cells is mixed with the library of pMHC Multiplexers, and incubated. Magnetic beads are coated with anti-CD8 antibodies. The mixture of cells and pMHC Multiplexers from above are added to the anti-CD8-coated beads and is incubated. Then the CD8+ T cells, bound to the anti-CD8-coated beads, are removed from the solution, transferred to another tube, and washed a few times in physiological buffer while on the magnetic beads. The pMHC Multiplexers that bind the CD8+ T cells after several washes will be those pMHC Multiplexers that carry pMHC complexes able to bind T cell receptors of the immobilized T cells. Sequencing of the DNA comprised within the bound pMHC Multiplexers will reveal the identity of the peptides of these pMHC complexes. These peptides are (potential) peptide epitopes.

In a preferred embodiment, individual wells of a microtiter-plate each comprise multiple copies of a unique pMHC Multiplexer, where the pMHC Multiplexer is a dendritic cell displaying pMHC complexes on its surface and comprising within its interior the DNA encoding the peptide component of said pMHC complexes. The pMHC Multiplexer of each of the wells may be screened, and potential peptide epitopes identified, by any of the following approaches:
Approach 1: Detection by flow cytometry. A cell sample comprising T cells is added to each of the wells comprising a unique pMHC Multiplexer in multiple copies, and is incubated. Fluorescently labelled anti-CD8 antibodies and fluorescently labelled anti-dendritic cell antibodies are added, and incubation continued. Then flow sorting applied to the contents of each well, one at a time, is used to identify cytotoxic T cells (binds anti-CD8 antibodies) that recognize and bind specific pMHC Multiplexers (binds anti-dendritic cell antibodies). In case T cells that bind a pMHC Multiplexer are identified by the flow cytometry analysis, the DNA of the pMHC Multiplexer in the corresponding well is sequenced. This will reveal the identity of the peptide of the pMHC complexes (of the pMHC Multiplexer) that was recognized by the T cell receptors of the T cells. This peptide is a (potential) peptide epitope.
Approach 2: Differential proliferation of antigen-specific T cells. To each of the wells comprising each a unique pMHC Multiplexer, a T cell sample (e.g., blood sample) is added. Appropriate cytokines/interleukins and other effector molecules are added, in order to allow stimulation and proliferation of T cells that are activated by encounter with a pMHC complex (of a pMHC Multiplexer) that is capable of binding a T cell receptor and thereby stimulating the corresponding T cell. After a few days of proliferation, the wells that contain pMHC Multiplexers that recognize T cells will have more growth of T cells than those that do not. The DNA of the pMHC Multiplexers in those wells can be sequenced, to reveal the identity of the peptides of the pMHC complexes (of the pMHC Multiplexers) that were recognized by the T cell receptors of the T cells. These peptides are (potential) peptide epitopes.

In order to more accurately quantify T cells that bind pMHC Multiplexers of a certain specificity, and/or in order to more stringently isolate T cells that bind pMHC Multiplexers of a certain specificity, several types of pMHC Multiplexers may be added. Thus, in a preferred embodiment, a set of pMHC Multiplexers comprising a set of specificities (e.g., contains 10, 100, or 1000 different peptides bound in 10, 100, or 1000 pMHC complexes, respectively) and comprising each a dendritic cell, may be mixed with a set of pMHC Dextramers comprising the same set of specificities (i.e. contains 10, 100, or 1000 different peptides bound in 10, 100, or 1000 pMHC complexes, respectively), and mix this set of reagents with a cell sample comprising T cells.

Antigen-specific T cell quantification or isolation may now be done as described above, e.g., by flow cytometry. In order to attain a higher accuracy, the two sets of reagents (dendrite cell-based and dextran-based reagents) may be labelled with different fluorophores.

“Application 7”: Identification of Peptide Epitopes Using pMHC Multiplexers that Each Carry a Sense and an Anti-Sense DNA Tag.

In one embodiment of the present invention, the invention is used to rapidly produce a large number (N) of different pMHC Multiplexers, where N is the number of unique peptides (p) of the pMHC Multiplexers and N may be any number between 2 and 1012, and where DNA is the encoding molecule.

Further, the DNA tags are designed as described above, i.e. to each unique peptide corresponds two DNA molecules, one comprising a sense region and the other containing an antisense region, where the sense and antisense region of a pMHC Multiplexer pair can anneal to each other but not to any other sense or antisense regions of another pMHC Multiplexer pair. See (FIG. 21).

Following their production, e.g., by the process described above, they are pooled into one solution (if they are not already in one solution), to generate a composition (also called a library) of a large number of pairs of pMHC Multiplexers. This solution is added to a sample containing T cells, such as e.g., a blood sample, a biopsy from a tumor of a human cancer patient, or a bone marrow sample, thus producing a mixture of cells and pMHC Multiplexers.

Once the cell sample has been incubated with the pMHC Multiplexers, the T cells that become bound by the pMHC Multiplexers may be identified, quantified or further manipulated, as follows.
Isolation and identification of pMHC complex-T cell receptor pairs by flow cytometry. The mixture of pMHC Multiplexers and cells from above are incubated, and then polymerase(s), fluorescent-labelled dNTPs, and other components necessary for extension are added. The double-stranded DNA regions resulting from the annealing of sense- and antisense-DNA strands of two pMHC Multiplexers bound to two neighboring TCRs are now extended from their 3′-ends, thereby forming an extended double stranded region, further increasing the combined binding strength of the two pMHC Multiplexers, and also thereby fluorescently labelling the cells that bound pMHC Multiplexers in this way.
Then the cells are sorted by flow sorting using a flow cytometer; all cells carrying the pMHC Multiplexer-specific label (i.e. the fluorescent label(s) that was introduced during extension), and optionally other markers of the target cells (e.g., anti-CD8 or anti-CD4, labelled with fluorophores that emit light at a wavelength different from those fluorochromes that were attached to the dNTPs) can then be isolated. Optionally, a PCR reaction is performed at this step, amplifying the extended DNA strands. Sequencing is performed on the encoding molecules (e.g., DNA) isolated with the isolated cells; the sequencing is preferably done using a primer that anneals to the part of the DNA duplex that was formed during extension. Thus, the criteria for a DNA molecule to be sequenced is that it i) was part of a pMHC Multiplexer that could bind to a TCR on a T cell, ii) bound close to another pMHC Multiplexer on the same T cell, iii) the sense and antisense of the two pMHC Multiplexers annealed and were extended during the extension, iv) the cell that the two pMHC Multiplexers bound to was isolated in the flow sorting step, and v) the extension process generated an extended DNA strand reaching past the annealing site of the sequencing primer, and vi) the primer used for sequencing was able to bind to the extended DNA strand. Together, these criteria make up a relatively robust system for detection of pMHC Multiplexers capable of binding to antigen-specific T cells.
From the sequencing of the encoding DNA the identity of the pMHC complex of a pMHC Multiplexer that bound the antigen-specific T cell can be deduced, from knowledge of which DNA tags were attached to which peptides (p) during the generation of the pMHC Multiplexers.

In a preferred embodiment of the invention, the pMHC Multiplexers are used in a screening of patient samples, as follows.

Using a library of e.g., 960 unique pMHC Multiplexers, a patient sample such as blood or tumor material, can be screened for e.g., cancer-specific epitopes by the use of flow sorting, as follows:
Step a: The blood is treated using standard procedures, and the library comprising 960 DNA-tagged pMHC Multiplexers is added under standard conditions, and incubation proceeds for approximately 30 minutes. Extension using fluorescently labelled dNTPs as described above.
Step b: Flow sorting is used to enrich (collect) those T cells that carry fluorescence above a certain level.
Step c: The encoding molecule of the pMHC Multiplexers are sequenced—e.g., the DNA molecules of the pMHC Multiplexers attached to the collected cells are amplified by PCR, after addition of external primers, using standard techniques, and the amplified DNA-tags are then sequenced. From the knowledge of which DNA-tags were attached to which peptides in the pMHC Multiplexer library, the identity of the peptides in the pMHC Multiplexers that bound the T cells and therefore were collected, can now be deduced from the sequence of the recovered and amplified DNA molecules. These peptides represent potential patient- and disease-specific epitopes.

The screening thus provides information about potential disease-specific epitopes of the patient from which the blood- or tumor sample was taken. This information can be used as a diagnostics tool, as well as a means for designing disease- and patient-specific vaccines and therapeutic treatments.

In a preferred embodiment the screening process involves solely repeated washing steps of the cells. A library (collection) of pMHC Multiplexers (prepared as described above) are incubated with a cell sample, screened, and potential peptide epitopes identified, by any of the following approaches:
Approach 1: Immobilization on anti-CD8 antibody-coated beads. A cell sample comprising T cells is mixed with the library of pMHC Multiplexers, and incubated. Magnetic beads are coated with anti-CD8 antibodies. The mixture of cells and pMHC Multiplexers from above are added to the anti-CD8-coated beads, and is incubated. Then extension from the annealed sense and antisense strands is performed, as described above. This increases the stability of binding of the pMHC Multiplexers that have become non-covalently linked (through an extended DNA duplex) to another (identical) pMHC Multiplexer on the surface of a T cell. Then the CD8+ T cells, bound to the anti-CD8-coated beads, are removed from the solution, transferred to another tube, and washed a few times in physiological buffer while on the magnetic beads. The pMHC Multiplexers that bind the CD8+ T cells after several washes will preferentially be those pMHC Multiplexers that i) carry pMHC complexes able to bind T cell receptors of the immobilized T cells, and ii) have become attached to one or more other pMHC Multiplexers thereby achieving increased half-life of binding. Sequencing of the DNA comprised within the bound pMHC Multiplexers will reveal the identity of the peptides of these pMHC complexes. These peptides are (potential) peptide epitopes.
Approach 3: Cell precipitation by centrifugation. A cell sample comprising T cells is mixed with the library of pMHC Multiplexers and is incubated. Then extension from the annealed sense and antisense strands is performed, as described above. This increases the stability of binding of the pMHC Multiplexers that have become non-covalently linked (through an extended DNA duplex) to another (identical) pMHC Multiplexer on the surface of a T cell. Then the solution is centrifuged. The cell precipitate will contain the pMHC Multiplexers that were bound to T cells. The cells may be resuspended and re-centrifuged a few times, to wash the cells. The pMHC Multiplexers that bind the T cells after several washes will preferentially be those pMHC Multiplexers that i) carry pMHC complexes able to bind T cell receptors of the immobilized T cells, and ii) have become attached to one or more other pMHC Multiplexers thereby achieving increased half-life of binding. Sequencing of the DNA comprised within the bound pMHC Multiplexers will reveal the identity of the peptides of these pMHC complexes. These peptides are (potential) peptide epitopes.

In a preferred embodiment, individual wells of a microtiter-plate each comprise multiple copies of a unique pMHC1 Multiplexer, where the pMHC1 Multiplexer is prepared as described above. The pMHC Multiplexer of each of the wells may be screened, and potential peptide epitopes identified, by differential proliferation of antigen-specific T cells, as follows.

To each of the wells comprising each a unique pMHC1 Multiplexer, a T cell sample (e.g., blood sample) is added. Extension from the sense/antisense duplex is preformed, as described above. Appropriate cytokines/interleukins and other effector molecules are added, in order to allow stimulation and proliferation of T cells that are activated by encounter with a pMHC1 complex (of a pMHC1 Multiplexer) that is capable of binding a T cell receptor and thereby stimulating the corresponding T cell. After a few days of proliferation, the wells that contain pMHC1 Multiplexers that recognize T cells will have more growth of T cells than those that do not. The DNA of the pMHC1 Multiplexers in those wells can be sequenced, to reveal the identity of the peptides of the pMHC1 complexes (of the pMHC1 Multiplexers) that were recognized by the T cell receptors of the T cells. These peptides are (potential) peptide epitopes.

“Application 8”: A Process for the Detection of Antigen-Specific T Cells, Involving PMHC Multiplexers and Microtiter Plates, where Each pMHC Multiplexer Comprises a Streptavidin to which is Attached 4 pMHC Complexes

In one embodiment of the present invention, the invention is used to rapidly produce a large number (N) of different pMHC Multiplexers, where N may be any number between 2 and 105. Following their production they are pooled into one solution (if they are not already in one solution), to generate a composition (also called a library) of a large number of pMHC Multiplexers. This solution may be added to a sample containing T cells, such as e.g., a blood sample, a biopsy from a tumor of a human cancer patient, or a bone marrow sample, allowing for e.g., parallel analysis and possible detection or isolation of one or more antigen-specific T cells. Once the cell sample has been incubated with the pMHC Multiplexers, the T cells that become bound by the pMHC Multiplexers may be identified, quantified or further manipulated, as follows.
Selection by centrifugation. The incubation mixture of cells and pMHC Multiplexers from above may be centrifuged, to form a cell pellet. After the supernatant has been removed, the cell pellet may optionally be resuspended in physiological buffer, and centrifuged again, and the supernatant removed. Optionally, the washing process can be repeated one or more times. After one or more washes, most of the pMHC Multiplexers isolated with the cell pellet will be pMHC Multiplexers that bind antigen-specific T cells of the cell sample. Sequencing the encoding molecule component (e.g., the DNA) of a recovered pMHC Multiplexer will reveal the identity of the peptide (p) of the pMHC component carried by that pMHC Multiplexer, in turn identifying the binding specificity of the T cell of the original cell sample that bound to this pMHC Multiplexer.
Isolation and identification of pMHC complex-T cell receptor pairs by flow cytometry. The pMHC Multiplexers of the incubation mixture of cells and pMHC Multiplexers from above may be labelled with fluorescent (e.g., PE-labelled) anti-bodies against part of the pMHC Multiplexer, e.g., by addition of anti-alpha antibody if the pMHC Multiplexers carry MHC2 complexes. Then the cells are sorted by flow sorting using a flow cytometer; all cells carrying the pMHC Multiplexer-specific label (e.g., PE) (and optionally other markers of the target cells) can then be isolated. By sequencing the encoding molecule (e.g., DNA) of the pMHC Multiplexer the identity of the peptide component of the pMHC Multiplexer is revealed, in turn revealing the binding specificity of the T cells that were isolated. By single cell sequencing of the isolated T cells along with sequencing of the encoding molecule of the pMHC Multiplexer that bound to it, it is possible to identify both the pMHC complex and the corresponding T cell receptor that bound it.

In a preferred embodiment of the invention, the pMHC Multiplexers are used in a screening of patient samples, as follows.

Using a library of e.g., 960 unique pMHC Multiplexers, a patient sample such as blood or tumor material, can be screened for e.g., cancer-specific epitopes by the use of flow sorting, as follows:
Step a: The blood is treated using standard procedures, and the library comprising 960 DNA-tagged pMHC Multiplexers is added under standard conditions, and incubation proceeds for approximately 30 minutes.
Step b: Flow sorting is used to enrich (collect) those T cells that bind a significant number of pMHC Multiplexers.
Step c: The encoding molecule of the pMHC Multiplexers are sequenced—e.g., if the encoding molecule is a DNA the DNA molecules of the pMHC Multiplexers attached to the collected cells are amplified by PCR, after addition of external primers, using standard techniques, and the amplified DNA-tags are then sequenced. From the knowledge of which DNA-tags were attached to which peptides in the pMHC Multiplexer library, the identity of the peptides in the pMHC Multiplexers that bound the T cells and therefore were collected, can now be deduced from the sequence of the recovered and amplified DNA molecules. These peptides represent potential patient- and disease-specific epitopes.

The screening thus provides information about potential disease-specific epitopes of the patient from which the blood- or tumor sample was taken. This information can be used as a diagnostics tool, as well as a means for designing disease- and patient-specific vaccines and therapeutic treatments.

In a preferred embodiment the pMHC Multiplexer comprises a MHC Dextramer and a DNA-tag, where the DNA encodes the peptide (p) component of said pMHC complexes, and where further the Dextramer carries one or more fluorochromes, here one or more PE molecules. A library (collection) of such pMHC Multiplexers may be screened, and potential peptide epitopes identified, by any of the following approaches:
Approach 1: Detection by flow cytometry. A cell sample comprising T cells is mixed with the library of pMHC Multiplexers, and incubated. Fluorescently (e.g., APC)-labelled anti-CD8 is added, and incubation continued. Then flow sorting is used to isolate cytotoxic T cells (binds anti-CD8 antibodies) that recognize and bind specific pMHC Multiplexers. Following the isolation of CD8+, pMHC Multiplexer+ T cells, the DNA of the pMHC Multiplexers is sequenced. This will reveal the identity of the peptides of the pMHC complexes (of the pMHC Multiplexers) that were recognized by the T cell receptors of the T cells. These peptides are (potential) peptide epitopes.
Approach 2: Detection by immobilization on anti-CD8 antibody-coated beads. A cell sample comprising T cells is mixed with the library of pMHC Multiplexers, and incubated. Magnetic beads are coated with anti-CD8 antibodies. The mixture of cells and pMHC Multiplexers from above are added to the anti-CD8-coated beads, and is incubated. Then the CD8+ T cells, bound to the anti-CD8-coated beads, are removed from the solution, transferred to another tube, and washed a few times in physiological buffer while on the magnetic beads. The pMHC Multiplexers that bind the CD8+ T cells after several washes will be those pMHC Multiplexers that carry pMHC complexes able to bind T cell receptors of the immobilized T cells. Sequencing of the DNA comprised within the bound pMHC Multiplexers will reveal the identity of the peptides of these pMHC complexes. These peptides are (potential) peptide epitopes.
Approach 3: Cell precipitation by centrifugation. A cell sample comprising T cells is mixed with the library of pMHC Multiplexers and is incubated. Then the solution is centrifuged. The cell precipitate will contain the pMHC Multiplexers that were bound to T cells. The cells may be resuspended and re-centrifuged a few times, to wash the cells. The pMHC Multiplexers that bind the T cells after several washes will be those pMHC Multiplexers that carry pMHC complexes able to bind T cell receptors of the T cells. Sequencing of the DNA comprised within the pMHC Multiplexers will reveal the identity of the peptides of these pMHC complexes. These peptides are (potential) peptide epitopes.

In a preferred embodiment, individual wells of a microtiter-plate each comprise multiple copies of a unique pMHC Multiplexer, where the pMHC Multiplexer comprises a streptavidin to which is attached 4 pMHC complexes. The pMHC Multiplexer of each of the wells may be screened, and potential peptide epitopes identified, by any of the following approaches:
Approach 1: Detection by flow cytometry. A cell sample comprising T cells is added to each of the wells comprising a unique pMHC Multiplexer in multiple copies, and is incubated. Fluorescently labelled anti-CD8 antibodies and fluorescently labelled anti-MHC2 protein antibodies are added, and incubation continued. Then flow sorting applied to the contents of each well, one at a time, is used to identify cytotoxic T cells (binds anti-CD8 antibodies) that recognize and bind specific pMHC Multiplexers (binds anti-MHC2 protein antibodies). In case T cells are identified by the flow cytometry analysis, the DNA of the pMHC Multiplexer in the corresponding well is sequenced. This will reveal the identity of the peptide of the pMHC complex (of the pMHC Multiplexer) that was recognized by the T cell receptors of the T cells. This peptide is a (potential) peptide epitope.
Approach 2: Differential proliferation of antigen-specific T cells. To each of the wells comprising each a unique pMHC Multiplexer, a T cell sample (e.g., blood sample) is added. Appropriate cytokines/interleukins and other effector molecules are added, in order to allow stimulation and proliferation of T cells that are activated by encounter with a pMHC complex (of a pMHC Multiplexer) that is capable of binding a T cell receptor and thereby stimulating the corresponding T cell. After a few days of proliferation, the wells that contain pMHC Multiplexers that recognize T cells will have more growth of T cells than those that do not. The DNA of the pMHC Multiplexers in those wells can be sequenced, to reveal the identity of the peptides of the pMHC complexes (of the pMHC Multiplexers) that were recognized by the T cell receptors of the T cells. These peptides are (potential) peptide epitopes.
   “Application 9”: Detection of Antigen-Specific T Cells Using pMHC Multiplexers Each Comprising a SP1 Dodecamer Displaying Up to 12 pMHC Complexes

In one embodiment of the present invention, the invention is used to rapidly produce a large number (N) of different pMHC Multiplexers, where N may be any number between 2 and 104. Following their production they are pooled into one solution (if they are not already in one solution), to generate a composition (also called a library) of a large number of pMHC Multiplexers. This solution is added to a sample containing T cells, such as e.g., a blood sample, a biopsy from a tumor of a human cancer patient, or a bone marrow sample, allowing for e.g., parallel analysis and possible detection or isolation of one or more antigen-specific T cells.

Once the cell sample has been incubated with the pMHC Multiplexers, the T cells that become bound by the pMHC Multiplexers may be identified, quantified or further manipulated, as follows.
Selection by centrifugation. The incubation mixture of cells and pMHC Multiplexers from above may be centrifuged, to form a cell pellet. After the supernatant has been removed, the cell pellet may optionally be resuspended in physiological buffer, and centrifuged again, and the supernatant removed. Optionally, the washing process can be repeated one or more times. After one or more washes, most of the pMHC Multiplexers isolated with the cell pellet will be pMHC Multiplexers that bind antigen-specific T cells of the cell sample. Sequencing the encoding molecule component (e.g., the DNA) of a recovered pMHC Multiplexer will reveal the identity of the peptide (p) of the pMHC component carried by that pMHC Multiplexer, in turn identifying the binding specificity of the T cell of the original cell sample that bound to this pMHC Multiplexer.
Isolation and identification of pMHC complex-T cell receptor pairs by flow cytometry. The pMHC Multiplexers of the incubation mixture of cells and pMHC Multiplexers from above may be labelled with fluorescent (e.g., PE-labelled) anti-bodies against part of the pMHC Multiplexer, e.g., by addition of anti-HC antibody if the pMHC Multiplexers carry MHC1 complexes. Then the cells are sorted by flow sorting using a flow cytometer; all cells carrying the pMHC Multiplexer-specific label (e.g., PE) (and optionally other markers of the target cells) can then be isolated. By sequencing the encoding molecule (e.g., DNA) of the pMHC Multiplexer the identity of the peptide component of the pMHC Multiplexer is revealed, in turn revealing the binding specificity of the T cells that were isolated. By single cell sequencing of the isolated T cells along with sequencing of the encoding molecule of the pMHC Multiplexer that bound to it, it is possible to identify both the pMHC complex and the corresponding T cell receptor that bound it.

In a preferred embodiment of the invention, the pMHC Multiplexers are used in a screening of patient samples, as follows.

Using a library of e.g., 960 unique pMHC Multiplexers, a patient sample such as blood or tumor material, can be screened for e.g., cancer-specific epitopes by the use of flow sorting, as follows:
Step a: The blood is treated using standard procedures, and the library comprising 960 DNA-tagged pMHC Multiplexers is added under standard conditions, and incubation proceeds for approximately 30 minutes.
Step b: Flow sorting is used to enrich (collect) those T cells that bind a significant number of pMHC Multiplexers.
Step c: The encoding molecule of the pMHC Multiplexers are sequenced—e.g., if the encoding molecule is a DNA, the DNA molecules of the pMHC Multiplexers attached to the collected cells are amplified by PCR, after addition of external primers, using standard techniques, and the amplified DNA-tags are then sequenced. From the knowledge of which DNA-tags were attached to which peptides in the pMHC Multiplexer library, the identity of the peptides in the pMHC Multiplexers that bound the T cells and therefore were collected, can now be deduced from the sequence of the recovered and amplified DNA molecules. These peptides represent potential patient- and disease-specific epitopes.

The screening thus provides information about potential disease-specific epitopes of the patient from which the blood- or tumor sample was taken. This information can be used as a diagnostics tool, as well as a means for designing disease- and patient-specific vaccines and therapeutic treatments.

In a preferred embodiment the pMHC Multiplexer comprises a SP1 dodecamer displaying up to 12 pMHC complexes and comprising the DNA encoding the peptide component (p) of said pMHC complexes. A library (collection) of such pMHC Multiplexers may be screened, and potential peptide epitopes identified, by any of the following approaches:
Approach 1: Detection by flow cytometry. A cell sample comprising T cells is mixed with a library of pMHC1 Multiplexers and incubated. Fluorescently labelled (e.g., PE-labelled) anti-CD8 antibodies and fluorescently labelled (e.g., APC-labelled) anti-heavy chain (HC) antibodies are added, and incubation continued. Then flow sorting is used to isolate cytotoxic T cells (binds anti-CD8 antibodies) that recognize and bind specific pMHC Multiplexers (binds anti-heavy chain (HC) antibodies). Following the isolation of CD8+, pMHC Multiplexer+ T cells, the DNA of the pMHC Multiplexers is sequenced. This will reveal the identity of the peptides of the pMHC complexes (of the pMHC Multiplexers) that were recognized by the T cell receptors of the T cells. These peptides are (potential) peptide epitopes.
Approach 2: Detection by immobilization on anti-CD8 antibody-coated beads. A cell sample comprising T cells is mixed with the library of pMHC Multiplexers and incubated. Magnetic beads are coated with anti-CD8 antibodies. The mixture of cells and pMHC Multiplexers from above are added to the anti-CD8-coated beads, and is incubated. Then the CD8+ T cells, bound to the anti-CD8-coated beads, are removed from the solution, transferred to another tube, and washed a few times in physiological buffer while on the magnetic beads. The pMHC Multiplexers that bind the CD8+ T cells after several washes will be those pMHC Multiplexers that carry pMHC complexes able to bind T cell receptors of the immobilized T cells. Sequencing of the DNA comprised within the bound pMHC Multiplexers will reveal the identity of the peptides of these pMHC complexes. These peptides are (potential) peptide epitopes.
Approach 3: Cell precipitation by centrifugation. A cell sample comprising T cells is mixed with the library of pMHC Multiplexers and is incubated. Then the solution is centrifuged. The cell precipitate will contain the pMHC Multiplexers that were bound to T cells. The cells may be resuspended and re-centrifuged a few times, to wash the cells. The pMHC Multiplexers that bind the T cells after several washes will be those pMHC

Multiplexers that carry pMHC complexes able to bind T cell receptors of the T cells. Sequencing of the DNA comprised within the pMHC Multiplexers will reveal the identity of the peptides of these pMHC complexes. These peptides are (potential) peptide epitopes.

As described above, SP1-based pMHC Multimers may be produced by the present invention, by simply excluding the step of DNA attachment included in the SP1-based pMHC Multiplexer production. Such SP1-based pMHC Multimers may be used in the same applications as can be done with MHC Tetramers. Typical labels used in such experiments include fluorochromes, antibodies, elements (particularly rare earth metals like lanthanide). One such application of such SP1-based pMHC Multimers is described immediately below: In this example, the SP1-based pMHC Multimer resulting from excluding the DNA and replacing the DNA with a PE fluorescent label, as described in Comment 9 above, is used in a flow cytometry-based detection of a particular antigen-specific T cell.

Step 1: First the SP1-based pMHC Multimer is labelled with the PE fluorochrome, e.g., by incubating the SP1-based pMHC Multimer with a N-hydroxysuccinimide (NHS) ester-functionalised PE at a pH of e.g., 9 for 30 minutes, to allow amino groups on the SP1-based pMHC Multimer to react with the NHS ester, thereby forming a covalent bond between the pMHC Multimer and the PE fluorochrome.

Step 2: The PE-labelled SP1-based pMHC Multimer is incubated with a blood sample comprising T cells.
Step 3: A flow analysis is performed on the incubated mixture, and the cells that carry PE-fluorescence are detected as positives, those that do not carry PE-fluorescence are detected as positive.

The number of positives will therefore be the number of antigen-specific T cells in the blood sample that recognize the unique pMHC complex of the pMHC Multimer.

In a preferred embodiment, individual wells of a microtiter-plate each comprise multiple copies of a unique pMHC1 Multiplexer, where the pMHC1 Multiplexer is a SP1-based pMHC1 Multiplexer displaying pMHC1 complexes and carrying the DNA encoding the peptide component (p) of said pMHC1 complexes. The pMHC Multiplexer of each of the wells may be screened, and potential peptide epitopes identified, by any of the following approaches:
Approach 1: Detection by flow cytometry. A cell sample comprising T cells is added to each of the wells comprising a unique pMHC Multiplexer in multiple copies, and is incubated. Fluorescently labelled anti-CD8 antibodies and fluorescently labelled anti-HC antibodies are added, and incubation continued. Then flow sorting applied to the contents of each well, one at a time, is used to identify cytotoxic T cells (binds anti-CD8 antibodies) that recognize and bind specific pMHC Multiplexers (binds anti-HC antibodies). In case T cells are identified by the flow cytometry analysis, the DNA of the pMHC Multiplexer in the corresponding well is sequenced. This will reveal the identity of the peptide of the pMHC complexes (of the pMHC Multiplexer) that was recognized by the T cell receptors of the T cells. This peptide is a (potential) peptide epitope.
Approach 2: Differential proliferation of antigen-specific T cells. To each of the wells comprising each a unique pMHC1 Multiplexer, a T cell sample (e.g., blood sample) is added. Appropriate cytokines/interleukins and other effector molecules are added, in order to allow stimulation and proliferation of T cells that are activated by encounter with a pMHC1 complex (of a pMHC1 Multiplexer) that is capable of binding a T cell receptor and thereby stimulating the corresponding T cell. After a few days of proliferation, the wells that contain pMHC1 Multiplexers that recognize T cells will have more growth of T cells than those that do not. The DNA of the pMHC1 Multiplexers in those wells can be sequenced, to reveal the identity of the peptides of the pMHC1 complexes (of the pMHC1 Multiplexers) that were recognized by the T cell receptors of the T cells. These peptides are (potential) peptide epitopes.

### Example Screening, Sorting or Modification Processes.

This table lists some of the approaches that may be applied for the screening, sorting or modification of antigen-specific T cells using pMHC Multiplexers of this invention.
Flow cytometry sorting, also called flow sorting
Manual or automated sorting
Bead sorting
Magnetic bead sorting.
Immobilization in microtiter plate or other surface
Centrifugation to precipitate T cells, and with them the bound pMHC Multiplexers.
Immunoprecipitation.
Proliferation of cells bound to pMHC Multiplexers.
Killing of cells bound to pMHC Multiplexers.
Identification of T cell receptor/pMHC pairs.

### ASPECTS

The following Aspects list preferred embodiments of the invention.
Aspect 1: A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, where the unique DNA molecule is of non-mammalian origin.
Aspect 2: A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, where the unique DNA molecule is of bacterial origin.
Aspect 3: A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, where the unique DNA molecule is of yeast origin.
Aspect 4: A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, where the unique DNA molecule comprises continuous sequences of more than 100 bp that are of non-mammalian origin.
Aspect 5: A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, where the unique DNA molecule comprises continuous sequences of more than 100 bp that are of bacterial origin.
Aspect 6: A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, where the unique DNA molecule comprises continuous sequences of more than 100 bp that are of yeast origin.
Aspect A1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect A2: A composition of two or more pMHC Multiplexers, each comprising an encoding molecule such as a DNA or RNA molecule, functionally linked to a peptide (p) that is encoded by said encoding molecules, and where said peptide is in complex with an MHC complex, thereby making up a pMHC complex.
Aspect A3: The composition of Aspect A2 where the encoding molecule is mechanically linked to the pMHC complex.
Aspect A4: The composition of Aspect A2 or Aspect A3 where the pMHC complex is chemically linked to a phage or virus coat protein.
Aspect A5: The composition of Aspect A4 where the phage is filamentous phage M13 and the coat protein is a fusion protein of the Acid Peptide and the pIII coat protein, or the coat protein is a fusion protein of the Base Peptide and the pIII coat protein, or the coat protein is a fusion protein of the Acid Peptide and the pVIII coat protein, or the coat protein is a fusion protein of the Base Peptide and the pVIII coat protein.
Aspect A6: The composition of Aspect A4 where the pMHC complex is chemically linked to a phage or virus coat protein by way of a non-covalent link.
Aspect A7: The process of making a pMHC Multiplexer, involving the following steps:
   i) Preparing a collection of phagemids that all carry a DNA sequence encoding the Acid Peptide in reading frame with and N-terminal to the pIII coat protein of phage M13, and where each of the phagemids carry a unique DNA sequence leading to the expression of a unique peptide of between 7 and 25 amino acid residues;
   ii Introducing the collection of phagemids of step (i) into the cells of a growing E. coli culture, by e.g., transformation;
   iii) Adding Helper Phage, to produce phage particles displaying the Acid Peptide on pIII coat protein;
   iv) Transferring aliquots of the supernatant to the wells of a microtiter-plate comprising growing E. coli cultures, in an amount corresponding to on average 0.1-0.4 phage particles per aliquot;
   v) Adding Helper Phage, to produce phage particles displaying the Acid Peptide on pIII coat protein;
   vi) Partially lysing the cells, to release peptides encoded by the phagemid;
   vii) Adding Base Peptide-Heavy Chain fusion protein;
   viii) Adding beta2M protein;
   ix) Denaturing the beta2M and Base Peptide-Heavy Chain fusion protein;
   x) Renaturing the proteins;
   xi) Adding redox buffer and then change to more oxidizing conditions, to form the Acid Peptide-Base Peptide dimer, thereby chemically attaching pMHC complexes to the phage particles,
      thereby producing pMHC Multiplexers.
Aspect A8: A MHC Multimer comprising two or more streptavidin molecules.
Aspect A9: The MHC Multimer of Aspect A8 additionally comprising a DNA-tag.
Aspect A10: A screening process involving the pMHC Multiplexer, the MHC Multimer, or the composition of any of Aspects A1-A9, where the screening process involves flow cytometry or centrifugation.
Aspect B1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect B2: A composition of two or more pMHC Multiplexers of Aspect B1.
Aspect B3: The composition of Aspect B2 where the encoding molecule is mechanically linked to the pMHC complex.
Aspect B4: The composition of Aspect B2 or Aspect B3 where the pMHC complex is attached by way of a chemical link to a phage or virus coat protein.
Aspect B5: The composition of Aspect B4 where the phage is filamentous phage M13 and the chemical link is a type of covalent bond that is not found in natural peptides made up of the 20 natural amino acids.
Aspect B6: The process of making a pMHC Multiplexer, involving the following steps:
   i) Preparing a collection of two or more phage genomes where each genome copy carry a DNA sequence encoding a promoter controlling the transcription of a DNA encoding a fusion-protein of a signal peptide and a unique peptide epitope, where said peptide epitope is capable of complexing with a MHC1 or MHC2 complex when it is not attached to the signal peptide;
   ii) Introducing the collection of phage genomes of step (i) into the cells of a growing E. coli culture, by e.g., transformation, and growing the E. coli cells for several generations;
   iii) Transferring aliquots of the supernatant, comprising on average less than 1 phage particle, to individual wells of a microtiter-plate comprising growing E. coli cultures;
   iv) Growing the E. coli cultures, to produce phage particles;
   v) Partially lysing the cells, to release peptides encoded by the phage genome and optionally releasing phage particles into the periplasm;
   vi) Optionally remove cells and cell debris by e.g., centrifugation and transfer of the supernatant to wells of another microtiter-plate;
   vii) To the solution comprising the phage and peptide X, add a compound comprising at least two reactive groups (a) and (x), where (a) is capable of reacting with an amino acid residue of the phage coat protein, and where (x) is capable of reacting with the reactive group (y);
   viii) Add MHC1 complexes that have been modified on at least one surface-exposed amino acid residue with a moiety (y) capable of reacting with (x), and allow reaction of (x) and (y), to covalently link the pMHC1 complexes to the phage coat;
   ix) Denaturing the proteins of the MHC complexes attached to the phage coat;
   x) Renaturing the proteins, thereby allowing the formation of pMHC1 complexes, where the peptide component is the peptide X present in the same well, thereby forming in each well a unique pMHC Multiplexer.
Aspect B7: The process of Aspect B6 where the reactive groups (x) and (y) are a triple bond and an azide, respectively.
Aspect B8: A MHC Multimer comprising two or more streptavidin molecules and a peptide epitope originating from a bacterial genome.
Aspect B9: The MHC Multimer of Aspect B8 additionally comprising a DNA-tag.
Aspect B10: A screening process involving the pMHC Multiplexer, the MHC Multimer, or the composition of any of Aspects B1-B9, where the screening process involves flow cytometry or centrifugation.
Aspect C1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect C2: A composition of two or more pMHC Multiplexers of Aspect C1.
Aspect C3: The composition of Aspect C2 where the encoding molecule is mechanically linked to the pMHC complex.
Aspect C4: The composition of Aspect C2 or Aspect C3 where the pMHC complex is chemically linked to a phage or virus coat protein.
Aspect C5: The composition of Aspect C4 where the phage is filamentous phage M13 and the coat protein is linked to the peptide encoded by the encoding molecule.
Aspect C6: The composition of any of claims 2-5 where the pMHC complex is chemically linked to a phage or virus coat protein by way of a covalent link.
Aspect C7: The process of making a collection of pMHC Multiplexers, involving the following steps:
   i) Preparing a collection of phagemids that all carry a DNA sequence encoding a peptide (p) in reading frame with and N-terminal to the pVIII coat protein of phage M13, and where each of the DNA sequences encode a unique peptide (p) of between 7 and 25 amino acid residues;
   ii) Introducing the collection of phagemids of step (i) into the cells of a growing E. coli culture, by e.g., transformation;
   iii) Adding Helper Phage, to produce phage particles displaying the peptide (p) on pVIII coat protein;
      thereby producing a collection of pMHC Multiplexers.
Aspect C8: A MHC Multimer comprising two or more streptavidin molecules.
Aspect C9: The MHC Multimer of Aspect C8 additionally comprising a DNA-tag.
Aspect C10: A screening process involving the pMHC Multiplexer, the MHC Multimer, or the composition of any of Aspects C1-C9, where the screening process involves flow cytometry or centrifugation.
Aspect D1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect D2: A composition of two or more pMHC Multiplexers of Aspect D1.
Aspect D3: The composition of Aspect D2 where the encoding molecule is mechanically linked to the pMHC complex.
Aspect D4: The composition of Aspect D2 or D3 where the pMHC complex is chemically linked to a phage or virus coat protein.
Aspect D5: The composition of Aspect D4 where the phage is filamentous phage M13 and the coat protein is pIII or pVIII.
Aspect D6: The composition of any of claims 2-5 where the peptide (p) of the pMHC complex is chemically linked to a phage or virus coat protein by way of a covalent link.
Aspect D7: The process of making a collection of pMHC Multiplexers, involving the following steps:
   i) Preparing a collection of phagemids that all carry a DNA sequence encoding a peptide (p) in reading frame with a dimerization domain X, and where each of the DNA sequences encode a unique peptide (p) of between 7 and 25 amino acid residues, and carrying a DNA sequence encoding a dimerization domain Y fused to the pVIII coat protein, where the X and Y dimerization domains are capable of dimerizing to each other;
   ii) Introducing the collection of phagemids of step (i) into the cells of a growing E. coli culture, by e.g., transformation;
   iii) Adding Helper Phage, to produce phage particles displaying the peptide (p) on a phage coat protein;
   iv) Adding empty MHC2 complexes;
      thereby producing a collection of pMHC Multiplexers.
Aspect D8: A MHC Multimer comprising 5 or more identical pMHC complexes.
Aspect D9: The MHC Multimer of Aspect D8 additionally comprising a DNA-tag.
Aspect D10: A screening process involving the pMHC Multiplexer, the MHC Multimer, or the composition of any of Aspects D1-D9, where the screening process involves flow cytometry or centrifugation.
Aspect E1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect E2: A composition of two or more pMHC Multiplexers, each comprising an encoding molecule such as a DNA or RNA molecule, functionally linked to a peptide (p) that is encoded by said encoding molecules, and where said peptide is in complex with an MHC complex, thereby making up a pMHC complex.
Aspect E3: The composition of Aspect E2 where the encoding molecule is mechanically linked to the pMHC complex.
Aspect E4: The composition of Aspect E2 or Aspect E3 where the pMHC complex is chemically linked to a phage or virus coat protein or is linked to a human cell such as a dendritic cell.
Aspect E5: The composition of Aspect E4 where the phage is filamentous phage M13 and the coat protein is a fusion protein of the HC and the pVIII coat protein, or the coat protein is a fusion protein of the beta2M and the pVIII coat protein, or the coat protein is a fusion protein of the Acid Peptide and the pVIII coat protein, or the coat protein is a fusion protein of the Base Peptide and the pVIII coat protein.
Aspect E6: The composition of Aspect E4 where the pMHC complex is chemically linked to a phage or virus coat protein or is linked to a dendritic cell by way of a non-covalent link.
Aspect E7: The process of making a pMHC Multiplexer, involving the following steps:
   i) Preparing a collection of encoding molecules, capable of being transcribed and/or translated into peptides or proteins, where each encoding molecule encodes the peptide of the pMHC complex being displayed in multiple copies in the a final MHC Multiplexer, or where each encodes the precursor peptide or precursor protein of the peptide of the pMHC complex being displayed in multiple copies in a final MHC Multiplexer; and where the encoding molecules may be a collection of RNA molecules or a collection of DNA molecules, where the DNA molecules may be made by synthetic chemistry or may be made by enzymatic means such as by reverse transcription of an mRNA, followed by amplification e.g., by PCR, and where the DNA may be e.g., a wild type virus or a genetically modified recombinant virus e.g., belonging to the following group of viruses: adenovirus, retrovirus, herpes simplex virus, vaccinia virus, influenza virus, and alpha virus; and where the RNA may be a collection of mRNA molecules purified from a cell extract such as a cell extract from a cancer patient, e.g., a cell extract of the cells from a biopsy from the tumor of a cancer patient, or alternatively, cDNA may be prepared from a cell extract, amplified by e.g., PCR and then transcribed into mRNA, or mRNA may be made by transcription from (optionally recombinant) viral DNA or other vector DNA such as plasmids, or may be made from transcription of single-stranded or double-stranded oligonucleotides prepared synthetic chemistry;
   ii) Introducing the collection of encoding molecules into a dendritic cell or a precursor of a dendritic cell, under conditions ensuring that one cell only receives one or more copies of the same encoding molecule, thereby generating a collection of cells, each comprising one or more copies of an encoding molecule, by e.g., electroporation, infection by e.g., virus, phagocytosis of another cell, e.g., a monocyte or bacteria, uptake of lipid nanoparticles or vesicles or other similar entities, or uptake of small lipid-comprising carriers, infection by e.g., bacterium, or transfection, e.g., using liposomes such as DOTAP;
   iii) Adding activating or inhibiting molecule(s);
   iv) Incubating;
   v) Allowing the transcription and/or translation of said encoding molecules;
   vi) Allowing the partial degradation and/or modification and complexation of peptide (p) with MHC complex, to form pMHC complex, and allowing the transfer of the pMHC complex onto the surface of the dendritic cell, thereby resulting in a display of the peptide in complex with MHC protein;
      where steps ii), iii), iv), v), and vi) may be performed in any order, thereby producing pMHC Multiplexers.
Aspect E8: A MHC Multimer comprising two or more streptavidin molecules.
Aspect E9: A MHC Multimer comprising a DNA-tag.
Aspect E10: A screening process involving the pMHC Multiplexer, the MHC Multimer, or the composition of any of Aspects E1-E9, where the screening process involves flow cytometry or centrifugation.
Aspect F1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect F2: A composition of two or more pMHC Multiplexers, each comprising an encoding molecule such as a DNA or RNA molecule, functionally linked to a peptide (p) that is encoded by said encoding molecules, and where said peptide is in complex with an MHC complex, thereby making up a pMHC complex.
Aspect F3: The composition of Aspect F2 where the encoding molecule is mechanically linked to the pMHC complex.
Aspect F4: The composition of Aspect F2 or Aspect F3 where the pMHC complex is chemically linked to a phage or virus coat protein.
Aspect F5: The composition of Aspect F4 where the phage is filamentous phage M13 and the coat protein is a fusion protein of a Pentamer subunit and the pIII coat protein, or the coat protein is a fusion protein of a Pentamer subunit and the pVIII coat protein.
Aspect F6: The composition of Aspect F4 where the pMHC complex is chemically linked to a phage or virus coat protein or is linked to a dendritic cell, by way of a non-covalent link.
Aspect F7: The process of making a pMHC Multiplexer, involving the following steps:
   i) Preparing a collection of encoding molecules, capable of being transcribed and/or translated into peptides or proteins, where each encoding molecule encodes the peptide of the pMHC complex being displayed in multiple copies in the a final MHC Multiplexer, or where each encodes the precursor peptide or precursor protein of the peptide of the pMHC complex being displayed in multiple copies in a final MHC Multiplexer; and where the encoding molecules may be a collection of RNA molecules or a collection of DNA molecules, where the DNA molecules may be made by synthetic chemistry or may be made by enzymatic means such as by reverse transcription of an mRNA, followed by amplification e.g., by PCR, and where the DNA may be e.g., a wild type virus or a genetically modified recombinant virus e.g., belonging to the following group of viruses: adenovirus, retrovirus, herpes simplex virus, vaccinia virus, influenza virus, and alpha virus; and where the RNA may be a collection of mRNA molecules purified from a cell extract such as a cell extract from a cancer patient, e.g., a cell extract of the cells from a biopsy from the tumor of a cancer patient, or alternatively, cDNA may be prepared from a cell extract, amplified by e.g., PCR and then transcribed into mRNA, or mRNA may be made by transcription from (optionally recombinant) viral DNA or other vector DNA such as plasmids, or may be made from transcription of single-stranded or double-stranded oligonucleotides prepared synthetic chemistry;
   ii) Introducing the collection of encoding molecules into a dendritic cell or a precursor of a dendritic cell, under conditions ensuring that one cell only receives one or more copies of the same encoding molecule, thereby generating a collection of cells, each comprising one or more copies of an encoding molecule, by e.g., electroporation, infection by e.g., virus, phagocytosis of another cell, e.g., a monocyte or bacteria, uptake of lipid nanoparticles or vesicles or other similar entities, or uptake of small lipid-comprising carriers, infection by e.g., bacterium, or transfection, e.g., using liposomes such as DOTAP;
   iii) Adding activating or inhibiting molecule(s);
   iv) Incubating;
   v) Allowing the transcription and/or translation of said encoding molecules;
   vi) Allowing the partial degradation and/or modification and complexation of peptide (p) with MHC complex, to form pMHC complex, and allowing the transfer of the pMHC complex onto the surface of the dendritic cell, thereby resulting in a display of the peptide in complex with MHC protein;
   where steps ii), iii), iv), v), and vi) may be performed in any order, thereby producing pMHC Multiplexers.
Aspect F8: A MHC Multimer comprising five or more pMHC complexes.
Aspect F9: A MHC Multiplexer comprising five or more pMHC complexes.
Aspect F10: A screening process involving the pMHC Multiplexer, the MHC Multimer, or the composition, of any of Aspects F1-F9, where the screening process involves flow cytometry or centrifugation.
Aspect G1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect G2: A composition of two or more pMHC Multiplexers, each comprising an encoding molecule such as a DNA or RNA molecule, functionally linked to a peptide (p) that is encoded by said encoding molecules, and where said peptide is in complex with an MHC complex, thereby making up a pMHC complex.
Aspect G3: The composition of Aspect G2 where the encoding molecule is mechanically linked to the pMHC complex.
Aspect G4: The composition of Aspect G2 or Aspect G3 where the pMHC complex is chemically linked to a phage or virus coat protein.
Aspect G5: The composition of Aspect G4 where the phage is filamentous phage M13 and where the pMHC complexes are displayed on a SP1 dodecamer protein scaffold.
Aspect G6: The composition of Aspect G4 where the pMHC complex is chemically linked to a phage or virus coat protein or is linked to a dendritic cell, by way of a non-covalent link.
Aspect G7: The process of making a pMHC Multiplexer, involving the following steps:
   i) Preparing a collection of encoding molecules, capable of being transcribed and/or translated into peptides or proteins, where each encoding molecule encodes the peptide of the pMHC complex being displayed in multiple copies in the a final MHC Multiplexer, or where each encodes the precursor peptide or precursor protein of the peptide of the pMHC complex being displayed in multiple copies in a final MHC Multiplexer; and where the encoding molecules may be a collection of RNA molecules or a collection of DNA molecules, where the DNA molecules may be made by synthetic chemistry or may be made by enzymatic means such as by reverse transcription of an mRNA, followed by amplification e.g., by PCR, and where the DNA may be e.g., a wild type virus or a genetically modified recombinant virus e.g., belonging to the following group of viruses: adenovirus, retrovirus, herpes simplex virus, vaccinia virus, influenza virus, and alpha virus; and where the RNA may be a collection of mRNA molecules purified from a cell extract such as a cell extract from a cancer patient, e.g., a cell extract of the cells from a biopsy from the tumor of a cancer patient, or alternatively, cDNA may be prepared from a cell extract, amplified by e.g., PCR and then transcribed into mRNA, or mRNA may be made by transcription from (optionally recombinant) viral DNA or other vector DNA such as plasmids, or may be made from transcription of single-stranded or double-stranded oligonucleotides prepared synthetic chemistry;
   ii) Introducing the collection of encoding molecules into a dendritic cell or a precursor of a dendritic cell, under conditions ensuring that one cell only receives one or more copies of the same encoding molecule, thereby generating a collection of cells, each comprising one or more copies of an encoding molecule, by e.g., electroporation, infection by e.g., virus, phagocytosis of another cell, e.g., a monocyte or bacteria, uptake of lipid nanoparticles or vesicles or other similar entities, or uptake of small lipid-comprising carriers, infection by e.g., bacterium, or transfection, e.g., using liposomes such as DOTAP;
   iii) Adding activating or inhibiting molecule(s);
   iv) Incubating;
   v) Allowing the transcription and/or translation of said encoding molecules;
   vi) Allowing the partial degradation and/or modification and complexation of peptide (p) with MHC complex, to form pMHC complex, and allowing the transfer of the pMHC complex onto the surface of the dendritic cell, thereby resulting in a display of the peptide in complex with MHC protein;
   where steps ii), iii), iv), v), and vi) may be performed in any order, thereby producing pMHC Multiplexers.
Aspect G8: A MHC Multimer comprising 10 or more pMHC complexes.
Aspect G9: A MHC Multiplexer comprising 10 or more pMHC complexes.
Aspect G10: A screening process involving the pMHC Multiplexer, the MHC Multimer, or the composition, of any of Aspects G1-G9, where the screening process involves flow cytometry or centrifugation.
Aspect H1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect H2: A composition of two or more pMHC Multiplexers, each comprising an encoding molecule such as a DNA or RNA molecule, functionally linked to a peptide (p) that is encoded by said encoding molecules, and where said peptide is in complex with an MHC complex, thereby making up a pMHC complex.
Aspect H3: The composition of Aspect H2 where the encoding molecule is mechanically linked to the pMHC complex.
Aspect H4: The composition of Aspect H2 or Aspect H3 where the pMHC complex is chemically linked to a phage or virus coat protein.
Aspect H5: The composition of Aspect H4 where the phage is filamentous phage M13 and the coat protein is a fusion protein of streptavidin and the pIII coat protein, or the coat protein is a fusion protein of streptavidin and the pVIII coat protein.
Aspect H6: The composition of Aspect H4 where the pMHC complex is chemically linked to a phage or virus coat protein or is linked to a dendritic cell, by way of a non-covalent link.
Aspect H7: The process of making a pMHC Multiplexer, involving the following steps:
   i) Preparing a collection of encoding molecules, capable of being transcribed and/or translated into peptides or proteins, where each encoding molecule encodes the peptide of the pMHC complex being displayed in multiple copies in the a final MHC Multiplexer, or where each encodes the precursor peptide or precursor protein of the peptide of the pMHC complex being displayed in multiple copies in a final MHC Multiplexer; and where the encoding molecules may be a collection of RNA molecules or a collection of DNA molecules, where the DNA molecules may be made by synthetic chemistry or may be made by enzymatic means such as by reverse transcription of an mRNA, followed by amplification e.g., by PCR, and where the DNA may be e.g., a wild type virus or a genetically modified recombinant virus e.g., belonging to the following group of viruses: adenovirus, retrovirus, herpes simplex virus, vaccinia virus, influenza virus, and alpha virus; and where the RNA may be a collection of mRNA molecules purified from a cell extract such as a cell extract from a cancer patient, e.g., a cell extract of the cells from a biopsy from the tumor of a cancer patient, or alternatively, cDNA may be prepared from a cell extract, amplified by e.g., PCR and then transcribed into mRNA, or mRNA may be made by transcription from (optionally recombinant) viral DNA or other vector DNA such as plasmids, or may be made from transcription of single-stranded or double-stranded oligonucleotides prepared synthetic chemistry;
   ii) Introducing the collection of encoding molecules into a dendritic cell or a precursor of a dendritic cell, under conditions ensuring that one cell only receives one or more copies of the same encoding molecule, thereby generating a collection of cells, each comprising one or more copies of an encoding molecule, by e.g., electroporation, infection by e.g., virus, phagocytosis of another cell, e.g., a monocyte or bacteria, uptake of lipid nanoparticles or vesicles or other similar entities, or uptake of small lipid-comprising carriers, infection by e.g., bacterium, or transfection, e.g., using liposomes such as DOTAP;
   iii) Adding activating or inhibiting molecule(s);
   iv) Incubating;
   v) Allowing the transcription and/or translation of said encoding molecules;
   vi) Allowing the partial degradation and/or modification and complexation of peptide (p) with MHC complex, to form pMHC complex, and allowing the transfer of the pMHC complex onto the surface of the dendritic cell, thereby resulting in a display of the peptide in complex with MHC protein;
   where steps ii), iii), iv), v), and vi) may be performed in any order, thereby producing pMHC Multiplexers.
Aspect H8: A MHC Multimer comprising 10 or more pMHC complexes.
Aspect H9: A MHC Multiplexer comprising 10 or more pMHC complexes.
Aspect H10: A screening process involving the pMHC Multiplexer, the MHC Multimer, or the composition, of any of Aspects H1-H9, where the screening process involves flow cytometry or centrifugation.
Aspect I1: A pMHC Multiplexer comprising an encoding molecule that is a DNA, functionally linked to a peptide (p) that is encoded by said encoding molecule, and where said peptide is in complex with an MHC complex, thereby making up a pMHC complex.
Aspect I2: A composition of two or more pMHC Multiplexers, each comprising an encoding molecule that is a DNA, functionally linked to a peptide (p) that is encoded by said encoding molecule, and where said peptide is in complex with an MHC complex, thereby making up a pMHC complex.
Aspect I3: The composition of Aspect I2 where the encoding molecule is mechanically linked to the pMHC complex.
Aspect I4: The composition of Aspect I2 or 3 where the pMHC complex is chemically linked to a phage or virus coat protein.
Aspect I5: The composition of Aspect I4 where the phage is filamentous phage M13 and the coat protein is a fusion protein of the Acid Peptide and the pIII coat protein, or the coat protein is a fusion protein of the Base Peptide and the pIII coat protein, or the coat protein is a fusion protein of the Acid Peptide and the pVIII coat protein, or the coat protein is a fusion protein of the Base Peptide and the pVIII coat protein.
Aspect I6: The composition of Aspect I4 where the pMHC complex is chemically linked to a phage or virus coat protein by way of a non-covalent link.
Aspect I7: The process of making a pMHC Multiplexer, involving the following steps:
   i) Preparing a collection of phagemids that all carry a DNA sequence encoding the Acid Peptide in reading frame with and N-terminal to the pIII coat protein of phage M13, and where each of the phagemids carry a unique DNA sequence leading to the expression of a unique peptide of between 7 and 25 amino acid residues;
   ii) Preparing a DNA vector such as a plasmid that comprises a sequence encoding the beta2M protein and a fusion protein, Base Peptide-HC protein;
   iii) Introducing the collection of phagemids and the vector of step (i) and (ii) into the cells of a growing E. coli culture, by e.g., transformation;
   iv) Adding Helper Phage, to produce intracellular phage particles displaying the Acid Peptide on pIII coat protein;
   v) Allowing the assembly of the Base Peptide-HC with the beta2M and the unique peptide, to form the pMHC complex thereof;
   vi) Allowing the assembly of the Acid-Base dimer, thereby producing intracellular phage particles displaying a pMHC complex on the pIII coat protein;
   vii) Adding redox buffer and then change to more oxidizing conditions, to form the Acid Peptide-Base Peptide dimer, thereby covalently attaching the pMHC complex to the phage coat protein, thereby producing pMHC Multiplexers.
Aspect I8: A MHC Multimer carrying at least five identical pMHC complexes.
Aspect I9: The MHC Multimer of Aspect I8 additionally comprising a DNA-tag.
Aspect I10: A screening process involving the pMHC Multiplexer, the MHC Multimer, or the composition of any of Aspects I1-I9, where the screening process involves flow cytometry or centrifugation.
Aspect J1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect J2: A composition of two or more pMHC Multiplexers, each comprising an encoding molecule such as a DNA or RNA molecule, functionally linked to a peptide (p) that is encoded by said encoding molecule, and where said peptide is in complex with an MHC complex, thereby making up a pMHC complex.
Aspect J3: The composition of Aspect J2 where the encoding molecule is mechanically linked to the pMHC complex.
Aspect J4: The composition of Aspect J2 or Aspect J3 where the pMHC complex is chemically linked to a cell outer surface protein.
Aspect J5: The composition of Aspect J4 where the cell is a dendritic cell.
Aspect J6: The composition of Aspect J4 where the pMHC complex is chemically linked to a cell outer surface protein by way of a non-covalent link.
Aspect J7: The process of making a pMHC Multiplexer, involving the following steps:
   i) Preparing a collection of encoding molecules, capable of being transcribed and/or translated into peptides or proteins;
   ii) Introducing the collection of encoding molecules into a dendritic cell or a precursor of a dendritic cell by e.g., electroporation or infection;
   iii) Adding activating or inhibiting molecule(s);
   iv) Incubating;
   v) Allowing the transcription and/or translation of said encoding molecules;
   vi) Allowing the partial degradation and/or modification and complexation of peptide (p) with MHC complex, to form pMHC complex, displayed on a dendritic cell;
   where steps ii), iii), iv), and v) may be performed in any order, thereby producing pMHC Multiplexers.
Aspect J8: A composition of two or more MHC Multiplexers comprising each a dextran molecule, and two or more MHC Multiplexers each comprising one dendritic cell.
Aspect J9: A composition of ten or more, such as 100 or more, such as 1000 or more, such as 10000 or more MHC Multiplexers comprising each a dextran molecule, and ten or more, such as 100 or more, such as 1000 or more, such as 10000 or more MHC Multiplexers each comprising one dendritic cell.
Aspect J10: The composition of Aspect J8 or Aspect J9, where each of the MHC Multiplexers comprising each a dextran molecule are bound to a T cell.
Aspect K1. A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect K2. A pair of pMHC Multiplexers of Aspect K1 where the encoding molecule is a single-stranded oligonucleotide and where the 3′-terminus of the encoding molecule of one pMHC Multiplexer consists of at least 3 nucleotides that is complementary to at least 3 nucleotides of the 3′-terminus of the encoding molecule of the other pMHC Multiplexer.
Aspect K3. A composition of two or more, such as at least 10 or more, such as at least 100 or more, such as at least 1000 or more, such as at least 10000 or more, such as at least 100000 or more, such as at least 1000000 or more, such as at least 10000000 or more, such as at least 100000000 or more, such as at least 1000000000 or more, such as at least 10000000000 or more, such as at least 100000000000 or more pairs of pMHC Multiplexers of Aspect K2.

The pMHC Multiplexer or the pair or the composition of any of claims 1-3 where each pMHC Multiplexer comprises two or more, such as 3 or more, such as 4 or more, such as 5 or more, such as 6 or more, such as 7 or more, such as 8 or more, such as 9 or more, such as 10 or more encoding molecules.
Aspect K4. The pMHC Multiplexer or the pair or the composition of any of claims 1-3 where each pMHC Multiplexer comprises a MHC Tetramer or a MHC Dextramer or a MHC Streptamer or a MHC Pentamer.
Aspect K5. A method for the detection or isolation of an antigen-specific T cell consisting of the following steps:
   i) Providing one or more T cells,
   ii) Providing one or more pairs of pMHC Multiplexers of Aspect K2,
   iii) Allowing the one or more pairs of pMHC Multiplexers to bind to the one or more T cells, and allowing any pair of pMHC Multiplexers bound to the same T cell to form a duplex by having their encoding molecules form a duplex,
   iv) Extending each of the oligonucleotides of the duplex from the 3′-end in a template-dependent manner, optionally incorporating labelled dNTPs into the extended DNA strand,
   v) Optionally, determining the degree of incorporation of dNTPs by determining the amount of incorporated label,
   vi) Determining the identity of the pMHC Multiplexers bound to a T cell, by one of the processes (a), (b), or (c):
      a. Performing flow sorting, to isolate labelled T cells, followed by sequencing of the encoding molecules bound to the isolated cells, thereby determining the identity of the pMHC complexes of the pMHC Multiplexer that was bound to a T cell,
      b. Adding primers that anneal to the 3′-ends of the oligonucleotides, and performing a PCR reaction, optionally incorporating labelled dNTPs, and measuring the amount of label attached to the pMHC Multiplexers or sequencing the PCR products thereby determining the identity of the pMHC complexes of the pMHC Multiplexer that was bound to a T cell,
      c. Sequencing the encoding molecules of the pMHC Multiplexers thereby determining the identity of the pMHC complexes of the pMHC Multiplexer that was bound to a T cell,
         thereby identifying the pMHC-binding specificity of the T cell that was bound to said pMHC Multiplexer.
Aspect K6. The method of Aspect K5 where the extension of step (iv) is performed enzymatically by a polymerase or is done chemically in the absence of an enzyme.
Aspect K7. The method of Aspect K5 where the pMHC Multiplexer comprises a MHC Dextramer, a MHC Tetramer, a MHC Pentamer, or a MHC Streptamer.
Aspect K8. The method of Aspect K7 involving at least 10 pairs of pMHC Multiplexers, such as at least 100 pairs of pMHC Multiplexers, such as at least 1000 pairs of pMHC Multiplexers, such as at least 10000 pairs of pMHC Multiplexers, such as at least 100000 pairs of pMHC Multiplexers, such as at least 1000000 pairs of pMHC Multiplexers, such as at least 10000000 pairs of pMHC Multiplexers, such as at least 10000000 pairs of pMHC Multiplexers.
Aspect K9. A pair of pMHC Multiplexers where the encoding molecule of one pMHC Multiplexer comprises a sequence of at least three consecutive nucleotides that are complementary to a sequence of at least three consecutive nucleotides of the other pMHC Multiplexer.
Aspect K10: The method or pMHC Multiplexer or composition of any of Aspects K1-K8 where the encoding molecule is a DNA oligonucleotide of at least 10 nt, such as at least 15 nt, such as at least 20 nt, such as at least 25 nt, such as at least 30 nt, such as at least 50 nt, such as at least 80 nt.
Aspect L1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect L2: A composition of two or more pMHC Multiplexers, each comprising an encoding molecule such as a DNA or RNA molecule, functionally linked to a peptide (p) that is encoded by said encoding molecules, and where said peptide is in complex with an MHC complex, thereby making up a pMHC complex.
Aspect L3: The composition of Aspect L2 where the encoding molecule is directly linked to the peptide (p) of the pMHC complex.
Aspect L4: The composition of Aspect L2 or Aspect L3 where the pMHC complex is linked to a dextran molecule.
Aspect L5: The composition of Aspect L4 where the pMHC complex is linked to a dextran molecule that further comprises a fluorochrome.
Aspect L6: The composition of Aspect L2 or Aspect L3 where the pMHC complex is chemically linked to at least 5 other pMHC complexes.
Aspect L7: The pMHC Multiplexer comprising at least 5 pMHC complexes linked in series, where for all 5 pMHC complexes the peptide (p) of the pMHC complex is directly linked to at least one encoding molecule.
Aspect L8: A pMHC Multimer comprising two or more streptavidin molecules.
Aspect L9: The pMHC Multimer of Aspect L8 additionally comprising a DNA-tag.
Aspect L10: A screening process involving the pMHC Multiplexer, the MHC Multimer, or the composition of any of Aspects L1-L9, where the screening process involves flow cytometry or centrifugation.
Aspect M1: A pMHC Multiplexer comprising an encoding molecule such as a DNA or RNA molecule, functionally linked to a peptide (p) that is encoded by said encoding molecule, and where said peptide is in complex with an MHC complex, thereby making up a pMHC complex.
Aspect M2: A composition of two or more pMHC Multiplexers, each comprising an encoding molecule such as a DNA or RNA molecule, functionally linked to a peptide (p) that is encoded by said encoding molecules, and where said peptide is in complex with an MHC complex, thereby making up a pMHC complex.
Aspect M3: The composition of Aspect M2 where the pMHC Multiplexer comprises a SP1 protein.
Aspect M4: The composition of Aspect M2 or 3 where the pMHC complex is chemically linked to a SP1 protein.
Aspect M5: The composition of Aspect M4 where each SP1 subunit is attached to a pMHC complex by way of a non-covalent bond.
Aspect M6: The composition of Aspect M1-M5 where the encoding molecule is a DNA that is chemically linked to the SP1 protein and/or to the SP1 subunit.
Aspect M7: The process of making a pMHC Multiplexer, involving the following steps:
   i) Providing 1000 wells each comprising a dimerization domain Y, capable of binding to domain X, and attached to a unique pMHC complex;
   ii) Adding to each of the 1000 wells a unique DNA molecule, attached to a dimerization domain Y, capable of binding to domain X;
   iii) Adding to each of the 1000 wells a SP1 protein where each of the 12 subunits of the SP1 protein is attached to a dimerization domain X, capable of binding to a dimerization domain Y;
   where steps (i) to (iii) can be performed in any order;
   iv) Allowing the X and Y dimerization domain to form an XY dimer,
      thereby producing 1000 pMHC Multiplexers, each of which comprise a unique peptide (p) complexed to MHC protein, and comprising a unique DNA encoding said unique peptide (p).
Aspect M8: A MHC Multimer comprising 5 or more pMHC complexes, such as 10 or more pMHC complexes.
Aspect M9: The MHC Multimer of Aspect M8 further comprising a DNA-tag.
Aspect M10: A screening process involving the pMHC Multiplexer, the MHC Multimer, or the composition of any of Aspect M1-M9, where the screening process involves flow cytometry or centrifugation.

### Aspects N1-T17

Aspect N1. A structure comprising a pMHC complex linked to a DNA molecule.
Aspect O1. A structure comprising a pMHC complex linked to a DNA molecule of non-human origin and further comprising a Cell.
Aspect O2. A structure comprising a pMHC complex linked to a DNA molecule of non-human origin and further comprising a Filamentous phage.
Aspect O3. A structure comprising a pMHC complex linked to a DNA molecule of non-human origin and further comprising an M13 phage.
Aspect O4. A structure comprising a pMHC complex linked to a DNA molecule of non-human origin and further comprising a Phage particle comprising phagemid.
Aspect O5. A structure comprising a pMHC complex linked to a DNA molecule of non-human origin and further comprising a Dendritic cell.
Aspect O6. A structure comprising a pMHC complex linked to a DNA molecule of non-human origin and further comprising an E. coli cell.
Aspect O7. A structure comprising a pMHC complex linked to a DNA molecule of non-human origin and further comprising a Phage.
Aspect O8. A structure comprising a pMHC complex linked to a DNA molecule of non-human origin and further comprising a Bacterial cell.
Aspect O9. A structure comprising a pMHC complex linked to a DNA molecule of non-human origin and further comprising a Virus.
Aspect O10. A structure comprising a pMHC complex linked to a DNA molecule of non-human origin and further comprising a B cell.
Aspect O11. A structure comprising a pMHC complex linked to a DNA molecule of non-human origin and further comprising a Human cell.
Aspect O12. A structure comprising a pMHC complex linked to a DNA molecule of non-human origin and further comprising a Yeast cell.
Aspect O13. A structure comprising a pMHC complex linked to a DNA molecule of non-human origin and further comprising a Micelle.
Aspect O14. A structure comprising a pMHC complex linked to a DNA molecule of non-human origin and further comprising a Macrophage cell.
Aspect O15. A structure comprising a pMHC complex linked to a DNA molecule of non-human origin and further comprising a S. typhimurium cell.
Aspect O16. A structure comprising a pMHC complex linked to a DNA molecule of non-human origin and further comprising a B. subtilis cell.
Aspect O17. A structure comprising a pMHC complex linked to a DNA molecule of non-human origin and further comprising a S. cerevisiae cell.
Aspect O18. A structure comprising a pMHC complex linked to a DNA molecule of non-human origin and further comprising a S. pombe cell.
Aspect O19. A structure comprising a pMHC complex linked to a DNA molecule of non-human origin and further comprising a Fungal cell.
Aspect O20. A structure comprising a pMHC complex linked to a DNA molecule of non-human origin and further comprising an Aspergillus cell.
Aspect O21. A structure comprising a pMHC complex linked to a DNA molecule of non-human origin and further comprising an Antigen-presenting cell.
Aspect O22. A structure comprising a pMHC complex linked to a DNA molecule of non-human origin and further comprising a Professional antigen-presenting cell.
Aspect O23. A structure comprising a pMHC complex linked to a DNA molecule of non-human origin and further comprising a Non-professional antigen-presenting cell.
Aspect O24. A structure comprising a pMHC complex linked to a DNA molecule of non-human origin and further comprising a Nucleated cell.
Aspect O25. A structure comprising a pMHC complex linked to a DNA molecule of non-human origin and further comprising a Baculovirus particle.
Aspect O26. A structure comprising a pMHC complex linked to a DNA molecule of non-human origin and further comprising a Eukaryotic cell comprising membrane-spanning protein (tANCHOR).
Aspect O27. A structure comprising a pMHC complex linked to a DNA molecule of non-human origin and further comprising a Virus-like particle such as Adaptsvac.
Aspect P1. A structure comprising a pMHC complex linked to a DNA molecule of non-human origin where the pMHC complex comprises the A*02:01 allele.
Aspect P2. A structure comprising a pMHC complex linked to a DNA molecule of non-human origin where the pMHC complex comprises the C*07:01 allele.
Aspect P3. A structure comprising a pMHC complex linked to a DNA molecule of non-human origin where the pMHC complex comprises the A*01:01 allele.
Aspect P4. A structure comprising a pMHC complex linked to a DNA molecule of non-human origin where the pMHC complex comprises the A*03:01 allele.
Aspect P5. A structure comprising a pMHC complex linked to a DNA molecule of non-human origin where the pMHC complex comprises the C*07:02 allele.
Aspect P6. A structure comprising a pMHC complex linked to a DNA molecule of non-human origin where the pMHC complex comprises the C*04:01 allele.
Aspect P7. A structure comprising a pMHC complex linked to a DNA molecule of non-human origin where the pMHC complex comprises the B*44:02 allele.
Aspect P8. A structure comprising a pMHC complex linked to a DNA molecule of non-human origin where the pMHC complex comprises the B*07:02 allele.
Aspect P9. A structure comprising a pMHC complex linked to a DNA molecule of non-human origin where the pMHC complex comprises the B*08:01 allele.
Aspect P10. A structure comprising a pMHC complex linked to a DNA molecule of non-human origin where the pMHC complex comprises the C*05:01 allele.
Aspect P11. A structure comprising a pMHC complex linked to a DNA molecule of non-human origin where the pMHC complex comprises the HLA-DPA1 allele.
Aspect P12. A structure comprising a pMHC complex linked to a DNA molecule of non-human origin where the pMHC complex comprises the HLA-DRB1 allele.
Aspect P13. A structure comprising a pMHC complex linked to a DNA molecule of non-human origin where the pMHC complex comprises the HLA-DQB1 allele.
Aspect P14. A structure comprising a pMHC complex linked to a DNA molecule of non-human origin where the pMHC complex comprises the HLA-DPB1 allele.
Aspect P15. A structure comprising a pMHC complex linked to a DNA molecule of non-human origin where the pMHC complex comprises the HLA-DRB4 allele.
Aspect P16. A structure comprising a pMHC complex linked to a DNA molecule of non-human origin where the pMHC complex comprises the HLA-DRB3 allele.
Aspect P17. A structure comprising a pMHC complex linked to a DNA molecule of non-human origin where the pMHC complex comprises the HLA-DRB5 allele.
Aspect Q1. A structure comprising a pMHC complex linked to a DNA molecule of non-human origin where the peptide (p) of the pMHC complex is a Cancer-specific epitope.
Aspect Q2. A structure comprising a pMHC complex linked to a DNA molecule of non-human origin where the peptide (p) of the pMHC complex is a Skin cancer-specific epitope.
Aspect Q3. A structure comprising a pMHC complex linked to a DNA molecule of non-human origin where the peptide (p) of the pMHC complex is a Lung cancer-specific epitope.
Aspect Q4. A structure comprising a pMHC complex linked to a DNA molecule of non-human origin where the peptide (p) of the pMHC complex is a Prostate cancer-specific epitope.
Aspect Q5. A structure comprising a pMHC complex linked to a DNA molecule of non-human origin where the peptide (p) of the pMHC complex is a Breast cancer-specific epitope.
Aspect Q6. A structure comprising a pMHC complex linked to a DNA molecule of non-human origin where the peptide (p) of the pMHC complex is a Melanoma-specific epitope.
Aspect Q7. A structure comprising a pMHC complex linked to a DNA molecule of non-human origin where the peptide (p) of the pMHC complex is a Colorectal cancer-specific epitope.
Aspect Q8. A structure comprising a pMHC complex linked to a DNA molecule of non-human origin where the peptide (p) of the pMHC complex is a Kidney (renal) cancer-specific epitope.
Aspect Q9. A structure comprising a pMHC complex linked to a DNA molecule of non-human origin where the peptide (p) of the pMHC complex is a Bladder cancer-specific epitope.
Aspect Q10. A structure comprising a pMHC complex linked to a DNA molecule of non-human origin where the peptide (p) of the pMHC complex is a Non-Hodgkin's lymphoma-specific epitope.
Aspect Q11. A structure comprising a pMHC complex linked to a DNA molecule of non-human origin where the peptide (p) of the pMHC complex is a Carcinoma-specific epitope.
Aspect Q12. A structure comprising a pMHC complex linked to a DNA molecule of non-human origin where the peptide (p) of the pMHC complex is a Sarcoma-specific epitope.
Aspect Q13. A structure comprising a pMHC complex linked to a DNA molecule of non-human origin where the peptide (p) of the pMHC complex is a Lymphoma-specific epitope.
Aspect Q14. A structure comprising a pMHC complex linked to a DNA molecule of non-human origin where the peptide (p) of the pMHC complex is a Leukemia-specific epitope.
Aspect Q15. A structure comprising a pMHC complex linked to a DNA molecule of non-human origin where the peptide (p) of the pMHC complex is a Germ cell tumor-specific epitope.
Aspect Q16. A structure comprising a pMHC complex linked to a DNA molecule of non-human origin where the peptide (p) of the pMHC complex is a Blastoma-specific epitope.
Aspect Q17. A structure comprising a pMHC complex linked to a DNA molecule of non-human origin where the peptide (p) of the pMHC complex is a virus-specific epitope.
Aspect Q18. A structure comprising a pMHC complex linked to a DNA molecule of non-human origin where the peptide (p) of the pMHC complex is a Corona virus-specific epitope.
Aspect Q19. A structure comprising a pMHC complex linked to a DNA molecule of non-human origin where the peptide (p) of the pMHC complex is a HIV (Human immunodeficiency virus)-specific epitope.
Aspect Q20. A structure comprising a pMHC complex linked to a DNA molecule of non-human origin where the peptide (p) of the pMHC complex is a Bacterium-specific epitope.
Aspect Q21. A structure comprising a pMHC complex linked to a DNA molecule of non-human origin where the peptide (p) of the pMHC complex is a Hepatitis A virus-specific epitope.
Aspect Q22. A structure comprising a pMHC complex linked to a DNA molecule of non-human origin where the peptide (p) of the pMHC complex is a Hepatitis B virus-specific epitope.
Aspect Q23. A structure comprising a pMHC complex linked to a DNA molecule of non-human origin where the peptide (p) of the pMHC complex is a Hepatitis C virus-specific epitope.
Aspect Q24. A structure comprising a pMHC complex linked to a DNA molecule of non-human origin where the peptide (p) of the pMHC complex is a Hepatitis D Virus-specific epitope.
Aspect Q25. A structure comprising a pMHC complex linked to a DNA molecule of non-human origin where the peptide (p) of the pMHC complex is a Hepatitis E virus-specific epitope.
Aspect Q26. A structure comprising a pMHC complex linked to a DNA molecule of non-human origin where the peptide (p) of the pMHC complex is a Herpes simplex virus 1-specific epitope.
Aspect Q27. A structure comprising a pMHC complex linked to a DNA molecule of non-human origin where the peptide (p) of the pMHC complex is a Salmonella-specific epitope.
Aspect Q28. A structure comprising a pMHC complex linked to a DNA molecule of non-human origin where the peptide (p) of the pMHC complex is a Tuberculosis-specific epitope.
Aspect Q29. A structure comprising a pMHC complex linked to a DNA molecule of non-human origin where the peptide (p) of the pMHC complex is an E. coli-specific epitope.
Aspect Q30. A structure comprising a pMHC complex linked to a DNA molecule of non-human origin where the peptide (p) of the pMHC complex is an Epstein-Barr virus (EBV)-specific epitope.
Aspect Q31. A structure comprising a pMHC complex linked to a DNA molecule of non-human origin where the peptide (p) of the pMHC complex is a Diabetes-specific epitope.
Aspect Q32. A structure comprising a pMHC complex linked to a DNA molecule of non-human origin where the peptide (p) of the pMHC complex is a Rheumatoid arthritis-specific epitope.
Aspect Q33. A structure comprising a pMHC complex linked to a DNA molecule of non-human origin where the peptide (p) of the pMHC complex is a Peptide epitope consisting of less than 6 amino acid residues.
Aspect Q34. A structure comprising a pMHC complex linked to a DNA molecule of non-human origin where the peptide (p) of the pMHC complex is a Peptide epitope consisting of 6 amino acid residues.
Aspect Q35. A structure comprising a pMHC complex linked to a DNA molecule of non-human origin where the peptide (p) of the pMHC complex is a Peptide epitope consisting of 7 amino acid residues.
Aspect Q36. A structure comprising a pMHC complex linked to a DNA molecule of non-human origin where the peptide (p) of the pMHC complex is a Peptide epitope consisting of 8 amino acid residues.
Aspect Q37. A structure comprising a pMHC complex linked to a DNA molecule of non-human origin where the peptide (p) of the pMHC complex is a Peptide epitope consisting of 9 amino acid residues.
Aspect Q38. A structure comprising a pMHC complex linked to a DNA molecule of non-human origin where the peptide (p) of the pMHC complex is a Peptide epitope consisting of 10 amino acid residues.
Aspect Q39. A structure comprising a pMHC complex linked to a DNA molecule of non-human origin where the peptide (p) of the pMHC complex is a Peptide epitope consisting of 11 amino acid residues.
Aspect Q40. A structure comprising a pMHC complex linked to a DNA molecule of non-human origin where the peptide (p) of the pMHC complex is a Peptide epitope consisting of 12 amino acid residues.
Aspect Q41. A structure comprising a pMHC complex linked to a DNA molecule of non-human origin where the peptide (p) of the pMHC complex is a Peptide epitope consisting of 13 amino acid residues.
Aspect Q42. A structure comprising a pMHC complex linked to a DNA molecule of non-human origin where the peptide (p) of the pMHC complex is a Peptide epitope consisting of 14-17 amino acid residues.
Aspect Q43. A structure comprising a pMHC complex linked to a DNA molecule of non-human origin where the peptide (p) of the pMHC complex is a Peptide epitope consisting of 18-25 amino acid residues.
Aspect Q44. A structure comprising a pMHC complex linked to a DNA molecule of non-human origin where the peptide (p) of the pMHC complex is a Peptide epitope consisting of more than 25 amino acid residues.
Aspect Q45. A structure comprising a pMHC complex linked to a DNA molecule of non-human origin where the peptide (p) of the pMHC complex is a Peptide epitope comprising less than 6 amino acid residues.
Aspect Q46. A structure comprising a pMHC complex linked to a DNA molecule of non-human origin where the peptide (p) of the pMHC complex is a Peptide epitope comprising of 6 amino acid residues.
Aspect Q47. A structure comprising a pMHC complex linked to a DNA molecule of non-human origin where the peptide (p) of the pMHC complex is a Peptide epitope comprising of 7 amino acid residues.
Aspect Q48. A structure comprising a pMHC complex linked to a DNA molecule of non-human origin where the peptide (p) of the pMHC complex is a Peptide epitope comprising of 8 amino acid residues.
Aspect Q49. A structure comprising a pMHC complex linked to a DNA molecule of non-human origin where the peptide (p) of the pMHC complex is a Peptide epitope comprising of 9 amino acid residues.
Aspect Q50. A structure comprising a pMHC complex linked to a DNA molecule of non-human origin where the peptide (p) of the pMHC complex is a Peptide epitope comprising of 10 amino acid residues.
Aspect Q51. A structure comprising a pMHC complex linked to a DNA molecule of non-human origin where the peptide (p) of the pMHC complex is a Peptide epitope comprising of 11 amino acid residues.
Aspect Q52. A structure comprising a pMHC complex linked to a DNA molecule of non-human origin where the peptide (p) of the pMHC complex is a Peptide epitope comprising of 12 amino acid residues.
Aspect Q53. A structure comprising a pMHC complex linked to a DNA molecule of non-human origin where the peptide (p) of the pMHC complex is a Peptide epitope comprising of 13 amino acid residues.
Aspect Q54. A structure comprising a pMHC complex linked to a DNA molecule of non-human origin where the peptide (p) of the pMHC complex is a Peptide epitope comprising of 14-17 amino acid residues.
Aspect Q55. A structure comprising a pMHC complex linked to a DNA molecule of non-human origin where the peptide (p) of the pMHC complex is a Peptide epitope comprising of 18-25 amino acid residues.
Aspect Q56. A structure comprising a pMHC complex linked to a DNA molecule of non-human origin where the peptide (p) of the pMHC complex is a Peptide epitope comprising more than 25 amino acid residues.
Aspect Q57. A structure comprising a pMHC complex linked to a DNA molecule of non-human origin where the peptide (p) of the pMHC complex is a Peptide epitope with the sequence Glycyl-Methionine.
Aspect Q58. A structure comprising a pMHC complex linked to a DNA molecule of non-human origin where the peptide (p) of the pMHC complex is a Peptide epitope with the sequence Glycyl-Leucine.
Aspect Q59. A structure comprising a pMHC complex linked to a DNA molecule of non-human origin where the peptide (p) of the pMHC complex is a Peptide epitope with the sequence Glycyl-Cyclohexylalanine.
Aspect Q60. A structure comprising a pMHC complex linked to a DNA molecule of non-human origin where the peptide (p) of the pMHC complex is a Peptide epitope with the sequence Glycyl-Homoleucine.
Aspect Q61. A structure comprising a pMHC complex linked to a DNA molecule of non-human origin where the peptide (p) of the pMHC complex is a Clostridium botulinum-specific epitope.
Aspect Q62. A structure comprising a pMHC complex linked to a DNA molecule of non-human origin where the peptide (p) of the pMHC complex is a Vibrio cholera-specific epitope.
Aspect Q63. A structure comprising a pMHC complex linked to a DNA molecule of non-human origin where the peptide (p) of the pMHC complex is a Tetanus-specific epitope.
Aspect Q64. A structure comprising a pMHC complex linked to a DNA molecule of non-human origin where the peptide (p) of the pMHC complex is a Klebsiella-specific epitope.
Aspect Q65. A structure comprising a pMHC complex linked to a DNA molecule of non-human origin where the peptide (p) of the pMHC complex is a Staphylococcus-specific epitope.
Aspect R1. A structure comprising a pMHC complex linked to a DNA molecule of non-human origin where the pMHC complex is a pMHC class 1 complex.
Aspect R2. A structure comprising a pMHC complex linked to a DNA molecule of non-human origin where the pMHC complex is a pMHC class 2 complex.
Aspect R3. A structure comprising a pMHC complex linked to a DNA molecule of non-human origin where the pMHC complex is a pMHC-like complex.
Aspect R4. A structure comprising a pMHC complex linked to a DNA molecule of non-human origin where the pMHC complex is a Peptide-receptive pMHC class 1 complex.
Aspect R5. A structure comprising a pMHC complex linked to a DNA molecule of non-human origin where the pMHC complex is a Peptide-receptive pMHC class 2 complex.
Aspect R6. A structure comprising a pMHC complex linked to a DNA molecule of non-human origin where the pMHC complex is a pMHC complex where the peptide (p) is a UV-cleavable peptide.
Aspect S1. A structure comprising a pMHC complex linked to a DNA molecule where the structure further comprises a non-mammal fluorochrome label.
Aspect S2. A structure comprising a pMHC complex linked to a DNA molecule where the structure further comprises a non-human fluorochrome label.
Aspect S3. A structure comprising a pMHC complex linked to a DNA molecule where the structure further comprises a non-mammal chromophore label.
Aspect S4. A structure comprising a pMHC complex linked to a DNA molecule where the structure further comprises a non-human chromophore label.
Aspect S5. A structure comprising a pMHC complex linked to a DNA molecule where the structure further comprises a Rare element label.
Aspect S6. A structure comprising a pMHC complex linked to a DNA molecule where the structure further comprises a Fluorescein label.
Aspect S7. A structure comprising a pMHC complex linked to a DNA molecule where the structure further comprises a Dye label.
Aspect S8. A structure comprising a pMHC complex linked to a DNA molecule where the structure further comprises a Chromophore label.
Aspect S9. A structure comprising a pMHC complex linked to a DNA molecule where the structure further comprises a Fluorochrome label.
Aspect S10. A structure comprising a pMHC complex linked to a DNA molecule where the structure further comprises an APC label.
Aspect S11. A structure comprising a pMHC complex linked to a DNA molecule where the structure further comprises a Cy5 label.
Aspect S12. A structure comprising a pMHC complex linked to a DNA molecule where the structure further comprises a PE label.
Aspect S13. A structure comprising a pMHC complex linked to a DNA molecule where the structure further comprises a Gadolinium label.
Aspect S14. A structure comprising a pMHC complex linked to a DNA molecule where the structure further comprises an Europium label.
Aspect S15. A structure comprising a pMHC complex linked to a DNA molecule where the structure further comprises a rare earth metal label.
Aspect S16. A structure comprising a pMHC complex linked to a DNA molecule where the structure further comprises a Rhodamine label.
Aspect S17. A structure comprising a pMHC complex linked to a DNA molecule where the structure further comprises a FITC label.
Aspect S18. A structure comprising a pMHC complex linked to a DNA molecule where the structure further comprises a Green FP label.
Aspect S19. A structure comprising a pMHC complex linked to a DNA molecule where the structure further comprises a DNA tag label.
Aspect S20. A structure comprising a pMHC complex linked to a DNA molecule where the structure further comprises an RNA tag label.
Aspect S21. A structure comprising a pMHC complex linked to a DNA molecule where the structure further comprises a Fluorescent dye label.
Aspect S22. A structure comprising a pMHC complex linked to a DNA molecule where the structure further comprises an Alexa Fluor label.
Aspect S23. A structure comprising a pMHC complex linked to a DNA molecule where the structure further comprises a NovaFluor label.
Aspect S24. A structure comprising a pMHC complex linked to a DNA molecule where the structure further comprises a BODIPY FL label.
Aspect S25. A structure comprising a pMHC complex linked to a DNA molecule where the structure further comprises a Coumarin label.
Aspect S26. A structure comprising a pMHC complex linked to a DNA molecule where the structure further comprises a Cy3 label.
Aspect S27. A structure comprising a pMHC complex linked to a DNA molecule where the structure further comprises a DNA stain label.
Aspect S28. A structure comprising a pMHC complex linked to a DNA molecule where the structure further comprises a DAPI label.
Aspect S29. A structure comprising a pMHC complex linked to a DNA molecule where the structure further comprises a Propidium iodide label.
Aspect S30. A structure comprising a pMHC complex linked to a DNA molecule where the structure further comprises a SYTO 9 label.
Aspect S31. A structure comprising a pMHC complex linked to a DNA molecule where the structure further comprises a SYTOX Green label.
Aspect S32. A structure comprising a pMHC complex linked to a DNA molecule where the structure further comprises a TO-PRO-3 label.
Aspect S33. A structure comprising a pMHC complex linked to a DNA molecule where the structure further comprises a Qdot probe label.
Aspect S34. A structure comprising a pMHC complex linked to a DNA molecule where the structure further comprises a Brilliant Ultra Violet Dye label.
Aspect S35. A structure comprising a pMHC complex linked to a DNA molecule where the structure further comprises an RNA stain label.
Aspect S36. A structure comprising a pMHC complex linked to a DNA molecule where the structure further comprises a Fluorescent protein label.
Aspect S37. A structure comprising a pMHC complex linked to a DNA molecule where the structure further comprises a Cyan FP label.
Aspect S38. A structure comprising a pMHC complex linked to a DNA molecule where the structure further comprises a Red FP label.
Aspect S39. A structure comprising a pMHC complex linked to a DNA molecule where the structure further comprises a Protein tag label.
Aspect S40. A structure comprising a pMHC complex linked to a DNA molecule where the structure further comprises a Biarsenical tag label.
Aspect S41. A structure comprising a pMHC complex linked to a DNA molecule where the structure further comprises a Histidine tag label.
Aspect S42. A structure comprising a pMHC complex linked to a DNA molecule where the structure further comprises a FLAG tag label.
Aspect S43. A structure comprising a pMHC complex linked to a DNA molecule where the structure further comprises a Biotin label.
Aspect S44. A structure comprising a pMHC complex linked to a DNA molecule where the structure further comprises a Streptavidin label.
Aspect S45. A structure comprising a pMHC complex linked to a DNA molecule where the structure further comprises a DNA tag label.
Aspect S46. A structure comprising a pMHC complex linked to a DNA molecule where the structure further comprises a RNA tag label.
Aspect T1. A structure comprising a pMHC complex linked to a DNA molecule and further comprising a Dextramer scaffold.
Aspect T2. A structure comprising a pMHC complex linked to a DNA molecule and further comprising a Streptamer scaffold.
Aspect T3. A structure comprising a pMHC complex linked to a DNA molecule and further comprising a tetramer scaffold.
Aspect T4. A structure comprising a pMHC complex linked to a DNA molecule and further comprising a pentamer scaffold.
Aspect T5. A structure comprising a pMHC complex linked to a DNA molecule and further comprising a Streptavidin scaffold.
Aspect T6. A structure comprising a pMHC complex linked to a DNA molecule and further comprising a Dextran scaffold.
Aspect T7. A structure comprising a pMHC complex linked to a DNA molecule and further comprising a dimer scaffold.
Aspect T8. A structure comprising a pMHC complex linked to a DNA molecule and further comprising a trimer scaffold.
Aspect T9. A structure comprising a pMHC complex linked to a DNA molecule and further comprising a hexamer scaffold.
Aspect T10. A structure comprising a pMHC complex linked to a DNA molecule and further comprising a SP1-based scaffold.
Aspect T11. A structure comprising a pMHC complex linked to a DNA molecule and further comprising an IgG-based scaffold.
Aspect T12. A structure comprising a pMHC complex linked to a DNA molecule and further comprising a Fos-Jun dimer scaffold.
Aspect T13. A structure comprising a pMHC complex linked to a DNA molecule and further comprising a Pentameric coil-coil structure scaffold.
Aspect T14. A structure comprising a pMHC complex linked to a DNA molecule and further comprising a Streptactin scaffold.
Aspect T15. A structure comprising a pMHC complex linked to a DNA molecule and further comprising an IgM-based scaffold.
Aspect T16. A structure comprising a pMHC complex linked to a DNA molecule and further comprising a Polypeptide scaffold.
Aspect T17. A structure comprising a pMHC complex linked to a DNA molecule and further comprising a triplex DNA-based scaffold.

### Aspects W1-CC17

Aspect W1. A structure comprising a pMHC complex mechanically linked to a DNA molecule of non-human origin.
Aspect X1. A structure comprising a pMHC complex mechanically linked to a DNA molecule of non-human origin and further comprising a Cell.
Aspect X2. A structure comprising a pMHC complex mechanically linked to a DNA molecule of non-human origin and further comprising a Filamentous phage.
Aspect X3. A structure comprising a pMHC complex mechanically linked to a DNA molecule of non-human origin and further comprising an M13 phage.
Aspect X4. A structure comprising a pMHC complex mechanically linked to a DNA molecule of non-human origin and further comprising a Phage particle comprising phagemid.
Aspect X5. A structure comprising a pMHC complex mechanically linked to a DNA molecule of non-human origin and further comprising a Dendritic cell.
Aspect X6. A structure comprising a pMHC complex mechanically linked to a DNA molecule of non-human origin and further comprising an E. coli cell.
Aspect X7. A structure comprising a pMHC complex mechanically linked to a DNA molecule of non-human origin and further comprising a Phage.
Aspect X8. A structure comprising a pMHC complex mechanically linked to a DNA molecule of non-human origin and further comprising a Bacterial cell.
Aspect X9. A structure comprising a pMHC complex mechanically linked to a DNA molecule of non-human origin and further comprising a Virus.
Aspect X10. A structure comprising a pMHC complex mechanically linked to a DNA molecule of non-human origin and further comprising a B cell.
Aspect X11. A structure comprising a pMHC complex mechanically linked to a DNA molecule of non-human origin and further comprising a Human cell.
Aspect X12. A structure comprising a pMHC complex mechanically linked to a DNA molecule of non-human origin and further comprising a Yeast cell.
Aspect X13. A structure comprising a pMHC complex mechanically linked to a DNA molecule of non-human origin and further comprising a Micelle.
Aspect X14. A structure comprising a pMHC complex mechanically linked to a DNA molecule of non-human origin and further comprising a Macrophage cell.
Aspect X15. A structure comprising a pMHC complex mechanically linked to a DNA molecule of non-human origin and further comprising a S. typhimurium cell.
Aspect X16. A structure comprising a pMHC complex mechanically linked to a DNA molecule of non-human origin and further comprising a B. subtilis cell.
Aspect X17. A structure comprising a pMHC complex mechanically linked to a DNA molecule of non-human origin and further comprising a S. cerevisiae cell.
Aspect X18. A structure comprising a pMHC complex mechanically linked to a DNA molecule of non-human origin and further comprising a S. pombe cell.
Aspect X19. A structure comprising a pMHC complex mechanically linked to a DNA molecule of non-human origin and further comprising a Fungal cell.
Aspect X20. A structure comprising a pMHC complex mechanically linked to a DNA molecule of non-human origin and further comprising an Aspergillus cell.
Aspect X21. A structure comprising a pMHC complex mechanically linked to a DNA molecule of non-human origin and further comprising an Antigen-presenting cell.
Aspect X22. A structure comprising a pMHC complex mechanically linked to a DNA molecule of non-human origin and further comprising a Professional antigen-presenting cell.
Aspect X23. A structure comprising a pMHC complex mechanically linked to a DNA molecule of non-human origin and further comprising a Non-professional antigen-presenting cell.
Aspect X24. A structure comprising a pMHC complex mechanically linked to a DNA molecule of non-human origin and further comprising a Nucleated cell.
Aspect X25. A structure comprising a pMHC complex mechanically linked to a DNA molecule of non-human origin and further comprising a Baculovirus particle.
Aspect X26. A structure comprising a pMHC complex mechanically linked to a DNA molecule of non-human origin and further comprising a Eukaryotic cell comprising membrane-spanning protein (tANCHOR).
Aspect X27. A structure comprising a pMHC complex mechanically linked to a DNA molecule of non-human origin and further comprising a Virus-like particle such as Adaptsvac.
Aspect Y1. A structure comprising a pMHC complex mechanically linked to a DNA molecule of non-human origin where the pMHC complex comprises the A*02:01 allele.
Aspect Y2. A structure comprising a pMHC complex mechanically linked to a DNA molecule of non-human origin where the pMHC complex comprises the C*07:01 allele.
Aspect Y3. A structure comprising a pMHC complex mechanically linked to a DNA molecule of non-human origin where the pMHC complex comprises the A*01:01 allele.
Aspect Y4. A structure comprising a pMHC complex mechanically linked to a DNA molecule of non-human origin where the pMHC complex comprises the A*03:01 allele.
Aspect Y5. A structure comprising a pMHC complex mechanically linked to a DNA molecule of non-human origin where the pMHC complex comprises the C*07:02 allele.
Aspect Y6. A structure comprising a pMHC complex mechanically linked to a DNA molecule of non-human origin where the pMHC complex comprises the C*04:01 allele.
Aspect Y7. A structure comprising a pMHC complex mechanically linked to a DNA molecule of non-human origin where the pMHC complex comprises the B*44:02 allele.
Aspect Y8. A structure comprising a pMHC complex mechanically linked to a DNA molecule of non-human origin where the pMHC complex comprises the B*07:02 allele.
Aspect Y9. A structure comprising a pMHC complex mechanically linked to a DNA molecule of non-human origin where the pMHC complex comprises the B*08:01 allele.
Aspect Y10. A structure comprising a pMHC complex mechanically linked to a DNA molecule of non-human origin where the pMHC complex comprises the C*05:01 allele.
Aspect Y11. A structure comprising a pMHC complex mechanically linked to a DNA molecule of non-human origin where the pMHC complex comprises the HLA-DPA1 allele.
Aspect Y12. A structure comprising a pMHC complex mechanically linked to a DNA molecule of non-human origin where the pMHC complex comprises the HLA-DRB1 allele.
Aspect Y13. A structure comprising a pMHC complex mechanically linked to a DNA molecule of non-human origin where the pMHC complex comprises the HLA-DQB1 allele.
Aspect Y14. A structure comprising a pMHC complex mechanically linked to a DNA molecule of non-human origin where the pMHC complex comprises the HLA-DPB1 allele.
Aspect Y15. A structure comprising a pMHC complex mechanically linked to a DNA molecule of non-human origin where the pMHC complex comprises the HLA-DRB4 allele.
Aspect Y16. A structure comprising a pMHC complex mechanically linked to a DNA molecule of non-human origin where the pMHC complex comprises the HLA-DRB3 allele.
Aspect Y17. A structure comprising a pMHC complex mechanically linked to a DNA molecule of non-human origin where the pMHC complex comprises the HLA-DRB5 allele.
Aspect Y18. A structure comprising a pMHC complex mechanically linked to a DNA molecule of non-human origin where the pMHC complex comprises the HLA-DRB1*01:01 allele.
Aspect Y19. A structure comprising a pMHC complex mechanically linked to a DNA molecule of non-human origin where the pMHC complex comprises the HLA-DRB1*03:01 allele.
Aspect Y20. A structure comprising a pMHC complex mechanically linked to a DNA molecule of non-human origin where the pMHC complex comprises the HLA-DRB1*04:01 allele.
Aspect Y21. A structure comprising a pMHC complex mechanically linked to a DNA molecule of non-human origin where the pMHC complex comprises the HLA-DRB1*07:01 allele.
Aspect Y22. A structure comprising a pMHC complex mechanically linked to a DNA molecule of non-human origin where the pMHC complex comprises the HLA-DRB1*08:01 allele.
Aspect Y23. A structure comprising a pMHC complex mechanically linked to a DNA molecule of non-human origin where the pMHC complex comprises the HLA-DRB1*11:01 allele.
Aspect Y24. A structure comprising a pMHC complex mechanically linked to a DNA molecule of non-human origin where the pMHC complex comprises the HLA-DRB1*13:01 allele.
Aspect Y25. A structure comprising a pMHC complex mechanically linked to a DNA molecule of non-human origin where the pMHC complex comprises the HLA-DRB1*15:01 allele.
Aspect Y26. A structure comprising a pMHC complex mechanically linked to a DNA molecule of non-human origin where the pMHC complex comprises the HLA-DRB1*04:01 allele.
Aspect Y27. A structure comprising a pMHC complex mechanically linked to a DNA molecule of non-human origin where the pMHC complex comprises the HLA-DRB3*01:01 allele.
Aspect Y28. A structure comprising a pMHC complex mechanically linked to a DNA molecule of non-human origin where the pMHC complex comprises the HLA-DRB3*02:02 allele.
Aspect Y29. A structure comprising a pMHC complex mechanically linked to a DNA molecule of non-human origin where the pMHC complex comprises the HLA-DRB4*01:01 allele.
Aspect Y30. A structure comprising a pMHC complex mechanically linked to a DNA molecule of non-human origin where the pMHC complex comprises the HLA-DRB5*01:01 allele.
Aspect Y31. A structure comprising a pMHC complex mechanically linked to a DNA molecule of non-human origin where the pMHC complex comprises the HLA-DPA1*01:03 allele.
Aspect Y32. A structure comprising a pMHC complex mechanically linked to a DNA molecule of non-human origin where the pMHC complex comprises the HLA-DPA1*02:02 allele.
Aspect Y33. A structure comprising a pMHC complex mechanically linked to a DNA molecule of non-human origin where the pMHC complex comprises the HLA-DPB1*04:01 allele.
Aspect Y34. A structure comprising a pMHC complex mechanically linked to a DNA molecule of non-human origin where the pMHC complex comprises the HLA-DPB1*04:02 allele.
Aspect Y35. A structure comprising a pMHC complex mechanically linked to a DNA molecule of non-human origin where the pMHC complex comprises the HLA-DQA1*01:01 allele.
Aspect Y36. A structure comprising a pMHC complex mechanically linked to a DNA molecule of non-human origin where the pMHC complex comprises the HLA-DQB1*03:01 allele.
Aspect Y37. A structure comprising a pMHC complex mechanically linked to a DNA molecule of non-human origin where the pMHC complex comprises the HLA-DQB1*05:01 allele.
Aspect Y38. A structure comprising a pMHC complex mechanically linked to a DNA molecule of non-human origin where the pMHC complex comprises the HLA-DQ2.5 allele.
Aspect Y39. A structure comprising a pMHC complex mechanically linked to a DNA molecule of non-human origin where the pMHC complex comprises the HLA-A*01:01 allele.
Aspect Y40. A structure comprising a pMHC complex mechanically linked to a DNA molecule of non-human origin where the pMHC complex comprises the HLA-A*02:01 allele.
Aspect Y41. A structure comprising a pMHC complex mechanically linked to a DNA molecule of non-human origin where the pMHC complex comprises the HLA-A*02:03 allele.
Aspect Y42. A structure comprising a pMHC complex mechanically linked to a DNA molecule of non-human origin where the pMHC complex comprises the HLA-A*02:06 allele.
Aspect Y43. A structure comprising a pMHC complex mechanically linked to a DNA molecule of non-human origin where the pMHC complex comprises the HLA-A*02:07 allele.
Aspect Y44. A structure comprising a pMHC complex mechanically linked to a DNA molecule of non-human origin where the pMHC complex comprises the HLA-A*02:11 allele.
Aspect Y45. A structure comprising a pMHC complex mechanically linked to a DNA molecule of non-human origin where the pMHC complex comprises the HLA-A*02:19 allele.
Aspect Y46. A structure comprising a pMHC complex mechanically linked to a DNA molecule of non-human origin where the pMHC complex comprises the HLA-A*03:01 allele.
Aspect Y47. A structure comprising a pMHC complex mechanically linked to a DNA molecule of non-human origin where the pMHC complex comprises the HLA-A*11:01 allele.
Aspect Y48. A structure comprising a pMHC complex mechanically linked to a DNA molecule of non-human origin where the pMHC complex comprises the HLA-A*23:01 allele.
Aspect Y49. A structure comprising a pMHC complex mechanically linked to a DNA molecule of non-human origin where the pMHC complex comprises the HLA-A*24:02 allele.
Aspect Y50. A structure comprising a pMHC complex mechanically linked to a DNA molecule of non-human origin where the pMHC complex comprises the HLA-A*24:03 allele.
Aspect Y51. A structure comprising a pMHC complex mechanically linked to a DNA molecule of non-human origin where the pMHC complex comprises the HLA-A*24:07 allele.
Aspect Y52. A structure comprising a pMHC complex mechanically linked to a DNA molecule of non-human origin where the pMHC complex comprises the HLA-A*25:01 allele.
Aspect Y53. A structure comprising a pMHC complex mechanically linked to a DNA molecule of non-human origin where the pMHC complex comprises the HLA-A*26:01 allele.
Aspect Y54. A structure comprising a pMHC complex mechanically linked to a DNA molecule of non-human origin where the pMHC complex comprises the HLA-A*26:03 allele.
Aspect Y55. A structure comprising a pMHC complex mechanically linked to a DNA molecule of non-human origin where the pMHC complex comprises the HLA-A*29:02 allele.
Aspect Y56. A structure comprising a pMHC complex mechanically linked to a DNA molecule of non-human origin where the pMHC complex comprises the HLA-A*30:01 allele.
Aspect Y57. A structure comprising a pMHC complex mechanically linked to a DNA molecule of non-human origin where the pMHC complex comprises the HLA-A*30:02 allele.
Aspect Y58. A structure comprising a pMHC complex mechanically linked to a DNA molecule of non-human origin where the pMHC complex comprises the HLA-A*31:01 allele.
Aspect Y59. A structure comprising a pMHC complex mechanically linked to a DNA molecule of non-human origin where the pMHC complex comprises the HLA-A*32:01 allele.
Aspect Y60. A structure comprising a pMHC complex mechanically linked to a DNA molecule of non-human origin where the pMHC complex comprises the HLA-A*33:03 allele.
Aspect Y61. A structure comprising a pMHC complex mechanically linked to a DNA molecule of non-human origin where the pMHC complex comprises the HLA-A*34:02 allele.
Aspect Y62. A structure comprising a pMHC complex mechanically linked to a DNA molecule of non-human origin where the pMHC complex comprises the HLA-A*36:01 allele.
Aspect Y63. A structure comprising a pMHC complex mechanically linked to a DNA molecule of non-human origin where the pMHC complex comprises the HLA-A*66:01 allele.
Aspect Y64. A structure comprising a pMHC complex mechanically linked to a DNA molecule of non-human origin where the pMHC complex comprises the HLA-A*68:01 allele.
Aspect Y65. A structure comprising a pMHC complex mechanically linked to a DNA molecule of non-human origin where the pMHC complex comprises the HLA-A*68:02 allele.
Aspect Y66. A structure comprising a pMHC complex mechanically linked to a DNA molecule of non-human origin where the pMHC complex comprises the HLA-A*74:01 allele.
Aspect Y67. A structure comprising a pMHC complex mechanically linked to a DNA molecule of non-human origin where the pMHC complex comprises the HLA-B*07:02 allele.
Aspect Y68. A structure comprising a pMHC complex mechanically linked to a DNA molecule of non-human origin where the pMHC complex comprises the HLA-B*08:01 allele.
Aspect Y69. A structure comprising a pMHC complex mechanically linked to a DNA molecule of non-human origin where the pMHC complex comprises the HLA-B*13:01 allele.
Aspect Y70. A structure comprising a pMHC complex mechanically linked to a DNA molecule of non-human origin where the pMHC complex comprises the HLA-B*13:02 allele.
Aspect Y71. A structure comprising a pMHC complex mechanically linked to a DNA molecule of non-human origin where the pMHC complex comprises the HLA-B*14:01 allele.
Aspect Y72. A structure comprising a pMHC complex mechanically linked to a DNA molecule of non-human origin where the pMHC complex comprises the HLA-B*14:02 allele.
Aspect Y73. A structure comprising a pMHC complex mechanically linked to a DNA molecule of non-human origin where the pMHC complex comprises the HLA-B*15:01 allele.
Aspect Y74. A structure comprising a pMHC complex mechanically linked to a DNA molecule of non-human origin where the pMHC complex comprises the HLA-B*15:02 allele.
Aspect Y75. A structure comprising a pMHC complex mechanically linked to a DNA molecule of non-human origin where the pMHC complex comprises the HLA-B*15:03 allele.
Aspect Y76. A structure comprising a pMHC complex mechanically linked to a DNA molecule of non-human origin where the pMHC complex comprises the HLA-B*15:09 allele.
Aspect Y77. A structure comprising a pMHC complex mechanically linked to a DNA molecule of non-human origin where the pMHC complex comprises the HLA-B*15:10 allele.
Aspect Y78. A structure comprising a pMHC complex mechanically linked to a DNA molecule of non-human origin where the pMHC complex comprises the HLA-B*18:01 allele.
Aspect Y79. A structure comprising a pMHC complex mechanically linked to a DNA molecule of non-human origin where the pMHC complex comprises the HLA-B*27:02 allele.
Aspect Y80. A structure comprising a pMHC complex mechanically linked to a DNA molecule of non-human origin where the pMHC complex comprises the HLA-B*27:03 allele.
Aspect Y81. A structure comprising a pMHC complex mechanically linked to a DNA molecule of non-human origin where the pMHC complex comprises the HLA-B*27:05 allele.
Aspect Y82. A structure comprising a pMHC complex mechanically linked to a DNA molecule of non-human origin where the pMHC complex comprises the HLA-B*35:01 allele.
Aspect Y83. A structure comprising a pMHC complex mechanically linked to a DNA molecule of non-human origin where the pMHC complex comprises the HLA-B*35:03 allele.
Aspect Y84. A structure comprising a pMHC complex mechanically linked to a DNA molecule of non-human origin where the pMHC complex comprises the HLA-B*35:05 allele.
Aspect Y85. A structure comprising a pMHC complex mechanically linked to a DNA molecule of non-human origin where the pMHC complex comprises the HLA-B*35:08 allele.
Aspect Y86. A structure comprising a pMHC complex mechanically linked to a DNA molecule of non-human origin where the pMHC complex comprises the HLA-B*37:01 allele.
Aspect Y87. A structure comprising a pMHC complex mechanically linked to a DNA molecule of non-human origin where the pMHC complex comprises the HLA-B*38:01 allele.
Aspect Y88. A structure comprising a pMHC complex mechanically linked to a DNA molecule of non-human origin where the pMHC complex comprises the HLA-B*38:02 allele.
Aspect Y89. A structure comprising a pMHC complex mechanically linked to a DNA molecule of non-human origin where the pMHC complex comprises the HLA-B*39:01 allele.
Aspect Y90. A structure comprising a pMHC complex mechanically linked to a DNA molecule of non-human origin where the pMHC complex comprises the HLA-B*39:02 allele.
Aspect Y91. A structure comprising a pMHC complex mechanically linked to a DNA molecule of non-human origin where the pMHC complex comprises the HLA-B*39:06 allele.
Aspect Y92. A structure comprising a pMHC complex mechanically linked to a DNA molecule of non-human origin where the pMHC complex comprises the HLA-B*39:10 allele.
Aspect Y93. A structure comprising a pMHC complex mechanically linked to a DNA molecule of non-human origin where the pMHC complex comprises the HLA-B*40:01 allele.
Aspect Y94. A structure comprising a pMHC complex mechanically linked to a DNA molecule of non-human origin where the pMHC complex comprises the HLA-B*40:02 allele.
Aspect Y95. A structure comprising a pMHC complex mechanically linked to a DNA molecule of non-human origin where the pMHC complex comprises the HLA-B*41:01 allele.
Aspect Y96. A structure comprising a pMHC complex mechanically linked to a DNA molecule of non-human origin where the pMHC complex comprises the HLA-B*42:01 allele.
Aspect Y97. A structure comprising a pMHC complex mechanically linked to a DNA molecule of non-human origin where the pMHC complex comprises the HLA-B*42:02 allele.
Aspect Y98. A structure comprising a pMHC complex mechanically linked to a DNA molecule of non-human origin where the pMHC complex comprises the HLA-B*44:02 allele.
Aspect Y99. A structure comprising a pMHC complex mechanically linked to a DNA molecule of non-human origin where the pMHC complex comprises the HLA-B*44:03 allele.
Aspect Y100. A structure comprising a pMHC complex mechanically linked to a DNA molecule of non-human origin where the pMHC complex comprises the HLA-B*46:01 allele.
Aspect Y101. A structure comprising a pMHC complex mechanically linked to a DNA molecule of non-human origin where the pMHC complex comprises the HLA-B*49:01 allele.
Aspect Y102. A structure comprising a pMHC complex mechanically linked to a DNA molecule of non-human origin where the pMHC complex comprises the HLA-B*50:01 allele.
Aspect Y103. A structure comprising a pMHC complex mechanically linked to a DNA molecule of non-human origin where the pMHC complex comprises the HLA-B*51:01 allele.
Aspect Y104. A structure comprising a pMHC complex mechanically linked to a DNA molecule of non-human origin where the pMHC complex comprises the HLA-B*52:01 allele.
Aspect Y105. A structure comprising a pMHC complex mechanically linked to a DNA molecule of non-human origin where the pMHC complex comprises the HLA-B*53:01 allele.
Aspect Y106. A structure comprising a pMHC complex mechanically linked to a DNA molecule of non-human origin where the pMHC complex comprises the HLA-B*54:01 allele.
Aspect Y107. A structure comprising a pMHC complex mechanically linked to a DNA molecule of non-human origin where the pMHC complex comprises the HLA-B*55:01 allele.
Aspect Y108. A structure comprising a pMHC complex mechanically linked to a DNA molecule of non-human origin where the pMHC complex comprises the HLA-B*55:02 allele.
Aspect Y109. A structure comprising a pMHC complex mechanically linked to a DNA molecule of non-human origin where the pMHC complex comprises the HLA-B*56:01 allele.
Aspect Y110. A structure comprising a pMHC complex mechanically linked to a DNA molecule of non-human origin where the pMHC complex comprises the HLA-B*57:01 allele.
Aspect Y111. A structure comprising a pMHC complex mechanically linked to a DNA molecule of non-human origin where the pMHC complex comprises the HLA-B*57:02 allele.
Aspect Y112. A structure comprising a pMHC complex mechanically linked to a DNA molecule of non-human origin where the pMHC complex comprises the HLA-B*57:03 allele.
Aspect Y113. A structure comprising a pMHC complex mechanically linked to a DNA molecule of non-human origin where the pMHC complex comprises the HLA-B*58:01 allele.
Aspect Y114. A structure comprising a pMHC complex mechanically linked to a DNA molecule of non-human origin where the pMHC complex comprises the HLA-B*58:02 allele.
Aspect Y115. A structure comprising a pMHC complex mechanically linked to a DNA molecule of non-human origin where the pMHC complex comprises the HLA-B*81:01 allele.
Aspect Y116. A structure comprising a pMHC complex mechanically linked to a DNA molecule of non-human origin where the pMHC complex comprises the HLA-B*83:01 allele.
Aspect Y117. A structure comprising a pMHC complex mechanically linked to a DNA molecule of non-human origin where the pMHC complex comprises the HLA-C*01:02 allele.
Aspect Y118. A structure comprising a pMHC complex mechanically linked to a DNA molecule of non-human origin where the pMHC complex comprises the HLA-C*02:02 allele.
Aspect Y119. A structure comprising a pMHC complex mechanically linked to a DNA molecule of non-human origin where the pMHC complex comprises the HLA-C*03:02 allele.
Aspect Y120. A structure comprising a pMHC complex mechanically linked to a DNA molecule of non-human origin where the pMHC complex comprises the HLA-C*03:03 allele.
Aspect Y121. A structure comprising a pMHC complex mechanically linked to a DNA molecule of non-human origin where the pMHC complex comprises the HLA-C*03:04 allele.
Aspect Y122. A structure comprising a pMHC complex mechanically linked to a DNA molecule of non-human origin where the pMHC complex comprises the HLA-C*04:01 allele.
Aspect Y123. A structure comprising a pMHC complex mechanically linked to a DNA molecule of non-human origin where the pMHC complex comprises the HLA-*C*05:01 allele.
Aspect Y124. A structure comprising a pMHC complex mechanically linked to a DNA molecule of non-human origin where the pMHC complex comprises the HLA-C*06:02* allele.
Aspect Y125. A structure comprising a pMHC complex mechanically linked to a DNA molecule of non-human origin where the pMHC complex comprises the HLA-C*07:01* allele.
Aspect Y126. A structure comprising a pMHC complex mechanically linked to a DNA molecule of non-human origin where the pMHC complex comprises the HLA-C*07:02* allele.
Aspect Y127. A structure comprising a pMHC complex mechanically linked to a DNA molecule of non-human origin where the pMHC complex comprises the HLA-C*08:01* allele.
Aspect Y128. A structure comprising a pMHC complex mechanically linked to a DNA molecule of non-human origin where the pMHC complex comprises the HLA-C*08:02* allele.
Aspect Y129. A structure comprising a pMHC complex mechanically linked to a DNA molecule of non-human origin where the pMHC complex comprises the HLA-C*12:03* allele.
Aspect Y130. A structure comprising a pMHC complex mechanically linked to a DNA molecule of non-human origin where the pMHC complex comprises the HLA-C*14:02* allele.
Aspect Y131. A structure comprising a pMHC complex mechanically linked to a DNA molecule of non-human origin where the pMHC complex comprises the HLA-C*15:02* allele.
Aspect Y132. A structure comprising a pMHC complex mechanically linked to a DNA molecule of non-human origin where the pMHC complex comprises the HLA-C*16:01* allele.
Aspect Y133. A structure comprising a pMHC complex mechanically linked to a DNA molecule of non-human origin where the pMHC complex comprises the HLA-C*17:01* allele.
Aspect Y134. A structure comprising a pMHC complex mechanically linked to a DNA molecule of non-human origin where the pMHC complex comprises the HLA-C*18:01* allele.
Aspect Y135. A structure comprising a pMHC complex mechanically linked to a DNA molecule of non-human origin where the pMHC complex comprises the HLA-CW*03:04 allele.
Aspect Y136. A structure comprising a pMHC complex mechanically linked to a DNA molecule of non-human origin where the pMHC complex comprises the HLA-CW*06:02 allele.
Aspect Z1. A structure comprising a pMHC complex mechanically linked to a DNA molecule of non-human origin where the peptide (p) of the pMHC complex is a Cancer-specific epitope.
Aspect Z2. A structure comprising a pMHC complex mechanically linked to a DNA molecule of non-human origin where the peptide (p) of the pMHC complex is a Skin cancer-specific epitope.
Aspect Z3. A structure comprising a pMHC complex mechanically linked to a DNA molecule of non-human origin where the peptide (p) of the pMHC complex is a Lung cancer-specific epitope.
Aspect Z4. A structure comprising a pMHC complex mechanically linked to a DNA molecule of non-human origin where the peptide (p) of the pMHC complex is a Prostate cancer-specific epitope.
Aspect Z5. A structure comprising a pMHC complex mechanically linked to a DNA molecule of non-human origin where the peptide (p) of the pMHC complex is a Breast cancer-specific epitope.
Aspect Z6. A structure comprising a pMHC complex mechanically linked to a DNA molecule of non-human origin where the peptide (p) of the pMHC complex is a Melanoma-specific epitope.
Aspect Z7. A structure comprising a pMHC complex mechanically linked to a DNA molecule of non-human origin where the peptide (p) of the pMHC complex is a Colorectal cancer-specific epitope.
Aspect Z8. A structure comprising a pMHC complex mechanically linked to a DNA molecule of non-human origin where the peptide (p) of the pMHC complex is a Kidney (renal) cancer-specific epitope.
Aspect Z9. A structure comprising a pMHC complex mechanically linked to a DNA molecule of non-human origin where the peptide (p) of the pMHC complex is a Bladder cancer-specific epitope.
Aspect Z10. A structure comprising a pMHC complex mechanically linked to a DNA molecule of non-human origin where the peptide (p) of the pMHC complex is a Non-Hodgkin's lymphoma-specific epitope.
Aspect Z11. A structure comprising a pMHC complex mechanically linked to a DNA molecule of non-human origin where the peptide (p) of the pMHC complex is a Carcinoma-specific epitope.
Aspect Z12. A structure comprising a pMHC complex mechanically linked to a DNA molecule of non-human origin where the peptide (p) of the pMHC complex is a Sarcoma-specific epitope.
Aspect Z13. A structure comprising a pMHC complex mechanically linked to a DNA molecule of non-human origin where the peptide (p) of the pMHC complex is a Lymphoma-specific epitope.
Aspect Z14. A structure comprising a pMHC complex mechanically linked to a DNA molecule of non-human origin where the peptide (p) of the pMHC complex is a Leukemia-specific epitope.
Aspect Z15. A structure comprising a pMHC complex mechanically linked to a DNA molecule of non-human origin where the peptide (p) of the pMHC complex is a Germ cell tumor-specific epitope.
Aspect Z16. A structure comprising a pMHC complex mechanically linked to a DNA molecule of non-human origin where the peptide (p) of the pMHC complex is a Blastoma-specific epitope.
Aspect Z17. A structure comprising a pMHC complex mechanically linked to a DNA molecule of non-human origin where the peptide (p) of the pMHC complex is a virus-specific epitope.
Aspect Z18. A structure comprising a pMHC complex mechanically linked to a DNA molecule of non-human origin where the peptide (p) of the pMHC complex is a Corona virus-specific epitope.
Aspect Z19. A structure comprising a pMHC complex mechanically linked to a DNA molecule of non-human origin where the peptide (p) of the pMHC complex is a HIV (Human immunodeficiency virus)-specific epitope.
Aspect Z20. A structure comprising a pMHC complex mechanically linked to a DNA molecule of non-human origin where the peptide (p) of the pMHC complex is a Bacterium-specific epitope.
Aspect Z21. A structure comprising a pMHC complex mechanically linked to a DNA molecule of non-human origin where the peptide (p) of the pMHC complex is a Hepatitis A virus-specific epitope.
Aspect Z22. A structure comprising a pMHC complex mechanically linked to a DNA molecule of non-human origin where the peptide (p) of the pMHC complex is a Hepatitis B virus-specific epitope.
Aspect Z23. A structure comprising a pMHC complex mechanically linked to a DNA molecule of non-human origin where the peptide (p) of the pMHC complex is a Hepatitis C virus-specific epitope.
Aspect Z24. A structure comprising a pMHC complex mechanically linked to a DNA molecule of non-human origin where the peptide (p) of the pMHC complex is a Hepatitis D Virus-specific epitope.
Aspect Z25. A structure comprising a pMHC complex mechanically linked to a DNA molecule of non-human origin where the peptide (p) of the pMHC complex is a Hepatitis E virus-specific epitope.
Aspect Z26. A structure comprising a pMHC complex mechanically linked to a DNA molecule of non-human origin where the peptide (p) of the pMHC complex is a Herpes simplex virus 1-specific epitope.
Aspect Z27. A structure comprising a pMHC complex mechanically linked to a DNA molecule of non-human origin where the peptide (p) of the pMHC complex is a Salmonella-specific epitope.
Aspect Z28. A structure comprising a pMHC complex mechanically linked to a DNA molecule of non-human origin where the peptide (p) of the pMHC complex is a Tuberculosis-specific epitope.
Aspect Z29. A structure comprising a pMHC complex mechanically linked to a DNA molecule of non-human origin where the peptide (p) of the pMHC complex is an E. coli-specific epitope.
Aspect Z30. A structure comprising a pMHC complex mechanically linked to a DNA molecule of non-human origin where the peptide (p) of the pMHC complex is an Epstein-Barr virus (EBV)-specific epitope.
Aspect Z31. A structure comprising a pMHC complex mechanically linked to a DNA molecule of non-human origin where the peptide (p) of the pMHC complex is a Diabetes-specific epitope.
Aspect Z32. A structure comprising a pMHC complex mechanically linked to a DNA molecule of non-human origin where the peptide (p) of the pMHC complex is a Rheumatoid arthritis-specific epitope.
Aspect Z33. A structure comprising a pMHC complex mechanically linked to a DNA molecule of non-human origin where the peptide (p) of the pMHC complex is a Peptide epitope consisting of less than 6 amino acid residues.
Aspect Z34. A structure comprising a pMHC complex mechanically linked to a DNA molecule of non-human origin where the peptide (p) of the pMHC complex is a Peptide epitope consisting of 6 amino acid residues.
Aspect Z35. A structure comprising a pMHC complex mechanically linked to a DNA molecule of non-human origin where the peptide (p) of the pMHC complex is a Peptide epitope consisting of 7 amino acid residues.
Aspect Z36. A structure comprising a pMHC complex mechanically linked to a DNA molecule of non-human origin where the peptide (p) of the pMHC complex is a Peptide epitope consisting of 8 amino acid residues.
Aspect Z37. A structure comprising a pMHC complex mechanically linked to a DNA molecule of non-human origin where the peptide (p) of the pMHC complex is a Peptide epitope consisting of 9 amino acid residues.
Aspect Z38. A structure comprising a pMHC complex mechanically linked to a DNA molecule of non-human origin where the peptide (p) of the pMHC complex is a Peptide epitope consisting of 10 amino acid residues.
Aspect Z39. A structure comprising a pMHC complex mechanically linked to a DNA molecule of non-human origin where the peptide (p) of the pMHC complex is a Peptide epitope consisting of 11 amino acid residues.
Aspect Z40. A structure comprising a pMHC complex mechanically linked to a DNA molecule of non-human origin where the peptide (p) of the pMHC complex is a Peptide epitope consisting of 12 amino acid residues.
Aspect Z41. A structure comprising a pMHC complex mechanically linked to a DNA molecule of non-human origin where the peptide (p) of the pMHC complex is a Peptide epitope consisting of 13 amino acid residues.
Aspect Z42. A structure comprising a pMHC complex mechanically linked to a DNA molecule of non-human origin where the peptide (p) of the pMHC complex is a Peptide epitope consisting of 14-17 amino acid residues.
Aspect Z43. A structure comprising a pMHC complex mechanically linked to a DNA molecule of non-human origin where the peptide (p) of the pMHC complex is a Peptide epitope consisting of 18-25 amino acid residues.
Aspect Z44. A structure comprising a pMHC complex mechanically linked to a DNA molecule of non-human origin where the peptide (p) of the pMHC complex is a Peptide epitope consisting of more than 25 amino acid residues.
Aspect Z45. A structure comprising a pMHC complex mechanically linked to a DNA molecule of non-human origin where the peptide (p) of the pMHC complex is a Peptide epitope comprising less than 6 amino acid residues.
Aspect Z46. A structure comprising a pMHC complex mechanically linked to a DNA molecule of non-human origin where the peptide (p) of the pMHC complex is a Peptide epitope comprising of 6 amino acid residues.
Aspect Z47. A structure comprising a pMHC complex mechanically linked to a DNA molecule of non-human origin where the peptide (p) of the pMHC complex is a Peptide epitope comprising of 7 amino acid residues.
Aspect Z48. A structure comprising a pMHC complex mechanically linked to a DNA molecule of non-human origin where the peptide (p) of the pMHC complex is a Peptide epitope comprising of 8 amino acid residues.
Aspect Z49. A structure comprising a pMHC complex mechanically linked to a DNA molecule of non-human origin where the peptide (p) of the pMHC complex is a Peptide epitope comprising of 9 amino acid residues.
Aspect Z50. A structure comprising a pMHC complex mechanically linked to a DNA molecule of non-human origin where the peptide (p) of the pMHC complex is a Peptide epitope comprising of 10 amino acid residues.
Aspect Z51. A structure comprising a pMHC complex mechanically linked to a DNA molecule of non-human origin where the peptide (p) of the pMHC complex is a Peptide epitope comprising of 11 amino acid residues.
Aspect Z52. A structure comprising a pMHC complex mechanically linked to a DNA molecule of non-human origin where the peptide (p) of the pMHC complex is a Peptide epitope comprising of 12 amino acid residues.
Aspect Z53. A structure comprising a pMHC complex mechanically linked to a DNA molecule of non-human origin where the peptide (p) of the pMHC complex is a Peptide epitope comprising of 13 amino acid residues.
Aspect Z54. A structure comprising a pMHC complex mechanically linked to a DNA molecule of non-human origin where the peptide (p) of the pMHC complex is a Peptide epitope comprising of 14-17 amino acid residues.
Aspect Z55. A structure comprising a pMHC complex mechanically linked to a DNA molecule of non-human origin where the peptide (p) of the pMHC complex is a Peptide epitope comprising of 18-25 amino acid residues.
Aspect Z56. A structure comprising a pMHC complex mechanically linked to a DNA molecule of non-human origin where the peptide (p) of the pMHC complex is a Peptide epitope comprising more than 25 amino acid residues.
Aspect Z57. A structure comprising a pMHC complex mechanically linked to a DNA molecule of non-human origin where the peptide (p) of the pMHC complex is a Peptide epitope with the sequence Glycyl-Methionine.
Aspect Z58. A structure comprising a pMHC complex mechanically linked to a DNA molecule of non-human origin where the peptide (p) of the pMHC complex is a Peptide epitope with the sequence Glycyl-Leucine.
Aspect Z59. A structure comprising a pMHC complex mechanically linked to a DNA molecule of non-human origin where the peptide (p) of the pMHC complex is a Peptide epitope with the sequence Glycyl-Cyclohexylalanine.
Aspect Z60. A structure comprising a pMHC complex mechanically linked to a DNA molecule of non-human origin where the peptide (p) of the pMHC complex is a Peptide epitope with the sequence Glycyl-Homoleucine.
Aspect Z61. A structure comprising a pMHC complex mechanically linked to a DNA molecule of non-human origin where the peptide (p) of the pMHC complex is a Clostridium botulinum-specific epitope.
Aspect Z62. A structure comprising a pMHC complex mechanically linked to a DNA molecule of non-human origin where the peptide (p) of the pMHC complex is a Vibrio cholera-specific epitope.
Aspect Z63. A structure comprising a pMHC complex mechanically linked to a DNA molecule of non-human origin where the peptide (p) of the pMHC complex is a Tetanus-specific epitope.
Aspect Z64. A structure comprising a pMHC complex mechanically linked to a DNA molecule of non-human origin where the peptide (p) of the pMHC complex is a Klebsiella-specific epitope.
Aspect Z65. A structure comprising a pMHC complex mechanically linked to a DNA molecule of non-human origin where the peptide (p) of the pMHC complex is a Staphylococcus-specific epitope.
Aspect Z66. A structure comprising a pMHC complex mechanically linked to a DNA molecule of non-human origin where the peptide (p) of the pMHC complex is a Syphilis-specific epitope.
Aspect Z67. A structure comprising a pMHC complex mechanically linked to a DNA molecule of non-human origin where the peptide (p) of the pMHC complex is a Streptococcus-specific epitope.
Aspect Z68. A structure comprising a pMHC complex mechanically linked to a DNA molecule of non-human origin where the peptide (p) of the pMHC complex is a Rhinovirus-specific epitope.
Aspect Z69. A structure comprising a pMHC complex mechanically linked to a DNA molecule of non-human origin where the peptide (p) of the pMHC complex is a Respiratory Syncytial Virus-specific epitope.
Aspect Z70. A structure comprising a pMHC complex mechanically linked to a DNA molecule of non-human origin where the peptide (p) of the pMHC complex is an Influenza virus-specific epitope.
Aspect Z71. A structure comprising a pMHC complex mechanically linked to a DNA molecule of non-human origin where the peptide (p) of the pMHC complex is a Herpes simplex-specific epitope.
Aspect Z72. A structure comprising a pMHC complex mechanically linked to a DNA molecule of non-human origin where the peptide (p) of the pMHC complex is a Varicella zoster-specific epitope.
Aspect Z73. A structure comprising a pMHC complex mechanically linked to a DNA molecule of non-human origin where the peptide (p) of the pMHC complex is a Mumps orthorubulavirus-specific epitope.
Aspect Z74. A structure comprising a pMHC complex mechanically linked to a DNA molecule of non-human origin where the peptide (p) of the pMHC complex is a Human papillomavirus (HPV)-specific epitope.
Aspect Z75. A structure comprising a pMHC complex mechanically linked to a DNA molecule of non-human origin where the peptide (p) of the pMHC complex is a Measles morbillivirus-specific epitope.
Aspect Z76. A structure comprising a pMHC complex mechanically linked to a DNA molecule of non-human origin where the peptide (p) of the pMHC complex is a Rubivirus rubella-specific epitope.
Aspect Z77. A structure comprising a pMHC complex mechanically linked to a DNA molecule of non-human origin where the peptide (p) of the pMHC complex is a Fungus-specific epitope.
Aspect Z78. A structure comprising a pMHC complex mechanically linked to a DNA molecule of non-human origin where the peptide (p) of the pMHC complex is a Candida albicans-specific epitope.
Aspect Z79. A structure comprising a pMHC complex mechanically linked to a DNA molecule of non-human origin where the peptide (p) of the pMHC complex is a Cryptococcosis neoformans-specific epitope.
Aspect Z80. A structure comprising a pMHC complex mechanically linked to a DNA molecule of non-human origin where the peptide (p) of the pMHC complex is an Aspergillus fumigatus-specific epitope.
Aspect Z81. A structure comprising a pMHC complex mechanically linked to a DNA molecule of non-human origin where the peptide (p) of the pMHC complex is a Coccidioides immitis-specific epitope.
Aspect Z82. A structure comprising a pMHC complex mechanically linked to a DNA molecule of non-human origin where the peptide (p) of the pMHC complex is a Histoplasma capsulatum-specific epitope.
Aspect Z83. A structure comprising a pMHC complex mechanically linked to a DNA molecule of non-human origin where the peptide (p) of the pMHC complex is a Blastomycosis dermatitidis-specific epitope.
Aspect Z84. A structure comprising a pMHC complex mechanically linked to a DNA molecule of non-human origin where the peptide (p) of the pMHC complex is a Pneumocystis pneumonia-specific epitope.
Aspect Z85. A structure comprising a pMHC complex mechanically linked to a DNA molecule of non-human origin where the peptide (p) of the pMHC complex is a Protozoan-specific epitope.
Aspect Z86. A structure comprising a pMHC complex mechanically linked to a DNA molecule of non-human origin where the peptide (p) of the pMHC complex is a Plasmodium falciparum-specific epitope.
Aspect Z87. A structure comprising a pMHC complex mechanically linked to a DNA molecule of non-human origin where the peptide (p) of the pMHC complex is a Trypanosoma brucei-specific epitope.
Aspect Z88. A structure comprising a pMHC complex mechanically linked to a DNA molecule of non-human origin where the peptide (p) of the pMHC complex is a Trypanosoma cruzi-specific epitope.
Aspect Z89. A structure comprising a pMHC complex mechanically linked to a DNA molecule of non-human origin where the peptide (p) of the pMHC complex is a Leishmania donovani-specific epitope.
Aspect Z90. A structure comprising a pMHC complex mechanically linked to a DNA molecule of non-human origin where the peptide (p) of the pMHC complex is a Toxoplasmosis gondii-specific epitope.
Aspect Z91. A structure comprising a pMHC complex mechanically linked to a DNA molecule of non-human origin where the peptide (p) of the pMHC complex is a Cryptosporidium hominis-specific epitope.
Aspect Z92. A structure comprising a pMHC complex mechanically linked to a DNA molecule of non-human origin where the peptide (p) of the pMHC complex is a Microbe-specific epitope.
Aspect Z93. A structure comprising a pMHC complex mechanically linked to a DNA molecule of non-human origin where the peptide (p) of the pMHC complex is an Acinetobacter baumannii-specific epitope.
Aspect Z94. A structure comprising a pMHC complex mechanically linked to a DNA molecule of non-human origin where the peptide (p) of the pMHC complex is an Actinomyces israelii-specific epitope.
Aspect Z95. A structure comprising a pMHC complex mechanically linked to a DNA molecule of non-human origin where the peptide (p) of the pMHC complex is an Actinomyces gerencseriae-specific epitope.
Aspect Z96. A structure comprising a pMHC complex mechanically linked to a DNA molecule of non-human origin where the peptide (p) of the pMHC complex is a Propionibacterium propionicus-specific epitope.
Aspect Z97. A structure comprising a pMHC complex mechanically linked to a DNA molecule of non-human origin where the peptide (p) of the pMHC complex is an Adenoviridae-specific epitope.
Aspect Z98. A structure comprising a pMHC complex mechanically linked to a DNA molecule of non-human origin where the peptide (p) of the pMHC complex is a Trypanosoma brucei-specific epitope.
Aspect Z99. A structure comprising a pMHC complex mechanically linked to a DNA molecule of non-human origin where the peptide (p) of the pMHC complex is an Entamoeba histolytica-specific epitope.
Aspect Z100. A structure comprising a pMHC complex mechanically linked to a DNA molecule of non-human origin where the peptide (p) of the pMHC complex is An Anaplasma species-specific epitope.
Aspect AA1. A structure comprising a pMHC complex mechanically linked to a DNA molecule of non-human origin where the pMHC complex is a pMHC class 1 complex.
Aspect AA2. A structure comprising a pMHC complex mechanically linked to a DNA molecule of non-human origin where the pMHC complex is a pMHC class 2 complex.
Aspect AA3. A structure comprising a pMHC complex mechanically linked to a DNA molecule of non-human origin where the pMHC complex is a pMHC-like complex.
Aspect AA4. A structure comprising a pMHC complex mechanically linked to a DNA molecule of non-human origin where the pMHC complex is a Peptide-receptive pMHC class 1 complex.
Aspect AA5. A structure comprising a pMHC complex mechanically linked to a DNA molecule of non-human origin where the pMHC complex is a Peptide-receptive pMHC class 2 complex.
Aspect AA6. A structure comprising a pMHC complex mechanically linked to a DNA molecule of non-human origin where the pMHC complex is a pMHC complex where the peptide (p) is a UV-cleavable peptide.
Aspect BB1. A structure comprising a pMHC complex mechanically linked to a DNA molecule where the structure further comprises a non-mammal fluorochrome label.
Aspect BB2. A structure comprising a pMHC complex mechanically linked to a DNA molecule where the structure further comprises a non-human fluorochrome label.
Aspect BB3. A structure comprising a pMHC complex mechanically linked to a DNA molecule where the structure further comprises a non-mammal chromophore label.
Aspect BB4. A structure comprising a pMHC complex mechanically linked to a DNA molecule where the structure further comprises a non-human chromophore label.
Aspect BB5. A structure comprising a pMHC complex mechanically linked to a DNA molecule where the structure further comprises a Rare element label.
Aspect BB6. A structure comprising a pMHC complex mechanically linked to a DNA molecule where the structure further comprises a Fluorescein label.
Aspect BB7. A structure comprising a pMHC complex mechanically linked to a DNA molecule where the structure further comprises a Dye label.
Aspect BB8. A structure comprising a pMHC complex mechanically linked to a DNA molecule where the structure further comprises a Chromophore label.
Aspect BB9. A structure comprising a pMHC complex mechanically linked to a DNA molecule where the structure further comprises a Fluorochrome label.
Aspect BB10. A structure comprising a pMHC complex mechanically linked to a DNA molecule where the structure further comprises an APC label.
Aspect BB11. A structure comprising a pMHC complex mechanically linked to a DNA molecule where the structure further comprises a Cy5 label.
Aspect BB12. A structure comprising a pMHC complex mechanically linked to a DNA molecule where the structure further comprises a PE label.
Aspect BB13. A structure comprising a pMHC complex mechanically linked to a DNA molecule where the structure further comprises a Gadolinium label.
Aspect BB14. A structure comprising a pMHC complex mechanically linked to a DNA molecule where the structure further comprises an Europium label.
Aspect BB15. A structure comprising a pMHC complex mechanically linked to a DNA molecule where the structure further comprises a rare earth metal label.
Aspect BB16. A structure comprising a pMHC complex mechanically linked to a DNA molecule where the structure further comprises a Rhodamine label.
Aspect BB17. A structure comprising a pMHC complex mechanically linked to a DNA molecule where the structure further comprises a FITC label.
Aspect BB18. A structure comprising a pMHC complex mechanically linked to a DNA molecule where the structure further comprises a Green FP label.
Aspect BB19. A structure comprising a pMHC complex mechanically linked to a DNA molecule where the structure further comprises a DNA tag label.
Aspect BB20. A structure comprising a pMHC complex mechanically linked to a DNA molecule where the structure further comprises an RNA tag label.
Aspect BB21. A structure comprising a pMHC complex mechanically linked to a DNA molecule where the structure further comprises a Fluorescent dye label.
Aspect BB22. A structure comprising a pMHC complex mechanically linked to a DNA molecule where the structure further comprises an Alexa Fluor label.
Aspect BB23. A structure comprising a pMHC complex mechanically linked to a DNA molecule where the structure further comprises a NovaFluor label.
Aspect BB24. A structure comprising a pMHC complex mechanically linked to a DNA molecule where the structure further comprises a BODIPY FL label.
Aspect BB25. A structure comprising a pMHC complex mechanically linked to a DNA molecule where the structure further comprises a Coumarin label.
Aspect BB26. A structure comprising a pMHC complex mechanically linked to a DNA molecule where the structure further comprises a Cy3 label.
Aspect BB27. A structure comprising a pMHC complex mechanically linked to a DNA molecule where the structure further comprises a DNA stain label.
Aspect BB28. A structure comprising a pMHC complex mechanically linked to a DNA molecule where the structure further comprises a DAPI label.
Aspect BB29. A structure comprising a pMHC complex mechanically linked to a DNA molecule where the structure further comprises a Propidium iodide label.
Aspect BB30. A structure comprising a pMHC complex mechanically linked to a DNA molecule where the structure further comprises a SYTO 9 label.
Aspect BB31. A structure comprising a pMHC complex mechanically linked to a DNA molecule where the structure further comprises a SYTOX Green label.
Aspect BB32. A structure comprising a pMHC complex mechanically linked to a DNA molecule where the structure further comprises a TO-PRO-3 label.
Aspect BB33. A structure comprising a pMHC complex mechanically linked to a DNA molecule where the structure further comprises a Qdot probe label.
Aspect BB34. A structure comprising a pMHC complex mechanically linked to a DNA molecule where the structure further comprises a Brilliant Ultra Violet Dye label.
Aspect BB35. A structure comprising a pMHC complex mechanically linked to a DNA molecule where the structure further comprises an RNA stain label.
Aspect BB36. A structure comprising a pMHC complex mechanically linked to a DNA molecule where the structure further comprises a Fluorescent protein label.
Aspect BB37. A structure comprising a pMHC complex mechanically linked to a DNA molecule where the structure further comprises a Cyan FP label.
Aspect BB38. A structure comprising a pMHC complex mechanically linked to a DNA molecule where the structure further comprises a Red FP label.
Aspect BB39. A structure comprising a pMHC complex mechanically linked to a DNA molecule where the structure further comprises a Protein tag label.
Aspect BB40. A structure comprising a pMHC complex mechanically linked to a DNA molecule where the structure further comprises a Biarsenical tag label.
Aspect BB41. A structure comprising a pMHC complex mechanically linked to a DNA molecule where the structure further comprises a Histidine tag label.
Aspect BB42. A structure comprising a pMHC complex mechanically linked to a DNA molecule where the structure further comprises a FLAG tag label.
Aspect BB43. A structure comprising a pMHC complex mechanically linked to a DNA molecule where the structure further comprises a Biotin label.
Aspect BB44. A structure comprising a pMHC complex mechanically linked to a DNA molecule where the structure further comprises a Streptavidin label.
Aspect BB45. A structure comprising a pMHC complex mechanically linked to a DNA molecule where the structure further comprises a DNA tag label.
Aspect BB46. A structure comprising a pMHC complex mechanically linked to a DNA molecule where the structure further comprises a RNA tag label.
Aspect CC1. A structure comprising a pMHC complex mechanically linked to a DNA molecule and further comprising a Dextramer scaffold.
Aspect CC2. A structure comprising a pMHC complex mechanically linked to a DNA molecule and further comprising a Streptamer scaffold.
Aspect CC3. A structure comprising a pMHC complex mechanically linked to a DNA molecule and further comprising a tetramer scaffold.
Aspect CC4. A structure comprising a pMHC complex mechanically linked to a DNA molecule and further comprising a pentamer scaffold.
Aspect CC5. A structure comprising a pMHC complex mechanically linked to a DNA molecule and further comprising a Streptavidin scaffold.
Aspect CC6. A structure comprising a pMHC complex mechanically linked to a DNA molecule and further comprising a Dextran scaffold.
Aspect CC7. A structure comprising a pMHC complex mechanically linked to a DNA molecule and further comprising a dimer scaffold.
Aspect CC8. A structure comprising a pMHC complex mechanically linked to a DNA molecule and further comprising a trimer scaffold.
Aspect CC9. A structure comprising a pMHC complex mechanically linked to a DNA molecule and further comprising a hexamer scaffold.
Aspect CC10. A structure comprising a pMHC complex mechanically linked to a DNA molecule and further comprising a SP1-based scaffold.
Aspect CC11. A structure comprising a pMHC complex mechanically linked to a DNA molecule and further comprising an IgG-based scaffold.
Aspect CC12. A structure comprising a pMHC complex mechanically linked to a DNA molecule and further comprising a Fos-Jun dimer scaffold.
Aspect CC13. A structure comprising a pMHC complex mechanically linked to a DNA molecule and further comprising a Pentameric coil-coil structure scaffold.
Aspect CC14. A structure comprising a pMHC complex mechanically linked to a DNA molecule and further comprising a Streptactin scaffold.
Aspect CC15. A structure comprising a pMHC complex mechanically linked to a DNA molecule and further comprising an IgM-based scaffold.
Aspect CC16. A structure comprising a pMHC complex mechanically linked to a DNA molecule and further comprising a Polypeptide scaffold.
Aspect CC17. A structure comprising a pMHC complex mechanically linked to a DNA molecule and further comprising a triplex DNA-based scaffold.

### Aspects FF1-LL17

Aspect FF1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin.
Aspect GG1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Cell.
Aspect GG2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Filamentous phage.
Aspect GG3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an M13 phage.
Aspect GG4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Phage particle comprising phagemid.
Aspect GG5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Dendritic cell.
Aspect GG6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an E. coli cell.
Aspect GG7. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Phage.
Aspect GG8. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Bacterial cell.
Aspect GG9. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Virus.
Aspect GG10. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a B cell.
Aspect GG11. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Human cell.
Aspect GG12. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Yeast cell.
Aspect GG13. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Micelle.
Aspect GG14. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Macrophage cell.
Aspect GG15. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a S. typhimurium cell.
Aspect GG16. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a B. subtilis cell.
Aspect GG17. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a S. cerevisiae cell.
Aspect GG18. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a S. pombe cell.
Aspect GG19. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Fungal cell.
Aspect GG20. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an Aspergillus cell.
Aspect GG21. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an Antigen-presenting cell.
Aspect GG22. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Professional antigen-presenting cell.
Aspect GG23. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Non-professional antigen-presenting cell.
Aspect GG24. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Nucleated cell.
Aspect GG25. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Baculovirus particle.
Aspect GG26. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Eukaryotic cell comprising membrane-spanning protein (tANCHOR).
Aspect GG27. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Virus-like particle such as Adaptsvac.
Aspect HH1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the A*02:01 allele.
Aspect HH2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the C*07:01 allele.
Aspect HH3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the A*01:01 allele.
Aspect HH4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the A*03:01 allele.
Aspect HH5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the C*07:02 allele.
Aspect HH6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the C*04:01 allele.
Aspect HH7. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the B*44:02 allele.
Aspect HH8. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the B*07:02 allele.
Aspect HH9. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the B*08:01 allele.
Aspect HH10. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the C*05:01 allele.
Aspect HH11. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the HLA-DPA1 allele.
Aspect HH12. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the HLA-DRB1 allele.
Aspect HH13. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the HLA-DQB1 allele.
Aspect HH14. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the HLA-DPB1 allele.
Aspect HH15. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the HLA-DRB4 allele.
Aspect HH16. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin, and where the pMHC complex comprises the HLA-DRB3 allele.
Aspect II1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Cancer-specific epitope.
Aspect II2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Skin cancer-specific epitope.
Aspect II3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Lung cancer-specific epitope.
Aspect II4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Prostate cancer-specific epitope.
Aspect II5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Breast cancer-specific epitope.
Aspect II6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Melanoma-specific epitope.
Aspect II7. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Colorectal cancer-specific epitope.
Aspect II8. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Kidney (renal) cancer-specific epitope.
Aspect II9. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Bladder cancer-specific epitope.
Aspect II10. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Non-Hodgkin's lymphoma-specific epitope.
Aspect II11. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Carcinoma-specific epitope.
Aspect II12. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Sarcoma-specific epitope.
Aspect II13. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Lymphoma-specific epitope.
Aspect II14. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Leukemia-specific epitope.
Aspect II15. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Germ cell tumor-specific epitope.
Aspect II16. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Blastoma-specific epitope.
Aspect II17. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a virus-specific epitope.
Aspect II18. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Corona virus-specific epitope.
Aspect II19. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a HIV (Human immunodeficiency virus)-specific epitope.
Aspect II20. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Bacterium-specific epitope.
Aspect II21. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Hepatitis A virus-specific epitope.
Aspect II22. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Hepatitis B virus-specific epitope.
Aspect II23. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Hepatitis C virus-specific epitope.
Aspect II24. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Hepatitis D Virus-specific epitope.
Aspect II25. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Hepatitis E virus-specific epitope.
Aspect II26. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Herpes simplex virus 1-specific epitope.
Aspect II27. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Salmonella-specific epitope.
Aspect II28. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Tuberculosis-specific epitope.
Aspect II29. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is an E. coli-specific epitope.
Aspect II30. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is an Epstein-Barr virus (EBV)-specific epitope.
Aspect II31. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Diabetes-specific epitope.
Aspect II32. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Rheumatoid arthritis-specific epitope.
Aspect II33. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of less than 6 amino acid residues.
Aspect II34. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of 6 amino acid residues.
Aspect II35. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of 7 amino acid residues.
Aspect II36. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of 8 amino acid residues.
Aspect II37. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of 9 amino acid residues.
Aspect II38. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of 10 amino acid residues.
Aspect II39. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of 11 amino acid residues.
Aspect II40. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of 12 amino acid residues.
Aspect II41. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of 13 amino acid residues.
Aspect II42. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of 14-17 amino acid residues.
Aspect II43. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of 18-25 amino acid residues.
Aspect II44. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope consisting of more than 25 amino acid residues.
Aspect II45. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising less than 6 amino acid residues.
Aspect II46. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising of 6 amino acid residues.
Aspect II47. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising of 7 amino acid residues.
Aspect II48. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising of 8 amino acid residues.
Aspect II49. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising of 9 amino acid residues.
Aspect II50. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising of 10 amino acid residues.
Aspect II51. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising of 11 amino acid residues.
Aspect II52. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising of 12 amino acid residues.
Aspect II53. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising of 13 amino acid residues.
Aspect II54. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising of 14-17 amino acid residues.
Aspect II55. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising of 18-25 amino acid residues.
Aspect II56. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope comprising more than 25 amino acid residues.
Aspect II57. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope with the sequence Glycyl-Methionine.
Aspect II58. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope with the sequence Glycyl-Leucine.
Aspect II59. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope with the sequence Glycyl-Cyclohexylalanine.
Aspect II60. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Peptide epitope with the sequence Glycyl-Homoleucine.
Aspect II61. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Clostridium botulinum-specific epitope.
Aspect II62. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Vibrio cholera-specific epitope.
Aspect II63. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Tetanus-specific epitope.
Aspect II64. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Klebsiella-specific epitope.
Aspect II65. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the peptide (p) of the pMHC complex is a Staphylococcus-specific epitope.
Aspect JJ1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the pMHC complex is a pMHC class 1 complex.
Aspect JJ2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the pMHC complex is a pMHC class 2 complex.
Aspect JJ3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the pMHC complex is a pMHC-like complex.
Aspect JJ4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the pMHC complex is a Peptide-receptive pMHC class 1 complex.
Aspect JJ5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the pMHC complex is a Peptide-receptive pMHC class 2 complex.
Aspect JJ6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule of non-human origin and where the pMHC complex is a pMHC complex where the peptide (p) is a UV-cleavable peptide.
Aspect KK1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a non-mammal fluorochrome label.
Aspect KK2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a non-human fluorochrome label.
Aspect KK3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a non-mammal chromophore label.
Aspect KK4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a non-human chromophore label.
Aspect KK5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Rare element label.
Aspect KK6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Fluorescein label.
Aspect KK7. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Dye label.
Aspect KK8. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Chromophore label.
Aspect KK9. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Fluorochrome label.
Aspect KK10. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an APC label.
Aspect KK11. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Cy5 label.
Aspect KK12. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a PE label.
Aspect KK13. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Gadolinium label.
Aspect KK14. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an Europium label.
Aspect KK15. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a rare earth metal label.
Aspect KK16. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Rhodamine label.
Aspect KK17. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a FITC label.
Aspect KK18. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Green FP label.
Aspect KK19. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a DNA tag label.
Aspect KK20. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an RNA tag label.
Aspect KK21. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Fluorescent dye label.
Aspect KK22. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an Alexa Fluor label.
Aspect KK23. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a NovaFluor label.
Aspect KK24. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a BODIPY FL label.
Aspect KK25. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Coumarin label.
Aspect KK26. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Cy3 label.
Aspect KK27. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a DNA stain label.
Aspect KK28. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a DAPI label.
Aspect KK29. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Propidium iodide label.
Aspect KK30. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a SYTO 9 label.
Aspect KK31. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a SYTOX Green label.
Aspect KK32. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a TO-PRO-3 label.
Aspect KK33. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Qdot probe label.
Aspect KK34. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Brilliant Ultra Violet Dye label.
Aspect KK35. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an RNA stain label.
Aspect KK36. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Fluorescent protein label.
Aspect KK37. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Cyan FP label.
Aspect KK38. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Red FP label.
Aspect KK39. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Protein tag label.
Aspect KK40. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Biarsenical tag label.
Aspect KK41. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Histidine tag label.
Aspect KK42. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a FLAG tag label.
Aspect KK43. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Biotin label.
Aspect KK44. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Streptavidin label.
Aspect KK45. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a DNA tag label.
Aspect KK46. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a RNA tag label.
Aspect LL1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Dextramer scaffold.
Aspect LL2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Streptamer scaffold.
Aspect LL3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a tetramer scaffold.
Aspect LL4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a pentamer scaffold.
Aspect LL5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Streptavidin scaffold.
Aspect LL6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Dextran scaffold.
Aspect LL7. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a dimer scaffold.
Aspect LL8. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a trimer scaffold.
Aspect LL9. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a hexamer scaffold.
Aspect LL10. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a SP1-based scaffold.
Aspect LL11. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an IgG-based scaffold.
Aspect LL12. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Fos-Jun dimer scaffold.
Aspect LL13. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Pentameric coil-coil structure scaffold.
Aspect LL14. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Streptactin scaffold.
Aspect LL15. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises an IgM-based scaffold.
Aspect LL16. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a Polypeptide scaffold.
Aspect LL17. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex linked to a unique DNA molecule and further comprises a triplex DNA-based scaffold.

### Aspects OO1-UU17

Aspect OO1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule.
Aspect PP1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Cell.
Aspect PP2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Filamentous phage.
Aspect PP3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an M13 phage.
Aspect PP4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Phage particle comprising phagemid.
Aspect PP5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Dendritic cell.
Aspect PP6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an E. coli cell.
Aspect PP7. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Phage.
Aspect PP8. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Bacterial cell.
Aspect PP9. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Virus.
Aspect PP10. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a B cell.
Aspect PP11. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Human cell.
Aspect PP12. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Yeast cell.
Aspect PP13. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Micelle.
Aspect PP14. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Macrophage cell.
Aspect PP15. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a S. typhimurium cell.
Aspect PP16. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a B. subtilis cell.
Aspect PP17. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a S. cerevisiae cell.
Aspect PP18. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a S. pombe cell.
Aspect PP19. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Fungal cell.
Aspect PP20. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an Aspergillus cell.
Aspect PP21. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an Antigen-presenting cell.
Aspect PP22. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Professional antigen-presenting cell.
Aspect PP23. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Non-professional antigen-presenting cell.
Aspect PP24. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Nucleated cell.
Aspect PP25. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Baculovirus particle.
Aspect PP26. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Eukaryotic cell comprising membrane-spanning protein (tANCHOR).
Aspect PP27. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Virus-like particle such as Adaptsvac.
Aspect QQ1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the A*02:01 allele.
Aspect QQ2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the C*07:01 allele.
Aspect QQ3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the A*01:01 allele.
Aspect QQ4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the A*03:01 allele.
Aspect QQ5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the C*07:02 allele.
Aspect QQ6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the C*04:01 allele.
Aspect QQ7. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the B*44:02 allele.
Aspect QQ8. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the B*07:02 allele.
Aspect QQ9. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the B*08:01 allele.
Aspect QQ10. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the C*05:01 allele.
Aspect QQ11. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the HLA-DPA1 allele.
Aspect QQ12. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the HLA-DRB1 allele.
Aspect QQ13. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the HLA-DQB1 allele.
Aspect QQ14. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the HLA-DPB1 allele.
Aspect QQ15. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the HLA-DRB4 allele.
Aspect QQ16. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the pMHC complex comprises the HLA-DRB3 allele.
Aspect RR1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Cancer-specific epitope.
Aspect RR2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Skin cancer-specific epitope.
Aspect RR3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Lung cancer-specific epitope.
Aspect RR4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Prostate cancer-specific epitope.
Aspect RR5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Breast cancer-specific epitope.
Aspect RR6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Melanoma-specific epitope.
Aspect RR7. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Colorectal cancer-specific epitope.
Aspect RR8. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Kidney (renal) cancer-specific epitope.
Aspect RR9. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Bladder cancer-specific epitope.
Aspect RR10. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Non-Hodgkin's lymphoma-specific epitope.
Aspect RR11. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Carcinoma-specific epitope.
Aspect RR12. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Sarcoma-specific epitope.
Aspect RR13. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Lymphoma-specific epitope.
Aspect RR14. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Leukemia-specific epitope.
Aspect RR15. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Germ cell tumor-specific epitope.
Aspect RR16. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Blastoma-specific epitope.
Aspect RR17. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a virus-specific epitope.
Aspect RR18. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Corona virus-specific epitope.
Aspect RR19. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a HIV (Human immunodeficiency virus)-specific epitope.
Aspect RR20. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Bacterium-specific epitope.
Aspect RR21. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Hepatitis A virus-specific epitope.
Aspect RR22. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Hepatitis B virus-specific epitope.
Aspect RR23. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Hepatitis C virus-specific epitope.
Aspect RR24. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Hepatitis D Virus-specific epitope.
Aspect RR25. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Hepatitis E virus-specific epitope.
Aspect RR26. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Herpes simplex virus 1-specific epitope.
Aspect RR27. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Salmonella-specific epitope.
Aspect RR28. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Tuberculosis-specific epitope.
Aspect RR29. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is an E. coli-specific epitope.
Aspect RR30. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is an Epstein-Barr virus (EBV)-specific epitope.
Aspect RR31. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Diabetes-specific epitope.
Aspect RR32. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Rheumatoid arthritis-specific epitope.
Aspect RR33. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of less than 6 amino acid residues.
Aspect RR34. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of 6 amino acid residues.
Aspect RR35. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of 7 amino acid residues.
Aspect RR36. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of 8 amino acid residues.
Aspect RR37. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of 9 amino acid residues.
Aspect RR38. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of 10 amino acid residues.
Aspect RR39. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of 11 amino acid residues.
Aspect RR40. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of 12 amino acid residues.
Aspect RR41. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of 13 amino acid residues.
Aspect RR42. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of 14-17 amino acid residues.
Aspect RR43. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of 18-25 amino acid residues.
Aspect RR44. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope consisting of more than 25 amino acid residues.
Aspect RR45. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising less than 6 amino acid residues.
Aspect RR46. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising of 6 amino acid residues.
Aspect RR47. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising of 7 amino acid residues.
Aspect RR48. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising of 8 amino acid residues.
Aspect RR49. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising of 9 amino acid residues.
Aspect RR50. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising of 10 amino acid residues.
Aspect RR51. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising of 11 amino acid residues.
Aspect RR52. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising of 12 amino acid residues.
Aspect RR53. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising of 13 amino acid residues.
Aspect RR54. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising of 14-17 amino acid residues.
Aspect RR55. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising of 18-25 amino acid residues.
Aspect RR56. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope comprising more than 25 amino acid residues.
Aspect RR57. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope with the sequence Glycyl-Methionine.
Aspect RR58. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope with the sequence Glycyl-Leucine.
Aspect RR59. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope with the sequence Glycyl-Cyclohexylalanine.
Aspect RR60. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Peptide epitope with the sequence Glycyl-Homoleucine.
Aspect RR61. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Clostridium botulinum-specific epitope.
Aspect RR62. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Vibrio cholera-specific epitope.
Aspect RR63. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Tetanus-specific epitope.
Aspect RR64. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Klebsiella-specific epitope.
Aspect RR65. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin, and where the peptide (p) of the pMHC complex is a Staphylococcus-specific epitope.
Aspect SS1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin and where the pMHC complex is a pMHC class 1 complex.
Aspect SS2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin and where the pMHC complex is a pMHC class 2 complex.
Aspect SS3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin and where the pMHC complex is a pMHC-like complex.
Aspect SS4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin and where the pMHC complex is a Peptide-receptive pMHC class 1 complex.
Aspect SS5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin and where the pMHC complex is a Peptide-receptive pMHC class 2 complex.
Aspect SS6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule of non-human origin and where the pMHC complex is a pMHC complex where the peptide (p) is a UV-cleavable peptide.
Aspect TT1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a non-mammal fluorochrome label.
Aspect TT2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a non-human fluorochrome label.
Aspect TT3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a non-mammal chromophore label.
Aspect TT4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a non-human chromophore label.
Aspect TT5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Rare element label.
Aspect TT6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Fluorescein label.
Aspect TT7. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Dye label.
Aspect TT8. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Chromophore label.
Aspect TT9. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Fluorochrome label.
Aspect TT10. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an APC label.
Aspect TT11. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Cy5 label.
Aspect TT12. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a PE label.
Aspect TT13. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Gadolinium label.
Aspect TT14. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an Europium label.
Aspect TT15. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a rare earth metal label.
Aspect TT16. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Rhodamine label.
Aspect TT17. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a FITC label.
Aspect TT18. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Green FP label.
Aspect TT19. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a DNA tag label.
Aspect TT20. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an RNA tag label.
Aspect TT21. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Fluorescent dye label.
Aspect TT22. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an Alexa Fluor label.
Aspect TT23. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a NovaFluor label.
Aspect TT24. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a BODIPY FL label.
Aspect TT25. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Coumarin label.
Aspect TT26. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Cy3 label.
Aspect TT27. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a DNA stain label.
Aspect TT28. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a DAPI label.
Aspect TT29. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Propidium iodide label.
Aspect TT30. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a SYTO 9 label.
Aspect TT31. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a SYTOX Green label.
Aspect TT32. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a TO-PRO-3 label.
Aspect TT33. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Qdot probe label.
Aspect TT34. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Brilliant Ultra Violet Dye label.
Aspect TT35. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an RNA stain label.
Aspect TT36. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Fluorescent protein label.
Aspect TT37. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Cyan FP label.
Aspect TT38. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Red FP label.
Aspect TT39. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Protein tag label.
Aspect TT40. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Biarsenical tag label.
Aspect TT41. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Histidine tag label.
Aspect TT42. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a FLAG tag label.
Aspect TT43. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Biotin label.
Aspect TT44. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Streptavidin label.
Aspect TT45. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a DNA tag label.
Aspect TT46. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a RNA tag label.
Aspect UU1. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Dextramer scaffold.
Aspect UU2. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Streptamer scaffold.
Aspect UU3. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a tetramer scaffold.
Aspect UU4. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a pentamer scaffold.
Aspect UU5. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Streptavidin scaffold.
Aspect UU6. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Dextran scaffold.
Aspect UU7. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a dimer scaffold.
Aspect UU8. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a trimer scaffold.
Aspect UU9. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a hexamer scaffold.
Aspect UU10. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a SP1-based scaffold.
Aspect UU11. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an IgG-based scaffold.
Aspect UU12. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Fos-Jun dimer scaffold.
Aspect UU13. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Pentameric coil-coil structure scaffold.
Aspect UU14. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Streptactin scaffold.
Aspect UU15. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises an IgM-based scaffold.
Aspect UU16. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a Polypeptide scaffold.
Aspect UU17. A composition of more than 2 structures, such as more than 10 structures, such as more than 100 structures, such as more than 1,000 structures, such as more than 10,000 structures, such as more than 100,000 structures, such as more than 1,000,000 structures, where each structure comprises a unique pMHC complex mechanically linked to a unique DNA molecule and further comprises a triplex DNA-based scaffold.

### Aspects AAA1-CCC100

Aspect AAA1. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the A*02:01 allele.
Aspect AAA2. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the A*02:01 allele.
Aspect AAA3. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the A*02:01 allele.
Aspect AAA4. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the A*02:01 allele.
Aspect AAA5. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the A*02:01 allele.
Aspect AAA6. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the A*02:01 allele.
Aspect AAA7. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the A*02:01 allele.
Aspect AAA8. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the A*02:01 allele.
Aspect AAA9. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the A*02:01 allele.
Aspect AAA10. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the A*02:01 allele.
Aspect AAA11. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the A*02:01 allele.
Aspect AAA12. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the A*02:01 allele.
Aspect AAA13. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the A*02:01 allele.
Aspect AAA14. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the A*02:01 allele.
Aspect AAA15. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the A*02:01 allele.
Aspect AAA16. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the A*02:01 allele.
Aspect AAA17. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the A*02:01 allele.
Aspect AAA18. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the A*02:01 allele.
Aspect AAA19. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the A*02:01 allele.
Aspect AAA20. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the A*02:01 allele.
Aspect AAA21. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the C*07:01 allele.
Aspect AAA22. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the C*07:01 allele.
Aspect AAA23. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the C*07:01 allele.
Aspect AAA24. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the C*07:01 allele.
Aspect AAA25. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the C*07:01 allele.
Aspect AAA26. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the C*07:01 allele.
Aspect AAA27. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the C*07:01 allele.
Aspect AAA28. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the C*07:01 allele.
Aspect AAA29. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the C*07:01 allele.
Aspect AAA30. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the C*07:01 allele.
Aspect AAA31. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the C*07:01 allele.
Aspect AAA32. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the C*07:01 allele.
Aspect AAA33. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the C*07:01 allele.
Aspect AAA34. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the C*07:01 allele.
Aspect AAA35. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the C*07:01 allele.
Aspect AAA36. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the C*07:01 allele.
Aspect AAA37. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the C*07:01 allele.
Aspect AAA38. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the C*07:01 allele.
Aspect AAA39. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the C*07:01 allele.
Aspect AAA40. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the C*07:01 allele.
Aspect AAA41. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the A*01:01 allele.
Aspect AAA42. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the A*01:01 allele.
Aspect AAA43. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the A*01:01 allele.
Aspect AAA44. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the A*01:01 allele.
Aspect AAA45. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the A*01:01 allele.
Aspect AAA46. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the A*01:01 allele.
Aspect AAA47. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the A*01:01 allele.
Aspect AAA48. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the A*01:01 allele.
Aspect AAA49. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the A*01:01 allele.
Aspect AAA50. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the A*01:01 allele.
Aspect AAA51. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the A*01:01 allele.
Aspect AAA52. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the A*01:01 allele.
Aspect AAA53. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the A*01:01 allele.
Aspect AAA54. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the A*01:01 allele.
Aspect AAA55. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the A*01:01 allele.
Aspect AAA56. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the A*01:01 allele.
Aspect AAA57. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the A*01:01 allele.
Aspect AAA58. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the A*01:01 allele.
Aspect AAA59. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the A*01:01 allele.
Aspect AAA60. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the A*01:01 allele.
Aspect AAA61. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the A*03:01 allele.
Aspect AAA62. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the A*03:01 allele.
Aspect AAA63. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the A*03:01 allele.
Aspect AAA64. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the A*03:01 allele.
Aspect AAA65. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the A*03:01 allele.
Aspect AAA66. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the A*03:01 allele.
Aspect AAA67. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the A*03:01 allele.
Aspect AAA68. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the A*03:01 allele.
Aspect AAA69. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the A*03:01 allele.
Aspect AAA70. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the A*03:01 allele.
Aspect AAA71. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the A*03:01 allele.
Aspect AAA72. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the A*03:01 allele.
Aspect AAA73. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the A*03:01 allele.
Aspect AAA74. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the A*03:01 allele.
Aspect AAA75. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the A*03:01 allele.
Aspect AAA76. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the A*03:01 allele.
Aspect AAA77. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the A*03:01 allele.
Aspect AAA78. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the A*03:01 allele.
Aspect AAA79. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the A*03:01 allele.
Aspect AAA80. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the A*03:01 allele.
Aspect AAA81. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the C*07:02 allele.
Aspect AAA82. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the C*07:02 allele.
Aspect AAA83. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the C*07:02 allele.
Aspect AAA84. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the C*07:02 allele.
Aspect AAA85. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the C*07:02 allele.
Aspect AAA86. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the C*07:02 allele.
Aspect AAA87. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the C*07:02 allele.
Aspect AAA88. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the C*07:02 allele.
Aspect AAA89. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the C*07:02 allele.
Aspect AAA90. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the C*07:02 allele.
Aspect AAA91. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the C*07:02 allele.
Aspect AAA92. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the C*07:02 allele.
Aspect AAA93. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the C*07:02 allele.
Aspect AAA94. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the C*07:02 allele.
Aspect AAA95. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the C*07:02 allele.
Aspect AAA96. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the C*07:02 allele.
Aspect AAA97. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the C*07:02 allele.
Aspect AAA98. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the C*07:02 allele.
Aspect AAA99. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the C*07:02 allele.
Aspect AAA100. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the C*07:02 allele.
Aspect AAA101. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the C*04:01 allele.
Aspect AAA102. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the C*04:01 allele.
Aspect AAA103. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the C*04:01 allele.
Aspect AAA104. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the C*04:01 allele.
Aspect AAA105. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the C*04:01 allele.
Aspect AAA106. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the C*04:01 allele.
Aspect AAA107. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the C*04:01 allele.
Aspect AAA108. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the C*04:01 allele.
Aspect AAA109. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the C*04:01 allele.
Aspect AAA110. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the C*04:01 allele.
Aspect AAA111. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the C*04:01 allele.
Aspect AAA112. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the C*04:01 allele.
Aspect AAA113. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the C*04:01 allele.
Aspect AAA114. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the C*04:01 allele.
Aspect AAA115. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the C*04:01 allele.
Aspect AAA116. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the C*04:01 allele.
Aspect AAA117. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the C*04:01 allele.
Aspect AAA118. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the C*04:01 allele.
Aspect AAA119. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the C*04:01 allele.
Aspect AAA120. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the C*04:01 allele.
Aspect AAA121. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the B*44:02 allele.
Aspect AAA122. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the B*44:02 allele.
Aspect AAA123. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the B*44:02 allele.
Aspect AAA124. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the B*44:02 allele.
Aspect AAA125. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the B*44:02 allele.
Aspect AAA126. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the B*44:02 allele.
Aspect AAA127. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the B*44:02 allele.
Aspect AAA128. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the B*44:02 allele.
Aspect AAA129. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the B*44:02 allele.
Aspect AAA130. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the B*44:02 allele.
Aspect AAA131. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the B*44:02 allele.
Aspect AAA132. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the B*44:02 allele.
Aspect AAA133. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the B*44:02 allele.
Aspect AAA134. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the B*44:02 allele.
Aspect AAA135. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the B*44:02 allele.
Aspect AAA136. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the B*44:02 allele.
Aspect AAA137. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the B*44:02 allele.
Aspect AAA138. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the B*44:02 allele.
Aspect AAA139. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the B*44:02 allele.
Aspect AAA140. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the B*44:02 allele.
Aspect AAA141. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the B*07:02 allele.
Aspect AAA142. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the B*07:02 allele.
Aspect AAA143. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the B*07:02 allele.
Aspect AAA144. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the B*07:02 allele.
Aspect AAA145. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the B*07:02 allele.
Aspect AAA146. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the B*07:02 allele.
Aspect AAA147. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the B*07:02 allele.
Aspect AAA148. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the B*07:02 allele.
Aspect AAA149. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the B*07:02 allele.
Aspect AAA150. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the B*07:02 allele.
Aspect AAA151. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the B*07:02 allele.
Aspect AAA152. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the B*07:02 allele.
Aspect AAA153. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the B*07:02 allele.
Aspect AAA154. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the B*07:02 allele.
Aspect AAA155. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the B*07:02 allele.
Aspect AAA156. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the B*07:02 allele.
Aspect AAA157. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the B*07:02 allele.
Aspect AAA158. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the B*07:02 allele.
Aspect AAA159. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the B*07:02 allele.
Aspect AAA160. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the B*07:02 allele.
Aspect AAA161. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the B*08:01 allele.
Aspect AAA162. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the B*08:01 allele.
Aspect AAA163. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the B*08:01 allele.
Aspect AAA164. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the B*08:01 allele.
Aspect AAA165. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the B*08:01 allele.
Aspect AAA166. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the B*08:01 allele.
Aspect AAA167. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the B*08:01 allele.
Aspect AAA168. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the B*08:01 allele.
Aspect AAA169. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the B*08:01 allele.
Aspect AAA170. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the B*08:01 allele.
Aspect AAA171. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the B*08:01 allele.
Aspect AAA172. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the B*08:01 allele.
Aspect AAA173. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the B*08:01 allele.
Aspect AAA174. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the B*08:01 allele.
Aspect AAA175. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the B*08:01 allele.
Aspect AAA176. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the B*08:01 allele.
Aspect AAA177. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the B*08:01 allele.
Aspect AAA178. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the B*08:01 allele.
Aspect AAA179. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the B*08:01 allele.
Aspect AAA180. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the B*08:01 allele.
Aspect AAA181. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the C*05:01 allele.
Aspect AAA182. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the C*05:01 allele.
Aspect AAA183. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the C*05:01 allele.
Aspect AAA184. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the C*05:01 allele.
Aspect AAA185. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the C*05:01 allele.
Aspect AAA186. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the C*05:01 allele.
Aspect AAA187. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the C*05:01 allele.
Aspect AAA188. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the C*05:01 allele.
Aspect AAA189. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the C*05:01 allele.
Aspect AAA190. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the C*05:01 allele.
Aspect AAA191. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the C*05:01 allele.
Aspect AAA192. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the C*05:01 allele.
Aspect AAA193. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the C*05:01 allele.
Aspect AAA194. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the C*05:01 allele.
Aspect AAA195. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the C*05:01 allele.
Aspect AAA196. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the C*05:01 allele.
Aspect AAA197. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the C*05:01 allele.
Aspect AAA198. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the C*05:01 allele.
Aspect AAA199. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the C*05:01 allele.
Aspect AAA200. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the C*05:01 allele.
Aspect AAA201. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the HLA-DPA1 allele.
Aspect AAA202. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the HLA-DPA1 allele.
Aspect AAA203. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the HLA-DPA1 allele.
Aspect AAA204. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the HLA-DPA1 allele.
Aspect AAA205. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the HLA-DPA1 allele.
Aspect AAA206. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the HLA-DPA1 allele.
Aspect AAA207. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the HLA-DPA1 allele.
Aspect AAA208. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the HLA-DPA1 allele.
Aspect AAA209. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the HLA-DPA1 allele.
Aspect AAA210. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the HLA-DPA1 allele.
Aspect AAA211. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the HLA-DPA1 allele.
Aspect AAA212. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the HLA-DPA1 allele.
Aspect AAA213. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the HLA-DPA1 allele.
Aspect AAA214. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the HLA-DPA1 allele.
Aspect AAA215. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the HLA-DPA1 allele.
Aspect AAA216. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the HLA-DPA1 allele.
Aspect AAA217. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the HLA-DPA1 allele.
Aspect AAA218. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the HLA-DPA1 allele.
Aspect AAA219. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the HLA-DPA1 allele.
Aspect AAA220. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the HLA-DPA1 allele.
Aspect AAA221. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the HLA-DRB1 allele.
Aspect AAA222. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the HLA-DRB1 allele.
Aspect AAA223. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the HLA-DRB1 allele.
Aspect AAA224. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the HLA-DRB1 allele.
Aspect AAA225. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the HLA-DRB1 allele.
Aspect AAA226. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the HLA-DRB1 allele.
Aspect AAA227. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the HLA-DRB1 allele.
Aspect AAA228. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the HLA-DRB1 allele.
Aspect AAA229. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the HLA-DRB1 allele.
Aspect AAA230. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the HLA-DRB1 allele.
Aspect AAA231. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the HLA-DRB1 allele.
Aspect AAA232. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the HLA-DRB1 allele.
Aspect AAA233. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the HLA-DRB1 allele.
Aspect AAA234. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the HLA-DRB1 allele.
Aspect AAA235. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the HLA-DRB1 allele.
Aspect AAA236. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the HLA-DRB1 allele.
Aspect AAA237. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the HLA-DRB1 allele.
Aspect AAA238. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the HLA-DRB1 allele.
Aspect AAA239. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the HLA-DRB1 allele.
Aspect AAA240. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the HLA-DRB1 allele.
Aspect AAA241. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the HLA-DQB1 allele.
Aspect AAA242. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the HLA-DQB1 allele.
Aspect AAA243. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the HLA-DQB1 allele.
Aspect AAA244. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the HLA-DQB1 allele.
Aspect AAA245. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the HLA-DQB1 allele.
Aspect AAA246. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the HLA-DQB1 allele.
Aspect AAA247. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the HLA-DQB1 allele.
Aspect AAA248. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the HLA-DQB1 allele.
Aspect AAA249. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the HLA-DQB1 allele.
Aspect AAA250. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the HLA-DQB1 allele.
Aspect AAA251. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the HLA-DQB1 allele.
Aspect AAA252. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the HLA-DQB1 allele.
Aspect AAA253. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the HLA-DQB1 allele.
Aspect AAA254. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the HLA-DQB1 allele.
Aspect AAA255. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the HLA-DQB1 allele.
Aspect AAA256. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the HLA-DQB1 allele.
Aspect AAA257. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the HLA-DQB1 allele.
Aspect AAA258. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the HLA-DQB1 allele.
Aspect AAA259. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the HLA-DQB1 allele.
Aspect AAA260. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the HLA-DQB1 allele.
Aspect AAA261. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the HLA-DPB1 allele.
Aspect AAA262. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the HLA-DPB1 allele.
Aspect AAA263. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the HLA-DPB1 allele.
Aspect AAA264. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the HLA-DPB1 allele.
Aspect AAA265. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the HLA-DPB1 allele.
Aspect AAA266. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the HLA-DPB1 allele.
Aspect AAA267. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the HLA-DPB1 allele.
Aspect AAA268. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the HLA-DPB1 allele.
Aspect AAA269. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the HLA-DPB1 allele.
Aspect AAA270. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the HLA-DPB1 allele.
Aspect AAA271. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the HLA-DPB1 allele.
Aspect AAA272. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the HLA-DPB1 allele.
Aspect AAA273. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the HLA-DPB1 allele.
Aspect AAA274. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the HLA-DPB1 allele.
Aspect AAA275. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the HLA-DPB1 allele.
Aspect AAA276. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the HLA-DPB1 allele.
Aspect AAA277. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the HLA-DPB1 allele.
Aspect AAA278. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the HLA-DPB1 allele.
Aspect AAA279. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the HLA-DPB1 allele.
Aspect AAA280. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the HLA-DPB1 allele.
Aspect AAA281. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and where the pMHC complex comprises the HLA-DRB4 allele.
Aspect AAA282. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and where the pMHC complex comprises the HLA-DRB4 allele.
Aspect AAA283. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and where the pMHC complex comprises the HLA-DRB4 allele.
Aspect AAA284. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and where the pMHC complex comprises the HLA-DRB4 allele.
Aspect AAA285. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and where the pMHC complex comprises the HLA-DRB4 allele.
Aspect AAA286. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and where the pMHC complex comprises the HLA-DRB4 allele.
Aspect AAA287. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and where the pMHC complex comprises the HLA-DRB4 allele.
Aspect AAA288. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and where the pMHC complex comprises the HLA-DRB4 allele.
Aspect AAA289. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and where the pMHC complex comprises the HLA-DRB4 allele.
Aspect AAA290. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and where the pMHC complex comprises the HLA-DRB4 allele.
Aspect AAA291. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and where the pMHC complex comprises the HLA-DRB4 allele.
Aspect AAA292. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and where the pMHC complex comprises the HLA-DRB4 allele.
Aspect AAA293. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and where the pMHC complex comprises the HLA-DRB4 allele.
Aspect AAA294. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and where the pMHC complex comprises the HLA-DRB4 allele.
Aspect AAA295. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and where the pMHC complex comprises the HLA-DRB4 allele.
Aspect AAA296. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and where the pMHC complex comprises the HLA-DRB4 allele.
Aspect AAA297. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and where the pMHC complex comprises the HLA-DRB4 allele.
Aspect AAA298. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and where the pMHC complex comprises the HLA-DRB4 allele.
Aspect AAA299. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and where the pMHC complex comprises the HLA-DRB4 allele.
Aspect AAA300. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and where the pMHC complex comprises the HLA-DRB4 allele.
Aspect BBB1. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a non-mammal fluorochrome label.
Aspect BBB2. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a non-human fluorochrome label.
Aspect BBB3. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a non-mammal chromophore label.
Aspect BBB4. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a non-human chromophore label.
Aspect BBB5. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a Rare element label.
Aspect BBB6. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a Fluorescein label.
Aspect BBB7. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a Dye label.
Aspect BBB8. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a Chromophore label.
Aspect BBB9. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a Fluorochrome label.
Aspect BBB10. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and an APC label.
Aspect BBB11. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a Cy5 label.
Aspect BBB12. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Human cell and a PE label.
Aspect BBB13. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a Gadolinium label.
Aspect BBB14. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and an Europium label.
Aspect BBB15. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a rare earth metal label.
Aspect BBB16. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a Rhodamine label.
Aspect BBB17. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a FITC label.
Aspect BBB18. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Human cell and a Green FP label.
Aspect BBB19. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a non-mammal fluorochrome label.
Aspect BBB20. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a non-human fluorochrome label.
Aspect BBB21. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a non-mammal chromophore label.
Aspect BBB22. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a non-human chromophore label.
Aspect BBB23. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a Rare element label.
Aspect BBB24. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a Fluorescein label.
Aspect BBB25. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a Dye label.
Aspect BBB26. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a Chromophore label.
Aspect BBB27. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a mammalian cell and a Fluorochrome label.
Aspect BBB28. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and an APC label.
Aspect BBB29. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a Cy5 label.
Aspect BBB30. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a PE label.
Aspect BBB31. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a Gadolinium label.
Aspect BBB32. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and an Europium label.
Aspect BBB33. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a rare earth metal label.
Aspect BBB34. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a Rhodamine label.
Aspect BBB35. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a FITC label.
Aspect BBB36. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a mammalian cell and a Green FP label.
Aspect BBB37. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a non-mammal fluorochrome label.
Aspect BBB38. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a non-human fluorochrome label.
Aspect BBB39. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a non-mammal chromophore label.
Aspect BBB40. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a non-human chromophore label.
Aspect BBB41. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a Rare element label.
Aspect BBB42. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises an Antigen-presenting cell and a Fluorescein label.
Aspect BBB43. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a Dye label.
Aspect BBB44. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a Chromophore label.
Aspect BBB45. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a Fluorochrome label.
Aspect BBB46. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and an APC label.
Aspect BBB47. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a Cy5 label.
Aspect BBB48. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a PE label.
Aspect BBB49. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a Gadolinium label.
Aspect BBB50. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and an Europium label.
Aspect BBB51. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a rare earth metal label.
Aspect BBB52. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a Rhodamine label.
Aspect BBB53. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a FITC label.
Aspect BBB54. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an Antigen-presenting cell and a Green FP label.
Aspect BBB55. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a non-mammal fluorochrome label.
Aspect BBB56. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a non-human fluorochrome label.
Aspect BBB57. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Dendritic cell and a non-mammal chromophore label.
Aspect BBB58. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a non-human chromophore label.
Aspect BBB59. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Rare element label.
Aspect BBB60. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Fluorescein label.
Aspect BBB61. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Dye label.
Aspect BBB62. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Chromophore label.
Aspect BBB63. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Fluorochrome label.
Aspect BBB64. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and an APC label.
Aspect BBB65. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Cy5 label.
Aspect BBB66. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a PE label.
Aspect BBB67. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Gadolinium label.
Aspect BBB68. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and an Europium label.
Aspect BBB69. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a rare earth metal label.
Aspect BBB70. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Rhodamine label.
Aspect BBB71. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a FITC label.
Aspect BBB72. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Dendritic cell and a Green FP label.
Aspect BBB73. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a non-mammal fluorochrome label.
Aspect BBB74. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a non-human fluorochrome label.
Aspect BBB75. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a non-mammal chromophore label.
Aspect BBB76. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a non-human chromophore label.
Aspect BBB77. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a Rare element label.
Aspect BBB78. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a Fluorescein label.
Aspect BBB79. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a Dye label.
Aspect BBB80. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a Chromophore label.
Aspect BBB81. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a Fluorochrome label.
Aspect BBB82. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and an APC label.
Aspect BBB83. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a Cy5 label.
Aspect BBB84. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a PE label.
Aspect BBB85. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a Gadolinium label.
Aspect BBB86. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and an Europium label.
Aspect BBB87. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Macrophage cell and a rare earth metal label.
Aspect BBB88. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a Rhodamine label.
Aspect BBB89. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a FITC label.
Aspect BBB90. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Macrophage cell and a Green FP label.
Aspect BBB91. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a non-mammal fluorochrome label.
Aspect BBB92. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a non-human fluorochrome label.
Aspect BBB93. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a non-mammal chromophore label.
Aspect BBB94. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a non-human chromophore label.
Aspect BBB95. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a Rare element label.
Aspect BBB96. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a Fluorescein label.
Aspect BBB97. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a Dye label.
Aspect BBB98. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a Chromophore label.
Aspect BBB99. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a Fluorochrome label.
Aspect BBB100. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and an APC label.
Aspect BBB101. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a Cy5 label.
Aspect BBB102. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a PE label.
Aspect BBB103. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a Gadolinium label.
Aspect BBB104. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and an Europium label.
Aspect BBB105. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a rare earth metal label.
Aspect BBB106. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a Rhodamine label.
Aspect BBB107. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a FITC label.
Aspect BBB108. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Professional antigen-presenting cell and a Green FP label.
Aspect BBB109. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a non-mammal fluorochrome label.
Aspect BBB110. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a non-human fluorochrome label.
Aspect BBB111. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a non-mammal chromophore label.
Aspect BBB112. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a non-human chromophore label.
Aspect BBB113. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a Rare element label.
Aspect BBB114. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a Fluorescein label.
Aspect BBB115. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a Dye label.
Aspect BBB116. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Non-professional antigen-presenting cell and a Chromophore label.
Aspect BBB117. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a Fluorochrome label.
Aspect BBB118. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and an APC label.
Aspect BBB119. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a Cy5 label.
Aspect BBB120. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a PE label.
Aspect BBB121. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a Gadolinium label.
Aspect BBB122. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and an Europium label.
Aspect BBB123. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a rare earth metal label.
Aspect BBB124. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a Rhodamine label.
Aspect BBB125. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a FITC label.
Aspect BBB126. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Non-professional antigen-presenting cell and a Green FP label.
Aspect BBB127. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a non-mammal fluorochrome label.
Aspect BBB128. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a non-human fluorochrome label.
Aspect BBB129. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a non-mammal chromophore label.
Aspect BBB130. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a non-human chromophore label.
Aspect BBB131. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a B cell and a Rare element label.
Aspect BBB132. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a Fluorescein label.
Aspect BBB133. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a Dye label.
Aspect BBB134. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a Chromophore label.
Aspect BBB135. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a Fluorochrome label.
Aspect BBB136. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and an APC label.
Aspect BBB137. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a Cy5 label.
Aspect BBB138. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a PE label.
Aspect BBB139. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a Gadolinium label.
Aspect BBB140. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and an Europium label.
Aspect BBB141. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a rare earth metal label.
Aspect BBB142. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a Rhodamine label.
Aspect BBB143. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a FITC label.
Aspect BBB144. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a B cell and a Green FP label.
Aspect CCC1. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and a non-mammal fluorochrome.
Aspect CCC2. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and a non-mammal fluorochrome.
Aspect CCC3. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and a non-mammal fluorochrome.
Aspect CCC4. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and a non-mammal fluorochrome.
Aspect CCC5. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and a non-mammal fluorochrome.
Aspect CCC6. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and a non-mammal fluorochrome.
Aspect CCC7. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and a non-mammal fluorochrome.
Aspect CCC8. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and a non-mammal fluorochrome.
Aspect CCC9. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and a non-mammal fluorochrome.
Aspect CCC10. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and a non-mammal fluorochrome.
Aspect CCC11. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and a non-mammal fluorochrome.
Aspect CCC12. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and a non-mammal fluorochrome.
Aspect CCC13. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and a non-mammal fluorochrome.
Aspect CCC14. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and a non-mammal fluorochrome.
Aspect CCC15. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and a non-mammal fluorochrome.
Aspect CCC16. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and a non-mammal fluorochrome.
Aspect CCC17. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and a non-mammal fluorochrome.
Aspect CCC18. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and a non-mammal fluorochrome.
Aspect CCC19. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and a non-mammal fluorochrome.
Aspect CCC20. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and a non-mammal fluorochrome.
Aspect CCC21. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and a non-human fluorochrome.
Aspect CCC22. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and a non-human fluorochrome.
Aspect CCC23. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and a non-human fluorochrome.
Aspect CCC24. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and a non-human fluorochrome.
Aspect CCC25. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and a non-human fluorochrome.
Aspect CCC26. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and a non-human fluorochrome.
Aspect CCC27. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and a non-human fluorochrome.
Aspect CCC28. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and a non-human fluorochrome.
Aspect CCC29. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and a non-human fluorochrome.
Aspect CCC30. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and a non-human fluorochrome.
Aspect CCC31. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and a non-human fluorochrome.
Aspect CCC32. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and a non-human fluorochrome.
Aspect CCC33. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and a non-human fluorochrome.
Aspect CCC34. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and a non-human fluorochrome.
Aspect CCC35. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and a non-human fluorochrome.
Aspect CCC36. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and a non-human fluorochrome.
Aspect CCC37. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and a non-human fluorochrome.
Aspect CCC38. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and a non-human fluorochrome.
Aspect CCC39. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and a non-human fluorochrome.
Aspect CCC40. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and a non-human fluorochrome.
Aspect CCC41. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and a non-mammal chromophore.
Aspect CCC42. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and a non-mammal chromophore.
Aspect CCC43. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and a non-mammal chromophore.
Aspect CCC44. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and a non-mammal chromophore.
Aspect CCC45. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and a non-mammal chromophore.
Aspect CCC46. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and a non-mammal chromophore.
Aspect CCC47. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and a non-mammal chromophore.
Aspect CCC48. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and a non-mammal chromophore.
Aspect CCC49. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and a non-mammal chromophore.
Aspect CCC50. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and a non-mammal chromophore.
Aspect CCC51. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and a non-mammal chromophore.
Aspect CCC52. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and a non-mammal chromophore.
Aspect CCC53. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and a non-mammal chromophore.
Aspect CCC54. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and a non-mammal chromophore.
Aspect CCC55. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and a non-mammal chromophore.
Aspect CCC56. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and a non-mammal chromophore.
Aspect CCC57. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and a non-mammal chromophore.
Aspect CCC58. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and a non-mammal chromophore.
Aspect CCC59. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and a non-mammal chromophore.
Aspect CCC60. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and a non-mammal chromophore.
Aspect CCC61. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and a non-human chromophore.
Aspect CCC62. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and a non-human chromophore.
Aspect CCC63. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and a non-human chromophore.
Aspect CCC64. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and a non-human chromophore.
Aspect CCC65. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and a non-human chromophore.
Aspect CCC66. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and a non-human chromophore.
Aspect CCC67. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and a non-human chromophore.
Aspect CCC68. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and a non-human chromophore.
Aspect CCC69. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and a non-human chromophore.
Aspect CCC70. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and a non-human chromophore.
Aspect CCC71. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and a non-human chromophore.
Aspect CCC72. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and a non-human chromophore.
Aspect CCC73. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and a non-human chromophore.
Aspect CCC74. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and a non-human chromophore.
Aspect CCC75. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and a non-human chromophore.
Aspect CCC76. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and a non-human chromophore.
Aspect CCC77. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and a non-human chromophore.
Aspect CCC78. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and a non-human chromophore.
Aspect CCC79. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and a non-human chromophore.
Aspect CCC80. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and a non-human chromophore.
Aspect CCC81. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a non-mammalian Cell and a Rare element.
Aspect CCC82. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Filamentous phage and a Rare element.
Aspect CCC83. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle and a Rare element.
Aspect CCC84. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a M13 phage and a Rare element.
Aspect CCC85. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Phage particle comprising phagemid and a Rare element.
Aspect CCC86. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an E. coli cell and a Rare element.
Aspect CCC87. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Bacterial cell and a Rare element.
Aspect CCC88. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus particle and a Rare element.
Aspect CCC89. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Micelle and a Rare element.
Aspect CCC90. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. typhimurium cell and a Rare element.
Aspect CCC91. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a B. subtilis cell and a Rare element.
Aspect CCC92. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Yeast cell and a Rare element.
Aspect CCC93. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. cerevisiae cell and a Rare element.
Aspect CCC94. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a S. pombe cell and a Rare element.
Aspect CCC95. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Fungal cell and a Rare element.
Aspect CCC96. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises an Aspergillus cell and a Rare element.
Aspect CCC97. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Nucleated cell and a Rare element.
Aspect CCC98. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Baculovirus particle and a Rare element.
Aspect CCC99. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a Eukaryotic cell comprising membrane-spanning protein (tANCHOR) and a Rare element.
Aspect CCC100. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity further comprises a virus-like particle such as Adaptsvac and a Rare element.

### Aspect DDD1-DDD325

Aspect DDD1. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Cancer-specific epitope.
Aspect DDD2. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Skin cancer-specific epitope.
Aspect DDD3. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Lung cancer-specific epitope.
Aspect DDD4. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Prostate cancer-specific epitope.
Aspect DDD5. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Breast cancer-specific epitope.
Aspect DDD6. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Melanoma-specific epitope.
Aspect DDD7. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Colorectal cancer-specific epitope.
Aspect DDD8. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Kidney (renal) cancer-specific epitope.
Aspect DDD9. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Bladder cancer-specific epitope.
Aspect DDD10. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Non-Hodgkin's lymphoma-specific epitope.
Aspect DDD11. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Carcinoma-specific epitope.
Aspect DDD12. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Sarcoma-specific epitope.
Aspect DDD13. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Lymphoma-specific epitope.
Aspect DDD14. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Cell and a Leukemia-specific epitope.
Aspect DDD15. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Germ cell tumor-specific epitope.
Aspect DDD16. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Blastoma-specific epitope.
Aspect DDD17. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a virus-specific epitope.
Aspect DDD18. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Corona virus-specific epitope.
Aspect DDD19. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a HIV (Human immunodeficiency virus)-specific epitope.
Aspect DDD20. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Bacterium-specific epitope.
Aspect DDD21. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Hepatitis A virus-specific epitope.
Aspect DDD22. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Hepatitis B virus-specific epitope.
Aspect DDD23. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Hepatitis C virus-specific epitope.
Aspect DDD24. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Hepatitis D Virus-specific epitope.
Aspect DDD25. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Hepatitis E virus-specific epitope.
Aspect DDD26. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Herpes simplex virus 1-specific epitope.
Aspect DDD27. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Salmonella-specific epitope.
Aspect DDD28. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Tuberculosis-specific epitope.
Aspect DDD29. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Cell and an E. coli-specific epitope.
Aspect DDD30. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and an Epstein-Barr virus (EBV)-specific epitope.
Aspect DDD31. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Diabetes-specific epitope.
Aspect DDD32. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Rheumatoid arthritis-specific epitope.
Aspect DDD33. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of less than 6 amino acid residues.
Aspect DDD34. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of 6 amino acid residues.
Aspect DDD35. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of 7 amino acid residues.
Aspect DDD36. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of 8 amino acid residues.
Aspect DDD37. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of 9 amino acid residues.
Aspect DDD38. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of 10 amino acid residues.
Aspect DDD39. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of 11 amino acid residues.
Aspect DDD40. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of 12 amino acid residues.
Aspect DDD41. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of 13 amino acid residues.
Aspect DDD42. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of 14-17 amino acid residues.
Aspect DDD43. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of 18-25 amino acid residues.
Aspect DDD44. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope consisting of more than 25 amino acid residues.
Aspect DDD45. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising less than 6 amino acid residues.
Aspect DDD46. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising of 6 amino acid residues.
Aspect DDD47. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising of 7 amino acid residues.
Aspect DDD48. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising of 8 amino acid residues.
Aspect DDD49. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising of 9 amino acid residues.
Aspect DDD50. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising of 10 amino acid residues.
Aspect DDD51. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising of 11 amino acid residues.
Aspect DDD52. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising of 12 amino acid residues.
Aspect DDD53. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising of 13 amino acid residues.
Aspect DDD54. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising of 14-17 amino acid residues.
Aspect DDD55. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising of 18-25 amino acid residues.
Aspect DDD56. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope comprising more than 25 amino acid residues.
Aspect DDD57. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope with the sequence Glycyl-Methionine.
Aspect DDD58. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope with the sequence Glycyl-Leucine.
Aspect DDD59. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Cell and a Peptide epitope with the sequence Glycyl-Cyclohexylalanine.
Aspect DDD60. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Peptide epitope with the sequence Glycyl-Homoleucine.
Aspect DDD61. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Clostridium botulinum-specific epitope.
Aspect DDD62. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Vibrio cholera-specific epitope.
Aspect DDD63. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Tetanus-specific epitope.
Aspect DDD64. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Klebsiella-specific epitope.
Aspect DDD65. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Cell and a Staphylococcus-specific epitope.
Aspect DDD66. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Cancer-specific epitope.
Aspect DDD67. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Skin cancer-specific epitope.
Aspect DDD68. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Lung cancer-specific epitope.
Aspect DDD69. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Prostate cancer-specific epitope.
Aspect DDD70. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Breast cancer-specific epitope.
Aspect DDD71. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Melanoma-specific epitope.
Aspect DDD72. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Colorectal cancer-specific epitope.
Aspect DDD73. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Kidney (renal) cancer-specific epitope.
Aspect DDD74. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Filamentous phage and a Bladder cancer-specific epitope.
Aspect DDD75. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Non-Hodgkin's lymphoma-specific epitope.
Aspect DDD76. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Carcinoma-specific epitope.
Aspect DDD77. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Sarcoma-specific epitope.
Aspect DDD78. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Lymphoma-specific epitope.
Aspect DDD79. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Leukemia-specific epitope.
Aspect DDD80. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Germ cell tumor-specific epitope.
Aspect DDD81. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Blastoma-specific epitope.
Aspect DDD82. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a virus-specific epitope.
Aspect DDD83. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Corona virus-specific epitope.
Aspect DDD84. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a HIV (Human immunodeficiency virus)-specific epitope.
Aspect DDD85. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Bacterium-specific epitope.
Aspect DDD86. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Hepatitis A virus-specific epitope.
Aspect DDD87. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Hepatitis B virus-specific epitope.
Aspect DDD88. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Hepatitis C virus-specific epitope.
Aspect DDD89. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Filamentous phage and a Hepatitis D Virus-specific epitope.
Aspect DDD90. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Hepatitis E virus-specific epitope.
Aspect DDD91. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Herpes simplex virus 1-specific epitope.
Aspect DDD92. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Salmonella-specific epitope.
Aspect DDD93. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Tuberculosis-specific epitope.
Aspect DDD94. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and an E. coli-specific epitope.
Aspect DDD95. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and an Epstein-Barr virus (EBV)-specific epitope.
Aspect DDD96. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Diabetes-specific epitope.
Aspect DDD97. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Rheumatoid arthritis-specific epitope.
Aspect DDD98. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of less than 6 amino acid residues.
Aspect DDD99. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of 6 amino acid residues.
Aspect DDD100. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of 7 amino acid residues.
Aspect DDD101. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of 8 amino acid residues.
Aspect DDD102. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of 9 amino acid residues.
Aspect DDD103. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of 10 amino acid residues.
Aspect DDD104. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of 11 amino acid residues.
Aspect DDD105. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of 12 amino acid residues.
Aspect DDD106. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of 13 amino acid residues.
Aspect DDD107. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of 14-17 amino acid residues.
Aspect DDD108. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of 18-25 amino acid residues.
Aspect DDD109. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope consisting of more than 25 amino acid residues.
Aspect DDD110. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising less than 6 amino acid residues.
Aspect DDD111. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising of 6 amino acid residues.
Aspect DDD112. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising of 7 amino acid residues.
Aspect DDD113. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising of 8 amino acid residues.
Aspect DDD114. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising of 9 amino acid residues.
Aspect DDD115. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising of 10 amino acid residues.
Aspect DDD116. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Filamentous phage and a Peptide epitope comprising of 11 amino acid residues.
Aspect DDD117. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising of 12 amino acid residues.
Aspect DDD118. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising of 13 amino acid residues.
Aspect DDD119. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising of 14-17 amino acid residues.
Aspect DDD120. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising of 18-25 amino acid residues.
Aspect DDD121. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope comprising more than 25 amino acid residues.
Aspect DDD122. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope with the sequence Glycyl-Methionine.
Aspect DDD123. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope with the sequence Glycyl-Leucine.
Aspect DDD124. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope with the sequence Glycyl-Cyclohexylalanine.
Aspect DDD125. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Peptide epitope with the sequence Glycyl-Homoleucine.
Aspect DDD126. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Clostridium botulinum-specific epitope.
Aspect DDD127. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Vibrio cholera-specific epitope.
Aspect DDD128. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Tetanus-specific epitope.
Aspect DDD129. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Klebsiella-specific epitope.
Aspect DDD130. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Filamentous phage and a Staphylococcus-specific epitope.
Aspect DDD131. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Cancer-specific epitope.
Aspect DDD132. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Skin cancer-specific epitope.
Aspect DDD133. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Lung cancer-specific epitope.
Aspect DDD134. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Prostate cancer-specific epitope.
Aspect DDD135. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Breast cancer-specific epitope.
Aspect DDD136. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Melanoma-specific epitope.
Aspect DDD137. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises an M13 phage and a Colorectal cancer-specific epitope.
Aspect DDD138. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Kidney (renal) cancer-specific epitope.
Aspect DDD139. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Bladder cancer-specific epitope.
Aspect DDD140. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Non-Hodgkin's lymphoma-specific epitope.
Aspect DDD141. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Carcinoma-specific epitope.
Aspect DDD142. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Sarcoma-specific epitope.
Aspect DDD143. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Lymphoma-specific epitope.
Aspect DDD144. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Leukemia-specific epitope.
Aspect DDD145. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Germ cell tumor-specific epitope.
Aspect DDD146. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Blastoma-specific epitope.
Aspect DDD147. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a virus-specific epitope.
Aspect DDD148. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Corona virus-specific epitope.
Aspect DDD149. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a HIV (Human immunodeficiency virus)-specific epitope.
Aspect DDD150. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Bacterium-specific epitope.
Aspect DDD151. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Hepatitis A virus-specific epitope.
Aspect DDD152. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises an M13 phage and a Hepatitis B virus-specific epitope.
Aspect DDD153. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Hepatitis C virus-specific epitope.
Aspect DDD154. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Hepatitis D Virus-specific epitope.
Aspect DDD155. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Hepatitis E virus-specific epitope.
Aspect DDD156. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Herpes simplex virus 1-specific epitope.
Aspect DDD157. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Salmonella-specific epitope.
Aspect DDD158. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Tuberculosis-specific epitope.
Aspect DDD159. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and an E. coli-specific epitope.
Aspect DDD160. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and an Epstein-Barr virus (EBV)-specific epitope.
Aspect DDD161. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Diabetes-specific epitope.
Aspect DDD162. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Rheumatoid arthritis-specific epitope.
Aspect DDD163. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of less than 6 amino acid residues.
Aspect DDD164. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of 6 amino acid residues.
Aspect DDD165. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of 7 amino acid residues.
Aspect DDD166. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of 8 amino acid residues.
Aspect DDD167. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises an M13 phage and a Peptide epitope consisting of 9 amino acid residues.
Aspect DDD168. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of 10 amino acid residues.
Aspect DDD169. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of 11 amino acid residues.
Aspect DDD170. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of 12 amino acid residues.
Aspect DDD171. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of 13 amino acid residues.
Aspect DDD172. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of 14-17 amino acid residues.
Aspect DDD173. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of 18-25 amino acid residues.
Aspect DDD174. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope consisting of more than 25 amino acid residues.
Aspect DDD175. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising less than 6 amino acid residues.
Aspect DDD176. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising of 6 amino acid residues.
Aspect DDD177. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising of 7 amino acid residues.
Aspect DDD178. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising of 8 amino acid residues.
Aspect DDD179. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising of 9 amino acid residues.
Aspect DDD180. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising of 10 amino acid residues.
Aspect DDD181. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising of 11 amino acid residues.
Aspect DDD182. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising of 12 amino acid residues.
Aspect DDD183. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising of 13 amino acid residues.
Aspect DDD184. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising of 14-17 amino acid residues.
Aspect DDD185. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising of 18-25 amino acid residues.
Aspect DDD186. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope comprising more than 25 amino acid residues.
Aspect DDD187. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope with the sequence Glycyl-Methionine.
Aspect DDD188. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope with the sequence Glycyl-Leucine.
Aspect DDD189. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope with the sequence Glycyl-Cyclohexylalanine.
Aspect DDD190. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Peptide epitope with the sequence Glycyl-Homoleucine.
Aspect DDD191. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Clostridium botulinum-specific epitope.
Aspect DDD192. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Vibrio cholera-specific epitope.
Aspect DDD193. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Tetanus-specific epitope.
Aspect DDD194. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Klebsiella-specific epitope.
Aspect DDD195. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises an M13 phage and a Staphylococcus-specific epitope.
Aspect DDD196. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Cancer-specific epitope.
Aspect DDD197. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Skin cancer-specific epitope.
Aspect DDD198. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Lung cancer-specific epitope.
Aspect DDD199. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Prostate cancer-specific epitope.
Aspect DDD200. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Breast cancer-specific epitope.
Aspect DDD201. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Melanoma-specific epitope.
Aspect DDD202. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Colorectal cancer-specific epitope.
Aspect DDD203. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Kidney (renal) cancer-specific epitope.
Aspect DDD204. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Bladder cancer-specific epitope.
Aspect DDD205. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Non-Hodgkin's lymphoma-specific epitope.
Aspect DDD206. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Carcinoma-specific epitope.
Aspect DDD207. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Sarcoma-specific epitope.
Aspect DDD208. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Lymphoma-specific epitope.
Aspect DDD209. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Phage particle comprising phagemid and a Leukemia-specific epitope.
Aspect DDD210. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Germ cell tumor-specific epitope.
Aspect DDD211. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Blastoma-specific epitope.
Aspect DDD212. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a virus-specific epitope.
Aspect DDD213. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Corona virus-specific epitope.
Aspect DDD214. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a HIV (Human immunodeficiency virus)-specific epitope.
Aspect DDD215. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Bacterium-specific epitope.
Aspect DDD216. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Hepatitis A virus-specific epitope.
Aspect DDD217. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Hepatitis B virus-specific epitope.
Aspect DDD218. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Hepatitis C virus-specific epitope.
Aspect DDD219. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Hepatitis D Virus-specific epitope.
Aspect DDD220. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Hepatitis E virus-specific epitope.
Aspect DDD221. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Herpes simplex virus 1-specific epitope.
Aspect DDD222. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Salmonella-specific epitope.
Aspect DDD223. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Tuberculosis-specific epitope.
Aspect DDD224. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and an E. coli-specific epitope.
Aspect DDD225. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and an Epstein-Barr virus (EBV)-specific epitope.
Aspect DDD226. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Diabetes-specific epitope.
Aspect DDD227. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Rheumatoid arthritis-specific epitope.
Aspect DDD228. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of less than 6 amino acid residues.
Aspect DDD229. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of 6 amino acid residues.
Aspect DDD230. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of 7 amino acid residues.
Aspect DDD231. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of 8 amino acid residues.
Aspect DDD232. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of 9 amino acid residues.
Aspect DDD233. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of 10 amino acid residues.
Aspect DDD234. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of 11 amino acid residues.
Aspect DDD235. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of 12 amino acid residues.
Aspect DDD236. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of 13 amino acid residues.
Aspect DDD237. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of 14-17 amino acid residues.
Aspect DDD238. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of 18-25 amino acid residues.
Aspect DDD239. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope consisting of more than 25 amino acid residues.
Aspect DDD240. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising less than 6 amino acid residues.
Aspect DDD241. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising of 6 amino acid residues.
Aspect DDD242. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising of 7 amino acid residues.
Aspect DDD243. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising of 8 amino acid residues.
Aspect DDD244. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising of 9 amino acid residues.
Aspect DDD245. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising of 10 amino acid residues.
Aspect DDD246. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising of 11 amino acid residues.
Aspect DDD247. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising of 12 amino acid residues.
Aspect DDD248. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising of 13 amino acid residues.
Aspect DDD249. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Phage particle comprising phagemid and a Peptide epitope comprising of 14-17 amino acid residues.
Aspect DDD250. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising of 18-25 amino acid residues.
Aspect DDD251. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope comprising more than 25 amino acid residues.
Aspect DDD252. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope with the sequence Glycyl-Methionine.
Aspect DDD253. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope with the sequence Glycyl-Leucine.
Aspect DDD254. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope with the sequence Glycyl-Cyclohexylalanine.
Aspect DDD255. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Peptide epitope with the sequence Glycyl-Homoleucine.
Aspect DDD256. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Clostridium botulinum-specific epitope.
Aspect DDD257. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Vibrio cholera-specific epitope.
Aspect DDD258. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Tetanus-specific epitope.
Aspect DDD259. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Klebsiella-specific epitope.
Aspect DDD260. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Phage particle comprising phagemid and a Staphylococcus-specific epitope.
Aspect DDD261. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Cancer-specific epitope.
Aspect DDD262. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Skin cancer-specific epitope.
Aspect DDD263. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Lung cancer-specific epitope.
Aspect DDD264. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Prostate cancer-specific epitope.
Aspect DDD265. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Breast cancer-specific epitope.
Aspect DDD266. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Melanoma-specific epitope.
Aspect DDD267. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Colorectal cancer-specific epitope.
Aspect DDD268. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Kidney (renal) cancer-specific epitope.
Aspect DDD269. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Bladder cancer-specific epitope.
Aspect DDD270. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Dendritic cell and a Non-Hodgkin's lymphoma-specific epitope.
Aspect DDD271. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Carcinoma-specific epitope.
Aspect DDD272. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Sarcoma-specific epitope.
Aspect DDD273. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Lymphoma-specific epitope.
Aspect DDD274. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Leukemia-specific epitope.
Aspect DDD275. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Germ cell tumor-specific epitope.
Aspect DDD276. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Blastoma-specific epitope.
Aspect DDD277. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a virus-specific epitope.
Aspect DDD278. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Corona virus-specific epitope.
Aspect DDD279. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a HIV (Human immunodeficiency virus)-specific epitope.
Aspect DDD280. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Bacterium-specific epitope.
Aspect DDD281. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Hepatitis A virus-specific epitope.
Aspect DDD282. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Hepatitis B virus-specific epitope.
Aspect DDD283. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Hepatitis C virus-specific epitope.
Aspect DDD284. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Hepatitis D Virus-specific epitope.
Aspect DDD285. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Dendritic cell and a Hepatitis E virus-specific epitope.
Aspect DDD286. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Herpes simplex virus 1-specific epitope.
Aspect DDD287. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Salmonella-specific epitope.
Aspect DDD288. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Tuberculosis-specific epitope.
Aspect DDD289. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and an E. coli-specific epitope.
Aspect DDD290. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and an Epstein-Barr virus (EBV)-specific epitope.
Aspect DDD291. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Diabetes-specific epitope.
Aspect DDD292. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Rheumatoid arthritis-specific epitope.
Aspect DDD293. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of less than 6 amino acid residues.
Aspect DDD294. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of 6 amino acid residues.
Aspect DDD295. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of 7 amino acid residues.
Aspect DDD296. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of 8 amino acid residues.
Aspect DDD297. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of 9 amino acid residues.
Aspect DDD298. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of 10 amino acid residues.
Aspect DDD299. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of 11 amino acid residues.
Aspect DDD300. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of 12 amino acid residues.
Aspect DDD301. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of 13 amino acid residues.
Aspect DDD302. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of 14-17 amino acid residues.
Aspect DDD303. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of 18-25 amino acid residues.
Aspect DDD304. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope consisting of more than 25 amino acid residues.
Aspect DDD305. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising less than 6 amino acid residues.
Aspect DDD306. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule, and where each entity where each entity further comprises a Dendritic cell and a Peptide epitope comprising of 6 amino acid residues.
Aspect DDD307. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising of 7 amino acid residues.
Aspect DDD308. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising of 8 amino acid residues.
Aspect DDD309. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising of 9 amino acid residues.
Aspect DDD310. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising of 10 amino acid residues.
Aspect DDD311. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising of 11 amino acid residues.
Aspect DDD312. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising of 12 amino acid residues.
Aspect DDD313. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising of 13 amino acid residues.
Aspect DDD314. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising of 14-17 amino acid residues.
Aspect DDD315. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising of 18-25 amino acid residues.
Aspect DDD316. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope comprising more than 25 amino acid residues.
Aspect DDD317. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope with the sequence Glycyl-Methionine.
Aspect DDD318. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope with the sequence Glycyl-Leucine.
Aspect DDD319. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope with the sequence Glycyl-Cyclohexylalanine.
Aspect DDD320. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Peptide epitope with the sequence Glycyl-Homoleucine.
Aspect DDD321. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Clostridium botulinum-specific epitope.
Aspect DDD322. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Vibrio cholera-specific epitope.
Aspect DDD323. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Tetanus-specific epitope.
Aspect DDD324. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Klebsiella-specific epitope.
Aspect DDD325. A composition of more than 2 entities, such as more than 10 entities, such as more than 100 entities, such as more than 1,000 entities, such as more than 10,000 entities, such as more than 100,000 entities, such as more than 1,000,000 entities, where each entity contains one or more copies of a unique pMHC complex mechanically linked to one or more copies of a unique DNA molecule of non-human origin, and where each entity further comprises a Dendritic cell and a Staphylococcus-specific epitope.

### Definitions

Chemically linked: Chemically linked shall solely denote covalently linked and non-covalently linked molecules. Likewise, chemical link shall solely denote covalent link and non-covalent link.

Coat: The term “coat” shall here mean the external surface of viruses or phages

Coat protein: Shall mean the proteins on the external side of the phage or virus coat.

Direct link and directly linked: That the peptide (p) of the pMHC complex is directly linked to the encoding molecule (e.g., DNA or RNA or LNA) shall mean that no other part of the pMHC complex, i.e. beta2M, HC, alpha, or beta proteins, shall be comprised within the linker connecting the peptide (p) and the encoding molecule. That the peptide and encoding molecule are directly linked shall mean that there is a direct link between the peptide and encoding molecule, and thus that neither the beta2M, HC, alpha or beta proteins, or any part of them, are comprised within the linker connecting the peptide (p) and the encoding molecule.

Encoding molecule: Shall mean any type of molecule uniquely identifying the encoded peptide (p) of the pMHC Multiplexer and can thus be a polymer or a scaffolded molecule, including DNA or RNA oligonucleotides, or artificial oligonucleotides such as LNA, PNA, etc., or peptides, branched polymers or globular or scaffolded structures.

Fluorochrome: Used interchangeably with fluorophore.

Functionally linked: Functionally linked shall mean any form of linkage that keeps two molecules within a certain maximum distance from each other, i.e. the two molecules are kept within given spatial boundaries. Functionally linked molecules include chemically linked molecules as well as mechanically linked molecules.

Mechanically linked: Mechanically linked shall for the purpose of this invention mean that the molecules in question—e.g., an encoding molecule (e.g., a DNA molecule) and a peptide that is encoded by said encoding molecule—are held in the vicinity of each other by physical rather than chemical means. Thus, if two molecules are said to be mechanically linked it is a physical boundary that keep the molecules from separating. Example physical boundaries and the functionally linked molecules are: i) two molecules, e.g., a DNA and a peptide, are both kept within the boundary of a micelle. The separation of the DNA from the peptide to a distance larger than the micelle diameter would require breakage of the micelle wall; ii) a DNA kept within the boundaries of a cell, and a peptide chemically linked (covalently or non-covalently) to the surface of said cell. The DNA and peptide are not attached to each other, but nevertheless a separation of the DNA from the peptide to a distance larger than the cell diameter would require breakage of the cell membrane/wall, or a breakage of the chemical link that keeps the peptide associated with the cell surface; iii) a DNA kept within the boundaries of a phage or virus particle, or a cell, and a peptide chemically linked (covalently or non-covalently) to the surface of said phage, virus or cell. As in the previous example, a separation of the DNA from the peptide to a distance larger than the longest dimension of the phage, virus, or cell particle would require breakage of the coat/membrane of the virus, phage or cell, or a breakage of the chemical link that keeps the peptide associated with the phage/virus/cell surface; iv) catenanes made up of two or more interlocked macrocycles, e.g., a circular DNA molecule interlocked with a circular peptide molecule.

MHC and MHC-like complexes: The term “MHC complexes” shall include MHC1 protein (also called empty MHC1), MHC2 protein (also called empty MHC2), pMHC1 complex, pMHC2 complex, MHC-like complexes such as CD1a, CD1b, CD1c, CD1D, and other MHC-like proteins.

MHC Multimer: A complex comprising more than one MHC-complex and/or peptide-MHC (MHC) complex, held together by covalent or non-covalent bonds. In one embodiment a MHC multimer comprises 2 copies (dimer), 3 copies (trimer), 4 copies (tetramer; e.g., MHC Tetramer), 5 copies, (pentamer, e.g., MHC Pentamer), 6 copies (hexamer), 7 copies (heptamer), 8 copies (octamer), 9 copies (nonamer), 10 copies (decamer), 11 copies, 12 copies, 13 copies, 14 copies, 15 copies, 16 copies, 17 copies, 18 copies, 19 copies or 20 or more copies of MHC complexes. A non-exhaustive list of possible MHC multimers illustrates the possibilities. ‘n’ indicates the number of MHC complexes comprised in the multimer: a) n=2, Dimers, multimerization can be based on IgG scaffold, streptavidin with two MHC's, coiled-coil dimerization e.g., Fos-Jun dimerization, b) n=3, Trimers, multimerization can be based on streptavidin as scaffold with three MHC's, TNFα-MHC hybrids, triplex DNA-MHC conjugates or other trimer structures, c) n=4, Tetramers, multimerization can be based on streptavidin with all four binding sites occupied by MHC molecules or based on dimeric IgA, d) n=5, Pentamers, multimerization can take place around a pentameric coil-coil structure, e) n=6, Hexamers, f) n=7, Heptamers, g) n=8-12, Octa-dodecamers, multimerization can take place using Streptactin, h) n=10, Decamers, multimerization can take place using IgM, i) 1<n<100, Dextramers, as multimerization domain polymers such as polypeptide, polysaccharides and Dextrans can be used, and j) 1<n<100, where the multimerization domain is a cell and the cell with its displayed pMHC complexes make up the MHC Multimer.

MHC multimers thus include MHC-dimers, MHC-trimers, MHC-tetramers, MHC-pentamers, MHC-hexamers, MHC Dextramers, MHC Streptamers. Example organic molecule-based multimers include functionalized cyclic structures such as benzene rings where e.g., a benzene ring is functionalized and covalently linked to e.g., three MHC complexes; example polymer-based MHC multimers include MHC-dextramers (dextran to which a number of MHC-peptide complexes are covalently or non-covalently attached).

Multimer scaffold: A multimer scaffold shall mean a scaffold to which two or more pMHC complexes can be attached, to form a MHC Multimer. Multimer scaffolds thus include IgG, Fos-Jun dimer, streptavidin (SA), pentameric coil-coil structure, streptactin, IgM, polypeptide, and dextran, or a phage, virus or cell.

N: N is a number and shall for the purpose of this invention mean the number of pMHC Multiplexers in a collection, where said collection is kept as a solution in a well or a precipitate or on dry form. N can be any number between 2 and 10¹⁵, such as 2-10, 11-100, 101-1000, 1001-10000, 10001-100000, 100001-1000000, 1000001-10000000, 10000001-100000000, or 100000001-1000000000000000.

Non-mammal: Non-mammal shall for the purpose of this invention mean a product (e.g., a protein) that is not generated in the bodies of mammalian species. As an example, non-mammal fluorochromes are thus fluorochromes that are not naturally generated in mammalian species.

Non-human: Non-human shall for the purpose of this invention mean a product (e.g., a protein) that is not generated in the bodies of human species. As an example, non-human fluorochromes are thus fluorochromes that are not naturally generated in human species.

Of non-human origin: Of non-human origin shall refer to the nucleotide sequence of a DNA molecule or a RNA molecule or amino acid sequence of a peptide molecule and shall for the purpose of this invention mean that a continuous stretch of the DNA, RNA or peptide sequence making up more than 10% of the DNA molecule, RNA molecule or peptide molecule is not a part of the human genome sequence or proteome sequence.

Peptide-receptive: Peptide-receptive MHCI complexes are derivatives of normal MHCI complexes which are able to bind peptide epitopes after the MHCI complex is formed thus generating an antigen-specific pMHCI complex.

pMHC Multiplexer: A pMHC Multiplexer is a spatially limited composition of two different molecules, an encoding molecule (i.e. an RNA or DNA molecule), and an encoded peptide, where said encoded peptide is encoded by said encoding molecule. Furthermore, the peptide is complexed to a MHC complex and thus is part of an pMHC complex. An example pMHC Multiplexer is a phage particle or a cell carrying on its surface a number of identical pMHC complexes, where the peptide of the pMHC complexes is encoded by the DNA contained within the phage particle or cell.

Sequencing: Sequencing an encoding molecule is in the present invention to be understood as determining the identity of the encoding molecule. The identity of an encoding molecule may be determined by e.g., (i) determining its mass by e.g., mass spectroscopy, (ii) determining its migration rate in a column (such as an affinity column or size separation column) or in a gel (e.g., by gel electrophoresis), (iii) determining its ability to bind to specific molecules, e.g., binding to single-stranded oligonucleotides on a microchip, (iv) determining it nucleotide sequence by traditional sequencing using a polymerase, (v) determining its nucleotide sequence by Maxam-Gilbert sequencing, or determining its identity in any other way. In some cases it may be desirable to amplify the encoding molecule before sequencing, e.g., by performing PCR on a DNA.

Spatially limited composition: A spatially limited composition shall mean a composition of two molecules where the molecules are held within a given maximum distance from each other by a functional link during the entire time the composition is applied to a given process such as e.g., a screening procedure. The spatial limitation controlling the maximum distance between the molecules of the composition can be in the form of i) a linker molecule covalently or non-covalently linking two or more molecules of the composition, where the length of the linker molecule will determine the spatial boundaries of the composition, ii) a space within a micelle that comprises all the molecules of the composition, and where the diameter of the micelle determines the spatial boundaries of the composition, iii) a cell or phage or virus particle, where all the molecules of the composition are contained within said cell or phage or virus particle or are chemically (covalently or non-covalently) linked to the membrane or coat or outer physical boundary of said cell or phage or virus particle, where the maximum distance between the molecules of the composition is determined by the diameter or length of the cell or phage or virus particle plus the length of the linker molecule that connects a molecule of the composition to the surface of that cell or phage or virus.

Tag: Used interchangeably with label. A tag is a molecule such as a fluorochrome (e.g., PE, as used in flow cytometry), a DNA oligonucleotide, or a rare element (e.g., Gd).

Tagged: Used interchangeably with labelled. A molecule is said to be tagged if a tag has been functionally linked to it.

Well: Any type of container that contains a solution whereby the contents of this solution are kept separate from the contents of a solution in another well. Wells thus include vials, tubes, flasks, containers, wells of a microtiter plate, drops including drops in an array, e.g., drops on a microchip, and micelles.

### FIGURE LEGENDS

FIG. 1. Schematic drawing of a generic pMHC Multiplexer.

The structure of a pMHC Multiplexer is indicated. The circle symbolizes the outer boundaries of the pMHC Multiplexer that keeps the encoding molecule (straight line) from being separated from the encoded peptide (wavy line); the Y-shape symbolizes the MHC complex; the wavy line and the Y-shape together symbolizes the pMHC complex; and the dashed line symbolizes the functional link between the encoding molecule and the encoded peptide (and optionally the encoded MHC complex).

FIG. 2. Preparation and structure of a pMHC Multiplexer comprising a phage particle and one or more pMHC complexes attached to the phage coat.

The preparation of a phage-based pMHC2 Multiplexer is shown. The preparation involves the following general steps:

Step 1. Cloning of multiple DNA molecules into a phagemid. The DNA molecules carry a promoter upstream of a sequence encoding the Acid Peptide fused to the 5′-end of the gIII gene, and a promoter upstream of a sequence encoding a Signal Peptide fused to the 5′end of DNA encoding peptide X. The promoter and Signal Peptide of the various DNA molecules may be the same in all or different; but the sequence of the peptide X is unique to each DNA molecules. In the figure the signal peptide is the Tat signal peptide sequence. The phagemids are then transformed into E. coli cells, and cell growth is continued, Helper Phage is added and production and cell externalization of phages into the growth medium is taking place.

Step 2. Transfer culture supernatant to new flask. From this flask, transfer aliquots containing approximately on average 0.1-0.3 phage particles to a number of microtiter wells comprising growth medium, add Helper Phage and continue growth.

Step 3. Partly lyse cells to release peptide X from the periplasm.

Step 4. Add a fusion-protein consisting of Base Peptide fused to the N-terminus of HC, i.e. the Base Peptide-HC fusion protein, and add Beta2M, to each of the wells.

Step 5. Perform a mild denaturation e.g., by heating the contents of the wells to 50-60° C., and allow to slowly cool down to 20° C., to form peptide-MHC complexes.

Step 6. Allow dimerization of Acid Peptide (on phage) with Base Peptide (on MHC1), to effectuate the display of the pMHC complex on phage.

FIG. 3. Genetic maps of phagemids pA2, pGV1 and pGV2.

(A) Phagemid pA2 is a pVIII-based display vector in which transcription of gVIII is under the control of the arabinose-inducible pBAD promoter of E. coli (Fagerlund et al., 2008). The plasmid also produces the AraC transcriptional activator to turn on pBAD promoter. (B) Genetic map of Phagemid pGV1. Phagemid pGV1 encodes “Velcro” peptide Acid that was inserted in-between the EcoRI-BgIII sites of pA2. The plasmid produces a pVIII′-Acid-′pVIII sandwich protein fusion that will be displayed at the M13 phage surface as an Acid-pVIII fusion protein upon induction of transcription of the gVIII′::Acid::′gVIII gene by the addition of arabinose to the growth medium. (C) Genetic map of Phagemid pGV2. Phagemid pGV2 is a derivative of pGV1 containing the strong, IPTG-regulated pA1/O4 promoter upstream of a Tat-signal-peptide encoding gene that has been engineered such that epitope peptide-encoding DNA fragments inserted in-between unique ApaI and BgIII restriction sites generate tat::epitope peptide “in-frame” fusions such that Tat-Epitope peptides will be exported to the periplasm of E. coli. (D) Blow-up of the cloning region of pGV2 showing the DNA sequences of the regulatory features and the unique ApAI and BgIII restriction sites used to insert peptide-encoding DNA fragments.

FIG. 4. Preparation and structure of a pMHC Multiplexer comprising a phage particle and one or more pMHC complexes covalently attached to the phage coat. The preparation of a phage-based pMHC2 Multiplexer is shown. The preparation involves the following general steps:

Step 1. Cloning of multiple DNA sequences into a phage genome. The DNA sequences carry a promoter upstream of a sequence encoding a Signal Peptide fused to peptide X. The promoter and Signal Peptide of the various DNA sequences may be the same in all or different; but the sequence of the peptide X is unique to each DNA sequence. In the figure the (Tat) signal peptide sequence is shown upstream of peptide X.

Transformation of the DNA molecules into E. coli, and cell growth, supporting production and cell externalization of phages into the growth medium.

Step 2. Transfer culture supernatant to new flask. From this flask, transfer aliquots containing on average 0.1-0.3 phage particles to a number of microtiter wells, and continue growth.

Step 3. Partly lyse cells to release peptide X from the periplasm.

Step 4. Add a molecule that comprises two reactive groups, e.g., a carboxylic acid chloride moiety and a triple bond. Allow one of the reactive groups (here: the carboxylic acid chloride) to react with a functional group on the phage coat (here: amino group), to covalently link the triple bond to the phage coat. (Steps 3 and 4 may be performed in the opposite order).

In parallel: Before or in parallel with steps 3 and 4, an empty MHC2 complex is reacted with a compound that comprises two reactive groups, e.g., a carboxylic acid chloride and an azide moiety. Allow one of the reactive groups (here: the carboxylic acid chloride) to react with a functional group on the empty MHC2 complex (here: amino group), to covalently attach the azide moiety to the empty MHC2 complex.

Step 5: Mix the azide-modified empty MHC2 complexes with the triple bond-modified phage particles, to generate a covalent bond between the MHC2 complex and the phage coat.

Step 6: Allow peptide X present in the supernatant also comprising the phages, to bind to the empty MHC complex, to form the pMHC complex. Each of the wells now contain a unique pMHC Multiplexer.

FIG. 5. Preparation and structure of a pMHC Multiplexer comprising a phage particle and one or more pMHC complexes attached to the phage coat.

The preparation of a phage-based pMHC Multiplexer is shown. The preparation involves the following general steps:

(A). A phagemid construct is shown. The phagemid DNA carries a promoter upstream of a sequence encoding the Peptide X fused to a DNA sequence encoding a flexible linker fused to the 5′-end of the gVIII gene.

(B). Preparation of pMHC Multiplexer.

Following standard protocols, a number of different phagemid (each carrying a unique DNA sequence encoding variants of peptide X) are introduced into E. coli, grown and helper phage added along with other necessary reagents to produce phage particles displaying peptide X on their pVIII coat proteins. Then the cells are pelleted by centrifugation, and the supernatant (containing the phage particles) is transferred to a new flask. Optionally, the phages are PEG precipitated and resuspended in appropriate buffer. Then empty MHC2 is added, and incubation is performed, to form peptide-X-MHC2 complexes, displayed on the phage.

(C). Using a phage construct, where the peptide X is fused to pVIII, all of the pVIII coat proteins of the phage will be fusion proteins with peptide X. Upon addition of (limiting) amount of empty MHC, peptide X-MHC2 complexes will form on a fraction of the pVIII coat proteins of the phage.

FIG. 6. Genetic map of phage display vector fth1. The general scheme of the fth1 vector is shown to scale illustrating the phage genes (opened segments), the reconstituted intergenic region (IG) and the genes introduced between the wild-type pVIII and pIII genes (stippled segments). The blow-up below shows details of the pIX to the middle of pIII is provided (not to scale). The hatched segments represent the modified wild type pVIII 3′ end (violet) and the −35 box of the pIII promoter (cyan), the engineered gVIII is shown in magenta within which a light magenta box illustrates the “stuffer” DNA fragment containing ttrpA transcriptional terminator flanked by two SfiI restriction sites. The two restriction sites allow for the insertion of epitope-encoding DNA fragments fused in frame with the signal peptide-encoding part of gVIII such that the resulting gene encodes a pVIII′-epitope peptide-‘pVIII sandwich fusion protein where pVIII’ symbolizes the signal peptide of pVIII and ′pVIII symbolizes the rest of pVIII (PMID: 11353095).

FIG. 7. Pre-enrichment of peptide X-displaying phage particles capable of binding to a MHC2.

A library (collection) of 10¹⁰ phage particles each displaying a unique peptide is exposed to empty MHC2 complexes (here: DR1) immobilized on a column, by addition of the phage particles to said column, allowing for complex formation and then washing thoroughly. The bound phages, typically in a much smaller number than applied to the column, are then recovered. These may be mixed with (non-immobilized) MHC2 (e.g., DR1), to form pMHC Multiplexers that can be used in screening processes. Alternatively, the recovered (bound) phages may be amplified by standard means and then mixed with (non-immobilized) MHC2 (e.g., DR1), to form pMHC Multiplexers that can be used in screening processes.

FIG. 8. Phagemid construction and structure of a pMHC Multiplexer comprising a phage particle and one or more pMHC complexes attached to the phage coat.

Upper panel shows the DNA sequence of the phagemid used in Example 18. The phagemid encodes the Base Peptide-unique Peptide X-Base Peptide fusion, the SP-Acid Peptide-pVIII peptide fusion, and the Acid Peptide-SSG-Acid Peptide fusion peptide.

Lower panel left, shows the packaged phage particle.

Lower panel right, shows the phage particle to which has been added Empty MHC2 complexes, to form multiple pMHC2 complexes attached to phage.

FIG. 9. Pre-enrichment of peptide X-displaying phage particles capable of binding to a MHC2.

A library (collection) of 10¹⁰ phage particles each displaying a unique peptide is exposed to empty MHC2 complexes (here: DR1) immobilized on a column, by addition of the phage particles to said column, allowing for complex formation and then washing thoroughly. The bound phages, typically in a much smaller number than applied to the column, are then recovered. These may be mixed with (non-immobilized) MHC2 (e.g., DR1), to form pMHC Multiplexers that can be used in screening processes. Alternatively, the recovered (bound) phages may be amplified by standard means and then mixed with (non-immobilized) MHC2 (e.g., DR1), to form pMHC Multiplexers that can be used in screening processes.

FIG. 10. Preparation and structure of a pMHC Multiplexer comprising a phage particle and one or more pMHC complexes attached to the phage coat.

A: Preparation and structure of a pMHC Multiplexer comprising a phage particle where the phagemid encodes a HC-pVIII fusion protein.

B: Preparation and structure of a pMHC Multiplexer comprising a phage particle where the phagemid encodes an Acid Peptide-pVIII fusion protein.

FIG. 11. Genetic maps of phagemids and production plasmids.

The genetic maps of phagemids and plasmids used in the application is shown.

FIG. 12. Preparation and structure of a pMHC Multiplexer comprising a phage particle and one or more pMHC complexes attached to the phage coat.

The figure depicts how a pentamer scaffold may be employed to display 5 pMHC complexes on the surface of phage M13. The phagemid DNA encodes a peptide fusion of, from the N-terminal, signal peptide (SP), Acid Peptide, Pentamer subunit (Psub), and pIII. The same phagemid encodes a unique peptide X, capable of binding to the MHC1 or MHC2 complex or an MHC-like complex. Upon expression of these proteins and production of phage particles by standard techniques for filamentous phage production, the result is a phage particle displaying on its surface pMHC complexes on a pentamer scaffold (see lower right corner).

FIG. 13. Preparation and structure of a pMHC Multiplexer comprising a phage particle and one or more pMHC complexes attached to the phage coat.

The figure depicts how a SP1 dodecamer protein scaffold may be employed to display 11 pMHC complexes on the surface of phage M13.

FIG. 14. Production and structure of a pMHC Multiplexer, where the pMHC Multiplexer comprises a filamentous phage.

The figure depicts how a Tetramer scaffold may be employed to display 4 pMHC complexes on the surface of phage M13. An intracellular biotin ligase couples biotin to the AP peptide fused to the Heavy Chain (HC). Assembly of the tetrameric streptavidin protein, where one subunit is linked to pIII, allows the streptavidin tetramer to become attached to the phage coat. Complexation of beta2M, HC, and peptide X, leads to formation of pMHC. Upon expression of these proteins and production of phage particles by standard techniques for filamentous phage production, the result is a phage particle displaying on its surface pMHC complexes on a tetramer scaffold (see lower right corner).

FIG. 15. Display of one or two or three MHC Tetramers on the surface of the phage particle.

Here, fusion of 1, 2 or 3 streptavidin monomers to the pIII coat protein leads to formation of a monomer, dimer or trimer, respectively, of a MHC Tetramer, displayed on phage.

FIG. 16. Multiple MHC Tetramers displayed on phage.

AP-pVIII peptide fusion leads to display of multiple MHC Tetramers on the phage coat.

FIG. 17. Preparation and structure of a pMHC Multiplexer comprising a phage particle and two or more pMHC complexes attached to the phage coat.

FIG. 18. Maps of phagemids and plasmid. (A) through (G) are derivatives of phagemid pCANTAB5. (H) shows production vector plasmid pGV4 that produces MHC1 HC allele HLA-A*01010101 and human beta-2-microglobolin upon addition of IPTG to growing E. coli cells carrying the production plasmid.

FIG. 19. Synthetic DNA sequences used to construct the gIII′-(Acid-cys-spacer peptide)₆-′gIII gene of pGV6. (A) The six varied DNA sequences encoding identical Acid-cys peptides used to construct pGV6. The top line yields the sequence of the Acid-cis Peptide while the six following lines give the DNA sequence that have been varied according to the codon usage of E. coli. The substitutions (in red) are chosen such that the codon frequencies moved from the highest to the second highest. (B) DNA sequences of the six 10 aa spacer sequences inserted between the DNA sequences encoding identical Acid-cys peptides. Base pairs varying from the preceding spacer sequence are marked in red. (C) Sequence (upper strand shown) of the synthetic DNA fragment B encoding the six identical Acid-cys peptides (red letters) and spacer sequences (black letters) inserted into pGV5 to generate display vector pGV6. SfiI and NotI restriction sites shown with upper case letters.

FIG. 20. Preparation, structure and application of a pMHC Multiplexer comprising a cell and one or more pMHC complexes attached to the cell surface.

The preparation of a cell-based pMHC Multiplexer is shown. The preparation involves the following general steps: (i) Introduction of encoding molecule (here DNA) by e.g., virus infection into a cell appropriate for the present invention, (ii) expression of encoded molecule, (iii) display of the pMHC complex. The structure of the final pMHC Multiplexer is shown (center of the figure).

Two example applications of pMHC Multiplexers are depicted.

In the first example, the pMHC Multiplexer from above is added to a blood sample, and the mixture incubated. After one or more days of incubation bispecific antibodies are added. These bi-specific antibodies have binding specificity for both (i) a certain molecule X secreted from the cell of the pMHC Multiplexer upon interaction with a T cell or from a T cell upon interaction with an antigen-presenting cell, and for (ii) a receptor of the cell of the pMHC Multiplexer. Also, a fluorescent-labelled antibody with affinity for the secreted molecule X is added. After a further incubation time of 1-3 hours pMHC Multiplexers that fluoresce are collected by flow sorting or by manually collecting these under a microscope. Finally, the DNA of the pMHC Multiplexers that were collected are sequenced.

In the second example, a pMHC Multiplexer is generated as described above, and in addition a vector has been introduced into the cell (that will become part of the pMHC Multiplexer) where the vector carries an interleukin-responsive (interleukin-activated) promoter controlling the transcription of the GFP gene. The interleukin used is an interleukin whose expression goes up upon interaction between the pMHC complexes of the pMHC Multiplexer and the T cell receptors of a T cell. After incubation the pMHC Multiplexers that have interacted productively with a T cell will fluoresce and can be collected, and finally, the encoding molecules of the pMHC Multiplexers that were collected are sequenced or identified in another way.

FIG. 21. Structure of pMHC Multiplexer, and annealing of Sense and Antisense regions.

Upper panel:

In the two A-wells, pMHC Multiplexers all carry the same unique peptide (p) of the pMHC complex. In one A-well the encoding DNA molecule carries a Sense-region; in the other A-well the encoding DNA molecule carries an Antisense region. The Sense and Antisense regions of the A-wells are complementary and Sense and Antisense regions can thus form a DNA duplex.

In the two B-wells, pMHC Multiplexers all carry the same unique peptide (p) of the pMHC complex. In one B-well the encoding DNA molecule carries a Sense-region; in the other B-well the encoding DNA molecule carries an Antisense region. The Sense and Antisense regions of the B-wells are complementary and Sense and Antisense regions can thus form a DNA duplex.

In the two C-wells, pMHC Multiplexers all carry the same unique peptide (p) of the pMHC complex. In one C-well the encoding DNA molecule carries a Sense-region; in the other C-well the encoding DNA molecule carries an Antisense region. The Sense and Antisense regions of the C-wells are complementary and Sense and Antisense regions can thus form a DNA duplex.

The DNA of the A-wells, the DNA of the B-wells or the DNA of the C-wells cannot bind the DNA of the B-wells/C-wells, A-wells/C-wells, and A-wells/B-wells, respectively.

Central Panel:

Schematic of T cells, showing TCR receptors on the cell surface.

Lower panel:

Schematic showing pMHC Multiplexers of the A-wells bound to the TCRs of a T cell, their Sense- and Antisense regions annealing to each other (Left); and pMHC Multiplexers of the C-wells bound to the TCRs of a T cell, their Sense- and Antisense regions annealing to each other (Right).

FIG. 22. Detection of TCR-binding pMHC Multiplexers.

(a). Two pMHC Multiplexers

(b). Two pMHC Multiplexers where their Sense- and Antisense regions have annealed.

(c1). The DNA has been turned into double-stranded DNA along its entire length, by extension.

(c2). The DNA has been turned into double-stranded DNA along its entire length, thereby incorporating fluorophores by extension using fluorescent-labelled dNTPs.

(c3). Primers anneal to the recently extended outer part of the double-stranded DNA

(c4). Double-stranded PCR products have been generated.

(c5). Sequencing is performed.

(c6.) Extension or PCR is performed using fluorescent-labelled dNTPs (optionally after adding outer primers, in addition to those added after step (c1).

FIG. 23. A pMHC Multiplexer may contain any number (n) of pMHC complexes.

Upper panel: Two pMHC Multiplexers, each comprising n pMHC complexes, are bound to the TCR receptors of the same T cell.

Central and Lower panel: The Sense- and Antisense DNA tags anneal, are extended using fluorescent-labelled dNTPs, are optionally taken through a flow sorting process, and the pMHC Multiplexers attached to the (isolated) cells are sequenced.

FIG. 24. A pMHC Multiplexer comprising a MHC Tetramer.

Upper panel: Two pMHC Multiplexers, each comprising 3 pMHC complexes non-covalently bound to a streptavidin protein through a biotin attached to the MHC protein, and each comprising a biotinylated DNA bound to the streptavidin and annealing to the DNA of the pMHC Multiplexer bound to the neighboring TCR receptor of the same T cell.

Lower panel: The annealed duplex has been extended

FIG. 25. Structure of example pMHC Multiplexers of the invention.

A: Two pMHC complexes are shown. The left pMHC2 complex comprises a peptide X, encoded by the directly linked DNA X; the right pMHC2 complex comprises a peptide Y, encoded by the directly linked DNA Y.

B: A DNA-tagged pMHC dimer

C: A DNA-tagged pMHC Tetramer

D: A DNA-tagged pMHC Pentamer

E: A DNA-tagged pMHC Dextramer

F: A DNA-tagged pMHC Tetramer, where the peptides (p) of the pMHC complexes are pair-wise linked G: A DNA-tagged pMHC Tetramer, where all the peptides (p) are linked to each other.

FIG. 26. A DNA-tagged pMHC Multimer where the DNA oligonucleotide functions as both encoding molecule and multimer scaffold.

Each peptide is directly linked to two DNA oligonucleotides, and the bridging DNA is double-stranded.

FIG. 27. A stabilizing configuration of interlinked peptides (p).

A: Four peptides are interlinked, and each of the four peptides bind to a separate MHC complex, thereby increasing the total affinity of the peptides for the MHC Tetramer complex.

B: Five peptides are interlinked, and each of the five peptides bind to a separate MHC complex, thereby increasing the total affinity of the peptides for the MHC Dextramer complex.

FIG. 28. Production and structure of a dodecamer Acid Peptide-displaying scaffold comprising a SP1 protein.

A: A schematic of the dodecameric SP1 protein, showing the 12 N-termini protruding inwards from the ring-shaped molecule.

B: A schematic of a recombinant SP1 protein, where a flexible linker peptide has been fused to the N-terminus of the SP1 subunit.

C: A schematic of a recombinant SP1 protein comprising a flexible linker at the N-termini of the 12 subunits, as described in (B), where additionally the Acid Peptide has been fused at the N-terminus of the flexible linker.

FIG. 29. SP1-based pMHC Multimer.

A: A schematic of a recombinant SP1 protein, where the flexible linker described in (FIG. 28, B.) and the HC peptide has been fused to the N-terminus of the SP1 subunit.

B: A schematic of the recombinant SP1 protein, where beta2M, peptide (p), as well as the HC-flexible linker-SP1 subunit fusion peptide described in (A), all have complexed to form a SP1-based pMHC Multimer carrying 12 pMHC complexes.

FIG. 30. SP1-based pMHC Multimer comprising Acid-Base dimer.

A schematic of a recombinant SP1 protein comprising 12 subunits, each of which are fused to the Acid Peptide, and where each of the Acid Peptides is dimerized with a Base Peptide that is attached to a pMHC complex, thereby forming a SP1-based pMHC Multimer comprising 12 pMHC complexes.

FIG. 31. SP1-based pMHC Multiplexer comprising Acid-Base dimer.

A schematic of a recombinant SP1 protein comprising 12 subunits, each of which are fused to the Acid Peptide, and where each of the Acid Peptides is dimerized with a Base Peptide. Eleven of these Base Peptides are attached to a pMHC complex; one Base Peptide is attached to a DNA. Thus, this is a pMHC Multiplexer comprising the SP1 protein, and carrying a DNA tag.

### EXAMPLES

### Example 1. Display of pMHC Class 2 Complexes by Phage Particles Via pVIII Encoding Peptide X on a Phagemid

The method generates a M13-based Multiplexer displaying pMHC2 complexes attached to pVIII by Acid-Base dimerization. The method is divided into X Steps as delineated in the following.
Step 1. Phagemid vector pA2 (FIG. 3, A) is chosen to generate the acid::gVIII gene fusion.
Step 2. Synthesize a double stranded DNA fragment A:
5′-CCCCCGAATTC GGCGCGGCGCAGCTGGAAAAAGAACTGCAGGCGCTGGAAAAAGAAAACGCGCAGCTGGAATGGGAACTGCAGGCGCTG GAAAAAGAACTGGCGCAGGGCGGCTGCCCGGCGGGCGCGGGGGATCCCCCCC (upper strand sequence) that encodes Acid-GAAQLEKELQALEKENAQLEWELQALEKELAQGGCPAGA (O'Shea et al., 1993). The Acid-encoding DNA sequence was deduced by “reverse translation” of the Acid peptide sequence using E. coli codon usage.
Step 3. Cleave DNA fragment A from with EcoRI and BamHI restriction enzymes according to standard procedure (Kay et al., 1996).
Step 4. Cleave Phagemid pA2 DNA with EcoRI and BgIII restriction enzymes and purification of the vector DNA fragment according to standard procedure (Kay et al., 1996).
Step 5. Ligate DNA fragment A with the vector fragment of phagemid pA2 using standard procedure (BamHI and BgIII has compatible cohesive ends).
Step 6. Transform the ligated DNA to E. coli TG1 [Δ(lac-pro), supE, thi, hsdΔ5/F′ [traD36, proAB, laclq, lacZΔM15] competent cells, purification of phagemid DNA and DNA sequencing of the resulting recombinant phagemid by standard procedure (Kay et al., 1996, op sit.) The resulting phagemid, called pGV1, encodes a pBAD::gVIII_::Acid-p1::gVIII gene sandwich fusion as shown in FIG. 3, B.
Step 7. Construct phagemid pGV2. Synthesis of double stranded DNA fragment B 5′-GCATGC TTTATCAAAAAGAGTGTTGACTTGTGAGCGGATAACAATGATACTTAGATTCATCGAGAGGGACACGGCGAAAAATAAGG AGGAAAAAAAAATGGCGAATAACGATCTCTTTCAGGCATCACGTCGGCGTTTTCTGGCACAACTCGGCGGCTTAACCGTC GCCGGGATGCTGGGGCCCTCATTGTTAACGCCGCGACGTGCGACTGCG AGATCT GCATGC (upper strand) encoding
   (i) the synthetic LacI-regulated pA1/O4 promoter (Lanzer and Bujard, 1988);
   (ii) the SD8 Shine & Dalgarno sequence (Ringquist et al., 1992);
   (iii) the twin arginine signal peptide (Tat) of TorA of E. coli (Thomas et al., 2001) engineered, by silent mutation, to contain an ApaI restriction cloning site (GGGCCC) unique in pGV2 and appropriately designed such that epitope-encoding DNA fragments generate in-frame translational fusions between the Tat signal peptide and an epitope-encoding reading frame when inserted in-between the unique ApaI-BgIII restriction sites. The Tat peptide of TorA is the most efficient of 5 Tat signal-peptides that have been tested for their ability to export heterologous peptides (Matos et al., 2012).
Step 8. Cleave DNA fragment B with SphI restriction enzyme according to standard procedure (Kay et al., 1996).
Step 9. Cleave Phagemid pGV1 DNA with SphI and purification of the vector DNA fragment according to standard procedure (Kay et al., 1996).
Step 10. Ligate DNA fragment B with the pGV1 cleaved with SphI according to standard procedure (Kay et al., 1996).
Step 11. Transform the ligated DNA to electrocompetent cells of E. coli K-12 strain TG1, purification of pGV2 phagemid DNA and full DNA sequencing of two chosen recombinant clones were according to standard laboratory procedures (Kay et al., 1996).
Step 12. Prepare DNA of Phagemid pGV2 was prepared according to standard plasmid preparation procedure using E. coli K-12 strain TG1 (Kay et al., 1996).
Step 13. Prepare Helper Phage M13K07 (encoding kanamycin resistance) according to standard protocol (Kay et al., 1996).
Step 14. Ready vector DNA. DNA of phagemid pGV2 is cleaved with ApaI and BgIII, purified away from the small ApaI-BgIII DNA fragment of the vector and stored at −20 C according to standard laboratory procedure (Kay et al., 1996).
Step 15. Synthesis of DNA oligonucleotides encoding epitopes and control peptide. Synthesis of ApaI-BgIII DNA fragments encoding epitope peptides, such as e.g., a CMV positive control epitope, a negative control peptide or test peptides to be displayed in the phage Multiplexer. The DNA fragments are designed such that the epitope-encoding reading frames are “in-frame” with the tat signal peptide-encoding reading frame of pGV2 (FIG. 3, C).

The control epitope peptides
CMV99: MSIYVYALPLKMLNI,
CMV105 (ALPLKMLNIPSINVH),
HA307-19 (PKYVKQNTLKLAT)
   are appropriate positive control peptides that bind to MHC II (DR-1) complexes while
VMC99 (INLMKLPLAYVYISM)
   is an appropriate negative control peptide that has the inverse sequence of that of CMV99.

Synthesize double-stranded DNA ApaI-BgIII fragment CMV99-GV2 gggccc tca ttg tta acg ccg cga cgt geg act geg ATG AGC ATC TAT GTG TAT GCG CTG CCG CTG AAA ATG CTG AAC ATT UAA UAA AGATCT (upper sequence) here called DNA fragment C. Fragment C consists of (i) an ApaI site at its very 5′-end; (ii) a reading frame that encodes the last 12 aa of the TorA signal peptide (DNA sequence shown in lower case) fused to a reading frame encoding CMV99 (italic); (iii) two UAA UAA ochre stop codons (in bold) and (iv) a BgIII restriction site. When ligated with the ApaI-BgIII DNA fragment of the pGV2 vector, DNA fragment C will generate an in frame fusion that, when translated by the bacterial ribosomes, will lead to the production of a TorA signal-peptide fused to CMV99. The TAT transport system of E. coli cells of TG1 will transport the TorA-CMV99 peptide to the periplasm. During said transport, the TorA signal-peptide will be cleaved off such that CMV99 peptide ends up in the periplasm of the bacterial cell (i.e. outside the cell membrane but inside the outer membrane) thus facilitating further release of the peptide to the extracellular medium.

Synthesize, by a similar standard approach DNA fragments encoding TorA′-CMV105 TorA′-HA307-19 and TorA′-VMC99 peptides that generate in-frame fusions with the torA signal peptide-encoding reading frame of pGV2 to generate TorA′-peptide X fusions.

Synthesize, by a similar standard approach, DNA fragments encoding test epitope peptides that generate in-frame fusions with the torA signal peptide-encoding reading frame of pGV2 to generate TorA′-peptide X peptide fusions.
Step 16. Insert by ligation the synthetic DNA fragments generated in Step 15 with DNA of phagemid vector pGV2 cleaved with ApaI-BgIII according to standard cloning protocols (Kay et al., 1996).
Step 17. Prepare electrocompetent cells and transform E. coli strain TG1 with ligated DNA according to standard procedure (Kay et al., 1996).
Step 18. Generate pure phage populations. The TG1 cells and phage particles in the growth medium from step 17 are separated by centrifugation, by pelleting the cells. The supernatant comprising the phage particles is transferred to a new tube. This solution of phage particles is then diluted, and small aliquots of diluted phage particles are added to an appropriate number (here: 5000) of microtiter wells (or tubes or flasks) comprising growing TG1 cultures, where the aliquots each comprise approximately on average 0.1-0.3 phage particles. To each micro titer well (or tube or flask) is added Helper Phage, to initiate phage production and secretion. Optionally, arabinose is added in order to increase transcription of the gene encoding Acid-pIII fusion protein and optionally, IPTG is added to increase transcription of the genes encoding signal peptide-peptide X fusions.
Step 19. Allow for the release of peptides to the extracellular growth medium. After an appropriate period of growth (e.g., 4-5 h), the E. coli cells generated in Step 18 will produce phage particles that are secreted into the growth medium. Likewise, the signal peptide TorA-peptide X fusion peptides are produced, the TorA signal peptide is cleaved off and the resulting C-terminal end (peptide X) is transported into the periplasm. The E. coli cells are exposed to (partial) lysis, permeabilizing the outer membrane and resulting in release of the peptides from the periplasm into the growth medium.
Step 20. The cells and cell debris are pelleted by centrifugation and the supernatant of each of the micro titer wells (or tubes or flasks) is transferred to another micro titer well (or tube or flask).
Step 21. Optionally, protease inhibitor(s) and/or nuclease inhibitors are added-before, during or after (partial) lysis.
Step 22. A Solution containing a Base Peptide-Alfa Chain (Base-AC) fusion protein in complex with Beta-Chain (thus generating a Base-tagged MHC2 (DR-1) complex) are transferred into each of the micro titer wells to a final concentration preferably lower than the estimated concentration of peptide X in the individual well. Here, a Base Peptide variant is used that carries a cysteine, capable of forming a disulfide bond with Acid Peptide upon their complexation.
Step 23. The temperature is decreased to approximately 20° C., to allow complex formation between peptide X and the Base-tagged MHC2 (DR-1) complex, to potentially form the peptide X-MHC2 complex.
Step 24. Optionally, a redox buffer is added, to first reduce the cysteine in each of the Acid Peptide and the Base Peptide.
Step 25. Optionally, the oxidation/reduction status of the buffer is adjusted to allow disulfide bond formation between the cysteines of the Acid Peptide and the Base Peptide. As a result, the peptide X-MHC2 complex becomes covalently linked to the Acid Peptide-pVIII fusion protein of the phage coat.

### Example 2. Generation of 1000 Different pMHC Multiplexers, Comprising Phage Particles that Carry Multiple MHC2 Complexes Covalently Attached to the Phage Coat

In this example it is described how click chemistry can be used to covalently attach multiple pMHC2 and pMHC1 complexes to the phage coat.
Step a. A large number of DNA molecules (here: 1000 different DNA molecules), each of which carries a promoter upstream of a DNA sequence encoding a signal peptide (SP) in reading frame (and N-terminal to) a short peptide X, are cloned into the M13 genome. Each of the 1000 different DNA molecules carry a unique sequence encoding a unique peptide X. Thus, in (FIG. 4), “peptide X” represents a number of (here: 1000) different peptide X sequences, where each phage DNA carries a unique DNA sequence encoding the peptide X. Optionally, the expression of peptide X is very efficient in E. coli, leading to high amounts of peptide X in a cell containing the phage DNA. Optionally, the expression level of peptide X can be controlled by extracellular changes such as the addition of an activator molecule or other changes in the growth medium. The peptide can be any peptide of any length, e.g., a peptide comprising 7-11 amino acid residues.
Step b. The phage DNA constructs encoding a total of 1000 different peptide X sequences is introduced (e.g., by transformation) into an E. coli strain that is capable of supporting phage infection and amplification, and cell growth is performed for several cell generations. Phage particles are now produced and are being secreted out into the supernatant, i.e., into the growth medium.
Step c. The growth medium comprising the E. coli cells and phage particles are centrifuged, to pellet the cells. The supernatant comprising the phage particles is transferred to a new tube. Optionally, the phage particles are PEG-precipitated, and the pellet resuspended in an appropriate buffer. The solution of phage particles is then diluted, and small aliquots of diluted phage particles are added to a number (here: 5000) of microtiter wells (or tubes or flasks) comprising growing E. coli cultures (i.e., E. coli strains capable of supporting phage infection and amplification), where the aliquots each comprise on average 0.1-0.3 phage particles. Growth is continued.
Step d. Optionally, activator molecules are added in order to increase the expression of the signal peptide-peptide X fusion peptides.
Step e. Continued growth of E. coli and resultant phage particle production and secretion into the growth medium is performed. Likewise, the signal peptide (SP)-peptide X fusion peptide is expressed, the signal peptide is cleaved off and the resulting C-terminal end (peptide X) is transported into the periplasm.
Step f. The E. coli cells are exposed to (partial) lysis, permeabilizing the outer membrane, and resulting in release of the peptides from the periplasm into the growth medium. Many methods for the permeabilization of the outer membrane exist, including treatment with MAC13243 (described in Scientific Reports 7, Article number: 17629 (2017), title: “Increasing the permeability of Escherichia coli using MAC13243”) or by cold osmotic shock (described in BioTechniques 66: 171-178 (2019), title: “A robust fractionation method for protein subcellular localization studies in Escherichia coli”).
Step g. The (partially) lysed cells and cell debris are pelleted by centrifugation and the supernatant of each of the microtiter wells (or tubes or flasks) is transferred to another microtiter well (or tube or flask).
Step h. Optionally, protease inhibitor(s) and/or nuclease inhibitors are added-before, during or after (partial) lysis.
Step i. A compound comprising a terminal triple bond and a carboxylic acid chloride is added to the wells, at a concentration that leads to reaction of the acid chloride with 0.1%-1% of the amino groups of the phage coat proteins (and peptide X). After reaction about 0.1%-1% of the amino groups of the phage coat protein will thus have been modified and now carry a triple bond.
Step j. In a separate reaction, the HC protein, beta2M protein or an empty MHC1 complex, and/or alpha protein, beta protein or an empty MHC2 complex is modified with an azide moiety. The modification is done in a way that results in approximately 1 modification per protein. The modifying reaction may be performed by the addition of a compound that comprises a carboxylic acid chloride and a terminal azide moiety, at a concentration that results in the covalent linkage of on average one azide moiety per protein, by reaction of the acid chloride with an amino group on the protein. Alternatively, instead of the compound comprising an carboxylic acid chloride, it may comprise an amine or a thiol or some other reactive group capable of reacting with an amino acid side chain, and the coupling reaction must then be performed accordingly.
Step k. The azide-modified HC protein, beta2M protein or empty MHC1 complex, and/or the azide-modified alpha protein, beta protein or empty MHC2 complex of step (j) is added to the wells of (i).
Step l. The triple bond (covalently attached to a phage coat protein) and the azide moiety (covalently attached to a protein of the pMHC complex) will react, thereby covalently linking the pMHC complex to the phage coat protein.
Step m. If azide-modified HC protein, beta2M protein or empty MHC1 complex was added in the above step, the wells may now be exposed to (partly) denaturing conditions, e.g., heat (50-70° C.), urea or other denaturing conditions. Then the conditions are brought back to non-denaturing conditions, to allow refolding of the proteins and formation of pMHC complexes. If only empty MHC2 or empty MHC1 complex was added in the above step, the peptide may simply bind to the empty MHC2 complex without prior denaturation, thereby forming the pMHC complex.

The resulting product of the process described by the above steps (a-m), is thus approximately 1000 unique pMHC Multiplexers.

### Example 3. Display of pMHC Class 2 Complexes Using Epitope Peptide Fused to pVIII

A method to generate a phage-based Multiplexer based on the M13 phage vector fth1 that contains a wild type and an engineered version of gVIII is described. The engineered gVIII is used to express an epitope-pVIII fusion protein on the phage surface that binds empty MHC2. This method has the advantage of multi-valance display on the phage surface by coat protein pVIII. The Method is designed such that epitope copy-number can vary from 2 to >2000 and obviates the need for the use of helper phage. The Method can directly be adopted to other M13 phage vectors carrying two copies of gVIII. In the following, epitope, epitope peptide and peptide X are used synonymously.

The method is divided into a number of steps, as follows.
Step 1. Description of M13 phage vector for display via pVIII. The genetically stable phage vector fth1 (FIG. 6) contains two copies of gVIII. The engineered gVIII allows for the insertion of peptide X-encoding DNA fragments such that pVIII′-epitope peptide-linker peptide (SSGSSG)-′pVIII sandwich fusion proteins are produced after transcription and translation within the bacterial cell, and after signal peptide cleavage (pVIII′) and transport and incorporation into the phage surface, displayed as an epitope-SSGSSG-pVIII fusion on the phage surface. Transcription of gVIII is driven by Ptac allowing for control of transcription of fusion genes by the level of an inducer in the growth medium thereby controlling the final valence of the displayed peptide.
Step 2. Preparation of vector fth1 RF DNA according to standard procedure as described in the laboratory manual Phage Display of Peptides and Proteins by Kay, B. C., Winter, J., and McCafferty, J. (1996). In the following the term standard procedure refers to this handbook.
Step 3. Ready fth1 vector DNA for cloning of epitope-encoding DNA fragments. Vector DNA (100 μg) is cleaved with restriction enzyme SfiI, dephosphorylated by alkaline phosphatase to remove 5′ phosphates, and stored at −20 C for later use.
Step 4. Synthesis of DNA oligonucleotides encoding positive and negative control epitopes and library epitopes. All fusion proteins are constructed with a domain-breaking linker peptide SSGSSG between the epitope peptide and the ′pVIII part of the fusions, resulting in pVIII′-epitope-SSGSSG-′pVIII fusion proteins.

### Displayed MHC (HLA-DR1) Epitope Peptides:

CMV99: MSIYVYALPLKMLNI;
CMV105: ALPLKMLNIPSINVH,
HA307-19: PKYVKQNTLKLAT;
EBNA1 515-527: TSLYNLRRGTALA

### Control Negative “Nonsense” Epitope Peptide:

VMC99: INLMKLPLAYVYISM

The five said peptides as well as SSGSSG are reverse translated using E. coli codon usage and the resulting five dsDNA fragments encoding the five peptides as well as the SSGSSG peptide are synthesized, with appropriate overhangs compatible with the SfiI restriction sites of the fth1 vector.

In the construction of larger MHC Multiplexer libraries encoding, DNA fragments encoding the epitope peptide and SSGSSG are inserted as a mixture into the fth1 vector by the same procedure.
Step 5. Ligation of epitope-encoding DNA fragments. Ligation of the epitope-encoding DNA fragments generated in step 4 with the linearized fth1 vector fragment generated in Step 3 is done according to standard procedure. The phage constructs generate epitope-pVIII chimeric proteins expressed concomitantly with wild type pVIII and expressed on the phage surface of a valence of ca. 150/2700 copies per phage. To vary the valence of the Multiplexer, the displayed number of chimeric proteins is increased by increasing the concentration of IPTG-inducer in the medium.
Step 6. Preparation of electrocompetent E. coli cells and transformation is according to standard procedure.
Step 7. Amplification and harvest of library phage. Amplify the library by growing the culture from Step 6 above with aeration at 37° C. for 8-10 hr. Harvest the library phage as soon after the culture has reached stationary phase, as expressed peptides may be susceptible to proteolysis. The E. coli cells and phage particles in the growth medium are separated by centrifugation, by pelleting the cells. The supernatant comprising the phage particles is transferred to a new tube. PEG precipitation of the phages is performed, the supernatant is carefully removed, and the phage pellet is resuspended.
Step 8. Add empty MHC class II complexes to phage mixture displaying epitopes. Empty MHC2 complexes are added, and the peptides X displayed on the phage coat protein pVIII are allowed to bind to the empty MHC2, thereby forming the pMHC2 complex, displayed on phage. Thereby a large number of unique pMHC Multiplexers are prepared, each comprising a unique peptide X.

### Example 4. Pre-Enrichment of Phage Particles Displaying Peptide X on a Phage Coat Protein, and Capable of Binding to an Empty pMHC2 Complex, Thereby Forming a pMHC Multiplexer Complex

A library (collection) of 10¹⁰ phage particles each displaying a unique peptide is exposed to empty MHC2 complexes (here: DR1) immobilized on a column, by addition of the phage particles to said column, allowing for complex formation and then washing thoroughly (see FIG. 7). The bound phages, typically in a much smaller number than applied to the column, are then recovered. These may be mixed with (non-immobilized) MHC2 (e.g., DR1), to form pMHC Multiplexers that can be used in screening processes, as described above. Alternatively, the recovered (bound) phages may be amplified by standard means and then mixed with (non-immobilized) MHC2 (e.g., DR1), to form pMHC Multiplexers that can be used in screening processes, as described above.

### Example 5. This Example Describes the Generation of a pMHC1 Multiplexer Based on an Acid Peptide-pVIII Protein Fusion Outlined in (FIG. 10, B)

Step 1. Phagemid vector pA2 (FIG. 11) was chosen to generate the acid::gVIII fusion gene

Construction of a pA2-derived phagemid (pGV1, FIG. 11) producing an Acid peptide-pVIII fusion protein. The construction of pGV1 is described the next 5 steps.
Step 2. Synthesis of a double stranded DNA fragment A 5′-CCCCCGAATTC GGCGCGGCGCAGCTGGAAAAAGAACTGCAGGCGCTGGAAAAAGAAAACGCGCAGCTGGAATGGGAACTGCAGGCGCTG GAAAAAGAACTGGCGCAGGGCGGCTGCCCGGCGGGCGCGGGGGATCCCCCCC (upper strand sequence) that encodes Acid-cys peptide GAAQLEKELQALEKENAQLEWELQALEKELAQGGCPAGA (O'Shea et al., 1993). The Acid-cys peptide-encoding DNA sequence was generated by “reverse translation” of the Acid-cys Peptide sequence using E. coli codon usage.
Step 3. Cleavage of DNA fragment A with EcoRI and BamHI restriction enzymes.
Step 4. Cleavage of Phagemid pA2 DNA with EcoRI and BgIII restriction enzymes and purification of the vector DNA fragment by standard procedure.
Step 5. Ligation of DNA fragment A to the vector fragment of phagemid pA2 using standard procedure (BamHI and BgIII has compatible cohesive ends).
Step 6. Transformation of the ligated DNA to E. coli TG1 competent cells, purification of phagemid pGV1 DNA and sequencing of the resulting recombinant phagemid DNA by standard procedure. The resulting phagemid, called pGV1, encodes a pBAD::gVIII′::Acid::′gVIII gene fusion that can be activated by the addition of arabinose to the growth medium (FIG. 11).
Step 7. Insertion of DNA fragment B that encodes a multiple cloning region downstream of the synthetic, IPTG regulated pA1/O4 promoter and a ribosome binding site, in the unique SphI site (pos. 3917 to 3922) resulting in phagemid pGV3 (FIG. 11). The ribosome binding site (S & D) is positioned at a unique NcoI site that can be used to insert peptide-encoding DNA fragments such that the peptide-encoding reading frame will be translated by the bacterial ribosomes. A number of unique restriction sites useful for the directed cloning of DNA fragments into pGV3 is located downstream of the NcoI site (i.e. BgIII, HindIII, XhoI, StuI).

Upper strand of DNA fragment B: 5′-CCCCC GCATGC TTTATCAAAAAGAGTGTTGACTTGTGAGCGGATAACAATGATACTTAGATTCATCGAGAGGGACACGGCGAAAAATAAGG AGGAAAAAA CCATGG AGATCT AAGCTT CTCGAG AGGCCT GCATGC CCCCC
Step 8. Construction of a plasmid producing a Base-cys-MHC1 HC fusion protein and human beta-2-microglobulin to generate an MHC1 complex that can dimerize with Acid-cys-pVIII. The vector plasmid chosen for said production was pNDM220 that has an origin of replication compatible with phagemid pA2 and that contains the synthetic pA1/O4 promoter activated by IPTG (FIG. 11).

The extracellular domain of Human MHC1 HC allele HLA-A*01:01:01:01 is a 307 aa peptide with the following sequence:

MAVMAPRTLL LLLSGALALT QTWAGSHSMR
YFFTSVSRPG RGEPRFIAVG YVDDTQFVRF
DSDAASQKME PRAPWIEQEG PEYWDQETRN
MKAHSQTDRA NLGTLRGYYN QSEDGSHTIQ
IMYGCDVGPD GRFLRGYRQD AYDGKDYIAL
NEDLRSWTAA DMAAQITKRK WEAVHAAEQR
RVYLEGRCVD GLRRYLENGK ETLQRTDPPK
THMTHHPISD HEATLRCWAL GFYPAEITLT
WQRDGEDQTQ DTELVETRPA GDGTFQKWAA
VVVPSGEEQR YTCHVQHEGL PKPLTLRWEL
SSQPTIP

MHC1 HC allele HLA-A*01:01:01:01 is encoded by the following DNA sequence generated be reverse translation using E. coli standard codon usage:

5′-atggcggtgatggcgccgcgcaccctgctgctgctgctgagc
ggcgcgctggcgctgacccagacctggggggcagccatagcatgc
gctatttttttaccagcgtgagccgcccgggccgcggcgaaccgc
gctttattgcggtgggctatgtggatgatacccagtttgtgcgct
ttgatagcgatgcggcgagccagaaaatggaaccgcgcgcgccgt
ggattgaacaggaaggcccggaatattgggatcaggaaacccgca
acatgaaagcgcatagccagaccgatcgcgcgaacctgggcaccc
tgcgcggctattataaccagagcgaagatggcagccataccattc
agattatgtatggctgcgatgtgggcccggatggccgctttctgc
gcggctatcgccaggatgcgtatgatggcaaagattatattgcgc
tgaacgaagatctgcgcagctggaccgcggcggatatggcggcgc
agattaccaaacgcaaatgggaagcggtgcatgcggcggaacagc
gccgcgtgtatctggaaggccgctgcgtggatggcctgcgccgct
atctggaaaacggcaaagaaaccctgcagcgcaccgatccgccga
aaacccatatgacccatcatccgattagcgatcatgaagcgaccc
tgcgctgctgggcgctgggcttttatccggcggaaattaccctga
cctggcagcgcgatggcgaagatcagacccaggataccgaactgg
tggaaacccgcccggcgggcgatggcacctttcagaaatgggcgg
cggtggtggtgccgagcggcgaagaacagcgctatacctgccatg
tgcagcatgaaggcctgccgaaaccgctgaccctgcgctgggaac

tgagcagccagccgaccattccg taataa

while the Base-cys peptide GAAQLKKKLQALKKKNAQLKWKLQALKKKLAQGGCPAGA reverse translates to

5′-ggcgcggcgcagctgaaaaaaaaactgcaggcgctgaaaaaa
aaaaacgcgcagctgaaatggaaactgcaggcgctgaaaaaaaaa
ctggcgcagggcggctgcccggcgggcgcg

while the linker peptide GSSGSS reverse translates to

5′-ggcagcagcggcagcagc

while the extracellular form of human beta-2-microglobulin has the sequence IQRTPKIQVYSRHPAENGKSNFLNCYVSGFHPSDIEVDLLKNGERIEKVEHSDLSFSKDW SFYLLYYTEFTPTEKDEYACRVNHVTLSQPKIVKWDRDM that reverse translates to

5′-attcagcgcaccccgaaaattcaggtgtatagccgccatccg
gcggaaaacggcaaaagcaactttctgaactgctatgtgagcggc
tttcatccgagcgatattgaagtggatctgctgaaaaacggcgaa
cgcattgaaaaagtggaacatagcgatctgagctttagcaaagat
tggagcttttatctgctgtattataccgaatttaccccgaccgaa
aaagatgaatatgcgtgccgcgtgaaccatgtgaccctgagccag
ccgaaaattgtgaaatgggatcgcgatatg

Generated from the above DNA sequences, the upper strand of synthetic double-stranded DNA fragment C encodes BamHI, Shine & Dalgarno sequence, start-codon, Base-cys MHC1 HC (HLA-A*01:01:01:01), beta-2-microglobulin, two ochre stop-codons and EcoRI as follows:

5′-CCCCC GATCC GGAGGAAAAAAAAATG ggcgcggcgcagc
tgaaaaaaaaactgcaggcgctgaaaaaaaaaaacgcgcagctga
aatggaaactgcaggcgctgaaaaaaaaactggcgcagggcggct
gcccggcgggcgcg ggcagcagcggcagcagcatggcggtgatg
gcgccgcgcaccctgctgctgctgctgagcggcgcgctggcgctg
acccagacctggggggcagccatagcatgcgctatttttttacca
gcgtgagccgcccgggccgcggcgaaccgcgctttattgcggtgg
gctatgtggatgatacccagtttgtgcgctttgatagcgatgcgg
cgagccagaaaatggaaccgcgcgcgccgtggattgaacaggaag
gcccggaatattgggatcaggaaacccgcaacatgaaagcgcata
gccagaccgatcgcgcgaacctgggcaccctgcgcggctattata
accagagcgaagatggcagccataccattcagattatgtatggct
gcgatgtgggcccggatggccgctttctgcgcggctatcgccagg
atgcgtatgatggcaaagattatattgcgctgaacgaagatctgc
gcagctggaccgcggcggatatggcggcgcagattaccaaacgca
aatgggaagcggtgcatgcggcggaacagcgccgcgtgtatctgg
aaggccgctgcgtggatggcctgcgccgctatctggaaaacggca
aagaaaccctgcagcgcaccgatccgccgaaaacccatatgaccc
atcatccgattagcgatcatgaagcgaccctgcgctgctgggcgc
tgggcttttatccggcggaaattaccctgacctggcagcgcgatg
gcgaagatcagacccaggataccgaactggtggaaacccgcccgg
cgggcgatggcacctttcagaaatgggcggcggtggtggtgccga
gcggcgaagaacagcgctatacctgccatgtgcagcatgaaggcc
tgccgaaaccgctgaccctgcgctgggaactgagcagccagccga
ccattccg taataa
GGAGGAAAAAAAAATG
attcagcgcaccccgaaaattcaggtgtatagccgccatccggcg
gaaaacggcaaaagcaactttctgaactgctatgtgagcggcttt
catccgagcgatattgaagtggatctgctgaaaaacggcgaacgc
attgaaaaagtggaacatagcgatctgagctttagcaaagattgg
agcttttatctgctgtattataccgaatttaccccgaccgaaaaa
gatgaatatgcgtgccgcgtgaaccatgtgaccctgagccagccg
aaaattgtgaaatgggatcgcgatatg TAATAAGAATTC CCCC
C.

DNA Fragment C is inserted into expression vector pNDM220 to generate pGV4 (FIG. 11). E. coli cells carrying pGV3 and pGV4 can produce Acid-cys-pVIII fusion peptide, Base-cys-MHC1-HC fusion peptide and beta-2-microglobulin when both arabinose and IPTG are added to the growth medium.
Step 9. Cloning of three HLA-A*01:01:01:01 restricted plus one negative epitope peptide-encoding sequences into phagemid pGV3.

### HLA-A*0101 Binds

P54922|ADPRH_HUMAN: FVEENLQHWSY that
reverse translates to
5′-tttgtggaagaaaacctgcagcattggagctat
Q15119|PDK2_HUMAN: VSEVVKDAY that
reverse translates to
5′-gtgagcgaagtggtgaaagatgcgtat
Q04446|GLGB_HUMAN: LTDDDLLRY that
reverse translates to
5′-ctgaccgatgatgatctgctgcgctat

while

P54922_REV: YSWHQLNEEVF that reverse
translates to
5′-tatagctggcatcagctgaacgaagaagtgttt

is a negative nonsense peptide with the reverse sequence of that of P54922.

The following 4 synthetic DNA fragments flanked by BgIII and XhoI sites will produce the 4 desired peptides when ligated into pGV3 downstream of the IPTG-activated pA1/O4 promoter:

P54922-DNA:
5′-CCCCC AGATCTGGAGGAAAAAAAAATG tttgtgga
agaaaacctgcagcattggagctatTAATAA CTCGAG
CCCCC
Q15119:
5′-CCCCC AGATCT GGAGGAAAAAAAAATG gtgagcg
aagtggtgaaagatgcgtatTAATAA CTCGAG CCCCC
Q04446:
5′-CCCCC AGATCT GGAGGAAAAAAAAATG ctgaccg
atgatgatctgctgcgctatTAATAA CTCGAG CCCCC
P54922_REV:
5′-CCCCC AGATCT GGAGGAAAAAAAAATG tatagc
tggcatcagctgaacgaagaagtgtttTAATAA CTCGAG
CCCCC

These synthetic DNA fragments are cleaved with BgIII and XhoI and inserted into pGV3 resulting in phagemids pP54922, pQ15119, p Q04446 and pP54922_REV (FIG. 11). Similarly, a library of 1000 or more DNA fragments encoding epitope peptide X can be synthesized by an analogous approach to generate a library of phagemids.
Step 10. Transformation of production plasmid pGV4 into E. coli TG1 cells.
Step 11. Transformation of phagemids pP54922, pQ15119, pQ04446 and pP54922_REV into TG1/pGV4 cells thus generating three positive control strains and one negative control strain:
TG1/pGV4/pP54922
TG1/pGV4/pQ15119
TG1/pGV4/pQ04446
TG1/pGV4/pP54922_REV

Similarly, a library of up to 1000 test strains TG1/pGV4/pGV3-peptideX-encoding producing 1000 or more different X peptides (FIG. 10) are constructed.
Step 12. Bacterial cultures of the 4 control strains and two bacterial cultures with the phagemid library are grown in for 1-2 in nutrient medium before helper phage M13KO7, IPTG and arabinose are added and cell growth continued for several hours to allow for the production of phages displaying pMHC1 complexes via pVIII on their surface.
Step 13. The E. coli cells and phage particles in the growth medium are separated by centrifugation, by pelleting the cells. The supernatant comprising the phage particles is transferred to a new tube. For a further purification step, the phage particles are PEG precipitated and resuspended in 2×YT medium and finally filtered through a 0.45 μm filter before storage at 4° C.

### Example 6. Preparation of 1000 Unique MHC Multiplexers, Each Comprising a Pentamer Scaffold Comprising Up to 5 pMHC Complexes

In this example the filamentous phage M13 is used, see (FIG. 12), to generate a large number of different pMHC Multiplexers, each of which carry one or more copies of the tryptophan-zipper pentamer (see PNAS, vol. 101, p. 16156-16161). In this example, 1000 unique pMHC Multiplexers are produced:
Step a. An M13-derived phagemid is constructed carrying a promoter controlling a DNA sequence encoding a fusion peptide consisting of, from the N-terminal end, a signal peptide (SP), Acid peptide, tryptophan-zipper pentamer subunit (Psub), and pIII coat protein. Further, the phagemid carries a promoter controlling the transcription of a unique peptide X. 1000 unique plasmid constructs are generated: all phagemids carry the same peptide fusion SP-Acid-Psub-pIII, but each phagemid carries a unique peptide X (see FIG. 12). The gIII fusion gene is under the control of a promoter whose expression can be modified by the addition of a molecule to the growth medium. One such molecule may be IPTG, where the promoter is under the regulatory control of IPTG. Optionally, the production of peptide X is very efficient, leading to high amounts of peptide X in a bacterial cell carrying the phagemid. Optionally, the expression level of peptide X can be controlled by extracellular changes such as the addition of a molecule that stimulates transcription or translation of the gene encoding peptide X.
Step b. On the same phagemid, or on another vector in the same cell, is a DNA sequence encoding a Base Peptide-HC fusion protein, the beta2M protein, and an Acid Peptide-Pentamer subunit fusion.
Step c. The DNA constructs of step (a) and (b) are introduced (e.g., by transformation) into an E. coli strain that is capable of supporting phage infection and amplification, and cell growth is performed for several cell generations. Then Helper Phage is added, and phage amplification is performed for several cell generations. Phage particles, carrying one or more copies of the Pentamer that carries up to five pMHC complexes each, are produced and are being secreted out of the cell, i.e. into the growth medium. Optionally, protease inhibitor(s) and/or nuclease inhibitors are added.
Step d. The E. coli cells and phage particles in the growth medium are separated by centrifugation, by pelleting the cells. The supernatant comprising the phage particles is transferred to a new tube.
Step e. Optionally, the phage particles are PEG precipitated.

The resulting product of the process described by the above steps (a-e), is 1000 unique pMHC Multiplexers.

### Example 7. Preparation of SP1-Based pMHC Multiplexers

In this example, the filamentous phage M13 is used, to generate a large number of different pMHC Multiplexers, each of which carry one or more copies of the SP1 dodecamer protein scaffold. In this example, 1000 unique pMHC Multiplexers are produced:
Step a. A M13-derived phagemid is constructed carrying a promoter controlling a DNA sequence encoding a fusion peptide consisting of, from the N-terminal end, a Signal Peptide (SP), Acid peptide, and pIII coat protein. Further, the phagemid carries a promoter controlling the transcription of a unique peptide X. 1000 unique plasmid constructs are generated: all phagemids carry the same peptide fusion Signal Peptide (SP), Acid peptide, and pIII coat protein, but each phagemid carries a unique peptide X (see FIG. 13). The gIII fusion gene is under the control of a promoter whose expression can be modified by the addition of a molecule to the growth medium. One such molecule may be IPTG, where the promoter is under the regulatory control of IPTG. Optionally, the production of peptide X is very efficient, leading to high amounts of peptide X in a bacterial cell carrying the phagemid. Optionally, the expression level of peptide X can be controlled by extracellular changes such as the addition of a molecule that stimulates transcription or translation of the gene encoding peptide X.
Step b. On the same phagemid, or on another vector in the same cell, is a DNA sequence encoding an Acid Peptide-HC fusion protein, the beta2M protein, and a fusion peptide of, from the N-terminal, Base Peptide-Flexible linker peptide (e.g., SSGSSG)-SP1 subunit peptide.
Step c. The DNA constructs of step (a) and (b) are introduced (e.g., by transformation) into an E. coli strain that is capable of supporting phage infection and amplification, and cell growth is performed for several cell generations. Then Helper Phage is added, and phage amplification is performed for several cell generations. Phage particles, carrying one or more copies of the Sp1 protein scaffold that carries up to 12 pMHC complexes each, are produced and are being secreted out of the cell, i.e. into the growth medium. Optionally, protease inhibitor(s) and/or nuclease inhibitors are added.
Step d. The E. coli cells and phage particles in the growth medium are separated by centrifugation, by pelleting the cells. The supernatant comprising the phage particles is transferred to a new tube.
Step e. Optionally, the phage particles are PEG precipitated.

The resulting product of the process described by the above steps (a-e), is 1000 unique pMHC Multiplexers, each of which carries up to 12 pMHC complexes.

### Example 8. Display of One, Two or Three MHC Tetramers on the Phage Coat

In this example it is described how one may prepare phages that display a total of up to 12 pMHC complexes on one pIII coat protein of phage M13 (see FIG. 15).

The DNA constructs described in Example 22, and depicted in (FIG. 14), are prepared as described, except that the streptavidin (in the SP-SA-pIII peptide fusion on the phagemid) is replaced with a SA-short linker-SA peptide fusion, or a SA-short linker-SA-short linker-SA peptide fusion, to give, respectively, SP-SA-short linker-SA-pIII and SP-SA-short linker-SA-short linker-SA-pIII. This may result in the formation of, respectively, phage particles displaying 2 pMHC complexes (FIG. 15, B) and phage particles displaying 3 pMHC complexes (FIG. 15, C). The short linker can be e.g., 2-5 amino acid residues, e.g., the dipeptide SS.

### Example 9. Polyvalent Display of pMHC Complexes on pVIII of Phage M13

In this example it is described how multiple pMHC Tetramers can be displayed on the surface of phage M13 (see FIG. 16).
Step 1. A collection of e.g., 1000 phagemid molecules with DNA encoding a fusion peptide consisting of, from the N-terminus, SP-AP-pVIII, and each encoding a unique peptide X, both under the control of a promoter whose activity can be adjusted by cell-external stimuli or effector molecules, are prepared.
Step 2. On a separate vector, compatible with the phagemid, DNA encoding beta2M and a fusion of AP and HC, is introduced.
Step 3. The phagemids and vector of steps 1 and 2 are introduced into an E. coli strain by e.g., transformation, and growth is carried out for several generations.
Step 4. Helper Phage and other necessary components for efficient phage particle production are added, and growth and phage particle production is carried out for several generations.
Step 5. Produced phage particles are secreted into the growth medium by the bacteria. The phages and cells are separated by centrifugation, and the supernatant comprising the phage particles is transferred to another flask. Optionally, the phages are PEG precipitated and resuspended in a physiological buffer.

The phage particles displaying multiple pMHC complexes on their surfaces are pMHC Multiplexers: the DNA contained in the phage particle is mechanically linked to the pMHC complexes on the phage particle coat; and the peptide (p) of the pMHC complex is encoded by the DNA inside the phage.

In this example immediately above, the pIII coat protein may be used instead of the pVIII coat protein of M13.

### Example 10. Generation and Structure of a pMHC1 Multiplexer Based on an Acid-Cys Peptide-pIII Protein Fusion

This example describes the generation of a pMHC1 multiplexer based on an Acid-cys peptide-pIII protein fusion outlined in (FIG. 17) that is displayed on the phage surface via fusion to pIII. In particular, the method uses a repeat of Acid-cys peptides fused to pIII to increase the valency of the multiplexer.
Step 1. The example uses phagemid vector pCANTAB5 (FIG. 18, A) that encodes an engineered version of M13 gene gIII used here to generate gIII gene fusions that produce Acid-cys peptide-pIII fusion proteins.
Step 2. DNA fragment A 5′-GACGTC TTTATCAAAAAGAGTGTTGACITGTGAGCGGATAACAATGATACITAGATTCATCGAGAGGGACACG AGATCT GGTACC GTCGAC ATCTACATTACTGAGCTAAT AACAGGCCTGCTGGTAATCGCAGGCCUUUUUATTT GACGTC that encodes
   (i) The IPTG regulated pA1/O4 promoter,
   (ii) A multiple cloning region containing BgIII, KpnI and SalI restriction sites used to insert peptide-encoding DNA fragments that also include a ribosome recognition site upstream of the peptide-encoding reading frame,
   (iii) The tR2 transcriptional terminator of lambda to prevent transcriptional interference is inserted into the unique AatII site (pos. 70) of pCANTAB5 (FIG. 18, A), resulting in pGV5 (FIG. 18, B). This phagemid allows for transcription of epitope peptide-encoding reading frames inserted into the multiple cloning region downstream of the IPTG-regulated promoter (e.g., between BgIII and SalI restriction sites).
Step 3. Construction of a gIII variant gene of pCANTAB5 encoding a pIII′-[Acid-cys peptide-spacer]₆-′pIII fusion protein where [Acid-cys peptide-spacer]6 is a six times repeated Acid-cys peptide in which the Acid-cys peptide sequences are separated by 10 aa peptide spacers. pIII′ is the pIII signal peptide encoded by pCANTAB5 and ′pIII is the C-terminal, mature part of the pIII protein displayed at the tail of the phage particle.

The Acid-cys peptide is a 39 aa sequence N-GAAQLEKELQALEKENAQLEWELQALEKELAQGGCPAGA while the six spacer sequences (1 to 6) inserted between the Acid-cys repeats are SSGSSGSSPG, SSGSSGSPSG, SSGSSGPSSG, SSGSSPGSSG, SSGSPSGSSG, SSGPSSGSSG. The spacer sequences vary to avoid DNA repeats that can cause genetic instability.

The DNA sequences encoding the six identical Acid-cys sequences (1-6) are varied in accordance with the degeneracy of the genetic code such that the encoded peptides are identical but the DNA sequences encoding the Acid-cys sequences are different to reduce genetic instability due to their repetition. The six varied DNA sequences encoding Acid-cys peptides are shown in (FIG. 19, A) and the DNA sequences of the six 10 aa spacer sequences in (FIG. 19, B).

The sequence of synthetic DNA fragment B (FIG. 19, C) encoding SfiI-[Acid-cys peptide-spacer]6-pIII fusion-NotI is cleaved with SfiI and NotI and inserted into pGV5, resulting in pGV6 (FIG. 18, C). Phagemid pGV6 produces a pIII′-[Acid-cys peptide-spacer]6-′pIII fusion protein upon addition of IPTG to growing cells of an E. coli strain carrying pGV6.
Step 4. Construction of a plasmid producing a Base-cys-MHC1 HC fusion protein and human beta-2-microglobulin to generate an MHC1 complex that can dimerize with Acid-cys-pVIII. The vector plasmid chosen for said production was pNDM220 that has an origin of replication compatible with phagemid pA2 and that contains the synthetic pA1/O4 promoter activated by IPTG (FIG. 18, H).

The extracellular domain of Human MHC1 HC allele HLA-A*01:01:01:01 is a 307 aa peptide with the following sequence:

MAVMAPRTLL LLLSGALALT QTWAGSHSMR
YFFTSVSRPG RGEPRFIAVG YVDDTQFVRF
DSDAASQKME PRAPWIEQEG PEYWDQETRN
MKAHSQTDRA NLGTLRGYYN QSEDGSHTIQ
IMYGCDVGPD GRFLRGYRQD AYDGKDYIAL
NEDLRSWTAA DMAAQITKRK WEAVHAAEQR
RVYLEGRCVD GLRRYLENGK ETLQRTDPPK
THMTHHPISD HEATLRCWAL GFYPAEITLT
WQRDGEDQTQ DTELVETRPA GDGTFQKWAA
VVVPSGEEQR YTCHVQHEGL PKPLTLRWEL
SSQPTIP

MHC1 HC allele HLA-A*01:01:01:01 is encoded by the following DNA sequence generated be reverse translation using E. coli standard codon usage:

5′-atggcggtgatggcgccgcgcaccctgctgctgctgctgagc
ggcgcgctggcgctgacccagacctggggggcagccatagcatgc
gctatttttttaccagcgtgagccgcccgggccgcggcgaaccgc
gctttattgcggtgggctatgtggatgatacccagtttgtgcgct
ttgatagcgatgcggcgagccagaaaatggaaccgcgcgcgccgt
ggattgaacaggaaggcccggaatattgggatcaggaaacccgca
acatgaaagcgcatagccagaccgatcgcgcgaacctgggcaccc
tgcgcggctattataaccagagcgaagatggcagccataccattc
agattatgtatggctgcgatgtgggcccggatggccgctttctgc
gcggctatcgccaggatgcgtatgatggcaaagattatattgcgc
tgaacgaagatctgcgcagctggaccgcggcggatatggcggcgc
agattaccaaacgcaaatgggaagcggtgcatgcggcggaacagc
gccgcgtgtatctggaaggccgctgcgtggatggcctgcgccgct
atctggaaaacggcaaagaaaccctgcagcgcaccgatccgccga
aaacccatatgacccatcatccgattagcgatcatgaagcgaccc
tgcgctgctgggcgctgggcttttatccggcggaaattaccctga
cctggcagcgcgatggcgaagatcagacccaggataccgaactgg
tggaaacccgcccggcgggcgatggcacctttcagaaatgggcgg
cggtggtggtgccgagcggcgaagaacagcgctatacctgccatg
tgcagcatgaaggcctgccgaaaccgctgaccctgcgctgggaac
tgagcagccagccgaccattccg taataa

while the Base-cys peptide GAAQLKKKLQALKKKNAQLKWKLQALKKKLAQGGCPAGA reverse translates to

5′-ggcgcggcgcagctgaaaaaaaaactgcaggcgctgaaaaaa
aaaaacgcgcagctgaaatggaaactgcaggcgctgaaaaaaaaa
ctggcgcagggcggctgcccggcgggcgcg

while the linker peptide GSSGSS reverse translates to 5′-ggcagcagcggcagcagc while the extracellular form of human beta-2-microglobulin has the sequence IQRTPKIQVYSRHPAENGKSNFLNCYVSGFHPSDIEVDLLKNGERIEKVEHSDLSFSKDW SFYLLYYTEFTPTEKDEYACRVNHVTLSQPKIVKWDRDM that reverse translates to

5′-attcagcgcaccccgaaaattcaggtgtatagccgccatccg
gcggaaaacggcaaaagcaactttctgaactgctatgtgagcggc
tttcatccgagcgatattgaagtggatctgctgaaaaacggcgaa
cgcattgaaaaagtggaacatagcgatctgagctttagcaaagat
tggagcttttatctgctgtattataccgaatttaccccgaccgaa
aaagatgaatatgcgtgccgcgtgaaccatgtgaccctgagccag
ccgaaaattgtgaaatgggatcgcgatatg

Generated from the above DNA sequences, the upper strand of synthetic double-stranded DNA fragment C encodes BamHI, Shine & Dalgarno sequence, start-codon, Base-cys MHC1 HC (HLA-A*01:01:01:01), beta-2-microglobulin, two ochre stop-codons and EcoRI as follows:

5′-CCCCC GATCC GGAGGAAAAAAAAATG ggcgcggcgcagc
tgaaaaaaaaactgcaggcgctgaaaaaaaaaaacgcgcagctga
aatggaaactgcaggcgctgaaaaaaaaactggcgcagggcggct
gcccggcgggcgcg ggcagcagcggcagcagcatggcggtgatg
gcgccgcgcaccctgctgctgctgctgagcggcgcgctggcgctg
acccagacctgggcgggcagccatagcatgcgctatttttttacc
agcgtgagccgcccgggccgcggcgaaccgcgctttattgcggtg
ggctatgtggatgatacccagtttgtgcgctttgatagcgatgcg
gcgagccagaaaatggaaccgcgcgcgccgtggattgaacaggaa
ggcccggaatattgggatcaggaaacccgcaacatgaaagcgcat
agccagaccgatcgcgcgaacctgggcaccctgcgcggctattat
aaccagagcgaagatggcagccataccattcagattatgtatggc
tgcgatgtgggcccggatggccgctttctgcgcggctatcgccag
gatgcgtatgatggcaaagattatattgcgctgaacgaagatctg
cgcagctggaccgcggcggatatggcggcgcagattaccaaacgc
aaatgggaagcggtgcatgcggcggaacagcgccgcgtgtatctg
gaaggccgctgcgtggatggcctgcgccgctatctggaaaacggc
aaagaaaccctgcagcgcaccgatccgccgaaaacccatatgacc
catcatccgattagcgatcatgaagcgaccctgcgctgctgggcg
ctgggcttttatccggcggaaattaccctgacctggcagcgcgat
ggcgaagatcagacccaggataccgaactggtggaaacccgcccg
gcgggcgatggcacctttcagaaatgggcggcggtggtggtgccg
agcggcgaagaacagcgctatacctgccatgtgcagcatgaaggc
ctgccgaaaccgctgaccctgcgctgggaactgagcagccagccg
accattccg taataa
GGAGGAAAAAAAAATG
attcagcgcaccccgaaaattcaggtgtatagccgccatccggcg
gaaaacggcaaaagcaactttctgaactgctatgtgagcggcttt
catccgagcgatattgaagtggatctgctgaaaaacggcgaacgc
attgaaaaagtggaacatagcgatctgagctttagcaaagattgg
agcttttatctgctgtattataccgaatttaccccgaccgaaaaa
gatgaatatgcgtgccgcgtgaaccatgtgaccctgagccagccg
aaaattgtgaaatgggatcgcgatatg TAATAAGAATTC CCCC
C.

DNA Fragment C is inserted into expression vector pNDM220 to generate pGV4 (FIG. 18, H). E. coli cells carrying pGV3 and pGV4 can produce Acid-cys-pVIII fusion peptide, Base-cys-MHC1-HC fusion peptide and beta-2-microglobulin when both arabinose and IPTG are added to the growth medium.
Step 5. Cloning of three HLA-A*01:01:01:01 restricted plus one negative epitope peptide-encoding sequences into phagemid pGV6.

The MHC1 complex with the allele HLA-A*0101 binds

P54922|ADPRH_HUMAN: FVEENLQHWSY that
reverse translates to
5'-tttgtggaagaaaacctgcagcattggagctat
Q15119|PDK2_HUMAN:
VSEVVKDAY that reverse translates to
5'-gtgagcgaagtggtgaaagatgcgtat
Q04446|GLGB_HUMAN:
LTDDDLLRY that reverse translates to
5'-ctgaccgatgatgatctgctgcgctat

while

P54922_REV:
YSWHQLNEEVF that reverse translatesto
5'-tatagctggcatcagctgaacgaagaagtgttt

is a negative nonsense peptide with the reverse sequence of that of P54922 that does not bind to HLA-A*0101. The following 4 synthetic DNA fragments flanked by BgIII and SalI sites will produce the 4 desired peptides when ligated into pGV6 downstream of the IPTG-activated pA1/O4 promoter:

P54922-DNA:
5'-CCCCC AGATCT GGAGGAAAAAAAAATG tttgtg
gaagaaaacctgcagcattggagctatTAATAA GTCGA
CCCCCC
Q15119:
5'-CCCCC AGATCT GGAGGAAAAAAAAATG gtgagc
gaagtggtgaaagatgcgtatTAATAA GTCGAC CCCC
C
Q04446:
5'-CCCCC AGATCT GGAGGAAAAAAAAATG ctgacc
gatgatgatctgctgcgctatTAATAA GTCGAC CCCC
C
P54922_REV:
5'-CCCCC AGATCT GGAGGAAAAAAAAATG tatagc
tggcatcagctgaacgaagaagtgtttTAATAA GTCGA
CCCCCC

These synthetic DNA fragments are cleaved with BgIII and SalI and inserted into pGV6, resulting in phagemids pGV6-P54922, pGV6-Q15119, pGV6-Q04446 and p GV6-P54922_REV (FIG. 18, D, E, F, G).

Similarly, a library of 1000 or more DNA fragments encoding epitope peptide X may synthesized by an analogous approach to generate a library of phagemids producing a library of 1000 or more epitope peptides.
Step 6. Transformation of production plasmid pGV4 into E. coli TG1 cells to establish strain TG1/pGV4.
Step 7. Transformation of phagemids pGV6-P54922, p GV6-Q15119, p GV6-Q04446 and p GV6-P54922_REV into cells of strain TG1/pGV4 thus generating three positive control strains and one negative control strain:
TG1/pGV4/pGV6-P54922
TG1/pGV4/pGV6-Q15119
TG1/pGV4/pGV6-Q04446
TG1/pGV4/pGV6-P54922_REV

Similarly, a library of up to 1000 test strains TG1/pGV4/pGV6-peptideX-encoding producing 1000 or more different X peptides (FIG. 17) are constructed.
Step 8. Bacterial cultures of the 4 control strains and two bacterial cultures with the phagemid library are grown in for 1-2 in nutrient medium before helper phage M13KO7, IPTG and arabinose are added and cell growth continued for several hours to allow for the production of phages displaying pMHC1 complexes via pVIII on their surface.
Step 9. The E. coli cells and phage particles in the growth medium are separated by centrifugation, by pelleting the cells. The supernatant comprising the phage particles is transferred to a new tube. For a further purification step, the phage particles are PEG precipitated and resuspended in 2×YT medium and finally filtered through a 0.45 μm filter before storage at 4° C.

### Example 11. Production of a pMHC Multiplexer Comprising a Dendritic Cell, Encoding mRNA and DNA, and Displaying pMHC Complexes

DNA oligonucleotides (prepared by synthetic chemistry) or DNA fragments (prepared from vectors of e.g., E. coli) encoding 100 different peptides of length 8-30 amino acids residues, is cloned into an appropriate vector, e.g., pGEM-eGFP vector, upstream of the GFP gene, in a way so that transcription of the 100 genes encoding said 100 peptides is under control of a T7 bacteriophage promoter—i.e. 100 different vector constructs are made, each of which carries a unique DNA sequence encoding one of the 100 different peptides.

Double-stranded DNA of each of the 100 vectors is placed in a well of a microtiter plate, and in vitro transcription is performed using T7 RNA polymerase, according to standard procedures.

Peripheral blood mononuclear cells (PBMC) are used as a source of DC precursors and are isolated from leukapheresis products of healthy donors. DC are generated according to standard procedures. Optionally, the DC yield may be improved by employing one or more of the following: (i) RPMI 1640 medium supplemented with heat-inactivated plasma, penicillin, streptomycin, L-asparagine, L-glutamine, human GM-CSF, and IL-4. Incubation is carried out for 6 days. On days 2 and 4, medium containing the cytokine amount of day 0 is added. On day 6 the DCs are harvested, to be used for electroporation below.

To each of the 100 wells above, each containing multiple copies of a unique mRNA encoding a unique peptide, is added particles, e.g., metal particles such as gold particles, and incubation is carried out. After incubation the contents of the 100 wells are pooled and DCs (preparation described above) are electroporated with the pooled solution. Alternatively, the contents of each of the 100 wells are electroporated into 100 aliquots of the DCs (preparation described above), and then the electroporated DCs are pooled after electroporation. Electroporation is done using standard protocols and instruments; e.g., EQUIBIO EasyjecT Plus apparatus from EQUIBIO, UK. Thereafter, the cells are resuspended in prewarmed RPMI 1640 medium supplemented with AP, PS, AAG, GM-CSF, and IL-4. After 4 hours, the DCs are matured using a maturation cocktail comprising IL-6, IL-1B, TNF-α, and PGE₂. After a further 1-4 days, the DCs are harvested.

The resulting cells are pMHC Multiplexers.

### Example 12. Construction of a Pair of pMHC Multiplexers, Both Comprising the Same Peptide (p) of the pMHC Complex, and One pMHC Multiplexer Carrying a DNA with a Sense Region and the Other pMHC Multiplexer Carrying a DNA with an Antisense Region, where the Sense- and Antisense Regions are Complementary

Step 1. A biotinylated pMHC complex (e.g., biotinylated at the N-terminus of HC) is provided.
Step 2. Two biotinylated DNA oligonucleotides are provided; these are called DNA-Sense and DNA-Antisense, and have the following sequences (where the biotin molecule is linked to the 5′-end, as indicated with “biotin”, and where the Sense and Antisense regions have underlining and double underlining, respectively):

DNA-Sense:
biotin-5'-AGCGGCTGGATATGCGGATGGTCGCGTAG-3'
DNA-Antisense:
3'-GCATCCGTACGTAGCTGAGGCTACGTTGACGCG-5'-biotin

Step 3. To a microtiter well (Well A1) is added Streptavidin protein, pMHC complex from step (1), and DNA-Sense from step (2), at a molar ratio of 1:3:1, and appropriate buffer is added (i.e., a buffer capable of supporting binding of pMHC complex to TCR, and capable of supporting polymerase extension of DNA).

To a microtiter well (Well A2) is added Streptavidin protein, pMHC complex from step (1), and DNA-Sense from step (2), at a molar ratio of 1:3:1, and appropriate buffer is added (i.e., a buffer capable of supporting binding of pMHC complex to TCR, and capable of supporting polymerase extension of DNA).

Incubation is carried out for 30 minutes at RT.

The resulting product is a pair of pMHC Multiplexers carrying identical pMHC complexes, with one pMHC Multiplexer comprising a Sense-DNA and the other pMHC Multiplexer comprising an Anti-sense DNA, where the Sense and Antisense DNA are complementary sequences able of annealing to each other.

A schematic of the pair of pMHC Multiplexers is shown in (FIG. 24, Upper panel).

### Example 13. Production of Four pMHC Multiplexers

Here, the synthesis and final structure of a library consisting of 4 pMHC Multiplexers is described. The pMHC Multiplexers are applicable to the screening processes described above.
Step 1. The following peptides are synthesized by standard solid phase synthesis. All peptides comprise an terminal amino group as indicated:

Peptide 1:
MSIYVYALPLKMLNI-NH₂
Peptide 2:
ALPLKMLNIPSINVH-NH₂
Peptide 3:
PKYVKQNTLKLAT-NH₂
Peptide 4:
INLMKLPLAYVYISM-NH₂

Step 2. The following 8 DNA oligonucleotides are synthesized. The constant A sequence is underlined, and the constant B sequence has double underlining. Sequence A and sequence B are complementary. All 8 DNA oligonucleotides carry a N-hydroxy-succinimide ester at their 5′-terminus.

Sequence 1A:
5′-ATGGCAGTAGTCGAAAGTAC-3′
Sequence 1B:
3′-GATGATCATCAGCTTACATG-5′
Sequence 2A:
5′-GTGTGAGTCGTGCGTTCTCG-3′
Sequence 2B:
3′-GCGATTCAGCACGCAGTGCC-5′
Sequence 3A:
5′-CCAATGTGACCGTAGCGTAG-3′
Sequence 3B:
3′-CGAATCACTGGCATCGAGTC-5′
Sequence 4A:
5′-CACAGGCTGAGCTGAGGACA-3′
Sequence 4B:
3′-CACGTCGACTCGACTCCAGA-5′

Step 3. The peptides and DNA oligonucleotides are added to each of four wells, as indicated:
Well 1: Peptide 1, Sequence 1A, Sequence 1B
Well 2: Peptide 2, Sequence 2A, Sequence 2B
Well 3: Peptide 3, Sequence 3A, Sequence 3B
Well 4: Peptide 4, Sequence 4A, Sequence 4B
Step 4. A phosphate buffer pH 8 is added to all the wells, and the mixture is incubated at 50 degrees Celsius for 1 hour, to allow the amino groups of the peptides to react with the N-hydroxy-succinimide groups of the DNA oligonucleotides, thereby forming an amide bond between the encoding DNA and the encoded peptide (p).
Step 5. The temperature is decreased to 20 degrees Celsius, and biotinylated empty MHC2 complex and streptavidin is added, and incubation continued for 1 hour.

The resulting pMHC Multiplexer comprises a DNA-tagged pMHC2 Multimer, where the DNA tag encodes the peptide (p) of the pMHC2 complexes. This pMHC Multiplexer may be used in the screening or isolation procedures described above.

### Example 14. Preparation of a SP1-Based pMHC Multiplexer

This example describes the formation of a pMHC1 multiplexer using the dodecameric SP1 multimer protein and Acid Peptide-Base Peptide multimerization.
Step 1. Construction of a production plasmid capable of producing large amounts of a linker-SP1 fusion protein. A synthetic DNA fragment encoding, from the 5′-end, the following elements:
   (i) A BamHI restriction site
   (ii) A Shine & Dalgarno element and an ATG start-codon to allow for binding of mRNA to the ribosome
   (iii) The 12 amino acid Linker Peptide LP1: SSGPSSGSSGPS (12 amino acids)
   (iv) The 108 amino acid Aspen SP1 protein:

MATRTPKLVKHTLLTRFKDEITREQIDNYINDYTNLLD
LIPSMKSFNWGTDLGMESAELNRGYTHAFESTFESKSG
LQEYLDSAALAAFAEGFLPTLSQRLVIDYFLY
(108 amino acids)

(v) Two ochre stop-codons (TAA TAA)
(vi) The transcriptional terminator tR2 from phage lambda
(vii) An EcoRI restriction site.
   is inserted into the high-copy number vector plasmid pUC19 downstream of the Plac promoter of said vector plasmid. The resulting production plasmid is called pGV10. Plasmid pGV10 is transformed into E. coli strain C41 that that has been developed to produce large amounts of recombinant proteins. The L1-SP1 peptide is purified according to standard protocols.
Step 2. Construction of a production plasmid capable of producing large amounts of a Base-MHC1 HC fusion protein. A synthetic DNA fragment encoding, from the 5′-end, the following elements:
   (i) A BamHI restriction site
   (ii) A Shine & Dalgarno element and an ATG start-codon to allow for binding of mRNA to the ribosome
   (iii) The 33 amino acid Base Peptide: AQLKKKLQALKKKNAQLKWKLQALKKKLAQGGC
   (iv) The extracellular fragment of MHC1 HC (allele A*01:01):

MAVMAPRTLLLLLSGALALTQTWAGSHSMRYFFTSVSRPGRGEPRF
IAVGYVDDTQFVRFDSDAASQKMEPRAPWIEQEGPEYWDQETRNM
KAHSQTDRANLGTLRGYYNQSEDGSHTIQIMYGCDVGPDGRFLRG
YRQDAYDGKDYIALNEDLRSWTAADMAAQITKRKWEAVHAAEQRR
VYLEGRCVDGLRRYLENGKETLQRTDPPKTHMTHHPISDHEATLR
CWALGFYPAEITLTWQRDGEDQTQDTELVETRPAGDGTFQKWAAV
VVPSGEEQRYTCHVQHEGLPKPLTLRWELSSQPTIP

(v) The transcriptional terminator tR2 from phage lambda
(vi) An EcoRI restriction site.
   is inserted into the high-copy number vector plasmid pUC19 downstream of the Plac promoter of said vector plasmid, using standard protocols. The resulting production plasmid is called pGV11. Plasmid pGV11 is transformed into E. coli strain C41 and the Base peptide-HC fusion peptide is purified according to standard protocols.
Step 3. The Base Peptide-HC peptide from step 2 is added to each of 1000 micro titer wells, in a buffer appropriate for formation of a pMHC complex. Then beta2M peptide, expressed and purified according to standard procedures, is added. Finally, a unique peptide of 7-11 amino acid residues is added to each of the 1000 micro titer wells. The solutions are first denatured and then renatured, e.g., by heating followed by cooling, or by addition of 6-8 M urea followed by removal of the urea by e.g., dialysis, or by any other means that allow denaturation and renaturation. Upon renaturation, the Base Peptide-pMHC1 complex will have formed. Thus, each micro titer well now comprises a unique Base Peptide-pMHC complex. In 4 wells, instead of adding a unique peptide, the following control peptides are added:

P54922|ADPRH_HUMAN:
FVEENLQHWSY
Q15119|PDK2_HUMAN:
VSEVVKDAY
Q04446|GLGB_HUMAN:
LTDDDLLRY

that are three positive control peptides restricted by MHC1 allele A*01:01 while

P54922_REV:
YSWHQLNEEVF

is a negative control peptide.
Step 4. In a separate experiment, Base Peptide carrying a reactive group X at one terminus (e.g., a triple bond at the N-terminus), is added to each of 1000 (empty) microtiter wells, and appropriate buffer is added. Then a unique DNA molecule, carrying a reactive group Y capable of reacting with X (e.g., an azide, capable of reacting with a triple bond) is added to each of the 1000 microtiter wells. The reactive groups X and Y are brought to react, thereby forming in each well a unique Base Peptide-DNA conjugate.
Step 5. Each of the 1000 solutions of a unique Base Peptide-pMHC complex from step (3) is added to one of the 1000 solutions of a unique Base Peptide-DNA conjugate from step (4), at a molar ratio of
   “Base Peptide-pMHC”: “Base Peptide-DNA conjugate”
      of approximately 10:1. Each of the resulting 1000 solutions now comprise both a unique Base Peptide-pMHC and a unique Base Peptide-DNA conjugate.
Step 6. To each of the 1000 solutions of step (e) is added the purified Acid Peptide-SP1 fusion protein from step (a), and the 1000 solutions are incubated for 30 min. After dimerization of Acid Peptide and Base Peptide, multiple copies of a unique pMHC Multiplexer will have formed in each of the 1000 solutions, where the unique pMHC Multiplexer comprises a SP1 scaffold displaying a unique pMHC complex in multiple copies, and where the identity of the peptide (p) of the pMHC complex is encoded by the DNA molecule attached to the same pMHC Multiplexer, see (FIG. 31).

Thus, 1000 unique pMHC Multiplexers have been formed by the above steps (1) to (6). The 1000 unique pMHC Multiplexers may be used for the detection or the isolation of antigen-specific T cells.

### Example 15. Preparation of pMHC Multiplexers by Extracellular pMHC Formation and Attachment to Phage by Dimerization

This example describes the preparation of a spatial array (microtiter plate wells) of phage clones (each individual clone placed in the same well as the free peptide that its DNA encodes), followed by addition of MHC complex, formation of pMHC complex, and attachment of the formed pMHC complex to the phage particle through the formation of an acid-base dimer. The result is a pMHC Multiplexer where the pMHC complex is attached to the phage particle (comprising the DNA) that encodes the peptide.

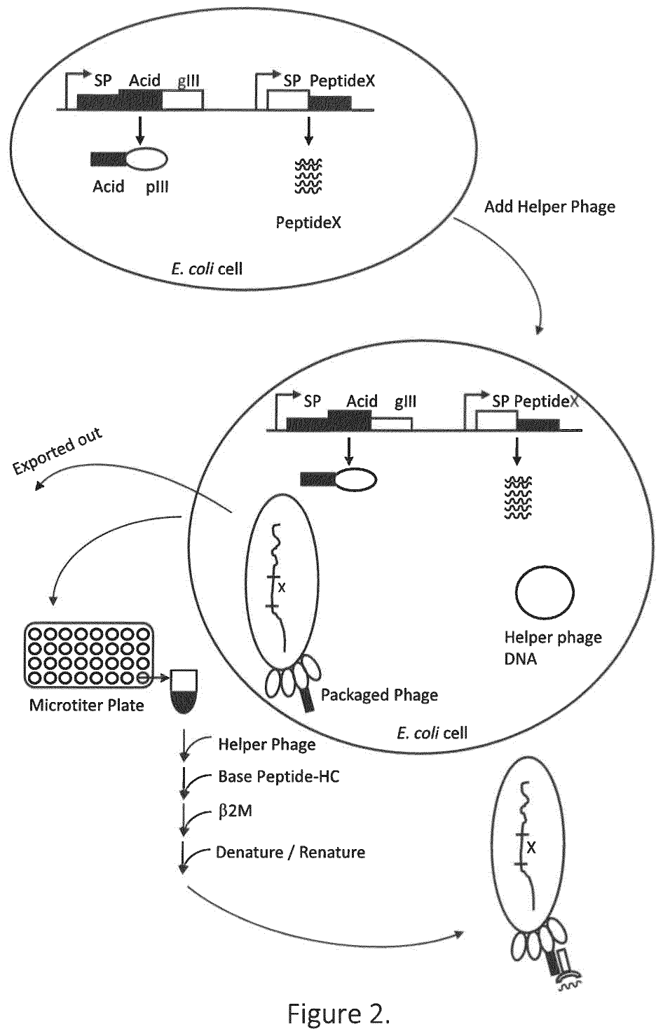

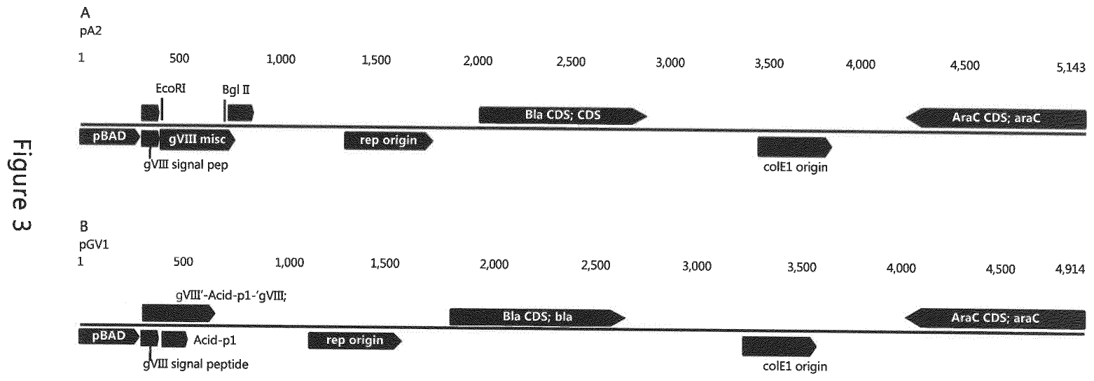

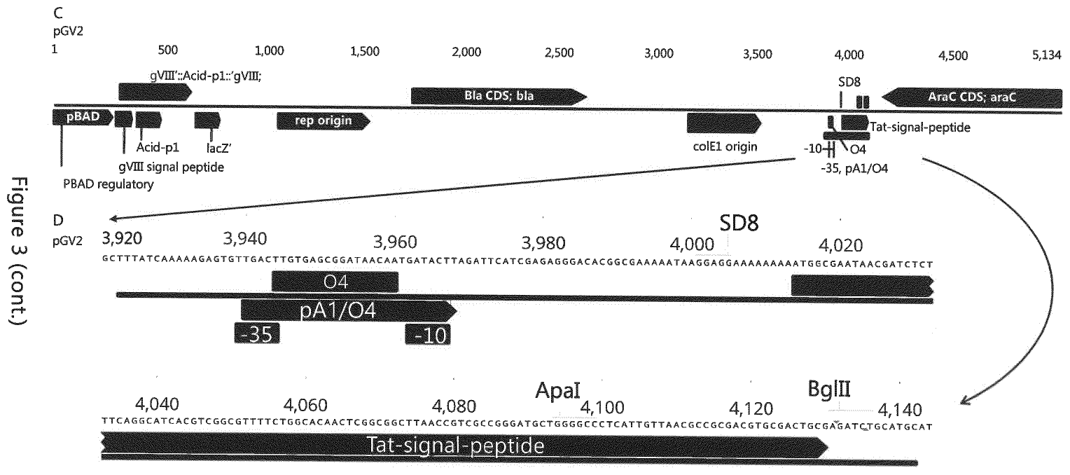

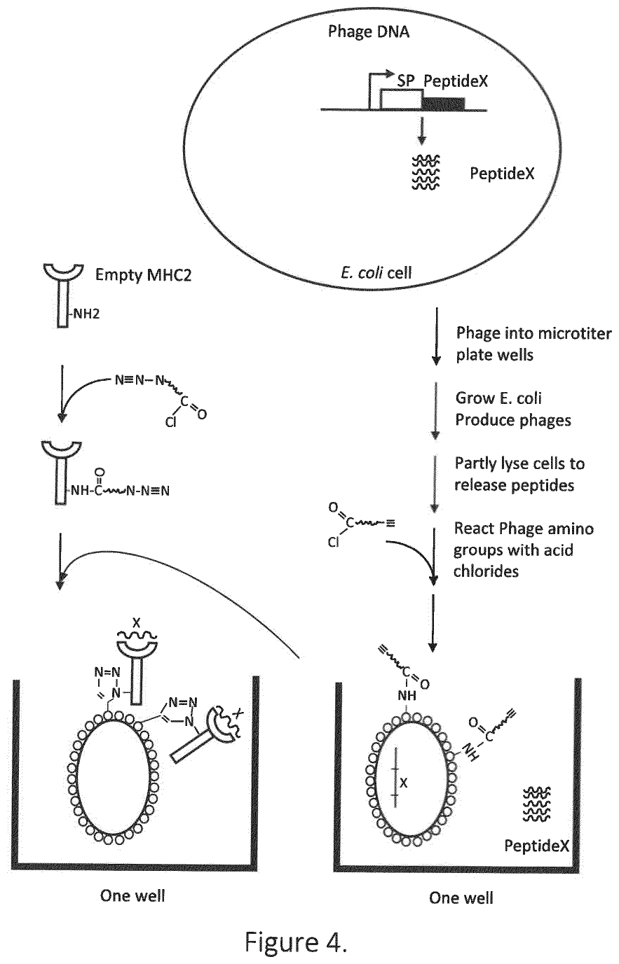

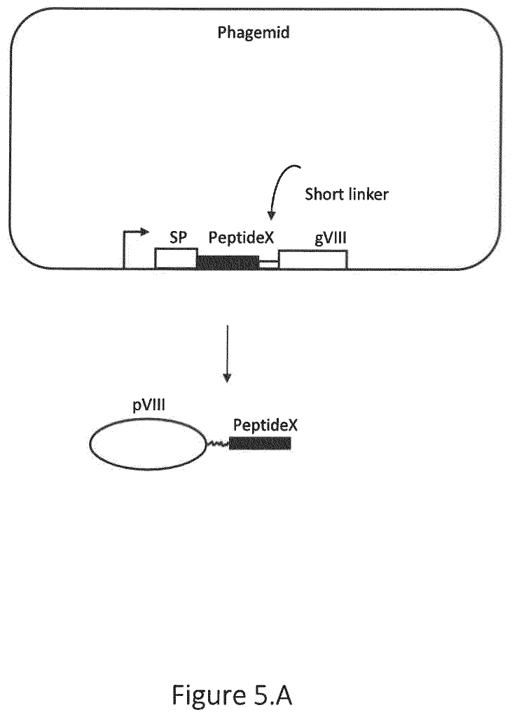

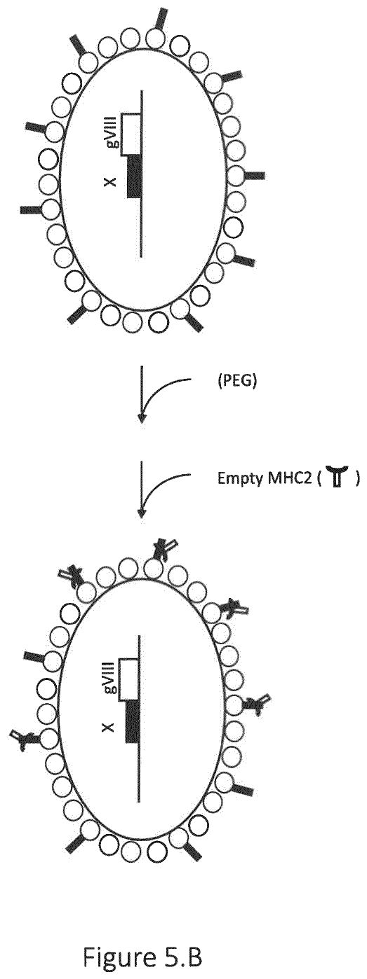

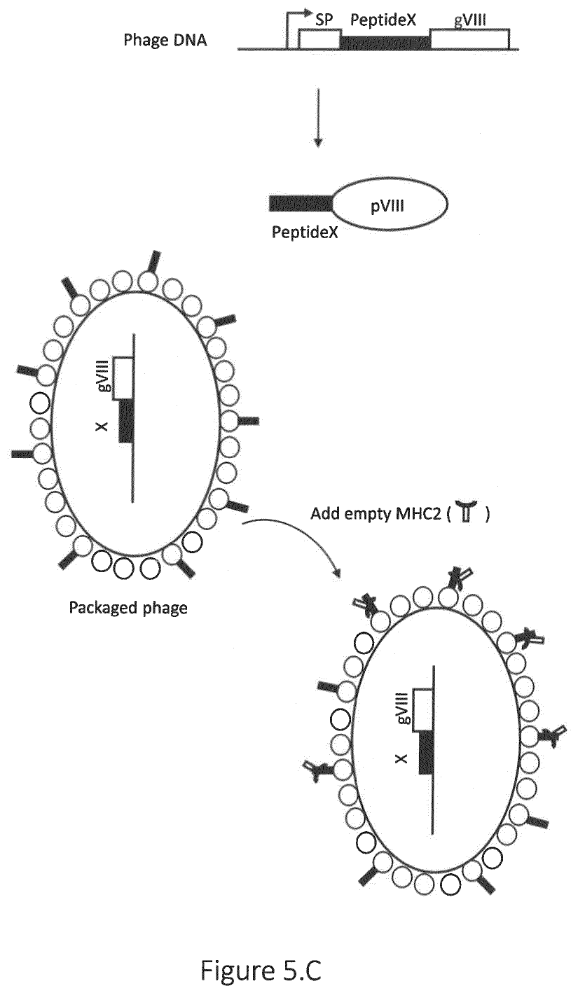

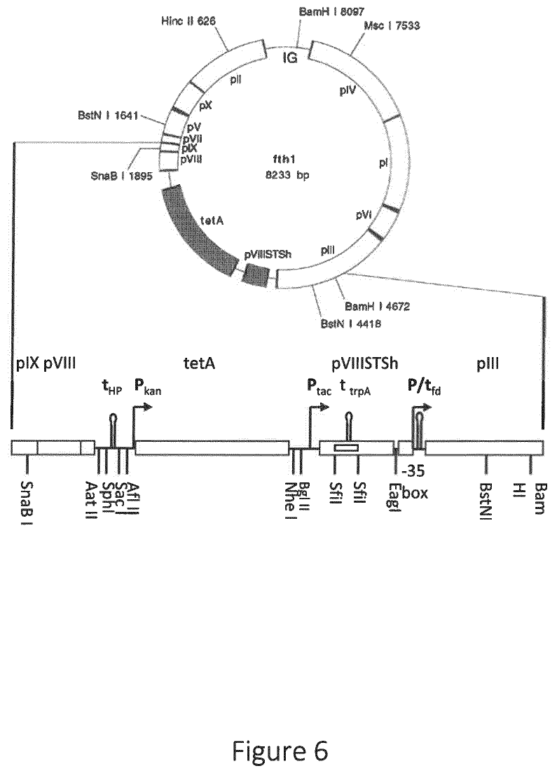

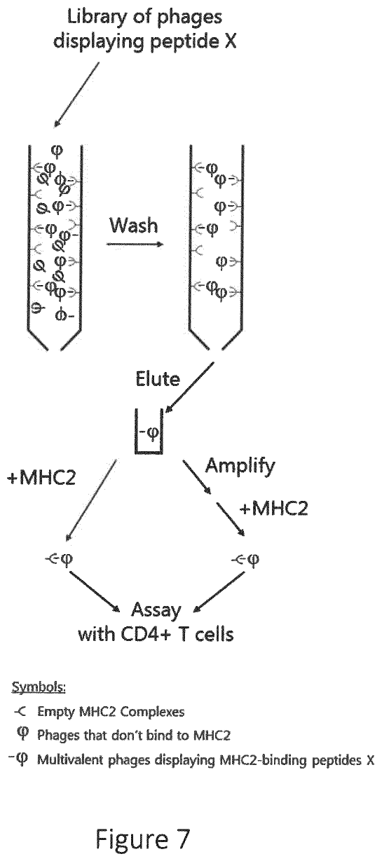

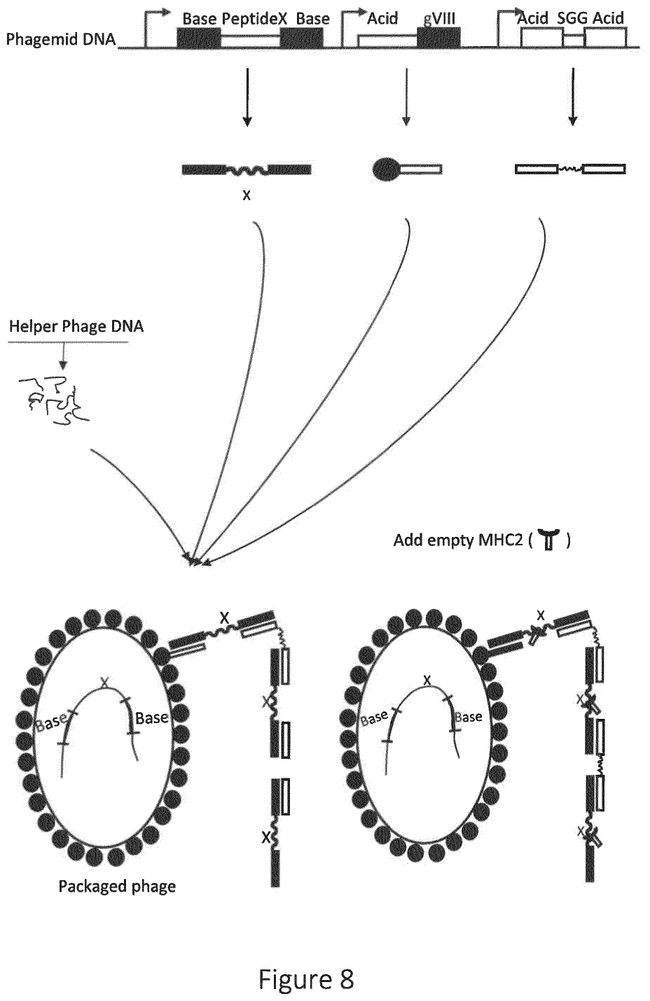

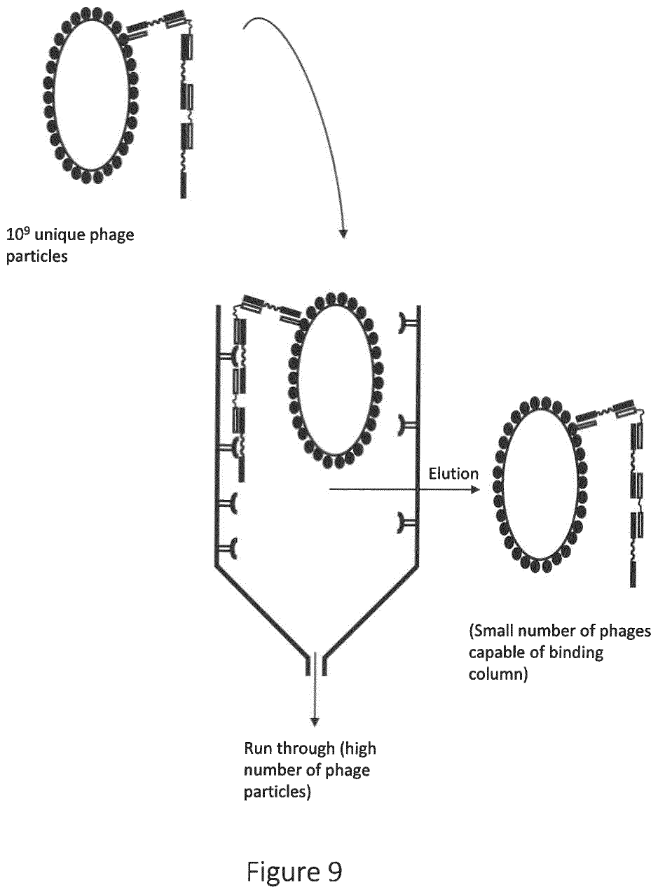

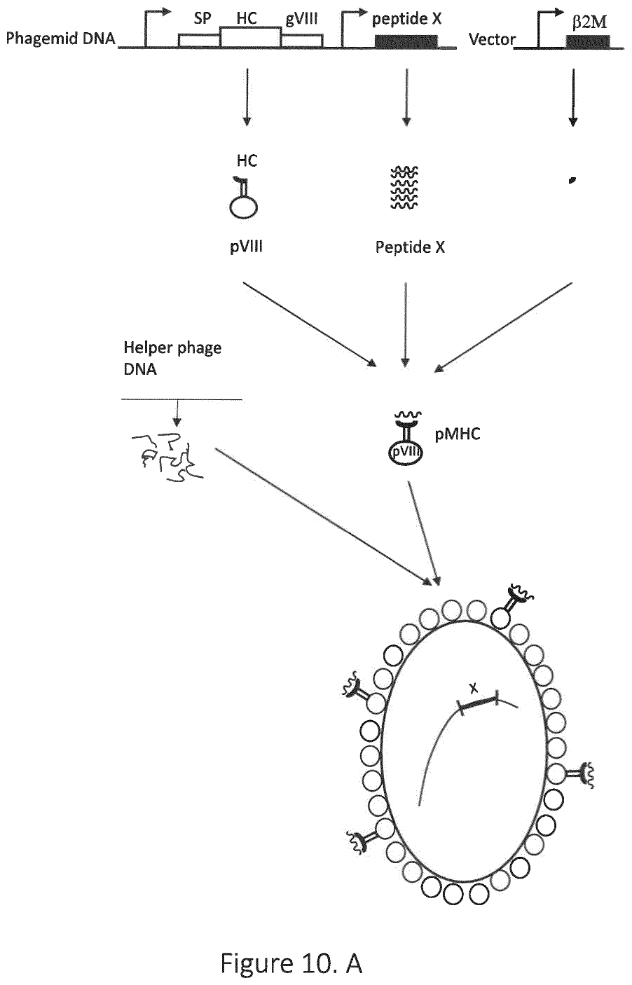

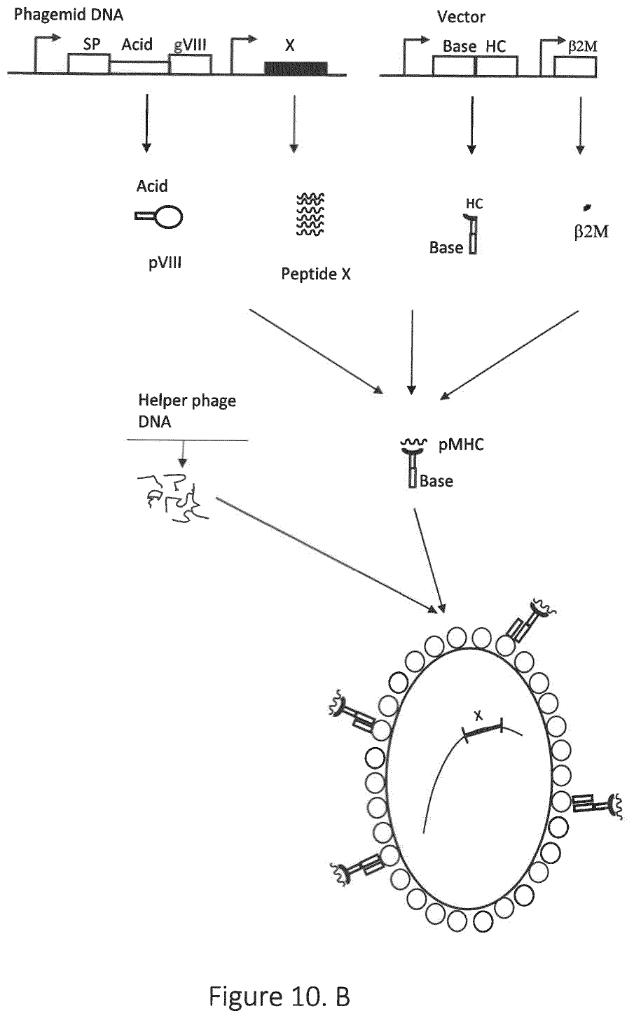

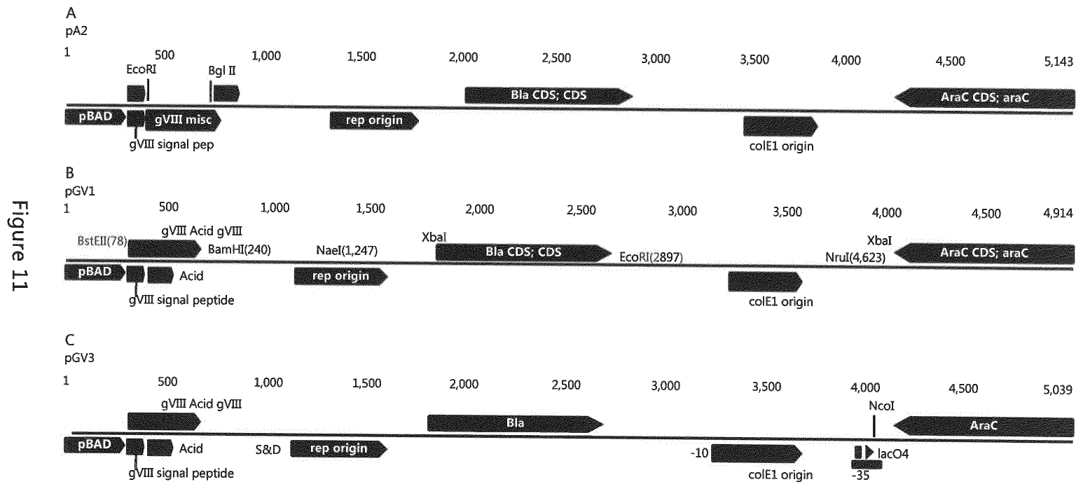

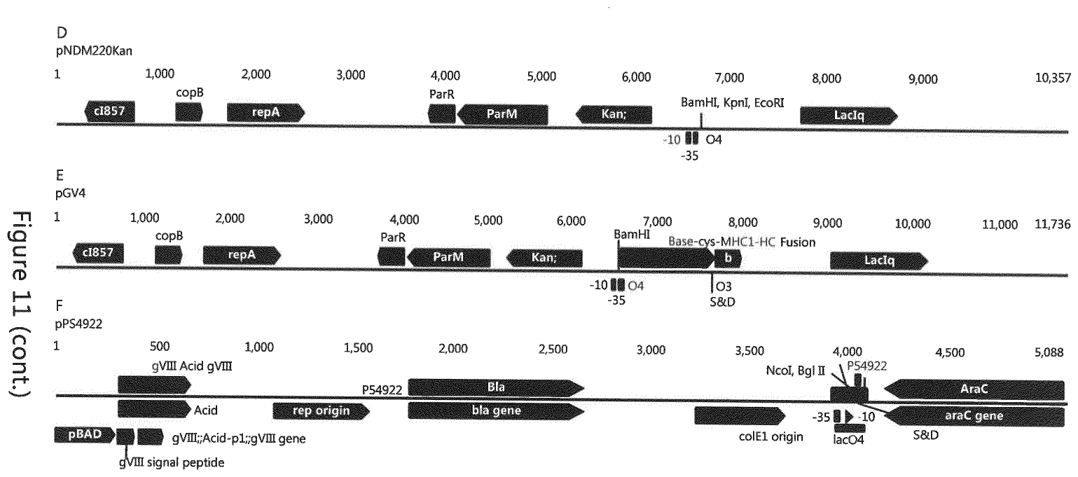

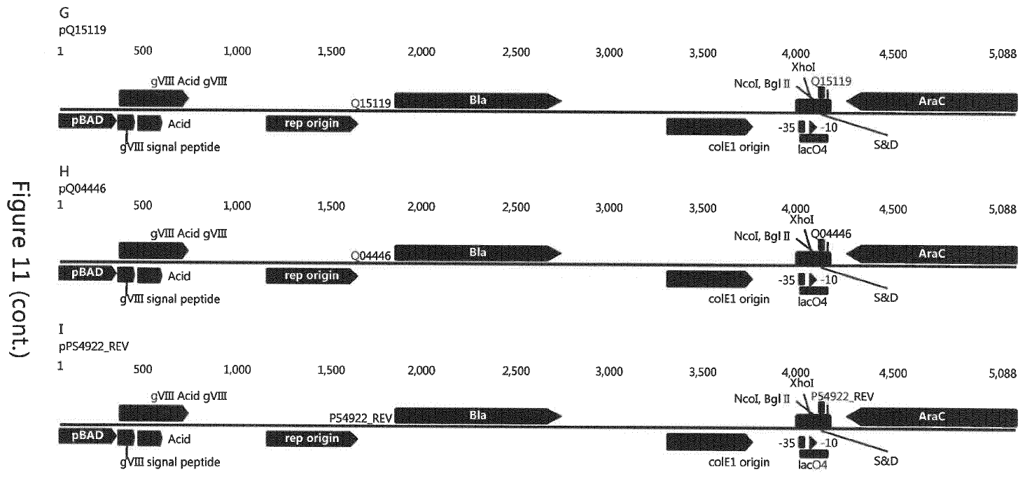

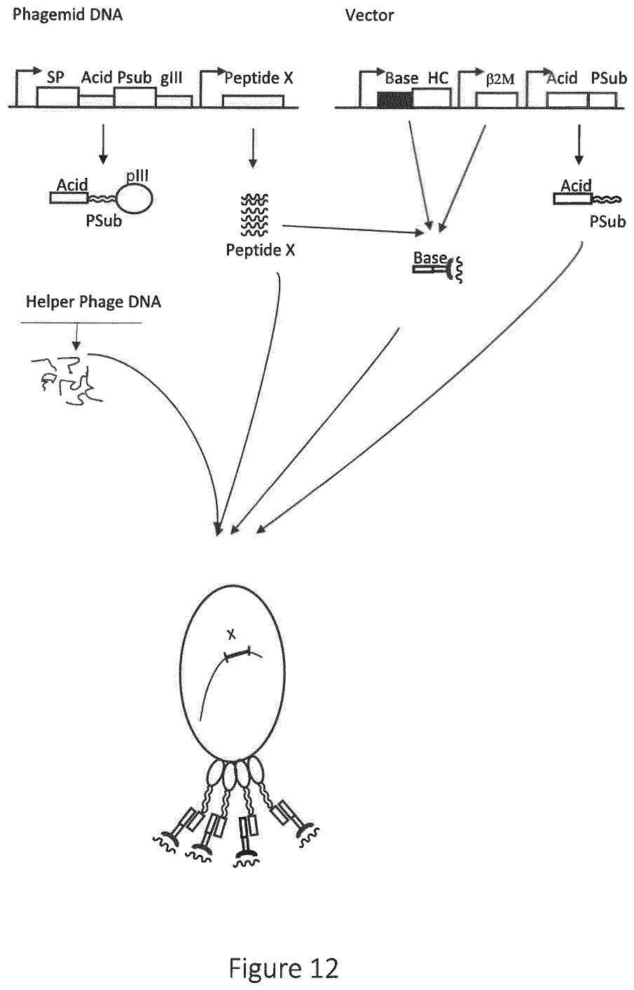

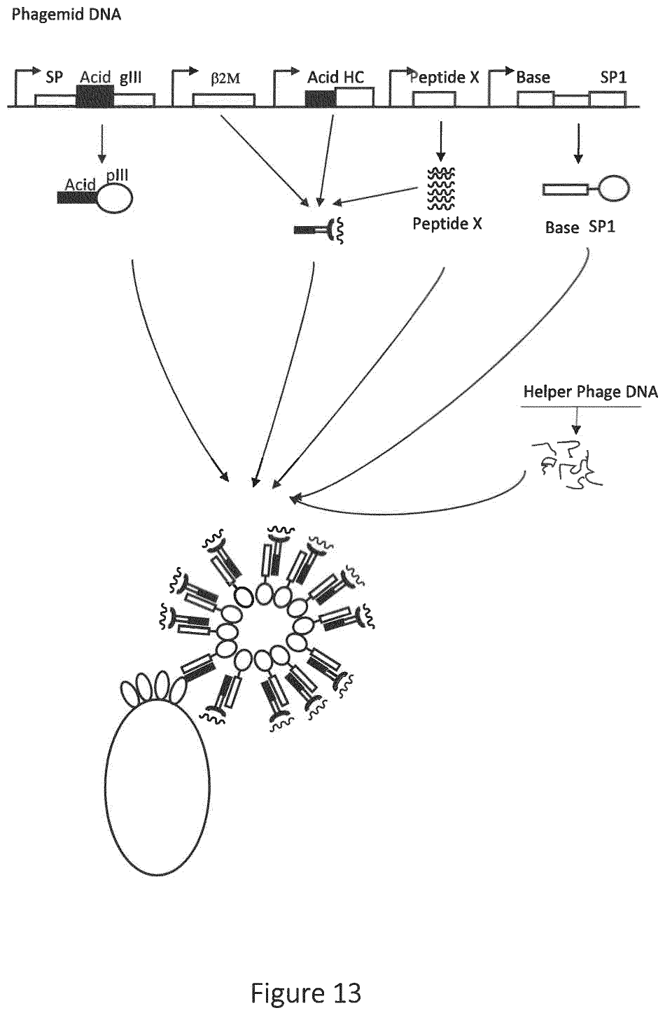

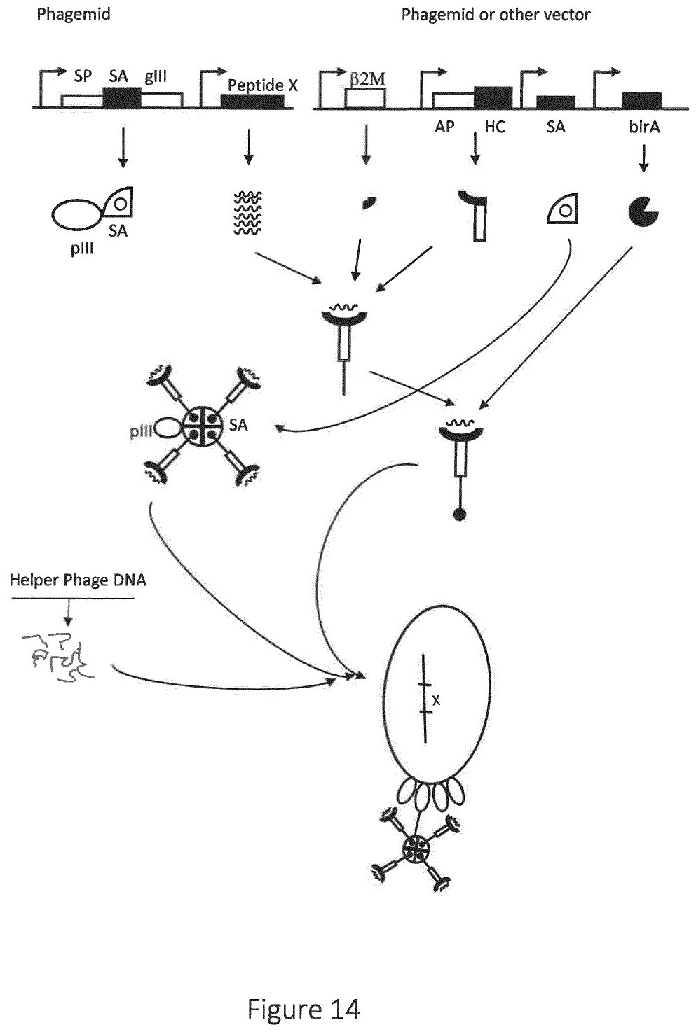

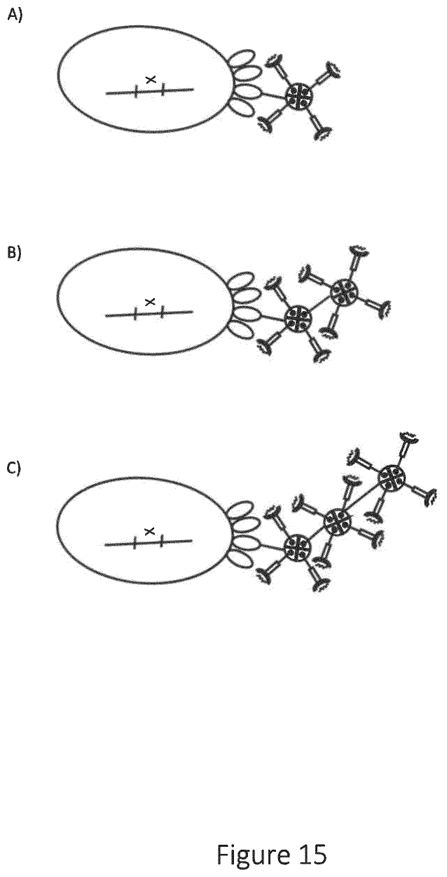

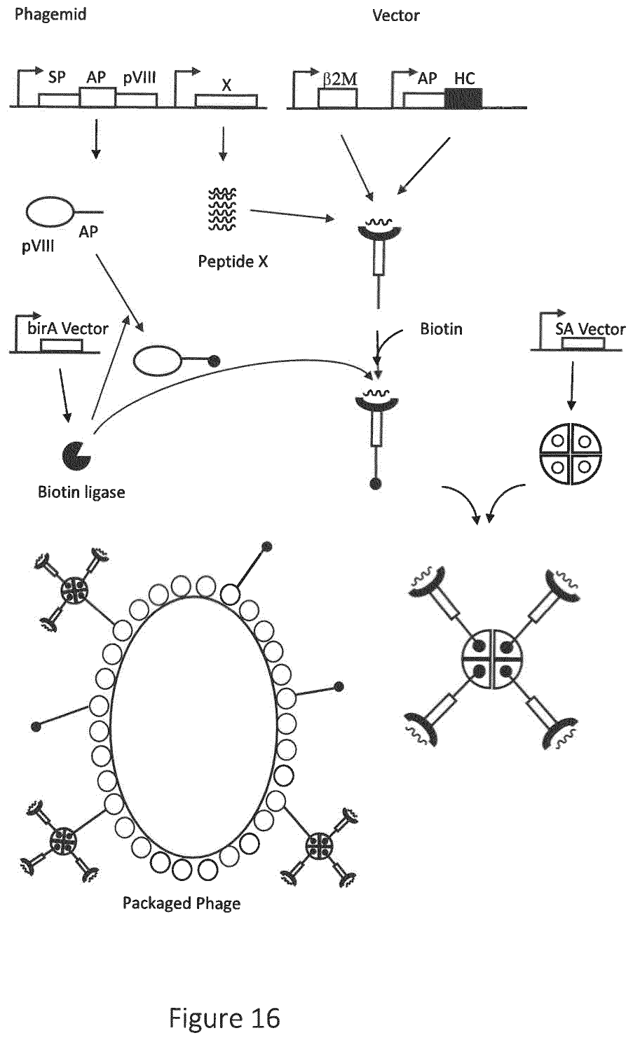

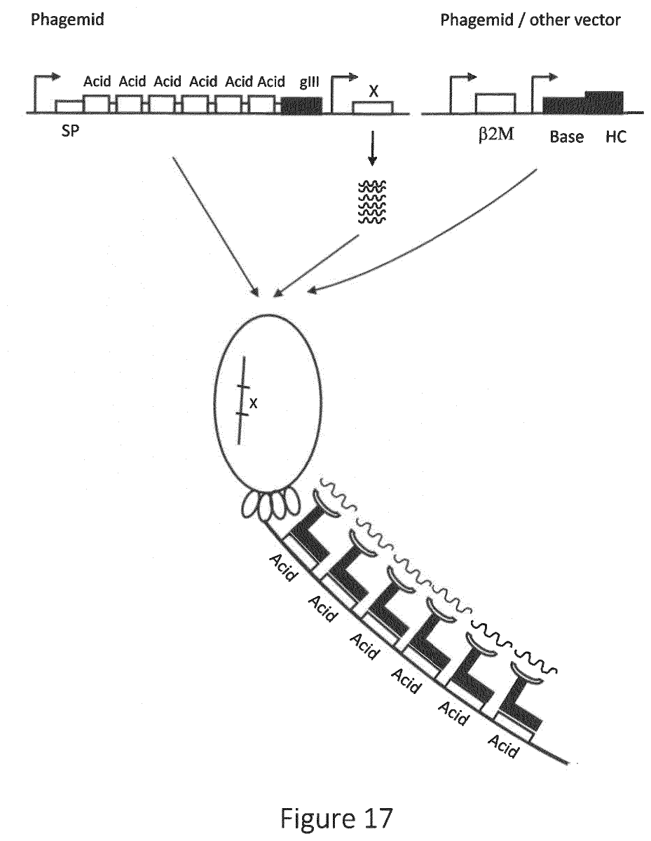

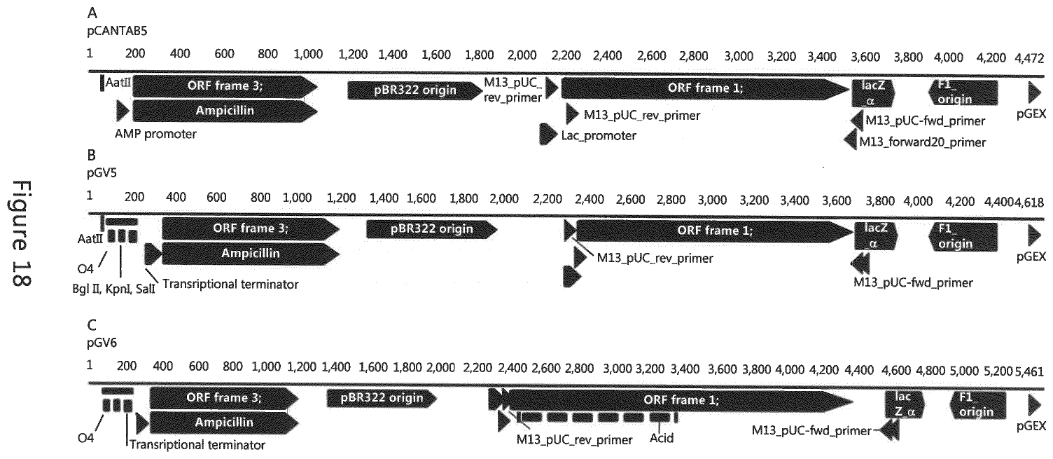

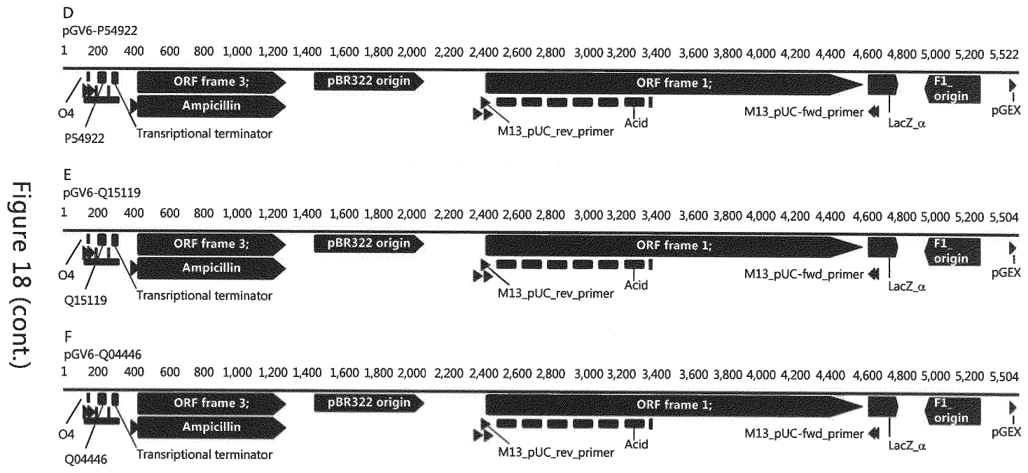

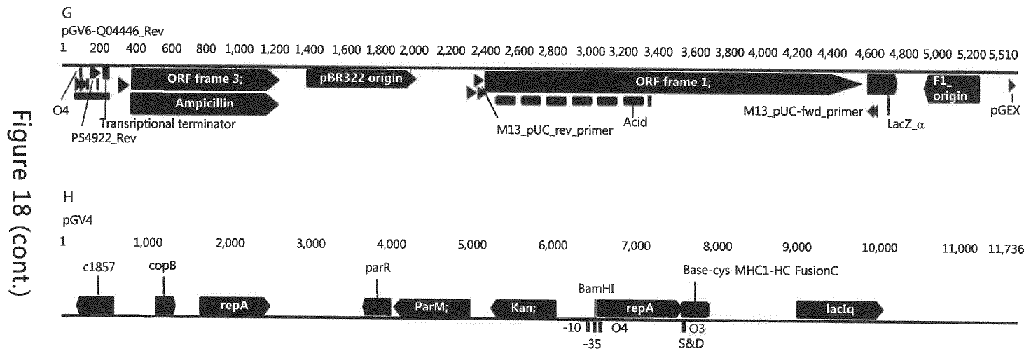

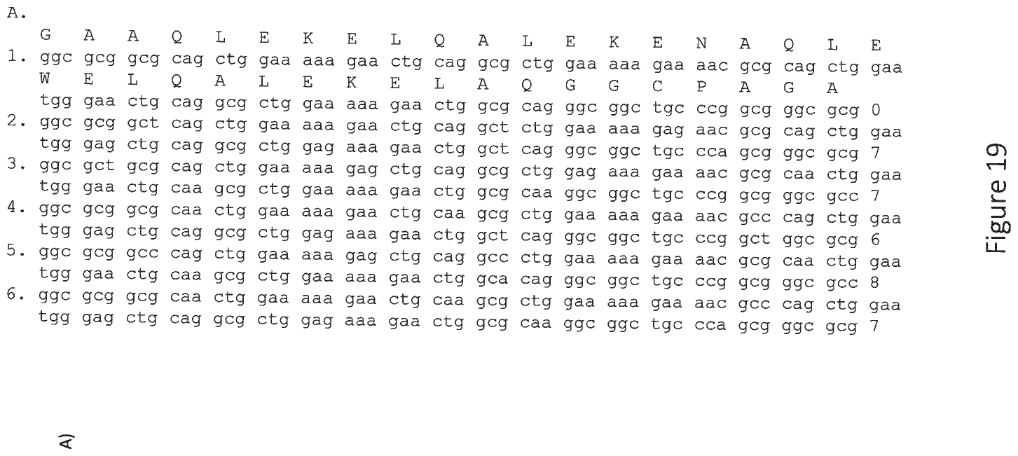

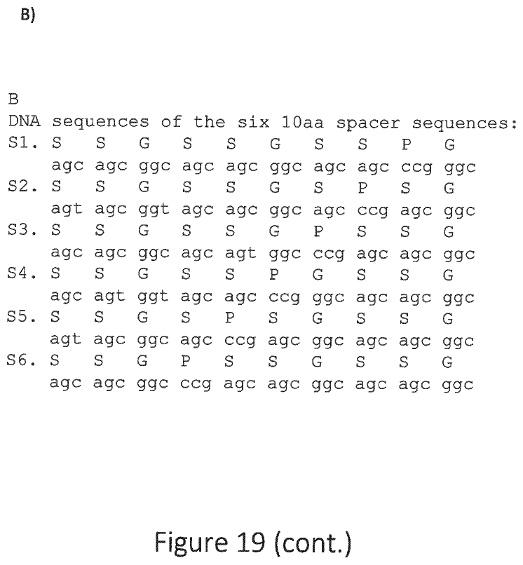

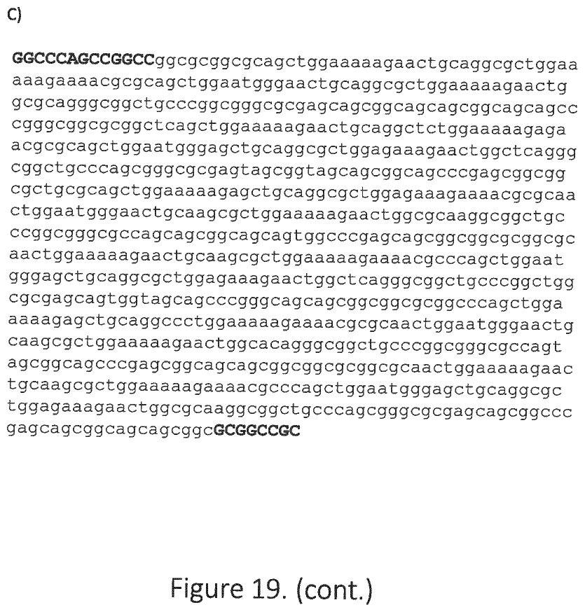

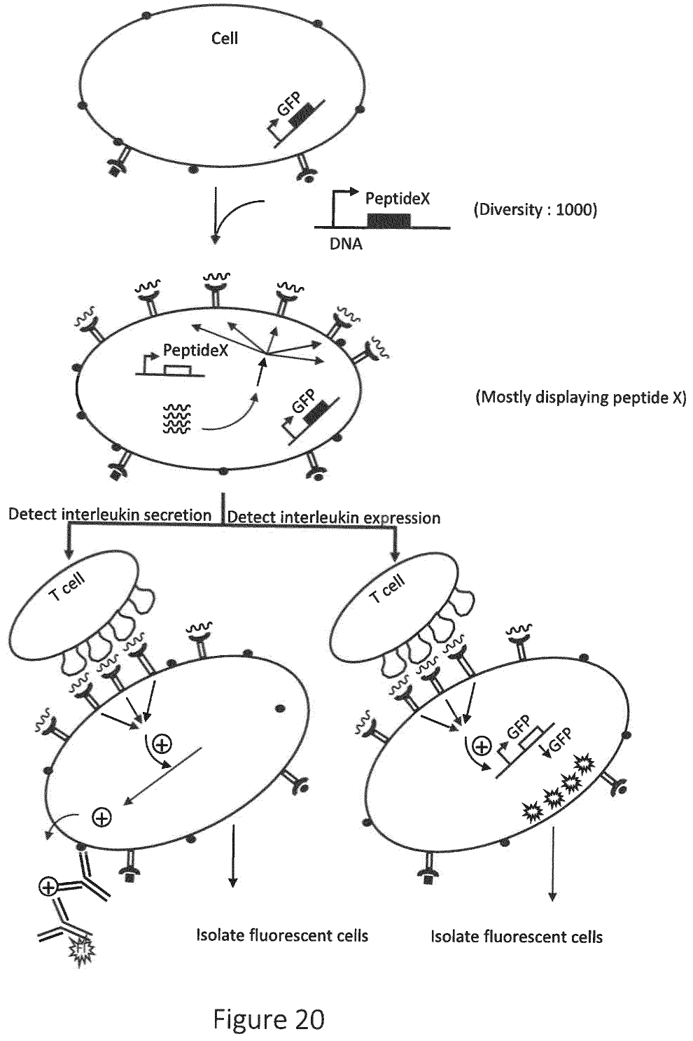

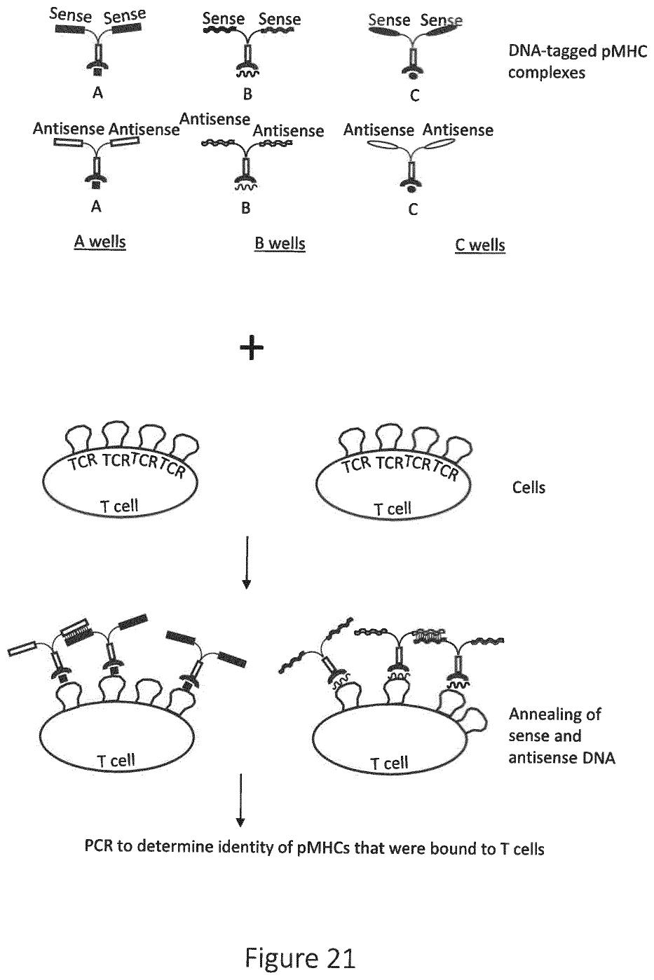

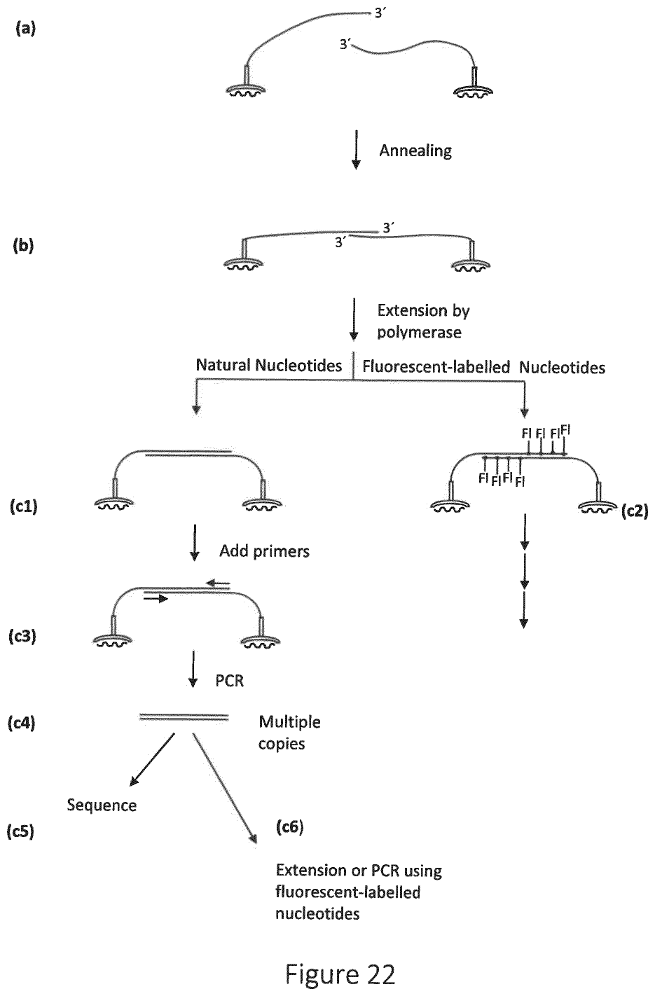

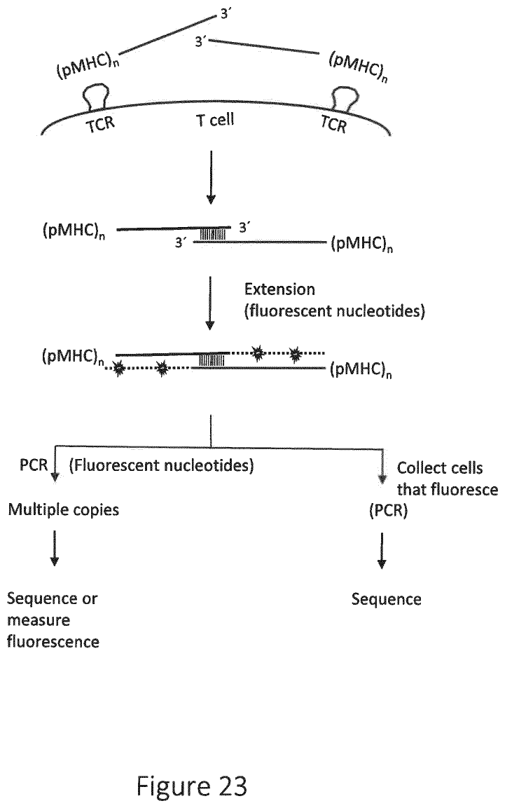

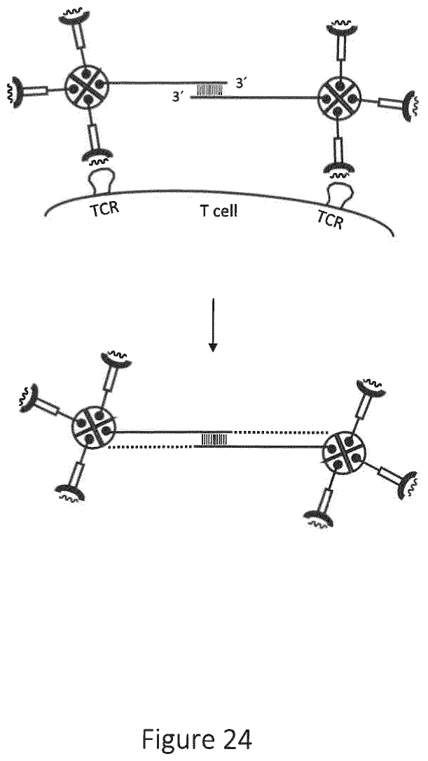

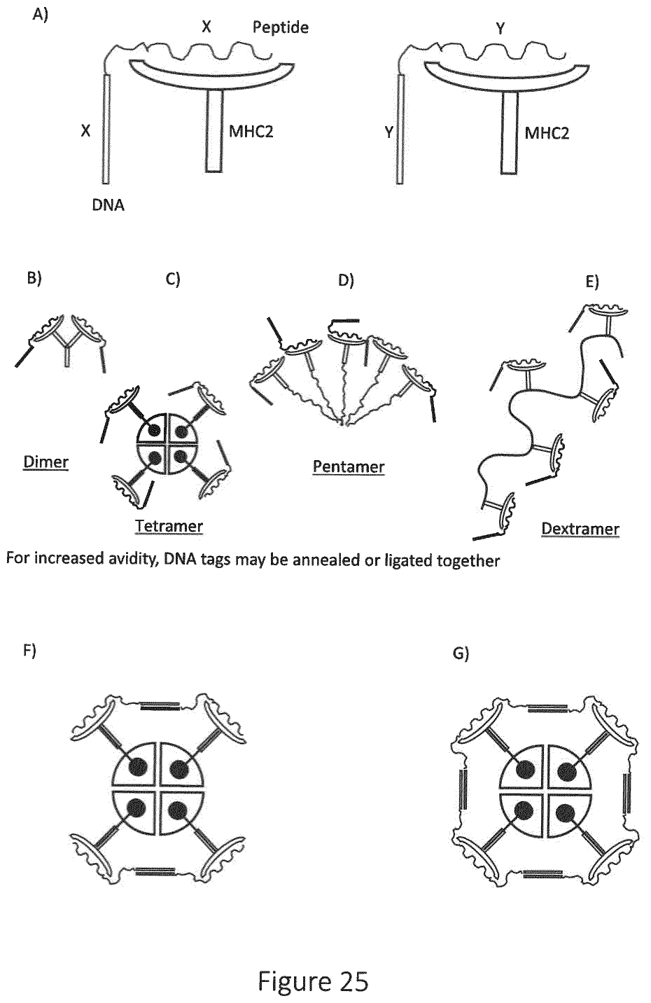

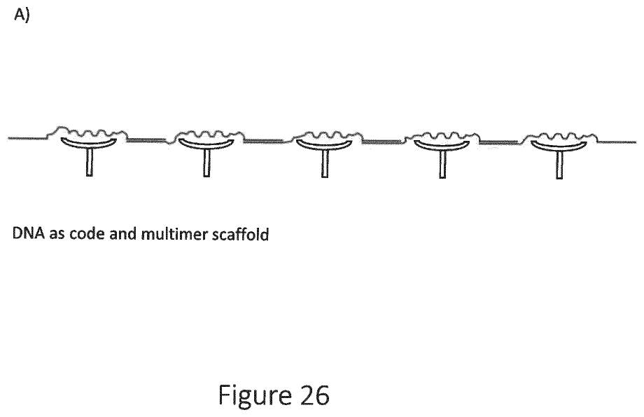

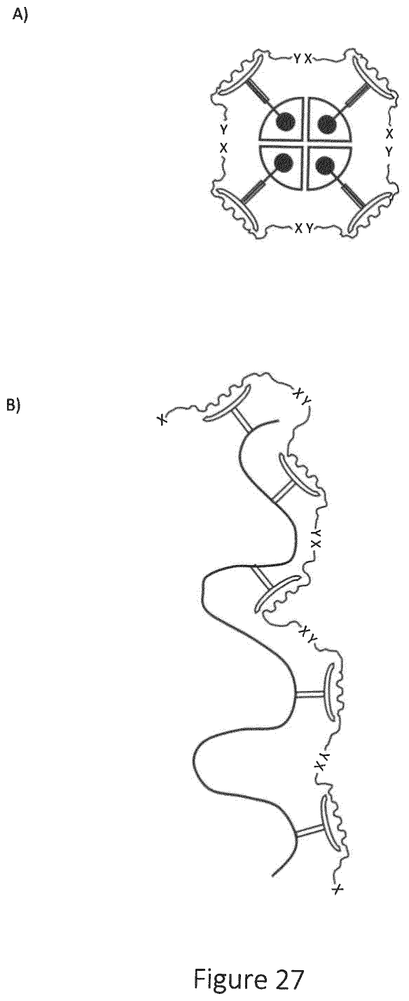

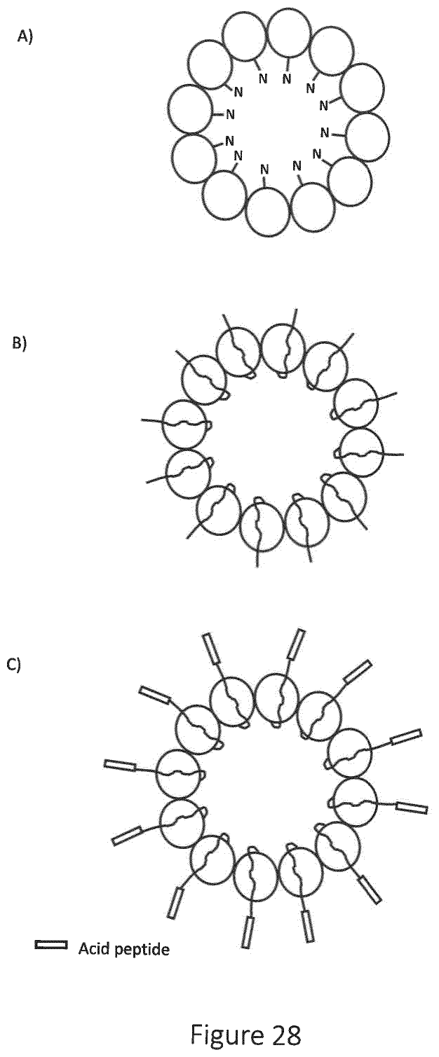

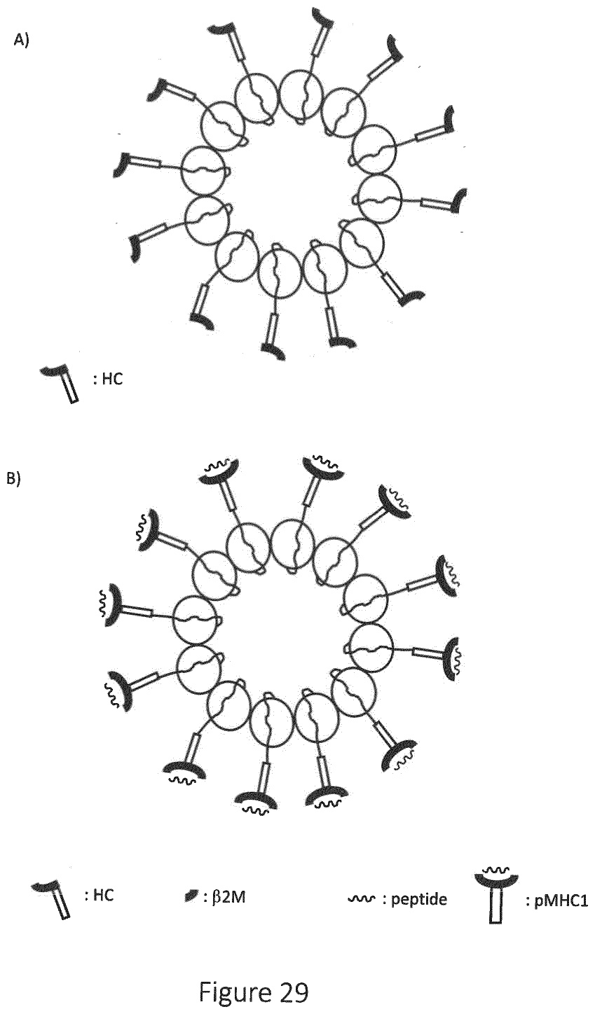

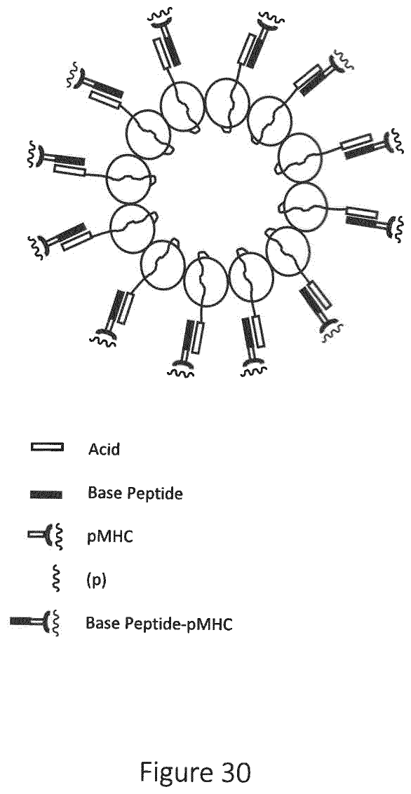

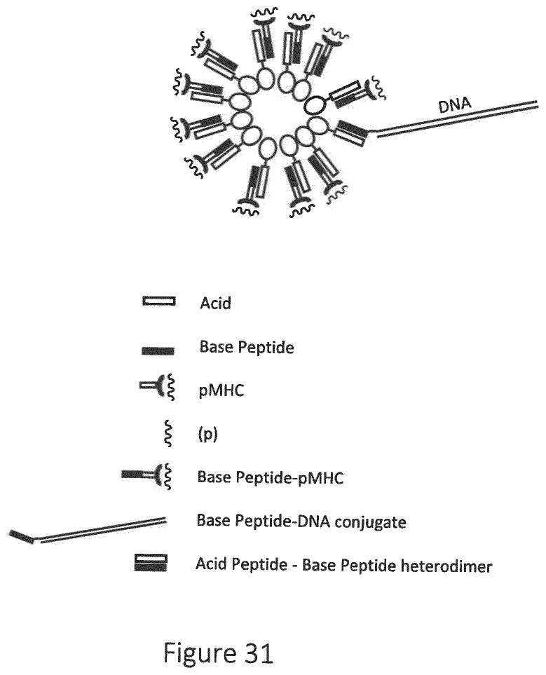

In a preferred embodiment of the invention, phage display is used as the display system. Specifically, the filamentous phage M13 is used, see (FIG. 2), to generate 1000 different pMHC Multiplexers:
Step a. On a phagemid, a DNA sequence is inserted encoding, from the N-terminal, Signal Peptide (SP) fused to Acid Peptide fused to the pIII coat protein. Here, an Acid Peptide variant is used that carries a cysteine, capable of forming a disulfide bond with Base Peptide upon their complexation. The SP-Acid-gIII fusion gene is under the control of a promoter whose expression can be modified by the addition of a molecule to the growth medium. One such molecule may be IPTG, where the promoter is under the regulatory control of IPTG.
Step b. On the same phagemid, a gene encoding a fusion of a signal peptide (SP) and a short peptide X is under the control of the same promoter as described above, or another promoter. Each phagemid copy encodes a specific peptide X; a collection of phagemids is used, here 1000 phagemids are present, where each phagemid carries a gene encoding a fusion of the signal peptide (SP) and a unique peptide sequence. Thus, in the figure, “peptide X” represents a number of (here: 1000) different peptide sequences, where each phagemid encodes a unique peptide sequence. Optionally, the production of peptide X is very efficient, leading to high amounts of peptide X in a bacterial cell carrying the phagemid. Optionally, the expression level of peptide X can be controlled by extracellular changes such as the addition of a molecule that stimulates transcription or translation of the gene encoding peptide X.
Step c. The phagemid constructs each encoding a unique peptide X sequence and all encoding the SP-Acid-pIII peptide fusion are introduced (e.g., by transformation) into an E. coli strain that is capable of supporting phage infection and amplification, and cell growth is performed for several cell generations. Then Helper Phage is added, and phage amplification is performed for several cell generations. Phage particles, some of which are carrying a copy of the Acid peptide, fused to the pIII coat protein, are now produced and are being secreted out to the supernatant, i.e. into the growth medium.
Step d. The E. coli cells and phage particles in the growth medium are separated by centrifugation, by pelleting the cells. The supernatant comprising the phage particles is transferred to a new tube. This solution of phage particles is then diluted, and small aliquots of diluted phage particles are added to a number (here: 5000) of microtiter wells (or tubes or flasks) comprising growing E. coli cultures (i.e. E. coli strains capable of supporting phage infection and amplification), where the aliquots each comprise approximately on average 0.1-0.3 phage particles. To each microtiter well (or tube or flask) is added Helper Phage, to initiate phage production and secretion.
Step e. Optionally, activator molecules are added in order to increase the expression of the Acid-pIII fusion protein and the signal peptide-peptide X fusion variants.
Step f. Growth of E. coli and resultant phage particle production and secretion into the growth medium is performed. Likewise, the signal peptide (SP)-peptide X fusion peptide is expressed, the signal peptide is cleaved off and the resulting C-terminal end (peptide X) is transported into the periplasm.
Step g. The E. coli cells are exposed to (partial) lysis, permeabilizing the outer membrane and resulting in release of the peptides from the periplasm into the growth medium. Many methods for the permeabilization of the outer membrane exist, including treatment with MAC13243 (described in Scientific Reports 7, Article number: 17629 (2017), title: “Increasing the permeability of Escherichia coli using MAC13243”) or by cold osmotic shock (described in BioTechniques 66: 171-178 (2019), title: “A robust fractionation method for protein subcellular localization studies in Escherichia coli”).
Step h. The cells and cell debris are pelleted by centrifugation and the supernatant of each of the microtiter wells (or tubes or flasks) is transferred to another microtiter well (or tube or flask).
Step i. Optionally, protease inhibitor(s) and/or nuclease inhibitors are added-before, during or after (partial) lysis.
Step j. The peptides of a solution comprising Base Peptide-Heavy Chain (Base-HC) fusion protein (consisting of the Heavy Chain protein, one of the monomers of the MHC1 dimeric complex, and the Base Peptide fused to the N-terminal end of HC) and Beta2M (B2M), are first denatured by increasing the temperature to preferably between 50° C. and 60° C. (or higher if desired), and then aliquoted into each of the microtiter wells to a final concentration preferably lower than the estimated concentration of peptide X in the individual well, and where the wells were kept at elevated temperature (e.g., 45° C.) for 30 seconds before addition of the solution. Here, a Base Peptide variant is used that carries a cysteine, capable of forming a disulfide bond with Acid Peptide upon their complexation.
Step k. The temperature of the solutions in the wells are kept at approximately 45° C. for 2 minutes.
Step l. The temperature is decreased to approximately 20° C., to allow complex formation between peptide X, HC and beta2M, to potentially form the peptide X-MHC1 complex.
Step m. Optionally, a redox buffer is added, to first reduce the cysteine in each of the Acid Peptide and the Base Peptide.
Step n. Optionally, the oxidation/reduction status of the buffer is adjusted to allow disulfide bond formation between the cysteines of the Acid Peptide and the Base Peptide. As a result, the peptide X-MHC1 complex becomes covalently linked to the Acid Peptide-pIII fusion protein of the phage coat.

The resulting product of the process described by the above steps (a-n), is 5000 wells in total comprising approximately 1000 unique pMHC Multiplexers.

### Comments:

In the experiment described above, one copy of a DNA sequence encoding the Acid Peptide is fused to the gIII gene encoding the pIII coat protein. As an alternative, a DNA encoding multiple copies of the Acid Peptide sequence can be fused to the gIII gene, resulting in phages displaying a peptide carrying multiple copies of the Acid Peptide repeat. After addition of the proteins of the MHC complex, folding of the pMHC complex and formation of the Acid Peptide/Base Peptide complex, the phage particle will carry multiple copies of the pMHC complex attached to its coat surface.
The DNA encoding the Signal Peptide (SP) encodes a peptide that is cleaved at or near the C-terminal end of the signal peptide sequence, in connection with its transport into the periplasm. Example signal peptides are Tat and Sec. In E. coli many signal peptides mediate the transport of peptides, to which they are fused, into the periplasm.
In the above experiment, a signal peptide is fused to the N-terminal of peptide X, in order to affect the transport of peptide X into the periplasm. This way, the cell lysis step does not need to affect the inner cell membrane of the E. coli cell, in order for the peptide transport into the growth medium to be effective. If a signal peptide is not fused to peptide X, it is preferable to do a more intensive cell lysis including the inner membrane, in order to release a high amount of peptide X into the growth medium.
In the above experiment, the Acid Peptide is fused to pIII, and Base Peptide is fused to HC. Alternatively, Base Peptide may be fused to pIII and Acid Peptide may be fused to HC.
In the above example, the Base Peptide is fused to the HC of the MHC1 complex, which leads to attachment of the MHC1 complex to the Acid Peptide. Alternatively, the Base Peptide may be fused to beta2M of the MHC1 complex, leading also to attachment of the MHC1 complex to the Acid Peptide.
In the above experiment, pMHC1 complexes were formed from HC and beta2M protein, and attached to the phage coat. Alternatively, pMHC2 complexes could be formed from alpha and beta protein, where either alpha or beta were fused to Base Peptide. The same protocol for denaturation may be used, or alternatively, the folded (empty) MHC2 complex may be added without the need for a denaturation step.
In the above experiment, denaturation was brought about using increased temperature. Alternatively, (partial) unfolding may be mediated by the addition of urea, ethanol, or any other agent or treatment that leads to (partial) unfolding of the HC and Beta2M protein. Preferably, the treatment should be done in a way that ensures that the phage particle is not (fully) disintegrated.
Instead of adding a Base Peptide-HC fusion protein, a Base Peptide-tagged MHC Multimer (such as a Base Peptide-tagged MHC Tetramer, a Base Peptide-tagged MHC Dextramer or a Base Peptide-tagged MHC Pentamer, SP1-based MHC Multimer), may be added. The resulting product will then be a MHC Multimer attached to the phage coat protein. The preparation of a Base Peptide-tagged MHC Multimer is here exemplified for the preparation of a Base Peptide-tagged MHC Tetramer: First a Base Peptide, biotinylated at one end, is mixed with about 1 equivalent of a streptavidin tetramer (SA), to allow formation of the Base Peptide-SA complex. Then 5-10 equivalents (relative to the SA tetramer) of beta2M protein and biotinylated HC protein is added, and incubated. This will lead to formation of Base Peptide-tagged MHC Tetramer, which can then be added to the phage particle carrying one or more copies of the Acid Peptide, to make the phage display an MHC Tetramer.
In the experiment described above, the individual proteins of the MHC1 complex were added, denatured and then renatured, to form the pMHC1 complex. Alternatively, an active exchange process may be applied. In this approach, a (relatively labile) p*MHC complex (where p* is a peptide that binds with modest affinity to the MHC1 complex, and where the p*MHC complex carries a Base Peptide sequence) is added to all microtiter wells (or tubes or flasks), instead of the HC and beta2M protein. Then, instead of denaturation, a so-called helper-ligand is added, capable of intermittent binding to the p*MHC1 complex. The helper-ligand mediates the active replacement of the p* peptide with peptide X from the solution, resulting in formation of the peptide X-MHC1 complex.
In the above example, the pMHC complex becomes attached to the pIII coat protein. Alternatively, the Acid Peptide could have been fused to the pVIII coat protein, which would have resulted in the pMHC complex becoming attached to the pVIII coat protein. Alternatively, the pMHC complex could have been attached to any other coat protein of the filamentous phage.
The above example uses a system including both phagemid and helper phage. This ensures that only a fraction of the pIII coat proteins or of the pVIII coat proteins of the phage particle will display the Acid Peptide, and hence only a fraction of the pIII coat proteins or of the pVIII coat proteins of the phage particle will display a pMHC complex after its attachment to the Acid Peptide.
Alternatively, one may fuse the Acid Peptide to the pIII protein or pVIII protein encoded by the phage DNA using a phage where the gene encoding pIII or pVIII is duplicated, thus avoiding the use of a phagemid and a helper phage. Hence, in this example, a phagemid would not be included, and the peptide X should be encoded by the phage DNA, and not the phagemid DNA as was done in the above example.
In a preferred embodiment of the invention, an E. coli strain is used with deletions or mutations in genes encoding proteases, such as proteases in the membranes or periplasm, in order to increase the average life-time of the expressed peptides.
As an alternative, the above procedure can be changed so as to not attach the pMHC complex to the phage (e.g., use a phagemid that does not carry a gene encoding Acid Peptide-pIII peptide fusion). The resulting product of the process described above will then be 1000 wells each comprising a unique pMHC complex and a unique DNA.
By appropriate modification, any other type of filamentous or non-filamentous phage, and any virus may be used in this invention. Thus, any appropriate coat protein on any phage or virus can be used in the invention.
Likewise, any membrane protein or other type of molecule associated with the extracellular membrane surface of a prokaryotic or eukaryotic cell may be used as the display carrier of the pMHC complex.

### Example 16. Preparation of pMHC Multiplexers by Extracellular pMHC Formation and Attachment to Phage by Click Chemistry

This example describes the preparation of a spatial array (microtiter plate wells) of phage clones (each individual clone placed in the same well as the free peptide that its DNA encodes), followed by addition of MHC complex, formation of pMHC complex, and attachment of the formed pMHC complex to the phage particle by click chemistry. The result is a pMHC Multiplexer where the pMHC complex is attached to the phage particle (comprising the DNA) that encodes the peptide.

In a preferred embodiment of the invention, phage display is used as the display system. Specifically, the filamentous phage M13 is used, see (FIG. 4), to generate 1000 different pMHC Multiplexers:
Step a. A large number of DNA molecules (here: 1000 different DNA molecules), each of which carries a promoter upstream of a DNA sequence encoding a signal peptide (SP) in reading frame (and N-terminal to) a short peptide X, are cloned into the M13 genome. Each of the 1000 different DNA molecules carry a unique sequence encoding a unique peptide X. Thus, in (FIG. 4), “peptide X” represents a number of (here: 1000) different peptide X sequences, where each phage DNA carries a unique DNA sequence encoding the peptide X. Optionally, the expression of peptide X is very efficient in E. coli, leading to high amounts of peptide X in a cell containing the phage DNA. Optionally, the expression level of peptide X can be controlled by extracellular changes such as the addition of an activator molecule or other changes in the growth medium.
Step b. The phage DNA constructs encoding a total of 1000 different peptide X sequences is introduced (e.g., by transformation) into an E. coli strain that is capable of supporting phage infection and amplification, and cell growth is performed for several cell generations. Phage particles are now produced and are being secreted out into the supernatant, i.e. into the growth medium.
Step c. The growth medium comprising the E. coli cells and phage particles are centrifuged, to pellet the cells. The supernatant comprising the phage particles is transferred to a new tube. Optionally, the phage particles are PEG-precipitated, and the pellet resuspended in an appropriate buffer. This solution of phage particles is then diluted, and small aliquots of diluted phage particles are added to a number (here: 5000) of microtiter wells (or tubes or flasks) comprising growing E. coli cultures (i.e. E. coli strains capable of supporting phage infection and amplification), where the aliquots each comprise on average 0.1-0.3 phage particles. Growth is continued.
Step d. Optionally, activator molecules are added in order to increase the expression of the signal peptide-peptide X fusion peptides.
Step e. Continued growth of E. coli and resultant phage particle production and secretion into the growth medium is performed. Likewise, the signal peptide (SP)-peptide X fusion peptide is expressed, the signal peptide is cleaved off and the resulting C-terminal end (peptide X) is transported into the periplasm.
Step f. The E. coli cells are exposed to (partial) lysis, permeabilizing the outer membrane, and resulting in release of the peptides from the periplasm into the growth medium. Many methods for the permeabilization of the outer membrane exist, including treatment with MAC13243 (described in Scientific Reports 7, Article number: 17629 (2017), title: “Increasing the permeability of Escherichia coli using MAC13243”) or by cold osmotic shock (described in BioTechniques 66: 171-178 (2019), title: “A robust fractionation method for protein subcellular localization studies in Escherichia coli”).
Step g. The (partially) lysed cells and cell debris are pelleted by centrifugation and the supernatant of each of the microtiter wells (or tubes or flasks) is transferred to another microtiter well (or tube or flask).
Step h. Optionally, protease inhibitor(s) and/or nuclease inhibitors are added-before, during or after (partial) lysis.
Step i. A compound comprising a terminal triple bond and a carboxylic acid chloride is added to the wells, at a concentration that leads to reaction of the acid chloride with 0.1%-1% of the amino groups of the phage coat proteins. After reaction, about 0.1%-1% of the amino groups of the phage coat protein will thus have been modified and now carry a triple bond.
Step j. In a separate reaction, the HC protein, beta2M protein or an empty MHC1 complex, and/or alpha protein, beta protein or an empty MHC2 complex is modified with an azide moiety. The modification is done in a way that results in approximately 1 modification per protein. The modifying reaction may be performed by the addition of a compound that comprises a carboxylic acid chloride and a terminal azide moiety, at a concentration that results in the covalent linkage of on average one azide moiety per protein.
Step k. The azide-modified HC protein, beta2M protein or empty MHC1 complex, and/or the azide-modified alpha protein, beta protein or empty MHC2 complex of step (j) is added to the wells of (i).
Step l. The triple bond (covalently attached to a phage coat protein) and the azide moiety (covalently attached to a protein of the pMHC complex) will react, thereby covalently linking the pMHC complex to the phage coat protein.
Step m. If azide-modified HC protein, beta2M protein or empty MHC1 complex was added in the above step, the wells are now exposed to (partly) denaturing conditions, e.g., heat (50-70° C.), urea or other denaturing conditions. Then the conditions are brought back to non-denaturing conditions, to allow refolding of the proteins and formation of pMHC complexes. If only empty MHC2 complex was added in the above step, the peptide may simply bind to the empty MHC2 complex without prior denaturation, thereby forming the pMHC2 complex.

The resulting product of the process described by the above steps (a-m), is thus approximately 1000 unique pMHC Multiplexers.

### Comments:

The DNA molecules that are cloned into the phage genome encodes a Signal Peptide (SP), fused to the N-terminal of variant sequences of the peptide X. In connection with its transport into the periplasm, the SP-peptide X fusion peptide is cleaved at or near the fusion point of the Signal Peptide and peptide X. As a result, the peptide X variants (without the Signal Peptide) are released into the periplasm.
Example signal peptides are Tat and Sec. In E. coli many signal peptides mediate the transport of peptides, to which they are fused, into the periplasm.
In the above experiment, a signal peptide is fused to the N-terminal of peptide X, in order to affect the transport of peptide X into the periplasm. This way, the cell lysis step does not need to affect the inner cell membrane of the E. coli cell, in order for the peptide transport into the growth medium to be effective. However, in a preferred embodiment of the invention, the phage DNA does not carry a signal peptide in fusion with the peptide X variants. In this case it is preferable to do a more intensive cell lysis including the inner membrane; lysis should be adjusted to allow the release of peptide X from the cytoplasm, as the peptide X may not be present in the periplasm in high amounts until (partial) lysis has been performed.
In the above experiment, the phage coat is modified with triple bonds and the pMHC complex is modified with azide. Alternatively, the coat may be modified with the azide and the pMHC complex modified with the triple bond; the result will also in this case be that the triple bond and the azide moiety will react, thereby covalently linking the pMHC complex to the phage coat protein.
In the above experiment, denaturation was brought about using increased temperature. Alternatively, (partial) unfolding may be mediated by the addition of urea, ethanol, or any other agent or treatment that leads to (partial) unfolding of the HC, beta2M, and/or MHC1 protein, or alpha, beta, and/or MHC2 proteins. Preferably, the treatment should be done in a way that ensures that the phage particle is not (fully) disintegrated.
In the above experiment, the MHC1, MHC2 or their individual components (HC, beta2M; alpha, beta) were azide-modified and added to the triple bond-modified phages. Alternatively, MHC1 Multimers or MHC2 Multimers (e.g., MHC1 Dextramers, MHC2 Tetramers), or their individual components (Multimer scaffold (e.g., streptavidin or dextran) attached to one of the components of the MHC protein (HC, beta2M; alpha, beta)) may be modified by azide, and then added to the triple bond-modified phages—optionally with the additional addition of the other component of the MHC protein (HC, beta2M; alpha, beta). The resulting product will then be a MHC Multimer covalently attached to the phage coat.
In the experiment described above, the MHC1 complex or the individual proteins of the MHC1 complex were added, denatured and then renatured, to form the pMHC1 complex. Alternatively, an active exchange process may be applied. In this approach, a (relatively labile) p*MHC complex (where p* is a peptide that binds with modest affinity to the MHC1 complex, and where the p*MHC complex carries a Base Peptide sequence) is added, instead of the HC, beta2M, or MHC1 complex. Then, instead of denaturation, a so-called helper-ligand is added, capable of intermittent binding to the p*MHC1 complex. The helper-ligand mediates the active replacement of the p* peptide with peptide X from the solution, resulting in formation of the peptide X-MHC1 complex.
In the above example in step (j), the HC protein, beta2M protein or an empty MHC1 complex (without peptide epitope), and/or alpha protein, beta protein or an empty MHC2 complex (without peptide epitope) is modified with an azide moiety, under conditions that results in approximately 1 modification per protein. As an alternative, a MHC Multimer scaffold (e.g., streptavidin tetramer, streptavidin-coated dextran, SP1 scaffold) to which is bound multiple copies of HC protein, beta2M protein, empty MHC1 complex, alpha protein, beta protein, or empty MHC2 complex, and then the scaffold or any of the associated peptides or proteins is modified by an azide moiety, under conditions that results in approximately 1 modification per scaffold with associated peptides or proteins. Then, in in step (l) the triple bond (covalently attached to a phage coat protein) and the azide moiety (covalently attached to the scaffold or one of the associated peptides or proteins will react, thereby covalently linking the MHC Multimer scaffold to the phage coat protein.
In the above example, the pMHC complex may become attached to any solvent-exposed coat protein of the phage particle.
As an alternative, the above procedure can be changed so as to not attach the pMHC complex to the phage (i.e. skipping steps (i-j), simply add empty MHC complexes without chemical modification in step (k), and skip step (l)). The resulting product of the process described by the above steps (a-m) will then be 1000 wells each comprising a unique pMHC complex and a unique DNA.
By appropriate modification, any other type of filamentous or non-filamentous phage, and any virus may be used in this invention. Thus, any phage or virus can be used in the invention.
Likewise, by appropriate modification, any type of cell, including human cell, can be used instead of the phage particle. In this case the peptide X would be carried by a DNA vector such as a plasmid or from the genome of the cell.

### Example 17. Preparation of pMHC Multiplexers by Addition of MHC2 and Fusion of Peptide X to Coat Protein

This example describes the formation of phage particles, each displaying a unique peptide on its surface, followed by the addition of empty MHC2, and if the displayed peptide is an epitope with affinity for this particular allele of MHC2, the possibility of pMHC2 formation, and thereby, formation of phage particles, each displaying a unique pMHC2 complex on its surface, and carrying the DNA that encodes the peptide of the pMHC complex. Thus, pMHC Multiplexers have been formed.

In a preferred embodiment of the invention, phage display is used as the display system. Specifically, the filamentous phage M13 is used, see (FIG. 5), to generate 1000 different pMHC Multiplexers:
Step a. 1000 M13 phagemid constructs are prepared, by cloning 1000 DNA sequences into, respectively, 1000 copies of a M13 phagemid, where the cloned DNA sequences each carries a promoter controlling the expression of a peptide fusion comprising, from the N-terminus: Signal Peptide (SP) fused to Unique peptide X fused to a short linker peptide (e.g., 3-10 amino acids residues, e.g., SSGSSG) fused to the pVIII coat protein of phage M13. Thus, 1000 different phagemid molecules are prepared, each of which carry a unique DNA sequence encoding a unique peptide X sequence, and thus, in (FIG. 5, A), “peptide X” represents a number of (here: 1000) different peptide sequences, where each phagemid encodes a unique peptide sequence. Optionally, the expression level of the fusion peptide comprising unique peptide X, linker peptide, and pVIII coat protein can be controlled by extracellular changes such as the addition of a molecule that stimulates or lowers transcription or translation of the gene encoding peptide X.
Step b. The 1000 phagemid constructs are introduced (e.g., by transformation) into an E. coli strain that is capable of supporting phage infection and amplification, and cell growth is performed for several cell generations. Then Helper Phage is added, and phage amplification is performed for several cell generations. Appropriate activators and repressors of cell growth and expression are added. Phage particles carrying multiple copies of the unique peptide X-linker peptide-pVIII coat fusion and therefore displaying multiple copies of peptide X on their coats, are now produced and are being secreted out to the supernatant, i.e. into the growth medium.
Step c. The E. coli cells and phage particles in the growth medium are separated by centrifugation, by pelleting the cells. The supernatant comprising the phage particles is transferred to a new tube. PEG precipitation of the phages is performed and the phage pellet is resuspended.
Step d. Empty MHC2 complexes are added, and the peptide X displayed on the phage coat protein pVIII is allowed to bind to the empty MHC2, thereby forming the pMHC2 complex, displayed on phage. Thereby up to 1000 unique pMHC multiplexers have been prepared, each comprising a unique peptide X.

### Comments:

In the above example, the pMHC2 complex becomes attached to the pVIII coat protein. Alternatively, the peptide X-flexible linker fusion peptide could have been fused to the pIII coat protein, which would have resulted in the pMHC2 complex becoming attached to the pIII coat protein. Alternatively, the pMHC2 complex could have been attached to any other coat protein of the filamentous phage.
The above example uses a system including both phagemid and helper phage, where the peptide X-linker peptide fusion peptide is fused to the pVIII coat protein. This ensures that only a fraction of the pVIII coat proteins of the phage particle will display the peptide X-flexible linker fusion peptide, and hence only a fraction of the pVIII coat proteins will display a pMHC2 complex.
Alternatively, one may fuse the peptide X-linker peptide fusion peptide to one of two pVIII coat proteins of a phage genome that carries two gVIII genes, thus avoiding the use of a phagemid and a helper phage to separately control expression of wildtype pVIII and peptide X-linker peptide-pVIII, thereby ensuring that only a fraction of the pVIII coat proteins display the fusion peptide.
As a second alternative, one may fuse the peptide X-linker peptide fusion peptide to the one pVIII coat protein encoded by a phage genome, wherefore all pVIII coat proteins of the phage particle will display the fusion peptide (see FIG. 5, C).
In either of the three set-ups described immediately above, the number of pMHC complexes displayed by the phage particle may be controlled by the amount of empty MHC2 that is being added (see FIG. 5, B and C)
In a preferred embodiment of the invention, an E. coli strain is used with deletions or mutations in genes encoding proteases, such as proteases in the membranes or periplasm.
By appropriate modification, any other type of filamentous or non-filamentous phage, and any virus may be used in this invention. Thus, any appropriate coat protein on any phage or virus can be used in the invention.
Likewise, any membrane protein or other type of molecule associated with the extracellular membrane surface of a prokaryotic or eukaryotic cell may be used as the display carrier of the pMHC complex.

### Example 18. Preparation of pMHC Multiplexers by Addition of MHC2 and Acid-Base-Assembled Peptide Multimer Display

This example describes the formation of pMHC Multiplexers, by first intracellular (in E. coli) formation of a larger complex of non-covalently linked peptides each comprising a central portion that encodes potential epitopes for the MHC2 complex, and phage propagation. The complex of peptides becomes displayed on the surface of the phages produced, empty MHC2 is added, and upon pMHC complex formation a library of pMHC Multiplexers have been produced.

In a preferred embodiment of the invention, phage display is used as the display system. Specifically, the filamentous phage M13 is used, see (FIG. 8), to generate 1000 different pMHC Multiplexers:
Step a. 1000 M13 phagemid constructs are prepared, by cloning 1000 DNA sequences into, respectively, 1000 copies of a M13 phagemid, where the cloned DNA sequences each carries a promoter controlling the expression of a peptide fusion comprising, from the N-terminus: Base peptide fused to a unique, 24 amino acid residue peptide X fused to Base peptide, and where the cloned DNA sequences also carries a promoter controlling the expression of a peptide fusion comprising, from the N-terminus: Signal Peptide (SP) fused to Acid Peptide fused to pVIII coat protein of phage M13. Thus, 1000 different phagemid molecules are prepared, each of which carry a unique DNA sequence encoding a unique peptide X sequence, and thus, in (FIG. 8), “peptide X” represents a number of (here: 1000) different peptide sequences, where each phagemid encodes a unique peptide sequence. Optionally, the expression level of the fusion peptides can be controlled by extracellular changes such as the addition of a molecule that stimulates or lowers transcription or translation of the gene encoding peptide X.
Step b. Additionally, a DNA carrying a promoter controlling the transcription of a DNA sequence encoding a fusion peptide, from the N-terminus: Acid Peptide in fusion with a short peptide linker (SSG) in fusion with Acid Peptide, is cloned into each of the phagemids of step a.
Step c. The 1000 phagemid constructs are introduced (e.g., by transformation) into an E. coli strain that is capable of supporting phage infection and amplification, and cell growth is performed for several cell generations. Then Helper Phage is added, and phage amplification is performed for several cell generations. Appropriate activators and repressors of cell growth and expression are added. Phage particles carrying multiple copies of the Acid Peptide-pVIII and therefore displaying multiple copies of Acid Peptide-pVIII on their coats, are now produced.

Also, the Base Peptide-Peptide X-Base Peptide fusion peptide, as well as the Acid Peptide-SSG-Acid Peptide fusion peptide, is expressed. As a result, several Base Peptide-Peptide X-Base Peptide fusion peptides and several Acid Peptide-SSG-Acid Peptide fusion peptides will have formed, some of which will further have complexed to the Acid Peptide of the Acid-pVIII fusion protein in the phage coat, as shown in (FIG. 8). These complexes are held together by the interaction between Acid Peptides and Base Peptides, a relatively strong interaction.

Upon phage packaging termination, the phages are being secreted out to the supernatant, i.e. into the growth medium.
Step d. The E. coli cells and phage particles in the growth medium are separated by centrifugation, by pelleting the cells. The supernatant comprising the phage particles is transferred to a new tube. PEG precipitation of the phages is performed, the supernatant removed, and the phage pellet is resuspended.
Step e. Empty MHC2 complexes are added, and the peptide X displayed on the phage coat protein pVIII is allowed to bind to the empty MHC2, thereby forming the pMHC2 complex, displayed on phage. Thereby up to 1000 unique pMHC multiplexers have been prepared, each comprising one or more pMHC complexes, each comprising a unique peptide X encoded by the DNA inside the phage.

### Comments:

In the above example, the Acid Peptide-pVIII fusion peptide is carried on the phagemid. This fusion peptide can also be carried on the Helper Phage or on a separate plasmid in the same cell.
If cysteine-comprising variants of the Acid Peptide and Base Peptide variants are used, the Acid Peptide-Base Peptide dimers may be stabilized by forming a disulfide bond between the Acid Peptide and the Base Peptide.
In the above example, the Acid peptide is fused to the pVIII coat protein. Alternatively, the Acid Peptide may be fused to the pIII coat protein, thereby reducing the pMHC display valency of the phage.
The above example uses a system including both phagemid and helper phage. This ensures that only a fraction of the pVIII coat proteins of the phage particle will display the Acid Peptide, and hence only a fraction of the pVIII coat proteins will display a pMHC2 complex.
Alternatively, one may fuse the DNA sequence encoding the Acid Peptide to one of two genes encoding pVIII coat proteins of a phage genome (that carries two gVIII genes), thus avoiding the use of both a phagemid and a helper phage.
As a second alternative, one may fuse the Acid Peptide to the one pVIII coat protein encoded by a phage genome, wherefore all pVIII coat proteins of the phage particle will display the Acid Peptide.
In all of the set-ups described above, the number of pMHC complexes displayed by phage particle may be adjusted by the amount of empty MHC2 that is being added.
In a preferred embodiment of the invention, an E. coli strain is used with deletions or mutations in genes encoding proteases.
By appropriate modification, any other type of filamentous or non-filamentous phage, and any virus may be used in this invention. Thus, any appropriate coat protein on any phage or virus can be used in the invention.
Likewise, any membrane protein or other type of molecule associated with the extracellular membrane surface of a prokaryotic or eukaryotic cell may be used as the display carrier of the pMHC complex.

### Example 19. pMHC Multiplexers by Intracellular pMHC Formation

In this example, the three components of the pMHC complex—the peptide (p), the heavy chain and the b2M peptides—are produced internally in the E. coli cell. The heavy chain is further fused to the pVIII coat protein. As a result, the resulting phage particles display pMHC1 complexes on their surface.

In a preferred embodiment of the invention, phage display is used as the display system. Specifically, the filamentous phage M13 is used, see (FIG. 10), to generate a large number of different pMHC Multiplexers. In this example, 1000 unique pMHC Multiplexers are produced:
Step a. On a phagemid, a DNA sequence encoding, from the N-terminal of the peptide, the Signal Peptide (SP) fused to the heavy chain (HC) component of the MHC1 protein fused to pVIII. The SP-HC-gVIII fusion gene is under the control of a promoter whose expression can be modified by the addition of a molecule to the growth medium. One such molecule may be IPTG, where the promoter is under the regulatory control of IPTG.
Step b. On the same phagemid, a gene encoding a short peptide X is under the control of the same promoter as described above, or another promoter. Each phagemid copy encodes a unique peptide X; a collection of phagemids is used, here 1000 phagemids are present, where each phagemid carries a gene encoding a unique peptide sequence. Thus, in the figure, “peptide X” represents a number of (here: 1000) different peptide sequences, where each phagemid encodes a unique peptide sequence. Optionally, the production of peptide X is very efficient, leading to high amounts of peptide X in a bacterial cell carrying the phagemid. Optionally, the expression level of peptide X can be controlled by extracellular changes such as the addition of a molecule that stimulates transcription or translation of the gene encoding peptide X. On a vector, either the same phagemid as described above or another vector as depicted in (FIG. 10, A), a gene encoding beta2M is cloned.
Step c. The phagemid constructs each encoding a unique peptide X sequences and all carrying the SP-HC-pVIII gene fusion, as well as the vector carrying the beta2M, are introduced (e.g., by transformation) into an E. coli strain that is capable of supporting phage infection and amplification, and cell growth is performed for several cell generations. Then Helper Phage is added, and phage amplification is performed for several cell generations. Phage particles, carrying several copies of the assembled pVIII-pMHC1 complexes, are produced and are being secreted out of the cell, i.e. into the growth medium. Optionally, protease inhibitor(s) and/or nuclease inhibitors are added.
Step d. The E. coli cells and phage particles in the growth medium are separated by centrifugation, by pelleting the cells. The supernatant comprising the phage particles is transferred to a new tube.
Step e. Optionally, the phage particles are PEG precipitated.

The resulting product of the process described by the above steps (a-e), is 1000 unique pMHC Multiplexers.

### Comments:

In the experiment described above, HC is fused to the gVIII gene encoding the pVIII coat protein, carried by the phagemid, the unique peptide X is also carried by the phagemid, and the gene encoding beta2M protein is carried by another vector. Alternatively, beta2M may be fused to the gene encoding pVIII and carried on the phagemid, while HC is carried on another vector.
In the experiment above or in the point immediately above, HC or beta2M is fused to the gene encoding pVIII. Alternatively, the HC or beta2M protein may be fused to a gene encoding pIII.
As a further alternative (see FIG. 10), encoded by the phagemid the Acid Peptide may be fused to pVIII, Base Peptide is fused to HC, peptide X is encoded by the phagemid, and beta2M is encoded by phagemid, Helper Phage or another vector in the cell. Upon Acid Peptide-Base Peptide formation, the pMHC1 complex will become non-covalently attached to pVIII, and thus, will become displayed on the surface of the phage particle.
As a further alternative, encoded by the phagemid the Acid Peptide may be fused to pVIII, Base Peptide is fused to beta2M, peptide X is encoded by the phagemid, and HC is encoded by phagemid, Helper Phage or another vector in the cell. Upon Acid Peptide-Base Peptide formation, the pMHC1 complex will become non-covalently attached to pVIII, and thus, will become displayed on the surface of the phage particle.
In the above example, pMHC1 complexes were formed from HC and beta2M protein, and attached to the phage coat. Alternatively, pMHC2 complexes could be formed from alpha and beta protein, where either alpha or beta were fused to Base Peptide or to pVIII or PIII. And as a result, the phage particles would display pMHC2 complexes.
The above example uses a system including both phagemid and helper phage. This ensures that only a fraction of the pIII coat proteins or of the pVIII coat proteins of the phage particle will display a pMHC complex. Alternatively, the coat fusion proteins may be carried by a phage genome, in which case all of the pIII coat proteins or all of the pVIII coat proteins will display a pMHC complex.
In a preferred embodiment of the invention, an E. coli strain is used with deletions or mutations in genes encoding proteases, such as proteases in the membranes or periplasm, in order to increase the average life-time of the expressed peptides.
By appropriate modification, any other type of filamentous or non-filamentous phage, and any virus may be used in this invention. Thus, any appropriate coat protein on any phage or virus can be used in the invention.

Likewise, any membrane protein or other type of molecule associated with the extracellular membrane surface of a prokaryotic or eukaryotic cell may be used as the display carrier of the pMHC complex.

### Example 20. pMHC Multiplexers by Intracellular Pentamer Formation

This example describes how phage particles carrying MHC Pentamers can be produced. The result is pMHC Multiplexers displaying MHC Pentamers.

In a preferred embodiment of the invention, phage display is used as the display system. Specifically, the filamentous phage M13 is used, see (FIG. 12), to generate a large number of different pMHC Multiplexers, each of which carry one or more copies of the tryptophan-zipper pentamer. In this example, 1000 unique pMHC Multiplexers are produced:
Step a. An M13-derived phagemid is constructed carrying a promoter controlling a DNA sequence encoding a fusion peptide consisting of, from the N-terminal end, a signal peptide (SP), Acid peptide, tryptophan-zipper pentamer subunit (Psub), and pIII coat protein. Further, the phagemid carries a promoter controlling the transcription of a unique peptide X. 1000 unique plasmid constructs are generated: all phagemids carry the same peptide fusion SP-Acid-Psub-pIII, but each phagemid carries a unique peptide X (see FIG. 12). The gIII fusion gene is under the control of a promoter whose expression can be modified by the addition of a molecule to the growth medium. One such molecule may be IPTG, where the promoter is under the regulatory control of IPTG. Optionally, the production of peptide X is very efficient, leading to high amounts of peptide X in a bacterial cell carrying the phagemid. Optionally, the expression level of peptide X can be controlled by extracellular changes such as the addition of a molecule that stimulates transcription or translation of the gene encoding peptide X.
Step b. On the same phagemid, or on another vector in the same cell, is a DNA sequence encoding a Base Peptide-HC fusion protein, the beta2M protein, and an Acid Peptide-Pentamer subunit fusion.
Step c. The DNA constructs of step (a) and (b) are introduced (e.g., by transformation) into an E. coli strain that is capable of supporting phage infection and amplification, and cell growth is performed for several cell generations. Then Helper Phage is added, and phage amplification is performed for several cell generations. Phage particles, carrying one or more copies of the Pentamer that carries up to five pMHC complexes each, are produced and are being secreted out of the cell, i.e. into the growth medium. Optionally, protease inhibitor(s) and/or nuclease inhibitors are added.
Step d. The E. coli cells and phage particles in the growth medium are separated by centrifugation, by pelleting the cells. The supernatant comprising the phage particles is transferred to a new tube.
Step e. Optionally, the phage particles are PEG precipitated.

The resulting product of the process described by the above steps (a-e), is 1000 unique pMHC Multiplexers.

### Comments:

The tryptophan-zipper Pentamer is described in the paper “Atomic structure of a tryptophan-zipper pentamer”, PNAS, 101, p. 16156-16161.
In the experiment described above, HC is fused to the Base Peptide. Alternatively, beta2M may be fused to the Base Peptide.
In the experiment above or in the point immediately above, HC or beta2M is fused to the Base peptide. Alternatively, the HC or beta2M protein may be fused to Acid Peptide, and then the PSub should be fused to Base Peptide. Other heterodimers than Acid Peptide-Base Peptide may be used.
As a further alternative, the gIII gene encoding the M13 pIII coat protein, may be replaced by the gVIII gene encoding the M13 pVIII coat protein.
In the above example, pMHC1 complexes were formed from HC and beta2M protein, and attached to the phage coat. Alternatively, pMHC2 complexes could be formed from alpha and beta protein, where either alpha or beta were fused to Base Peptide. And as a result, the phage particles would display pMHC2 complexes.
In the above example, the Acid Peptide-Psub peptide fusion was expressed in the E. coli cell, leading to display of a pMHC Pentamer on the phage particle surface. By constructing a DNA vector encoding Psub-Acid Peptide-Psub peptide the display valence of pMHC may be increased to nine.
The above example uses a system including both phagemid and helper phage. This ensures that only a fraction of the pIII coat proteins or of the pVIII coat proteins of the phage particle will display a pMHC complex. Alternatively, the coat fusion proteins may be carried by a phage genome, in which case all of the pIII coat proteins or all of the pVIII coat proteins will display a pMHC complex.
In a preferred embodiment of the invention, an E. coli strain is used with deletions or mutations in genes encoding proteases, such as proteases in the membranes or periplasm, in order to increase the average life-time of the expressed peptides.
By appropriate modification, any other type of filamentous or non-filamentous phage, and any virus or may be used in this invention. Thus, any appropriate coat protein on any phage or virus can be used in the invention.
Likewise, any membrane protein or other type of peptide associated with the extracellular membrane surface of a prokaryotic or eukaryotic cell may be used as the display carrier of the pMHC complex.

### Example 21. pMHC Multiplexers by Intracellular SP1 Multimer Formation

This example describes how phage particles carrying SP1-based MHC Multimers can be produced. The result is pMHC Multiplexers displaying SP1-based MHC Multimers.

In a preferred embodiment of the invention, phage display is used as the display system. Specifically, the filamentous phage M13 is used, see (FIG. 13), to generate a large number of different pMHC Multiplexers, each of which carry one or more copies of the SP1 dodecamer protein scaffold. In this example, 1000 unique pMHC Multiplexers are produced:
Step a. A M13-derived phagemid is constructed carrying a promoter controlling a DNA sequence encoding a fusion peptide consisting of, from the N-terminal end, a Signal Peptide (SP), Acid peptide, and pIII coat protein. Further, the phagemid carries a promoter controlling the transcription of a unique peptide X. 1000 unique plasmid constructs are generated: all phagemids carry the same peptide fusion Signal Peptide (SP), Acid peptide, and pIII coat protein, but each phagemid carries a unique peptide X (see FIG. 13). The gIII fusion gene is under the control of a promoter whose expression can be modified by the addition of a molecule to the growth medium. One such molecule may be IPTG, where the promoter is under the regulatory control of IPTG. Optionally, the production of peptide X is very efficient, leading to high amounts of peptide X in a bacterial cell carrying the phagemid. Optionally, the expression level of peptide X can be controlled by extracellular changes such as the addition of a molecule that stimulates transcription or translation of the gene encoding peptide X.
Step b. On the same phagemid, or on another vector in the same cell, is a DNA sequence encoding an Acid Peptide-HC fusion protein, the beta2M protein, and a fusion peptide of, from the N-terminal, Base Peptide-Flexible linker peptide (e.g., SSGSSG)-SP1 subunit peptide.
Step c. The DNA constructs of step (a) and (b) are introduced (e.g., by transformation) into an E. coli strain that is capable of supporting phage infection and amplification, and cell growth is performed for several cell generations. Then Helper Phage is added, and phage amplification is performed for several cell generations. Phage particles, carrying one or more copies of the SP1 protein scaffold that carries up to 12 pMHC complexes each, are produced and are being secreted out of the cell, i.e. into the growth medium. Optionally, protease inhibitor(s) and/or nuclease inhibitors are added.
Step d. The E. coli cells and phage particles in the growth medium are separated by centrifugation, by pelleting the cells. The supernatant comprising the phage particles is transferred to a new tube.
Step e. Optionally, the phage particles are PEG precipitated.

The resulting product of the process described by the above steps (a-e), is 1000 unique pMHC Multiplexers, each of which carries up to 12 pMHC complexes.

### Comments:

The SP1 dodecamer protein is described in the paper “Protein scaffold engineering towards tunable surface attachment”, Angew. Chem. Int. Ed. 2009, vol 48, p. 9290-9294.
In the experiment described above, HC is fused to the Acid Peptide. Alternatively, beta2M may be fused to the Acid Peptide.
In the experiment above or in the point immediately above, HC or beta2M is fused to the Acid peptide. Alternatively, the HC or beta2M protein may be fused to Base Peptide, and then the Sp1 should be fused to Acid Peptide. Other heterodimers than Acid Peptide-Base Peptide may be used.
As a further alternative, the gIII gene encoding the M13 pIII coat protein, may be replaced by the gVIII gene encoding the M13 pVIII coat protein.
In the above example, pMHC1 complexes were formed from HC and beta2M protein, and attached to the phage coat. Alternatively, pMHC2 complexes could be formed from alpha and beta protein, where either alpha or beta were fused to Base Peptide. And as a result, the phage particles would display pMHC2 complexes.
The above example uses a system including both phagemid and helper phage. This ensures that only a fraction of the pIII coat proteins or of the pVIII coat proteins of the phage particle will display a pMHC complex. Alternatively, the coat fusion proteins may be carried by a phage genome, in which case all of the pIII coat proteins or all of the pVIII coat proteins will display a pMHC complex.
In a preferred embodiment of the invention, an E. coli strain is used with deletions or mutations in genes encoding proteases, such as proteases in the membranes or periplasm, in order to increase the average life-time of the expressed peptides.
By appropriate modification, any other type of filamentous or non-filamentous phage, and any virus or may be used in this invention. Thus, any appropriate coat protein on any phage or virus can be used in the invention.
Likewise, any membrane protein or other type of peptide associated with the extracellular membrane surface of a prokaryotic or eukaryotic cell may be used as the display carrier of the pMHC complex.

### Example 22. pMHC Multiplexers by Intracellular Tetramerₙ Formation

In this example, MHC Tetramers are being produced intracellularly in E. coli, and by fusion of pIII coat protein and streptavidin, the phage particles end up displaying MHC Multimers. The result is therefore pMHC Multiplexers displaying MHC Tetramers.

In a preferred embodiment of the invention, phage display is used as the display system. Specifically, the filamentous phage M13 is used, see (FIG. 14), to generate a large number of different pMHC Multiplexers, each of which carry one or more copies of the streptavidin Tetramer. In this example, 1000 unique pMHC Multiplexers are produced:
Step a. An M13-derived phagemid is constructed carrying a promoter controlling a DNA sequence encoding a fusion peptide consisting of, from the N-terminal end, a signal peptide (SP), streptavidin (SA), and pIII coat protein. Further, the phagemid carries a promoter controlling the transcription of a unique peptide X. 1000 unique plasmid constructs are generated: all phagemids carry the same peptide fusion SP-SA-pIII, but each phagemid carries a unique peptide X (see FIG. 14). The gIII fusion gene is under the control of a promoter whose expression can be modified by the addition of a molecule to the growth medium. One such molecule may be IPTG, where the promoter is under the regulatory control of IPTG. Optionally, the production of peptide X is very efficient, leading to high amounts of peptide X in a bacterial cell carrying the phagemid. Optionally, the expression level of peptide X can be controlled by extracellular changes such as the addition of a molecule that stimulates transcription or translation of the gene encoding peptide X.
Step b. On the same phagemid, or on another vector in the same cell, is a DNA sequence encoding an Acceptor Peptide-HC fusion protein, the beta2M protein, streptavidin, and biotin ligase (birA).
Step c. The DNA constructs of step (a) and (b) are introduced (e.g., by transformation) into an E. coli strain that is capable of supporting phage infection and amplification, and cell growth is performed for several cell generations. Then Helper Phage is added, and phage amplification is performed for several cell generations. Phage particles, carrying one or more copies of the Tetramer that carries up to four pMHC complexes each, are produced and are being secreted out of the cell, i.e. into the growth medium. Optionally, protease inhibitor(s) and/or nuclease inhibitors are added.
Step d. The E. coli cells and phage particles in the growth medium are separated by centrifugation, by pelleting the cells. The supernatant comprising the phage particles is transferred to a new tube.
Step e. Optionally, the phage particles are PEG precipitated.

The resulting product of the process described by the above steps (a-e), is 1000 unique pMHC Multiplexers, each comprising one or more Tetramers each carrying up to four pMHC complexes.

### Comments:

The Acceptor Peptide (AP) is a peptide sequence capable of being biotinylated in vivo by the biotin ligase.
In the experiment described above, HC is fused to the Acceptor Peptide (AP). Alternatively, beta2M may be fused to the Acceptor Peptide.
As a further alternative, the gIII gene encoding the M13 pIII coat protein, may be replaced by the gVIII gene encoding the M13 pVIII coat protein.
In the above example, pMHC1 complexes were formed from HC and beta2M protein, and attached to the phage coat. Alternatively, pMHC2 complexes could be formed from alpha and beta protein, where either alpha or beta were fused to AP. And as a result, the phage particles would display pMHC2 complexes.
The above example uses a system including both phagemid and helper phage. This ensures that only a fraction of the pIII coat proteins or of the pVIII coat proteins of the phage particle will display a pMHC complex. Alternatively, the coat fusion proteins may be carried by a phage genome, in which case all of the pIII coat proteins or all of the pVIII coat proteins will display a pMHC complex.
In a preferred embodiment of the invention, an E. coli strain is used with deletions or mutations in genes encoding proteases, such as proteases in the membranes or periplasm, in order to increase the average life-time of the expressed peptides.
By appropriate modification, any other type of filamentous or non-filamentous phage, and any virus or may be used in this invention. Thus, any appropriate coat protein on any phage or virus can be used in the invention.
Likewise, any membrane protein or other type of peptide associated with the extracellular membrane surface of a prokaryotic or eukaryotic cell may be used as the display carrier of the pMHC complex.

### Example 23. pMHC Multiplexers by Intracellular pMHC and Acid Poly-Repeat Scaffold

In this example, a poly-repeat peptide carrying multiple repeats of the Acid peptide sequence is generated. The poly-repeat is fused to pIII, and therefore the phage particles display the poly-repeat on their surface. Simultaneous intracellular pMHC complex formation (involving a heavy chain that is fused to the Base-peptide) results in the formation of a poly-pMHC structure that is displayed on phage, i.e., a pMHC Multiplexer has been generated.

In a preferred embodiment of the invention, phage display is used as the display system. Specifically, the filamentous phage M13 is used, see (FIG. 17), to generate 1000 different pMHC Multiplexers:
Step a. On a phagemid, a DNA sequence encoding, from the N-terminal end, signal peptide (SP) fused to a peptide comprising 6 Acid Peptide repeat sequences fused to the pIII coat protein. Here, an Acid Peptide variant is used that carries a cysteine, capable of forming a disulfide bond with Base Peptide, also carrying a cysteine, upon their complexation. The SP-Acid₆-gIII fusion gene is under the control of a promoter whose expression can be modified by the addition of a molecule to the growth medium. One such molecule may be IPTG, where the promoter is under the regulatory control of IPTG. See (FIG. 17).
Step b. On the same phagemid, a gene encoding a short peptide X is under the control of the same promoter as described above, or another promoter. Each phagemid copy encodes a specific peptide X; a collection of phagemids is used, here 1000 phagemids are present, where each phagemid carries a gene encoding a unique peptide sequence. Thus, in the figure, “peptide X” represents a number of (here: 1000) different peptide sequences, where each phagemid encodes a unique peptide sequence. Optionally, the production of peptide X is very efficient, leading to high amounts of peptide X in a bacterial cell carrying the plasmid. Optionally, the expression level of peptide X can be controlled by extracellular changes such as the addition of a molecule that stimulates transcription or translation of the gene encoding peptide X.
Step c. The same phagemid, or another vector such as a plasmid, carries a gene encoding beta2M and a gene encoding a protein-fusion of the Base Peptide and HC (see FIG. 17). Beta2M and HC are the two proteins of the MHC1 protein; the Base Peptide used in this construct comprises a cysteine capable of forming a disulfide bond with an Acid Peptide upon formation of a Acid-Base Peptide dimer.
Step d. The phagemid constructs encoding a total of 1000 unique peptide X sequences and all carrying the Acid₆-pIII gene fusion, and the vector carrying the beta2M and Base-HC fusion, are introduced (e.g., by transformation) into an E. coli strain that is capable of supporting phage infection and amplification, and cell growth is performed for several cell generations. Then Helper Phage is added, and phage amplification is performed for several cell generations. Phage particles, some of which are carrying a copy of the Acid₆ peptide, fused to the pIII coat protein, are produced. Meanwhile, the peptide X, the beta2M and the Base Peptide-HC fusion peptides are being expressed and folded, and now assemble to form a pMHC complex, where the HC component is fused to the Base Peptide. This Base Peptide may dimerize with one of the six Acid Repeats of the peptide attached to pIII protein. As a result, phage particles will form that carry up to six pMHC complexes attached to a pIII coat protein by way of Acid-Base dimerization. These phage particles are secreted out of the cell, i.e. into the growth medium. Optionally, protease inhibitor(s) and/or nuclease inhibitors are added—before, during or after (partial) lysis.
Step e. The E. coli cells and phage particles in the growth medium are separated by centrifugation, by pelleting the cells. The supernatant comprising the phage particles is transferred to a new tube.
Step f. Optionally, the phages are PEG precipitated.

The resulting phages represent 1000 unique pMHC Multiplexers, where each multiplexer comprises a phage, where the phage particle contains a unique DNA molecule that encodes the unique peptide (p) of the pMHC complex, and where the pMHC Multiplexer comprises a number of pMHC complexes attached to the Acid 6-pIII coat protein of the phage.

The 1000 unique pMHC Multiplexers may be used individually or as a library in the screening of antigen-specific T cells, as described below.

### Comments:

In the above experiment, the multiple Acid Peptide repeats are fused to pIII, and Base Peptide is fused to HC. Alternatively, multiple repeats of Base Peptide may be fused to pIII and Acid Peptide may be fused to HC.
In the above experiment, the Base Peptide is fused to the HC of the MHC1 complex, which leads to attachment of the MHC1 complex to the Acid Peptide. Alternatively, the Base Peptide may be fused to beta2M of the MHC1 complex, leading also to attachment of the MHC1 complex to the Acid Peptide.
In the above example, pMHC1 complexes were formed from HC and beta2M protein, and attached to the phage coat. Alternatively, pMHC2 complexes could be formed from alpha and beta protein, where either alpha or beta were fused to Base Peptide.
In the above example, the pMHC complex becomes attached to the pIII coat protein. Alternatively, the multiple repeats of Acid Peptide could have been fused to the pVIII coat protein, which would have resulted in the pMHC complexes becoming attached to the pVIII coat protein. Alternatively, the pMHC complexes could have been attached to any other coat protein of the filamentous phage.
The above example uses a system including both phagemid and helper phage. This ensures that only a fraction of the pIII coat proteins or of the pVIII coat proteins of the phage particle will display the Acid Peptide, and hence only a fraction of the pIII coat proteins or of the pVIII coat proteins of the phage particle will display a pMHC complex after its attachment to the Acid Peptide. Alternatively, one may fuse the multiple Acid Peptide repeats to the pIII protein or pVIII protein encoded by the phage DNA using a phage where the gene encoding pIII or pVIII is duplicated, thus avoiding the use of a phagemid and a helper phage. Hence, in this example, a phagemid would not be included, and the peptide X should be encoded by the phage DNA, and not the phagemid DNA as was done in the above example.
In a preferred embodiment of the invention, an E. coli strain is used with deletions or mutations in genes encoding proteases, such as proteases in the membranes or periplasm, in order to increase the average life-time of the expressed peptides.
By appropriate modification, any other type of filamentous or non-filamentous phage, and any virus may be used in this invention. Thus, any appropriate coat protein on any phage or virus can be used in the invention.
Likewise, any membrane protein or other type of molecule associated with the extracellular membrane surface of a prokaryotic or eukaryotic cell may be used as the display carrier of the pMHC complex.

### Example 24. pMHC Multiplexers by Artificially Encoded Dendritic Cell Display

DNA encoding potential epitopes are in this example introduced into dendritic cells by e.g. electroporation, and upon translation in the cells, some of the encoded peptides become displayed in the form of pMHC complexes on the surface of the dendritic cell. These “artificially programmed dendritic cells” (programmed to display the peptides encoded by the DNA that was introduced) can now be used as pMHC Multiplexers, for the detection, sorting or modification (e.g., stimulation, induction of apoptosis, or other) of antigen-specific T cells.

In a preferred embodiment of the invention, an antigen-presenting cell, such as a dendritic cell, is used as the display system, to generate a large number, i.e., at least 2, such as at least 10, such as at least 100, such as at least 1000, such as at least 10000, such as at least 100000, such as at least 1000000, such as at least 10000000 different pMHC Multiplexers. Immediately below, a general production method is described along with example subprocesses available for making the components of a pMHC Multiplexer of the invention. See also (FIG. 20).
Step a. A collection of encoding molecules (e.g., DNA or RNA), where each encodes the peptide of the pMHC complex being displayed in multiple copies in a MHC Multiplexer, or where each encodes the precursor peptide or precursor protein of the peptide of the pMHC complex being displayed in multiple copies in a MHC Multiplexer, are generated.

The encoding molecules may be a collection of RNA molecules or a collection of DNA molecules. The DNA molecules may be made by synthetic chemistry or may be made by enzymatic means such as by reverse transcription of an mRNA, followed by amplification e.g., by PCR. In a preferred embodiment the DNA comprises a promoter and the transcribed mRNA comprises sequences for translation, e.g., ribosome binding sites or other signal sequences for translation initiation.

The DNA may be e.g., a wildtype virus or a genetically modified recombinant virus e.g., belonging to the following group of viruses: adenovirus, retrovirus, herpes simplex virus, vaccinia virus, influenza virus, and alpha virus. The DNA may be double-stranded or single-stranded and may comprise the following number of nucleotides (for single-stranded DNA) or base pairs (for double-stranded DNA): 20-10000000, such as 20-1000000, such as 20-100000, such as 20-10000, such as 20-1000, such as 20-100, such as 20-70, such as 20-50, or may be 40-10000000, such as 40-1000000, such as 40-100000, such as 40-10000, such as 40-1000, such as 40-100, such as 40-70, such as 40-50.

The RNA may be a collection of mRNA molecules purified from a cell extract such as a cell extract from a cancer patient, e.g., a cell extract of the cells from a biopsy from the tumor of a cancer patient. Alternatively, cDNA may be prepared from a cell extract, amplified by e.g., PCR and then transcribed into mRNA, or mRNA may be made by transcription from (optionally recombinant) viral DNA or other vector DNA such as plasmids, or may be made from transcription of single-stranded or double-stranded oligonucleotides prepared synthetic chemistry.

The stability of the encoding molecules before or after introduction into the cells may be improved by several means, including (i) modification of the chemical structure of the encoding molecule, such as methylation and/or introduction of N1-methyl-pseudouridine and/or modification at the termini of the encoding molecules, or by circularizing the encoding molecules (ii) using cells that have been depleted for certain nucleases that have had certain genes encoding nucleases deleted or mutated to decrease or eliminate the corresponding nucleases' nucleolytic effects on the encoding molecule, and (iii) using cells that express proteins or produce other molecules that bind to the encoding molecule and thereby increase the stability of the encoding molecules.
Step b. The encoding molecules are introduced into a cell, under conditions ensuring that one cell only receives one or more copies of the same encoding molecule, thereby generating a collection of cells, each comprising one or more copies of an encoding molecule. The encoding molecules can be introduced into the cell by a number of methods, including (i) electroporation, (ii) infection by e.g., virus, (iii) phagocytosis of another cell, e.g., a monocyte or bacteria (iv) uptake of lipid-nanoparticles or vesicles or other similar entities, (v) small lipid-comprising carriers, (vi) infection by e.g., bacterium, and (vii) transfection, e.g., using liposomes such as DOTAP.

The encoding molecules may be introduced into cells from a variety of organisms, including humans and other mammals, rodents such as mice and rats, goats, birds, rabbits, guinea pigs, hamsters, farm animals (including pigs and sheep), dogs, primates (including apes, monkeys and chimpanzees), frogs, fish, cats and yeast.

The encoding molecules may be introduced into a variety of cell types, including dendritic cells (DCs), macrophages, B cells, thymic and other epithelial cells, endothelial cells, and any type of cell capable of displaying on its surface a pMHC complex, such as pMHC1, pMHC2, and MHC-like complexes. Cells capable of displaying such pMHC or MHC-like complexes on their surface are called antigen-presenting cells. Depending on the method for introduction of the encoding molecules, the cells may be treated in various ways to make the introduction of encoding molecules more efficient.
Step c. The encoding molecules may be expressed in the cells, to each generate the encoded peptide of the pMHC complex being displayed in multiple copies in a MHC Multiplexer, or to each generate the precursor peptide or precursor protein for the peptide of the pMHC complex being displayed in multiple copies in a MHC Multiplexer. Alternatively, the peptide (p) of the pMHC complex, or a precursor peptide or precursor protein for the peptide, may have been introduced into the cell before, during or after the introduction of the encoding molecule that encodes it (see comment below).
Step d. The peptide or a precursor peptide or precursor protein for the peptide (p) may be modified. Thus, optionally, the introduced encoding molecule and/or introduced or encoded peptide (p) of the MHC complex are transcribed and/or translated and/or (partially) proteolyzed and/or (partially) degraded and/or in any other way modified. Then the peptide (p) is complexed with MHC or MHC like complexes and is displayed on the surface of the cell.
Step e. Optionally, one or more stimulatory or inhibitory or modulating molecules are added to the collection of cells, and optionally, is allowed to incubate for one or more days.

Thus, the product of steps (a)-(e) is a number (N) of pMHC Multiplexers, where each pMHC Multiplexer comprises a cell (e.g., a dendritic cell) displaying on its surface a number of identical pMHC complexes, and where said cell comprises or is associated with an encoding molecule (e.g., DNA or RNA) that encodes the identity of said number of identical pMHC complexes.

The sequence of steps (a)-(e) can be in any order, as long as the end product is a cell displaying on its surface a pMHC complex where the identity of the pMHC complex is encoded by the encoding molecule associated with the cell. This is a pMHC Multiplexer.

In the following, two example applications of a collection of pMHC Multiplexers, prepared in a way similar to that described immediately above, are described. See also (FIG. 20).

In the first example, the pMHC Multiplexer from above is added to a blood sample, and the mixture incubated (lower part of FIG. 20, left). After one or more days of incubation bispecific antibodies are added. These bi-specific antibodies have binding specificity for both (i) a certain molecule X secreted from the cell of the pMHC Multiplexer upon interaction with a T cell or from a T cell upon interaction with an antigen-presenting cell, and for (ii) a receptor or membrane bound protein of the cell of the pMHC Multiplexer. Also, a fluorescent-labelled antibody with affinity for the secreted molecule X is added. After a further incubation time of 1-5 hours pMHC

Multiplexers that fluoresce are collected by flow sorting or by manually collecting these under a microscope. Finally, the DNA of the pMHC Multiplexers that were collected are sequenced.

In the second example, a pMHC Multiplexer is generated as described above, and in addition a vector has been introduced into the cell (that will become part of the pMHC Multiplexer) where the vector carries an interleukin-responsive (interleukin-activated) promoter controlling the transcription of the GFP gene (see lower part of FIG. 20, right). The interleukin used is an interleukin whose expression goes up upon interaction between the pMHC complexes of the pMHC Multiplexer and the T cell receptors of a T cell. After incubation the pMHC Multiplexers that have interacted productively with a T cell will fluoresce and can be collected, and finally, the encoding molecules of the pMHC Multiplexers that were collected are sequenced.

### Comments:

In step (b), the peptide (p) of the pMHC complex may be transported into the cell together with the encoding molecule that encodes it. As an example, the peptide and its encoding molecule can be placed in the same vesicle that is then taken up by a cell; or the peptide and its encoding molecule can be bound to the same particle (e.g., gold particle) that is being electroporated into a cell; or the peptide and its encoding molecule can be in the same solution that is injected into a cell; or the peptide and its encoding molecule may be present in a cell that is then phagocytosed by another cell. Under such circumstances where the peptide (p) of the pMHC complex is delivered to the cell together with its encoding molecule, the encoding molecule does not necessarily need to be able to template the synthesis of an mRNA or peptide—but it must be possible to determine its identity, e.g., by sequencing, mass spectrometry or any other means that can identify it uniquely.
In the experiment above, the encoding molecule was introduced into the cell. As an alternative, the encoding molecule may be attached covalently or noncovalently to the surface of the cell.

### Example 25. pMHC Multiplexers, Sense-Antisense, and PCR Detection of Antigen-Specific T Cells

In this example, each unique pMHC complex is tagged with either sense or antisense DNA, and the annealing of the sense and antisense strands of two pMHC complexes of the same specificity—in synergy with binding of the pMHC to T cell receptor—allows PCR detection of those pMHC complexes capable of specifically binding antigen-specific T cells.

In a preferred embodiment of the invention, each pMHC Multiplexer contains only one pMHC complex, a DNA molecule is attached to the pMHC Multiplexer, and the DNA molecule serves at least two purposes, i.e. i) a part of the DNA molecule (e.g., five nucleotides at the 3′-end) is capable of annealing to another pMHC Multiplexer comprising an identical peptide (p) of the pMHC complex, and ii) comprises a unique encoding sequence that specifies the identity of said peptide (p) of the pMHC complex. The two parts of the DNA that serve these two purposes may be identical, overlapping or non-overlapping. The two purposes may also be served by two DNA molecules attached to the same pMHC complex. The following procedure describes the preparation of three such pMHC Multiplexers; see (FIG. 21).
Step a. Three unique pMHC complexes (A, B, C) are each aliquoted into two wells.
Step b. For each of A, B, and C: To one of the two wells is added a DNA molecule (“Sense”), carrying a reactive group X (e.g., N-hydroxysuccinimide ester) at one end, thereby making it capable of reacting with a reactive group Y (e.g., amino group) on the pMHC complex. To the other of the two wells is added a DNA molecule (“Antisense”), carrying a reactive group X (e.g., N-hydroxysuccinimide ester) at one end, thereby making it capable of reacting with a reactive group Y (e.g., amino group) on the pMHC complex. The Sense and Antisense DNA molecules added to the two “A-wells” have complementary, short (e.g., 5 nt) sequences at their 3′-ends; DNA molecules of A are not significantly complementary to neither DNA molecules of B or C, i.e. DNA molecules of A cannot anneal to any significant extent to DNA molecules of B or C, under the conditions used.
Step c. For each of the six wells, the reaction of X with Y is carried out, thereby covalently linking the DNA molecules to the pMHC complexes. At this point there thus are six wells, each comprising a unique DNA molecule attached to a pMHC complex; the two DNA molecules present in A-wells are complementary, the two DNA molecules present in B-wells are complementary, the two DNA molecules present in C-wells are complementary; and A-wells comprise the same unique peptide (p_{A}), B-wells comprise the same unique peptide (p_{B}), and C-wells comprise the same unique peptide (p_{C}).

Thus, 6 unique pMHC Multiplexers have been formed by the above steps; pairwise, they have identical peptide (p) of the pMHC complex. The 3 unique pMHC Multiplexers may be used for detection or isolation of antigen-specific T cells as described below.

Using a similar approach, one may prepare libraries comprising more than 10, 100, 1000, or 10000 unique pMHC Multiplexers.
Step d. The contents of the 6 wells are pooled, and added to a sample containing T cells, e.g., a blood sample, and incubation is carried out for 30 minutes.
Step e. During the incubation, the two pMHC Multiplexers of one of the pairs of pMHC Multiplexers (i.e. pair A, pair B or pair C) may bind to the T cell receptors (TCRs) of a T cell. This may allow their attached DNA molecules to interact and potentially anneal to each other (i.e. Sense anneal to Antisense). See (FIG. 21, lower panel).

The annealing event of two identical pMHC complexes, carrying complementary sense and antisense DNA, can be detected by extension of the annealing DNA molecules, followed by PCR, sequencing, incorporation of fluorescent nucleotides, and/or isolation of fluorescent-labelled cells. Two of those approaches are described in the following; See (FIG. 22).

### Approach 1: Direct In Situ PCR Amplification and Sequencing (See FIG. 22, Left).

Approach 1, Step f. To the incubation mixture of step (e) is added an appropriate polymerases (e.g., Taq polymerase), dNTPs, and other reagents necessary to perform an extension and several cycles of PCR.
Approach 1, Step g. The annealed DNA duplexes are extended by incubating the mixture at 37 degrees Celsius for 15 minutes.
Approach 1, Step h. Outside primers are added and a PCR is performed, to produce multiple copies of the extended DNA strands that resulted from the annealing of sense and antisense DNA.
Approach 1, Step i. Finally, the amplified DNA molecules are sequenced (FIG. 22, C5). This will reveal which of the three input pMHC Multiplexers were able to bind to antigen-specific T cells, and therefore able to form annealed double-stranded DNA of sense/antisense DNA sequences. From a knowledge of the identity of the pMHC complexes and DNA molecules that were linked in step (c), it may be deduced which of the input pMHC Multiplexers are capable of binding to T cells of the cell sample through their pMHC complexes.

If the analysis was performed using one pair of pMHC Multiplexer at a time (i.e. if the pMHC Multiplexers had been used pair-wise, e.g., using the contents of the A-wells with one cell sample), the PCR reaction could have been performed with fluorescent-labelled dNTPs and fluorescence measurements used as read-out indicating the relative amount of antigen-specific T cells in the sample. See (FIG. 22, C6).

### Approach 2: Incorporation of Fluorescent Nucleotides, Cell Sorting and Sequencing of DNA Tags (See FIG. 22, Right).

Approach 2, Step f. To the incubation mixture of step (e) is added an appropriate polymerases (e.g., Taq polymerase), fluorescent-labelled dNTPs (at least on of the four dNTPs labelled with a fluorochrome), and other reagents necessary to perform an extension and several cycles of PCR.
Approach 2, Step g. Extension from the 3′end of the pairwise annealed DNA strands is performed, to generate a fluorescent-labelled double-stranded DNA connecting two pMHCs (see FIG. 22, C2). This will stabilize the link between the two pMHCs, and hence, stabilize the interaction of the two pMHCs with the two TCRs.
Approach 2, Step h. A flow cytometry analysis is performed on the cell sample. The fluorescent-labelled pMHC Multiplexers will label T cells to which they are bound. Optionally, fluorescence markers of CD8 cells, CD4 cells or other types of T cells may be labelled with another fluorochrome also. pMHC Multiplexer+ cells are now isolated, and the DNA of their associated pMHC Multiplexers are sequenced. From these sequences, and from a knowledge of which DNAs were attached to which peptides in step c, the identity of the peptide (p) in the pMHC Multiplexer that bound T cells can be deduced.

Thus, by either approach 1 or 2, or any other variation of these approaches, it can be deduced which pMHC Multiplexers recognize and bind to T cells of a given blood sample.

### Comments:

Comment 1: The preparation and analysis can be made with both MHC1, MHC2 and MHC-like complexes.
Comment 2: In a preferred embodiment, the complementary regions (sense- and antisense-regions) of the DNA tags attached to pMHC Multimers carrying identical peptide (p) are relatively short, such as 2-3, 4-5, 6-8, 9-12, 13-20 nt, or longer, in order to not efficiently anneal the sense and antisense regions in solution, but only when the corresponding pMHCs are both bound to a TCR on the same cell.

In the above example, each pMHC Multiplexer comprises only one pMHC complex. In a preferred embodiment, each pMHC Multiplexer comprises at least 2, such as at least 3, such as at least 4, such as at least 5, such as at least 6, such as at least 8, such as at least 10, such as at least 15, such as at least 20, such as at least 40, such as at least 100 or more pMHC complexes, all of which carry identical peptide (p) in the pMHC complex.

In a preferred embodiment, each pMHC Multiplexer of the above example, or a similar example involving 10, 100, 1000, 10000, 100000 or more unique pMHC multiplexers, comprises a MHC Tetramer, a MHC Dextramer, a MHC Streptamer, a MHC Pentamer, a MHC Multimer comprising a PROfusion display system or a Ribosome display system, or any other MHC Multimer. (FIG. 24) depicts the principle applied to pMHC Multiplexers comprising a MHC Tetramer.

Thus, the pMHC Multiplexer may contain any number of pMHC complexes and may comprise any kind of multimer scaffold. In (FIG. 23) this is depicted schematically. The term “(pMHC),” denotes any pMHC Multimer comprising n identical pMHC complexes. As may be seen, the procedures applied to the pMHC Multiplexers comprising only one pMHC complex (FIG. 21) and (FIG. 22) can be applied to pMHC Multiplexers containing multiple pMHC complexes as well.

### Example 26. DNA-Peptide Conjugates Used as MHC Multimer Scaffolds

Here, DNA-peptide conjugates, combined with MHC complexes, are used to construct stable DNA-tagged pMHC complexes. Thus generating pMHC Multiplexers.

In this example, the production and structure of a pMHC Multiplexer is described. The pMHC Multiplexer comprises several pMHC complexes, where the peptide (p) of the pMHC complex is directly linked to the DNA that encodes it, and where said DNA also comprises a part capable of annealing to another DNA molecule of the pMHC Multiplexer, thereby chemically linking the individual pMHC complexes of the pMHC Multiplexer.
Step a. 1000 unique peptides, each with a length of 20 amino acids residues, and comprising a primary amino group at one terminus, are synthesized using solid phase synthesis. Also, 2000 unique DNA oligonucleotides, each with a length of 20 nucleotides and comprising a N-hydroxysuccinimide ester at one terminus, are synthesized using solid phase synthesis. 1000 of these comprise a coding region of 10 nucleotides that is unique to each, and a 10-nucleotide constant region (A) that is the same in all. The remaining 1000 of the 2000 unique oligonucleotides comprise a coding region of 10 nucleotides that is unique to each, and a 10-nucleotide constant region (B) that is the same in all. A and B are complementary sequences and thus can anneal to each other.
Step b. To each of 1000 microtiter wells is added one of the unique peptides, one of the unique DNA oligonucleotides comprising the constant region (A), and one of the unique DNA oligonucleotides comprising the constant region (B). A phosphate buffer pH 9 is added, the temperature is increased to 70 degrees Celsius, and incubation is performed for 2 hours, during which the amino group of the peptide will react with a N-hydroxy-succinimide moiety of a DNA oligonucleotide.
Step c. The temperature is decreased to 25 degrees Celsius, thereby allowing the constant regions (A) to anneal to constant regions (B), to form a complex comprising 2 identical peptides, bridged by a double-stranded DNA comprising the constant sequence (A) on one strand and the constant (and complementary) sequence (B) on the other strand.
Step d. Empty, biotinylated MHC2 complexes are added to each of the 1000 wells, and incubation is continued at 25 degrees Celsius for 1 hr.
Step e. Streptavidin (SA) is added to each of the 1000 wells, and incubation is continued for 25 degrees Celsius for 1 hour.
Step f. The temperature is decreased to 4 degrees Celsius, and incubation is continued for at least 15 minutes.

The resulting product in each of the 1000 wells is thus a pMHC Multiplexer where the encoding molecule (here: DNA) is directly linked to the encoded peptide (p), and where up to 4 pMHC complexes are linked to a streptavidin tetramer (see FIG. 25, F).

### Comments:

If in step (b) only the DNA oligonucleotides comprising a constant region (A) is added, and no streptavidin is added in step (e), a DNA-tagged pMHC monomer as shown in (FIG. 25, A) is formed.
If in step (b) only the DNA oligonucleotides comprising a constant region (A) is added, and if a dimer capable of binding two biotinylated pMHC complexes is added instead of the streptavidin in step (e), a pMHC dimer as shown in (FIG. 25, B) is formed.
If in step (b) only the DNA oligonucleotides comprising a constant region (A) is added, a DNA-tagged pMHC tetramer as shown in (FIG. 25, C) is formed.
If in step (d) empty MHC2 complexes attached to a polypeptide capable of forming a pentamer with four identical polypeptides, and no streptavidin is added in step (e), a DNA-tagged pMHC Pentamer as shown in (FIG. 25, D) is formed.
If in step (b) only the DNA oligonucleotide with constant sequence (A) is added, and if a streptavidin-coated dextran is added instead of streptavidin in step (e), the DNA-tagged pMHC Dextramer shown in (FIG. 25, E) is formed.
If the 1000 peptides of step (a) are synthesized with an amino group at each terminus instead of just at one end, each peptide will be linked directly to two DNA oligonucleotides, and a DNA tagged Tetramer as shown in (FIG. 25, G) may be formed.
In analogous ways, pMHC Multiplexers comprising a pMHC dimer, a pMHC tetramer, a pMHC Pentamer, a pMHC Dextramer or any other pMHC Multimer may be formed, where all the peptides are linked directly to two DNA oligonucleotides, such as described above and shown in (FIG. 25, G) for a pMHC Multiplexer comprising a pMHC Tetramer.
If the 1000 peptides of step (a) are synthesized with an amino group at each terminus instead of just at one end, and no streptavidin is added in step (e), each peptide will be linked directly to two DNA oligonucleotides, and a pMHC Multiplexer comprising multiple pMHC complexes in series as shown in (FIG. 26, A) may be formed. In this pMHC Multiplexer the DNA oligonucleotides function as both the encoding molecule and the multimer scaffold.
By appropriate adjustments of the length of DNA and peptide, the synergy in binding and annealing temperature of constant sequence (A) for constant sequence (B) may be adjusted as desired.
A similar principle may also be applied in order to stabilize pMHC2 complexes that are unstable because of poor affinity of the peptide (p) for the MHC complex. Thus, if 1000 unique peptides are each synthesized in two variants, e.g., one variant with a reactive group X at both termini and one variant with a reactive group Y at both termini, and if each pair of unique peptide in two variant forms is added to a separate well, and the reactive group X is allowed to react with the reactive group Y, a polymer of the unique peptide will form. If then a streptavidin is added, a pMHC tetramer as shown in (FIG. 27, A) may form, and if instead a streptavidin-coated dextran is added, a pMHC Dextramer as shown in (FIG. 27, B) may form. It is to be understood from the above that the reactive group X (e.g., an amine) and the reactive group Y (e.g., a N-hydroxy-succinimide ester) are capable of reacting with each other under the conditions used, to form a covalent bond.

### Example 27. Preparation of SP1-Based pMHC Multiplexers

In this example it is described how SP1-based pMHC Multiplexers can be generated.

In a preferred embodiment of the invention, the SP1 protein is used as the display system. In this example, 1000 unique pMHC Multiplexers are produced:
Step a. By recombinant means, a gene encoding the Acid Peptide is fused to the 5′-end of a DNA sequence encoding a flexible linker peptide of approximately 5-15 amino acid residues (e.g., SSGPSSGSSGPS) which is fused to the 5′-end of the gene encoding the SP1 protein subunit. The DNA construct is inserted into a plasmid under the control of a promoter. The plasmid is transformed into an E. coli strain capable of expressing the fusion peptide in large amounts and capable of allowing the correct folding of the SP1 protein. The dodecameric Acid-SP1 fusion protein is now expressed and purified, according to standard protocols (the cloning, expression and purification is described in e.g., (Plant Physiol. 130 (2), 865-875 (2002); Biotechnol. Bioeng. 95 (1), 161-168 (2006); J. Biol. Chem. 279 (49), 51516-51523 (2004); Nano. Lett. 8 (2), 473-477 (2008)). The dodecameric SP1 wild-type protein is shown in (FIG. 28, A); the Acid-SP1 fusion protein is shown in (FIG. 28, C).
Step b. In a separate experiment a plasmid is constructed that carries a DNA sequence encoding a promoter controlling the transcription of a gene fusion consisting of, from the 5′-end: A gene encoding Base Peptide, fused to the 5′-end of the gene encoding heavy chain (HC). This plasmid is transformed into an E. coli strain, and the Base Peptide-HC peptide is expressed and purified, using standard protocols.
Step c. The Base Peptide-HC peptide from step (b) is added to each of 1000 microtiter wells, in a buffer appropriate for formation of a pMHC complex. Then beta2M peptide is added. Finally, a unique peptide of 7-11 amino acid residues is added to each of the 1000 microtiter wells. The solutions are first denatured and then renatured, e.g., by heating followed by cooling, or by addition of 6-8 M urea followed by removal of the urea by e.g., dialysis, or by any other means that allow denaturation and renaturation. Upon renaturation, the Base Peptide-pMHC complex will have formed. Thus, each microtiter well now comprises a unique Base Peptide-pMHC complex.
Step d. In a separate experiment, Base Peptide carrying a reactive group X at one terminus (e.g., a triple bond at the N-terminus), is added to each of 1000 (empty) microtiter wells, and appropriate buffer is added. Then a unique DNA molecule, carrying a reactive group Y capable of reacting with X (e.g., an azide, capable of reacting with a triple bond) is added to each of the 1000 microtiter wells. The reactive groups X and Y are brought to react, thereby forming in each well a unique Base Peptide-DNA conjugate.
Step e. Each of the 1000 solutions of a unique Base Peptide-pMHC complex from step (c) is added to one of the 1000 solutions of a unique Base Peptide-DNA conjugate from step (d), at a molar ratio of
   “Base Peptide-pMHC”: “Base Peptide-DNA conjugate”
      of approximately 10:1. Each of the resulting 1000 solutions now comprise both a unique Base Peptide-pMHC and a unique Base Peptide-DNA conjugate.
Step f. To each of the 1000 solutions of step (e) is added the purified Acid Peptide-SP1 fusion protein from step (a), and the 1000 solutions are incubated for 30 min.

After dimerization of Acid Peptide and Base Peptide, multiple copies of a unique pMHC Multiplexer will have formed in each of the 1000 solutions, where the unique pMHC Multiplexer comprises a SP1 scaffold displaying a unique pMHC complex in multiple copies, and where the identity of the peptide (p) of the pMHC complex is encoded by the DNA molecule attached to the same pMHC Multiplexer, see (FIG. 31).

Thus, 1000 unique pMHC Multiplexers have been formed by the above steps (a)-(f). The 1000 unique pMHC Multiplexers may be used for detection or isolation of antigen-specific T cells as described below. If desired, a fluorochrome such as fluorescein, PE, APC or any other fluorochrome may be attached covalently or non-covalently to the pMHC Multiplexer, to ease its detection in e.g., flow cytometry analysis.

### Comments:

Comment 1: In the above example, pMHC1 complexes were formed from HC and beta2M protein. Alternatively, pMHC2 complexes could be formed from alpha and beta protein, where either alpha or beta were fused to Base Peptide. And as a result, each pMHC Multiplexer would comprise multiple copies of pMHC2 complexes.
Comment 2: If in step (f) no DNA molecule is added (and therefore no Base Peptide-DNA conjugate generated), a pMHC Multimer (rather than a pMHC Multiplexer) will be generated by the above process. The generated SP1-based pMHC Multimers will each carry up to 12 pMHC molecules.
Comment 3: In the above example, the HC peptide is linked to the Base Peptide, and then beta2M is added. As an alternative, beta2M may be linked to Base Peptide, and then HC and peptide (p) added, to form the Base Peptide-pMHC complex.
Comment 4: The SP1 protein is highly stable, even near 100 degrees Celsius. It is therefore not a problem to denature the components of pMHC before renaturation (i.e. denature HC, beta2M, peptide or alpha, beta, peptide); the SP1 scaffold will not be affected.
Comment 5: Instead of using the Acid Peptide-Base Peptide dimerization as a means to attach the pMHC complex to the SP1, the pMHC complex may be directly linked to the SP1 subunit, e.g., by making a gene fusion between a gene encoding HC, beta2M, alpha or beta and a gene encoding the SP1 subunit. See (FIG. 29, B).
Comment 6: Any other heterodimer than Acid and Base may be used; preferably, the dimerization should be efficient and preferably be on dimeric form most of the time. In a preferred embodiment the heterodimer is covalently linked to allow for high stability.
Comment 7: Homodimers may also be used in place instead of the Acid Peptide-Base Peptide dimer.
Comment 8: In a preferred embodiment of the invention, an E. coli strain is used with deletions or mutations in genes encoding proteases, such as proteases in the membranes or periplasm, in order to increase the average life-time of the expressed peptides.
Comment 9: In a preferred embodiment of the invention, the DNA in the above example is replaced by a fluorescence label or an element label such as a rare earth element. Thereby the process will generate 1000 unique SP1-based pMHC Multimers, all of which are labelled with the same label.
Comment 10: Alternatively, each of the 1000 unique SP1-based pMHC Multimers can be labelled with a unique combination of fluorochromes, elements and or copy-number of each of said labels-thereby generating pMHC Multiplexers representing 1000 unique peptide (p) identities, each encoded by a unique combination of (unique number of copies of) said labels.

### Example 28. pMHC Multiplexer Formation by Intracellular SP1 Multimer Formation

This example explains how SP1-based pMHC Multiplexers may be prepared using the phage M13 as a scaffold.

In a preferred embodiment of the invention, phage display is used as the display system. Specifically, the filamentous phage M13 is used, see (FIG. 13), to generate a large number of different pMHC Multiplexers, each of which carry one or more copies of the SP1 dodecamer protein scaffold. In this example, 1000 unique pMHC Multiplexers are produced:
Step a. A M13-derived phagemid is constructed carrying a promoter controlling a DNA sequence encoding a fusion peptide consisting of, from the N-terminal end, a Signal Peptide (SP), Acid peptide, and pIII coat protein. Further, the phagemid carries a promoter controlling the transcription of a unique peptide X. 1000 unique plasmid constructs are generated: all phagemids carry the same peptide fusion Signal Peptide (SP), Acid peptide, and pIII coat protein, but each phagemid carries a unique peptide X (see FIG. 13). The gIII fusion gene is under the control of a promoter whose expression can be modified by the addition of a molecule to the growth medium. One such molecule may be IPTG, where the promoter is under the regulatory control of IPTG. Optionally, the production of peptide X is very efficient, leading to high amounts of peptide X in a bacterial cell carrying the phagemid. Optionally, the expression level of peptide X can be controlled by extracellular changes such as the addition of a molecule that stimulates transcription or translation of the gene encoding peptide X.
Step b. On the same phagemid, or on another vector in the same cell, is a DNA sequence encoding an Acid Peptide-HC fusion protein, the beta2M protein, and a fusion peptide of, from the N-terminal, Base Peptide-Flexible linker peptide (e.g., SSGSSG)-SP1 subunit peptide.
Step c. The DNA constructs of step (a) and (b) are introduced (e.g., by transformation) into an E. coli strain that is capable of supporting phage infection and amplification, and cell growth is performed for several cell generations. Then Helper Phage is added, and phage amplification is performed for several cell generations. Phage particles, carrying one or more copies of the SP1 protein scaffold that carries up to 12 pMHC complexes each, are produced and are being secreted out of the cell, i.e. into the growth medium. Optionally, protease inhibitor(s) and/or nuclease inhibitors are added.
Step d. The E. coli cells and phage particles in the growth medium are separated by centrifugation, by pelleting the cells. The supernatant comprising the phage particles is transferred to a new tube.
Step e. Optionally, the phage particles are PEG precipitated.

The resulting product of the process described by the above steps (a-e), is 1000 unique pMHC Multiplexers, each of which carries up to 12 pMHC complexes.

### Comments:

The SP1 dodecamer protein is described in the paper “Protein scaffold engineering towards tunable surface attachment”, Angew. Chem. Int. Ed. 2009, vol 48, p. 9290-9294.
In the experiment described above, HC is fused to the Acid Peptide. Alternatively, beta2M may be fused to the Acid Peptide.
In the experiment above or in the point immediately above, HC or beta2M is fused to the Acid peptide. Alternatively, the HC or beta2M protein may be fused to Base Peptide, and then the Sp1 should be fused to Acid Peptide. Other heterodimers than Acid Peptide-Base Peptide may be used.
As a further alternative, the gIII gene encoding the M13 pIII coat protein, may be replaced by the gVIII gene encoding the M13 pVIII coat protein.
In the above example, pMHC1 complexes were formed from HC and beta2M protein, and attached to the phage coat. Alternatively, pMHC2 complexes could be formed from alpha and beta protein, where either alpha or beta were fused to Base Peptide. And as a result, the phage particles would display pMHC2 complexes.
The above example uses a system including both phagemid and helper phage. This ensures that only a fraction of the pIII coat proteins or of the pVIII coat proteins of the phage particle will display a pMHC complex. Alternatively, the coat fusion proteins may be carried by a phage genome, in which case all of the pIII coat proteins or all of the pVIII coat proteins will display a pMHC complex.
In a preferred embodiment of the invention, an E. coli strain is used with deletions or mutations in genes encoding proteases, such as proteases in the membranes or periplasm, in order to increase the average life-time of the expressed peptides.
By appropriate modification, any other type of filamentous or non-filamentous phage, and any virus or may be used in this invention. Thus, any appropriate coat protein on any phage or virus can be used in the invention.
Likewise, any membrane protein or other type of peptide associated with the extracellular membrane surface of a prokaryotic or eukaryotic cell may be used as the display carrier of the pMHC complex.

### Example 30. Generation of Cell-Displayed Peptide Library by Fusion to the LamB Membrane Protein of E. coli

This example describes the cloning and display of a library of repeated polypeptide sequences fused to the LamB membrane protein. The work has been published by Stanley Brown (Metal-recognition by repeating polypeptides, Nature Biotechnology, 1997, Vol 15, pp 269-272. DOI 10.1038/nbt0397-269). This example is not an embodiment of the present invention but serves as an example for the various examples described below.

In this example, a library of small repeating polypeptides was displayed on the surface of E. coli as part of the maltodextrin porin, LamB. In brief, a plasmid encoding a modified version of the structural gene for LamB allowing for cloning of DNA fragments between codon 155 and 156 was constructed. The library of oligonucleotides encoding the repeating 14 amino acid polypeptides was synthesized by a rolling circle method using the template oligonucleotides described below. Three different libraries were constructed, transformed into a ΔLamB E. coli strain and pooled to generate a population of more than 10E6 clones.

The peptides libraries were constructed as described in more details in the following steps:
Step 1. Construction of expression vector.

A LamB expression vector, pSB2267, was constructed as described in Brown et al, 1997 (10.1038/nbt0397-269). Specifically, the DNA sequence at codon 155 and 156 was mutated to introduce PstI and XhoI sites used for cloning of the nucleotide libraries.
Step 2. Construction of nucleotide circles.

Template oligonucleotides containing 17 nucleotides of defined sequence and 25 random nucleotides (e.g. 5′-GCT CTG NNK NNK NNS GYT NNK NNS CTG NNK NNK NNS ATG CAT-3′) were circularized by annealing of an oligo complementary to the defined nucleotides and treatment with T4 DNA ligase. The resulting products were analyzed in polyacrylamide gels and circular molecules purified. These were either monomer or dimer circles.
Step 3. Generation of double-stranded DNA

The circular templates were annealed with oligonucleotides whose 3′ ends were complementary to the defined nucleotides of the circular template and whose 5′ portion included an XhoI recognition site within a primer site for later PCR amplification. (e.g. rolling circle primer 5′-CAGCCAGTTGCTCTCGAGGGACAGAGCATGCAT-3′). The rolling circle primers were extended around the circular template by the Sequenase form of T7 DNA polymerase. DNA polymerization was continued around the circular template many times using a single-stranded DNA binding protein, T4 gene 32 protein to generate the repeating oligonucleotides. An oligonucleotide containing a PCR primer sequence with a PstI recognition site at its 5′ end was annealed to the defined nucleotide now present in the repeating oligonucleotide and extended with Klenow fragment (e.g. PstI primer: 5′-GGTTCACAGGCTTGGTCTGCAGGCTCTG-3′).
Step 4. PCR amplification and cloning of rolling circle library

The repeating oligonucleotides containing PCR primer sites at both ends were amplified by PCR (PstI PCR primer: 5′-GGTTCACAGGCTTGGTCTGCAG-3′; XhoI PCR primer: 5′-CAGCCAGTTGCTCTCGAGGGA-3′). The resulting double-stranded repeating oligonucleotides was digested with PstI and XhoI, cloned into the expression vector pSB2267 and transformed into E. coli S2188.
Step 5. Growth procedure

Cultures were established at 30° C. in YT broth supplemented with 25 μg/ml chloramphenicol and transcription of the hybrid lamB genes induced by 2 mM IPTG. This procedure results in approximately 10,000 copies of the hybrid protein to be displayed on the surface of the bacterium. The induced cultures were diluted into M63 salt containing a final concentration of 75% Percoll. At this concentration, the bacteria were less dense than the solution.
Step 6. Enrichment of E. coli library

Metal was added at a concentration of 0.5-1 mg/ml and the bacteria allowed to adhere at room temperature. Following this incubation, the suspensions were centrifuged, the supernatants with the nonadhering bacteria discarded, and the metal with any adhering bacteria resuspended in YT broth supplemented with 25 g/ml chloramphenicol. The broth suspensions were incubated overnight, during which the bacteria multiplied under conditions that did not induce expression of the hybrid LamB protein. After each cycle of enrichment, aliquots of the saturated cultures were frozen at −80° C. with 15% glycerol and stored for later analysis.

The six steps process described above resulted in a library of millions of E. coli cells each displaying on their surface a unique repeating polypeptide fused to LamB. This library of pMHC Multiplexers is called “E. coli-based pMHC Multiplexers of Example 30”.

### Example 31. Generation of Up to 1 Million pMHC Multiplexers, by External Addition of Empty MHC 2 to Cells Displaying Peptides on their Surface

This is a modification of Example 30.

In this Example 31, pMHC Multiplexers are generated where the pMHC Multiplexers comprise an E. coli cell carrying a DNA tag encoding the peptide that is attached to the surface of the E. coli cell, and where multiple copies of the displayed peptide on the cell surface has been complexed to an empty MHC 2 complex, to form a cell displaying multiple copies of the pMHC complex.
Step 1. Library generation

Steps 1-5 of Example 30 are performed as described. This generates a large library of E. coli cells each displaying a repeating peptide epitope motive fused to LamB on its surface.
Step 2. pMHC2 Complex formation

MHC2 (DR1) are added at an appropriate concentration for pMHC complex formation, e.g., a concentration of 0.01 mg/ml, or 0.1 mg/ml, or 1 mg/ml, and the LamB-peptide epitope-MHC2 complex allowed to form at room temperature. Following this incubation, the suspensions is centrifuged, the supernatants with the non-adhering MHC discarded, and the bacteria with any adhering MHC2 resuspended.

The resulting product of Step 2 is a large collection of pMHC Multiplexers, each displaying a unique peptide-MHC2 (DR1) complex on its surface. This library of pMHC Multiplexers is called “E. coli-based pMHC Multiplexers of Example 31”.

### Example 32. Generation of Three pMHC Multiplexers, by External Addition of Empty MHC2 (Empty DR1) to Cells Each Displaying Multiple Copies of One of Three Different Peptide Epitopes

This is a modification of Example 31.

In this Example 32, three specific MHC2 peptide epitopes and a negative control are cloned in LamB and displayed on E. coli.
Step 1. Positive and negative control MHC2 epitopes

The control epitope peptides

CMV99:
MSIYVYALPLKMLNI,
CMV105:
ALPLKMLNIPSINVH,
HA307-19:
PKYVKQNTLKLAT

are appropriate positive control peptides that bind to MHC II (DR-1) complexes while

VMC99:
INLMKLPLAYVYISM

is an appropriate negative control peptide that has the inverse sequence of that of CMV99.
Step 2. Synthesis of DNA oligonucleotides encoding MHC2 peptide epitopes and negative control

The DNA fragments are designed such that the epitope-encoding reading frames are “in-frame” with the LamB protein. The DNA fragments is designed to contain a proximal PstI and a distal XhoI restriction site to allow an in-frame cloning of the epitope-encoding DNA between codon 155 and 156 of LamB as described in Example 30.
Step 3. pMHC2 complex formation

MHC2 (DR1) are added at a concentration of 0.01 mg/ml, or 0.1 mg/ml, or 1 mg/ml, and the LamB-peptide epitope-MHC2 complex allowed to form at room temperature. Following this incubation, the suspensions is centrifuged, the supernatants with the non-adhering MHC discarded, and the bacteria with any adhering MHC2 resuspended.

The resulting product of Step 3 is a collection of 3 individual positive control pMHC Multiplexers each displaying an individual peptide-MHC2 (DR1) complex on its surface as well as a negative control pMHC Multiplexer.

In the above example, the PstI-XhoI epitope-encoding DNA fragment can encode 2, 3 or multiple copies of the peptide epitope.

### Example 33. Generation of a Library of pMHC Multiplexers, Comprising Three Known Peptide Epitopes

This example is a modification of example 32. In Example 32, three pools of individual pMHC Multiplexers were constructed. In the present example, these three individual pools each containing a known peptide epitope are mixed into one pool.
Step 1. Construction of 3 individual pools of pMHC multiplexers.

This step is done as described in Steps 1-3 in Example 32.
Step 2. Individual pMHC multiplexers are mixed into one pool.

The resulting product is a pool of multiplexers which can contain an approximate equal amount of each of three multiplexers. This 3-member library of pMHC Multiplexers is called “E. coli-based pMHC Multiplexers of Example 32”.

Alternatively, the amount of one specific pMHC Multiplexer can be significantly higher than the others. Alternatively, the amount of one specific pMHC Multiplexer can also be significantly lower than the others.

### Example 34. Generation of a 100,000-Member Corona Virus-Specific pMHC Multiplexer Library

This is a modification of Example 31. Here, a large library of corona virus-specific pMHC Multiplexers is constructed. Coronaviruses including SARS-COV-2, belong to a family of positive-stranded RNA viruses termed Coronaviridae and have a large genome size between 27-31 kb. Specifically, the RNA genome of SARS-CoV-2 has 29,811 nucleotides, encoding for around 29 proteins. Double-stranded cDNA libraries can be constructed in several ways described in detail in the literature and can comprise the following steps:
Step 1. Purification of experimental RNA sample
Step 2. cDNA synthesis and purification
Step 3. DNA fragmentation
Step 4. End repair and dA-tailing
Step 5) Ligation of DNA adaptors and purification
Step 6) PCR amplification using adaptor-specific primers

The resulting cDNA libraries can be further fragmented to reflect the preferred epitope size for MHC2 (and MHC1). The adaptors used for ligation and PCR amplification can encode PstI/XhoI DNA restriction sites to allow cloning in the LamB protein expression plasmid described in Example 30.
Step 7. pMHC2 complex formation

MHC2 (DR1) are added at a concentration of 0.01 mg/ml, or 0.1 mg/ml, or 1 mg/ml, and the LamB-peptide epitope-MHC2 complex allowed to form at room temperature. Following this incubation, the suspensions is centrifuged, the supernatants with the non-adhering MHC discarded, and the bacteria with any adhering MHC2 resuspended.

The result of the example described above is a large library of (potentially) corona-specific peptide epitopes expressed and displayed on the surface of E. coli on the LamB protein. This Corona-directed library of pMHC Multiplexers is called “Corona-directed pMHC Multiplexers of Example 34”.

### Example 35 Generation of a Human Genome-Specific pMHC Multiplexer Library

This is a modification of Example 31. Here, a large library of human genome-specific pMHC Multiplexers is constructed. The human genome encodes for approximately

Double-stranded cDNA libraries can be constructed in several ways described in details in the literature and can comprise the following steps:
Step 1. Purification of experimental RNA sample
Step 2. cDNA synthesis and purification
Step 3. DNA fragmentation
Step 4. End repair and dA-tailing
Step 5) Ligation of DNA adaptors and purification
Step 6) PCR amplification using adaptor-specific primers

The resulting cDNA libraries can be further fragmented to reflect the preferred epitope size of MHC2 (and MHC1). The adaptors used for ligation and PCR amplification can comprise PstI/XhoI DNA restriction sites to allow cloning in the LamB protein expression plasmid as described in Example 31 and expressed as LamB protein fusions.

Alternatively, human cDNA libraries are commercially available, can be acquired and used for further DNA fragmentation, adaptor/restriction site ligation and amplification as described above from step 3 and forward.
Step 7. pMHC2 complex formation

MHC2 (DR1) are added at a concentration of 0.01 mg/ml, or 0.1 mg/ml, or 1 mg/ml, and the LamB-peptide epitope-MHC2 complex allowed to form at room temperature. Following this incubation, the suspensions is centrifuged, the supernatants with the non-adhering MHC discarded, and the bacteria with any adhering MHC2 resuspended.

The result of the example described above is a large library of human-specific peptide epitopes expressed and displayed on the surface of E. coli on the LamB protein. The library of pMHC Multiplexers is called “Human-directed pMHC Multiplexers of Example 35”.

### Example 36. pMHC Multiplexers by Extracellular pMHC Formation and Biological Attachment by Acid-Base Dimerization

This is a modification of Example 31. In this example, an acid peptide is displayed on the surface of E. coli as part of the maltodextrin porin, LamB. In brief, a plasmid encoding a modified version of the structural gene for LamB allowing for cloning of DNA fragments between codon 155 and 156 is used.
Step 1. DNA fragment encoding Acid peptide.

A DNA fragment is synthesized which encodes the Acid peptide with amino acid sequence GAAQLEKELQALEKENAQLEWELQALEKELAQGGCPAGA and with corresponding PstI and XhoI restriction sites positioned at each end to allow in-frame cloning of the DNA fragment with the LamB gene.
Step 2. Acid Peptide/LamB fusion and expression vector construction.

The Acid-peptide encoding DNA fragment is digested with PstI and XhoI and cloned into the expression vector pSB2267
Step 3. Construction of peptide epitope libraries

In the same expression vector but located at another place in the expression vector and under control of an independent promoter, a signal peptide-peptide X library is generated. This will generate peptides encoded by the peptide X library into the periplasm of E. coli
Step 4. Transformation of plasmid encoding Acid-peptide and peptide X library

The modified pSB2267 vector is transformed into E. coli S2188 and the transformed cells grown in bacterial growth medium.
Step 5. Growth of library in microtiter wells

The transformed E. coli cells are aliquoted into microtiter wells containing growth medium where each aliquot comprise approximate 0.1-0.3 transformed E. coli cell. The cells are grown at a permissible temperature such as 30° C. under shaking and hence aeration.
Step 6. Induction of Acid peptide and/or peptide X

A transcriptional inducer of the promoters expressing LamB-Acid Peptide fusion and/or peptide X can be applied allowing for production of an increased amount of the proteins in question.
Step 7.

A Solution containing a Base Peptide-Alfa Chain (Base-AC) fusion protein in complex with Beta-Chain (thus generating a Base-tagged MHC2 (DR-1) complex) are transferred into each of the micro titer wells to a final concentration preferably lower than the estimated concentration of peptide X in the individual well. Here, a Base Peptide variant is used that carries a cysteine, capable of forming a disulfide bond with Acid Peptide upon their complexation.
Step 8. The temperature is decreased to approximately 20° C., to allow complex formation between peptide X and the Base-tagged MHC2 (DR-1) complex, to potentially form the peptide X-MHC2 complex.
Step 9. Optionally, a redox buffer is added, to first reduce the cysteine in each of the Acid Peptide and the Base Peptide.
Step 10. Optionally, the oxidation/reduction status of the buffer is adjusted to allow disulfide bond formation between the cysteines of the Acid Peptide and the Base Peptide. As a result, the peptide X-MHC2 complex becomes covalently linked to the Acid Peptide-pVIII fusion protein of the phage coat.

The product of this example is a library of base-tagged-pMHC2 complexes fused to the LamB-acid-peptide fusion of a E. coli cells encoding and expressing the bound peptide. The library is called “pMHC Multiplexers of Example 36”.

The DNA fragment encoding Acid peptide can be constructed to encode an Acid peptide monomer, an Acid peptide dimer, an Acid peptide trimer, a tetramer, a pentamer, hexamer, heptamer, octamer and further multiples separated by a suitable spacer.

Base-tagged peptide-receptive MHC1 complexes can be employed.

The base-tag can be cloned in the LamB protein while the acid peptide can be fused to MHC1 and/or MHC2 to allow LamB/pMHC complex formation.

### Example 37. Screening of Antigen-Specific T Cells Using DNA-Tagged MHC Dextramers

This example describes Multi-parallel detection of antigen-responsive T cells in single samples. The work has been published by Bentzen et al. (Nature Biotechnology Vol 34, number 10, 2016. doi:10.1038/nbt.3662). This example is not an embodiment of the present invention but serves as an example for the various examples described below.

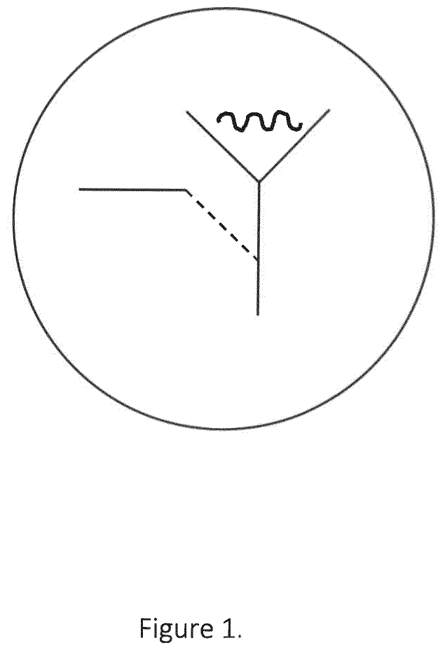

To allow complex assessments of T cell reactivity in limited biological samples, Bentzen et al developed a technology using DNA barcodes as tags for specific interactions between pMHC molecules and CD8 T cells. DNA barcoded MHC multimer reagents also carrying a common fluorescent label, phycoerythrin (PE), were generated on a dextran backbone as shown in FIG. 1a of the paper. This strategy enabled single-tube-based detection of pMHC responsive T cells using mixtures of >1000 distinct pMHC multimers where each specific pMHC molecule was associated with a given DNA barcode. MHC multimer-binding T cells were isolated using fluorescence-activated cell sorting (FACS) based on their PE fluorescence intensity, and the composition of the associated DNA barcodes were identified through amplification and high-throughput sequencing. Antigen-responsive T cells within the isolated T cell pool were identified based on the number of reads for a specific pMHC-associated barcode compared to the complete pMHC multimer library. The number of unique DNA sequences originating from each pMHC-associated barcode was assessed based on the incorporation of unique molecular identifiers (UMIs), allowing the clonal reduction of sequences from the amplified product. This revealed the number of specific pMHC multimers that interacted with T cells in the given cell sample. The DNA barcodes were designed from sets of unique 25mer oligonucleotides using previously published sequences described as having similar amplification properties while containing maximum diversity of their identification motifs (Xu et al, Proc. Natl. Acad. Sci. U.S.A 106, 2289-94 (2009)). To provide a system adaptable to large library screenings, Bentzen et al applied a combinatorial design of DNA barcodes as depicted in FIG. 1b and listed in supplementary Table 1-3 of their paper.

### Feasibility and Limit of Detection Using Large Libraries of DNA-Barcoded MHC Multimers for T Cell Analyses

To provide proof-of-feasibility for staining antigen-specific T cells in mixtures of >1000 different pMHC multimers, Bentzen et al compared detection of various T cell populations responsive to virus-derived peptides using DNA-barcoded MHC multimers or combinatorial fluorescently-labeled MHC multimers, respectively. They verified that PE labeled MHC multimers carrying DNA barcodes were able to bind specifically to their target T cell population even in excess of 999 irrelevant pMHC multimers, and that the DNA barcode associated with positive control reagents could be specifically recovered after isolation of MHC multimer binding cells (Supplementary FIG. 1a-d, Bentzen et al).

### Detection of Tumor-Reactive T Cells

Recent clinical success in cancer immunotherapy has led to great interest in examining T cell reactivity against cancer. To demonstrate the feasibility of using DNA-barcoded MHC multimers for the detection of tumor-restricted pMHC-specific T cells, Bentzen et al analyzed T cell reactivity among tumor-infiltrating lymphocytes (TILs) from melanoma patients against a previously described library of shared melanoma-associated peptides. Antigen-specificity was assessed within 11 expanded TIL products against an HLA-A*0201-restricted DNA-barcoded pMHC multimer library melanoma-associated peptides and 8 virus-derived peptides (Supplementary Table 8, Bentzen et al, 2016). They detected numerous T cell populations responsive to melanoma associated antigens in 8 of the 11 TIL products (FIG. 4A-B Bentzen et al, 2016). In comparison the same library of melanoma-associated epitopes was included for T cell screening in the healthy donor cohort resulting in very few detected responses

Detection of Tumor-Reactive T Cells from Small-Size Clinical Samples

One major advantage of multiplex technologies for T cell detection is the possibility of determining the composition of antigen-specific T cells in small biological samples without a need for lymphocyte expansion. Bentzen et al, (2016) utilized the high-throughput screening capacity of DNA-barcoded MHC multimers to study the dynamics of T cell responses in various samples from two patients with metastatic melanoma participating in a phase II trial, with adoptive cell therapy using in-vitro expanded TILs. They screened for T cell recognition towards a large library of virus- and shared melanoma-derived epitopes (328 barcoded pMHC multimers, Supplementary Table 9, Bentzen et al (2016)) in:
a) uncultured tumor fragments following enzymatic digest, i.e. unexpanded TILs (digest),
b) TILs in vitro expanded from small tumor fragments, dissected from the same metastatic lesion and
c) peripheral blood obtained before and approximately 1 month after infusion of expanded TILs. Although very few lymphocytes were available in the melanoma samples directly after enzymatic digest (18,000 and 48,000, respectively for MM01 and MM02), they detected numerous melanoma-associated T cell responses in these tissue samples.

### Flow Cytometry and Cell Sorting

Cells stained with DNA-barcoded multimers were sorted on a FACSAria (Aria, Aria-II or AriaFusion) (Becton Dickinson) into tubes containing 200 μL barcode-cytometry buffer (tubes were saturated with PBS+2% BSA in advance). Using FACSDiva software Bentzen et al (2016) gated on single, live CD8+ positive and ‘dump’ (CD4, 14, 16, 19 and 40) negative lymphocytes and sorted all multimer (PE) positive cells within this population. For the samples stained with antibodies for intracellular activation-markers and DNA-barcoded-multimers, they gated on single, live CD8+/CD3+ lymphocytes. In all ICS experiments they sorted the IFN-v/TNF-α double positive and the double negative population into separate tubes. The sorted cells were centrifuged 10 min, 5000 g, and the buffer was removed. The cell pellet was stored at −80° C. in a minimal amount of residual buffer (<20 μL).

### Example 38. Screening of Antigen-Specific T Cells Using Fluorescently Labelled pMHC Multiplexers

This is a modification of Example 37 (Screening of antigen-specific T cells using DNA-tagged MHC Dextramers). In this example, fluorescently labelled pMHC Multiplexers from the libraries generated in example 30, example 31, example 32, example 33, example 34 and example 35 above is screened for the capacity to bind epitope-specific T-cells. The specific libraries used are:
“E. coli-based pMHC Multiplexers of Example 30”
“E. coli-based pMHC Multiplexers of Example 31”
“E. coli-based pMHC Multiplexers of Example 32”
“Corona-directed pMHC Multiplexers of Example 34”
“Human-directed pMHC Multiplexers of Example 35”
“pMHC Multiplexers of Example 36”

pMHC Multiplexers will be labelled with fluorochrome-labelled anti-MHC1 antibody and/or fluorochrome-labelled anti-MHC2 antibody. This will label all pMHC multiplexers and hence the cells, e.g. T-cells, with the ability to bind the pMHC multiplexers.

### Flow Cytometry and Cell Sorting

T-cells stained with fluorochrome-labelled pMHC Multiplexers can be sorted on a FACS sorter (FACSAria (Aria, Aria-II or AriaFusion) (Becton Dickinson)) into tubes containing 200 μL buffer (tubes were saturated with PBS+2% BSA in advance). The sorted cells can be centrifuged 10 min, 5000 g, and the buffer removed. The cell pellet can be stored at −80° C. in a minimal amount of residual buffer (<20 μL).

For the libraries “E. coli-based pMHC Multiplexers of Example 30”, “E. coli-based pMHC Multiplexers of Example 31”, “E. coli-based pMHC Multiplexers of Example 32”, “Corona-directed pMHC Multiplexers of Example 34” and “Human-directed pMHC Multiplexers of Example 35”, the DNA fragment encoding the peptide epitope able to form the pMHC Multiplexer and able to bind T-cells can be amplified in a PCR reaction and sequenced using an appropriate sequencing platform.

For the library “pMHC Multiplexers of Example 36” and where the Acid peptide is inserted in the PstI/XhoI site a different set of PCR primers flanking the specific part encoding the peptide epitope library will be used. PCR amplification followed by DNA sequencing will identify the corresponding peptide epitopes.

### Example 39. Screening of Antigen-Specific T Cells Using Fluorescently Labelled pMHC Multiplexers

This is a modification of Example 38 (Screening of antigen-specific T cells using fluorescently labelled pMHC Multiplexers).

In this example, all pMHC Multiplexers and pMHC Multiplexer libraries described throughout this invention can be used as well, since fluorochrome-labelled anti-MHC1 antibody and fluorochrome-labelled anti-MHC2 antibody will label them all. Adequately labelled pMHC Multiplexer and T-cells complexes can be selected by FACS sorting as described in Example 38.

In another embodiment, the pMHC Multiplexer/T-cell complexes can be selected using a magnetic bead system, where magnetic microbeads can be specifically attached to MHC1 and/or MHC2 complexes, to relevant molecular or cellular structures on e.g. dendrit cells, phages or microbes.

### Example 40. Generation of pMHC1 Multiplexers by Extracellular pMHC1 Formation and Attachment of pMHC1 to Phage by Acid-Base Dimerization

This example is a modification of Example 1 (Display of pMHC Class 2 complexes by phage particles via pVIII encoding peptide X on a phagemid).

In this new example, the Multiplexers consists of extracellular pMHC1 complexes attached to phage by acid-base dimerization.
Steps 1-14 may be carried out as described in Example 1
Step 15 of Example 1, describing the cloning and expression of test epitopes and control peptides may be changed to reflect that shorter peptide of typically—but not restricted to—8-10 amino acids bind MHC1. Relevant MHC1 epitopes, design of corresponding oligonucleotides including adequately positioned DNA restriction sites are known to persons skilled in the art and need not be further described here.
Steps 16-21 may be carried out as described in Example 1.
Step 22. A Solution containing a Base Peptide-Heavy Chain (Base-HC) fusion protein in complex with β2m (thus generating a Base-tagged MHC1 complex) are transferred into each of the micro titer wells to a final concentration preferably lower than the estimated concentration of peptide X in the individual well. Here, a Base Peptide variant is used that carries a cysteine, capable of forming a disulfide bond with Acid Peptide upon their complexation.
Step 23. The temperature is decreased to approximately 20° C., to allow complex formation between peptide X and the Base-tagged MHC1 complex, to potentially form the peptide X-MHC1 complex.
Step 24. Optionally, a redox buffer is added, to first reduce the cysteine in each of the Acid Peptide and the Base Peptide.
Step 25. Optionally, the oxidation/reduction status of the buffer is adjusted to allow disulfide bond formation between the cysteines of the Acid Peptide and the Base Peptide. As a result, the peptide X-MHC1 complex becomes covalently linked to the Acid Peptide-pVIII fusion protein of the phage coat.

The final product is thus a number of pMHC1 Multiplexers, more specifically a number of phages displaying multiple copies of the same base peptide pMHC1 complex attached to the coat of the phage, and the phage carrying a DNA inside that encodes the peptide of the pMHC1 complex attached to the phage's coat. The collection of pMHC1 multiplexers are called “pMHC1 Multiplexers using base-peptide-MHC1 protein of Example 1”.

### Example 41. Generation of pMHC1 Multiplexers by Extracellular pMHC Formation from Peptide and Peptide-Receptive MHC1, and Attachment of pMHC1 to Phage by Acid-Base Dimerization

This example is a modification of Example 40

In this example, the pMHC Multiplexers comprise of pMHC1 complexes (e.g. HLA-A*02:01) formed extracellularly by complexation of peptide (p) and peptide-receptive MHC1 complexes, and attached to phage by acid-base dimerization. Peptide-receptive MHC1, also termed empty-loadable complexes are MHC class I molecules stabilized—in this specific example—by a disulfide bond to link the α1 and α2 helices close to the F pocket and are described in Saini et al., Sci. Immunol. 4, eaau9039 (2019). These disulfide-stabilized MHC class I molecules can be loaded with peptide in the multimerized form allowing for easy binding and display of different peptide epitopes and corresponding formation of functional pMHC1 complexes.

Peptide-receptive MHC1 complexes can be formed using other isotypes including but not limited to HLA-A (HLA-A), HLA-B (HLA-B), HLA-C(HLA-C), and some less polymorphic such as HLA-E (HLA-E), HLA-F (HLA-F), HLA-G (HLA-G)

### Protocol:

Steps 1-21. Are carried out as described in Example 40.
Step 22. Construction, expression, purification, solubilization and MHC-complex formation of a peptide-receptive (i.e. disulphide-stabilized) Heavy Chain (HC) are described in Saini et al., Sci. Immunol. 4, eaau9039 (2019). To construct a Base-peptide-disulphide-stabilized peptide-receptive Heavy chain (Base-HC), the corresponding DNA sequence of the base peptide (base peptide aa sequence AQLKKKLQALKKKNAQLKWKLQALKKKLAQ) is cloned in frame at the N-terminal part of the Heavy Chain.
Step 23. Production of MHC-I heavy chain and β2m

Base peptide fusions of peptide-receptive MHC-I heavy chains and native β2m can be produced in E. coli, as described in D. N. Garboczi, D. T. Hung, D. C. Wiley, HLA-A2-peptide complexes: Refolding and crystallization of molecules expressed in Escherichia coli and complexed with single antigenic peptides. Proc. Natl. Acad. Sci. U.S.A. 89, 3429-3433 (1992), and in S. K. Saini, E. T. Abualrous, A.-S. Tigan, K. Covella, U. Wellbrock, S. Springer, Not all empty MHC class I molecules are molten globules: Tryptophan fluorescence reveals a two-step mechanism of thermal denaturation. Mol. Immunol. 54, 386-396 (2013). Briefly, proteins are expressed in E. coli strain BL21(DE3)pLysS using pET series plasmids. Inclusion bodies containing expressed proteins can be harvested using sonication in lysis buffer followed by washing in detergent buffer and wash buffer and solubilizing the protein in 8 M urea buffer [8 M urea, 50 mM K·Hepes (pH 6.5), and 100 μM β-mercaptoethanol]. Proteins can be stored at −80° C. until they are to be used for in vitro folding.
Step 24. In vitro folding and purification of MHC-I molecules

Base peptide/peptide-receptive MHC-I heavy chain (HLA-A*02:01) fusion (1 μM) and β2m (2 μM) are diluted in a folding buffer [0.1 M tris (pH 8.0), 500 mM l-arginine-HCl, 2 mM EDTA, 0.5 mM oxidized glutathione, and 5 mM reduced glutathione] with 10 mM dipeptide GM (HLA-A*02:01) and incubated at 4° C. for 3 to 5 days, followed by concentrating folded proteins with 30-kDa cutoff membrane filters (Vivaflow 200; Sartorius). MHC-I monomers can be further purified by various chromatography methods and stored at −80° C. until further use.
Step 25. A Solution containing a Base Peptide fused to the peptide-receptive Heavy chain (Base-HC) fusion protein in complex with Beta-2M (thus generating a Base-tagged peptide-receptive MHC1 complex) are transferred into each of the micro titer wells of Step 18-21 described above and in Example 1 and Example 40 to a final concentration preferably lower than the estimated concentration of peptide X in the individual well after Step 20.
Step 26. The temperature is decreased to approximately 20° C., to allow complex formation between peptide X and the Base-tagged peptide-receptive MHC1 complex, to potentially form the peptide X-MHC1 complex.
Step 27. Optionally, other temperatures above or below 20° C. can be tested in steps of 5° C., e.g. 25° C., 30° C., 35° C. or 15° C., 10° C. or 5° C.
Step 28. Optionally, a redox buffer is added, to first reduce the cysteine in each of the Acid Peptide and the Base Peptide.
Step 29. Optionally, the oxidation/reduction status of the buffer is adjusted to allow disulfide bond formation between the cysteines of the Acid Peptide and the Base Peptide. As a result, the base peptide of the peptide X-MHC1 complex becomes covalently linked to the Acid Peptide-pVIII fusion protein of the phage coat.

Base peptide can be fused to Heavy chain, it can be fused to β2m, and it can be fused at to the N-terminus of HC or β2m, or it can be fused to the C-terminus of HC or β2m.

In this example, MHC1 isotype HLA-A*02:01 is used. Other isotypes can be used as well and include but are not limited to HLA-A (HLA-A), HLA-B (HLA-B), HLA-C(HLA-C), and some less polymorphic such as HLA-E (HLA-E), HLA-F (HLA-F), HLA-G (HLA-G).

The final product is thus a number of pMHC1 Multiplexers, more specifically a number of phages displaying multiple copies of the same base peptide pMHC1 complex attached to the coat of the phage, and the phage carrying a DNA inside that encodes the peptide of the pMHC1 complex attached to the phage's coat. The collection of pMHC1 multiplexers are called “pMHC1 Multiplexers using base-peptide-peptide-receptive MHC1 protein of Example 1”.

### Variations of the Above Protocol.

In Step 22, the Base peptide may alternatively be fused to the C-terminal of the HC.
In Step 22, a Base-peptide-peptide-receptive MHC1 complex may alternatively be prepared by fusion of the DNA encoding the base peptide (base peptide aa sequence AQLKKKLQALKKKNAQLKWKLQALKKKLAQ) in frame with B2M at the N- or C-terminal end of B2M, followed by purification of Base-peptide-B2M protein fusion, and complexation with the cysteine-stabilized peptide-receptive HC described above.
In Step **22**, DNA encoding a Base Peptide variant which carries a cysteine can be used, allowing the Base peptide to form a disulfide bond with a cysteine-containing Acid Peptide upon their complexation.

The following references were referred to in the general Description and in the Examples and Figures section:
Fagerlund, A., Myrset, A. H., and Kulseth, M. A. (2008). Construction and characterization of a 9-mer phage display pVIII-library with regulated peptide density. Applied microbiology and biotechnology 80, 925-936.
Kay, B. C., Winter, J., and McCafferty, J. (1996). Phage Display of Peptides and Proteins.
Lanzer, M., and Bujard, H. (1988). Promoters largely determine the efficiency of repressor action. Proceedings of the National Academy of Sciences of the United States of America 85, 8973-8977.
Matos, C. F., Branston, S. D., Albiniak, A., Dhanoya, A., Freedman, R. B., Keshavarz-Moore, E., and Robinson, C. (2012). High-yield export of a native heterologous protein to the periplasm by the tat translocation pathway in Escherichia coli. Biotechnology and bioengineering 109, 2533-2542.
O'Shea, E. K., Lumb, K. J., and Kim, P. S. (1993). Peptide ‘Velcro’: design of a heterodimeric coiled coil. Current biology: CB 3, 658-667.
Ringquist, S., Shinedling, S., Barrick, D., Green, L., Binkley, J., Stormo, G. D., and Gold, L. (1992). Translation initiation in Escherichia coli: sequences within the ribosome-binding site. Molecular microbiology 6, 1219-1229.
Thomas, J. D., Daniel, R. A., Errington, J., and Robinson, C. (2001). Export of active green fluorescent protein to the periplasm by the twin-arginine translocase (Tat) pathway in Escherichia coli. Molecular microbiology 39, 47-53.

1. A composition of 10 or more pMHC Multiplexers, such as 100 or more pMHC Multiplexers, wherein each pMHC Multiplexer comprises a unique encoding molecule of non-human origin, such as a DNA or RNA molecule, mechanically linked to a unique pMHC complex where the peptide (p) of the pMHC complex or the pMHC complex itself is encoded by said encoding molecule.

2. The composition according to claim 1 comprising 1,000 or more pMHC Multiplexers.

3. The composition according to claims 1-2 comprising 10,000 or more pMHC Multiplexers, such as 100,000 or more pMHC Multiplexers, such as 1,000,000 or more pMHC Multiplexers.

4. The composition according to claims 1-3, wherein each of the pMHC complexes is chemically linked to a phage or virus coat protein by way of a non-covalent link.

5. The composition according to claims 1-4, wherein each of the pMHC complexes is chemically linked to a phage, wherein the phage is filamentous phage M13 and the chemical link comprises a chemical entity, such as a tetrazole, which is not found in natural peptides made up of the 20 natural amino acids.

6. The composition according to any one of the preceding claims, wherein each pMHC complex is functionally linked to a human cell, such as a human dendritic cell; a yeast cell, or a bacterial cell.

7. The composition according to any one of the preceding claims, wherein the multiplexer comprises a bacterial cell, a yeast cell, a human cell, a dendritic cell, an antigen-presenting cell, a virus particle, or a phage particle.

8. The composition according to any one of the preceding claims, wherein the peptide (p) is a cancer-specific epitope, a virus-specific epitope, or a bacterium-specific epitope.

9. The composition according to any one of the preceding claims, wherein the composition of pMHC Multiplexers includes more than 100 unique peptides from the human genome, a viral genome, a bacterial genome or a fungal genome; or includes more than 100 random sequence peptides.

10. The composition according to any one of the preceding claims, wherein the unique peptide (p) comprises between 2 and 1000 amino acid residues, such as between 5 and 200 amino acid residues, such as between 6 and 60 amino acid residues, such as between 7 and 20 amino acid residues, such as between 7 and 11 amino acid residues.

11. The composition according to any one of the preceding claims, wherein the unique peptide (p) is derived from the proteomes of the following group of viruses: adenovirus, retrovirus, herpes simplex virus, vaccinia virus, or influenza virus.

12. The composition according to any one of the preceding claims, wherein the RNA is a collection of mRNA molecules purified from a cell extract such as a cell extract from a cancer patient, e.g. a cell extract of the cells from a biopsy from the tumor of a cancer patient, or alternatively, cDNA prepared from a cell extract, amplified by e.g. PCR and then transcribed into mRNA, or mRNA made by transcription from (optionally recombinant) viral DNA or other vector DNA such as plasmids, or made from transcription of single-stranded or double-stranded oligonucleotides prepared synthetic chemistry.

13. A process for making a composition of pMHC Multiplexers, comprising the following steps:
i) providing a collection of phage- or virus particles, each of which comprises a DNA or RNA molecule encoding a unique peptide (p);
ii) transferring one of the phage- or virus particles into each of several containers, such as into each of several wells of a microtiter plate, where each of the containers comprise cells, growth medium and other conditions appropriate for the amplification of the phage- or virus particles and expression of the unique peptides in said cells;
iii) adding a molecule comprising two reactive groups, A and X, where reactive group A is capable of forming a covalent bond to the surface of one of the phage- or virus particles, by reaction with a reactive group on the surface of one of the phage- or virus particles;
iv) allowing the reaction of reactive group A with surface of a phage- or virus particle;
v) optionally, partially lyse the cells;
vi) releasing the unique peptides of the cells into the growth medium of the container;
vii) adding beta2M and HC peptide, where one of the peptides carries a reactive group Y capable of reacting with reactive group X to form a covalent bond, or adding preformed MHC 1 complex carrying a reactive group Y capable of reacting with reactive group X to form a covalent bond; or adding alpha and beta subunits, where one of the subunits carries a reactive group Y capable of reacting with reactive group X to form a covalent bond, or adding preformed MHC 2 complex carrying a reactive group Y capable of reacting with reactive group X to form a covalent bond;
viii) optionally, introducing denaturing conditions in the containers;
ix) optionally, introducing renaturing conditions in the containers;
x) allowing the reaction between reactive groups X and Y;
xi) thereby leading to the formation of a pMHC complex and its chemical attachment to a phage particle by reaction of reactive group X with reactive group Y to form a covalent bond linking the pMHC complex to the surface of the phage- or virus particles, where the unique peptide (p) of the pMHC complex is encoded by the DNA or RNA comprised within the phage- or virus particle that the pMHC complex is attached to;
where the above steps may be done in any sequence,
thereby producing pMHC Multiplexers.

14. The process according to claim 13 wherein the reactive group X is a triple bond and reactive group Y is an azide, or wherein the reactive group Y is a triple bond and reactive group X is an azide.

15. A process for making a composition of pMHC Multiplexers, comprising the following steps:
i) providing a collection of phage- or virus particles, each of which comprises a DNA or RNA molecule encoding a unique peptide (p) and a protein fusion between a phage coat protein and a first dimerization domain peptide, such as the Acid Peptide;
ii) transferring one of the phage- or virus particles into each of several containers, such as into each of several wells of a microtiter plate, where each of the containers comprise cells, growth medium and other conditions appropriate for the amplification of the phage- or virus particles and expression of the unique peptides in said cells, thereby leading to, in each container, the generation of multiple copies of the unique peptide (p) and multiple copies of the phage- or virus particle that carries within it the DNA or RNA that encodes the unique peptide, where the phage- or virus particle displays on its surface the first dimerization domain peptide;
iii) optionally, partially lyse the cells;
iv) releasing the unique peptides into the growth medium;
v) adding the two proteins of a MHC complex-either separately or as a pre-formed complex-one of which is fused to a second dimerization domain peptide such as the Base peptide, capable of dimerizing with the first dimerization domain peptide;
vi) optionally, introducing denaturing conditions in each of the containers;
xii) optionally, introducing renaturing conditions in each of the containers;
xiii) allowing the first and second dimerization domain peptides to bind to each other and leading to the formation of a pMHC complex and chemical attachment of the pMHC complex to the phage- or virus particle,
where the above steps may be done in any sequence,
thereby producing pMHC Multiplexers.

16. A screening process involving the composition of pMHC Multiplexers according to any one of claims 1-12.

17. The screening process of claim 15 where the screening process involves flow cytometry or centrifugation or beads.

18. A process for making a composition of cell-based pMHC Multiplexers, comprising the following steps:
i) Preparing a collection of encoding molecules, capable of being transcribed and/or translated into peptides or proteins, where each encoding molecule encodes the peptide of the pMHC complex of the a final MHC Multiplexer, or where each encoding molecule encodes the precursor peptide or precursor protein of the peptide of the pMHC complex being displayed in multiple copies in a final MHC Multiplexer;
ii) introducing the collection of encoding molecules into dendritic cells or precursors of dendritic cells, thereby generating a collection of cells, each comprising one or more copies of an encoding molecule,
iii) allowing the transcription and/or translation of said encoding molecules;
iv) optionally, allowing the partial degradation and/or modification of peptide (p);
v) allowing complexation of peptide (p) with MHC complex, to form pMHC complex, and allowing the transfer of the pMHC complex onto the surface of the dendritic cell;
where steps ii), iii), iv), v), and vi) may be performed in any order, thereby producing a composition of pMHC Multiplexers.

19. A process for making a composition of pMHC Multiplexers, comprising the following steps:
i) preparing a collection of two or more phage genomes where each genome carries a DNA sequence encoding a promoter controlling the transcription of a DNA encoding a fusion-protein of a signal peptide and a unique peptide epitope, where said peptide epitope is capable of complexing with a MHC1 or MHC2 complex when it is not attached to the signal peptide;
ii) introducing the collection of phage genomes of step (i) into the cells of a growing E. coli culture, by e.g. transformation, and growing the E. coli cells for several generations;
iii) transferring aliquots of the supernatant, comprising on average less than 1 phage particle, to individual wells of a microtiter-plate comprising growing E. coli cultures;
iv) growing the E. coli cultures, to produce phage particles;
v) partially lysing the cells, to release peptides encoded by the phage genome and optionally releasing phage particles into the periplasm;
vi) optionally, removing cells and cell debris by e.g. centrifugation and transfer of the supernatant to wells of another microtiter-plate;
vii) adding a compound comprising at least two reactive groups (a) and (x), to the solution comprising the phage and peptide X, where (a) is capable of reacting with an amino acid residue of the phage coat protein, and where (x) is capable of reacting with a reactive group (y);
viii) adding MHC1 complexes that have been modified on at least one surface-exposed amino acid residue with a moiety (y) capable of reacting with (x), and allowing reaction of (x) and (y), to covalently link the pMHC1 complexes to the phage coat;
ix) denaturing the proteins of the MHC complexes attached to the phage coat;
x) renaturing the proteins, thereby allowing the formation of pMHC1 complexes, where the peptide component is the peptide X present in the same well, thereby forming in each well a unique pMHC Multiplexer.

20. The process according to claim 19 wherein the reactive group (x) is a triple bond and reactive group (y) is an azide, or wherein the reactive group (y) is a triple bond and reactive group (x) is an azide.

21. A process for making a collection of pMHC Multiplexers, comprising the following steps:
i) preparing a collection of phagemids that all carry a DNA sequence encoding a peptide (p) in reading frame with and N-terminal to the pk VIII coat protein of phage M13, and where each of the DNA sequences encode a unique peptide (p) of between 7 and 25 amino acid residues;
ii) introducing the collection of phagemids of step (i) into the cells of a growing E. coli culture, by e.g. transformation;
iii) adding Helper Phage, to produce phage particles displaying the peptide (p) on pVIII coat protein;
iv) adding a MHC complex, such as an peptide receptive MHC complex,
thereby producing a collection of pMHC Multiplexers.

22. A process for making a collection of pMHC Multiplexers, comprising the following steps:
i) preparing a collection of phagemids that all carry a DNA sequence encoding a peptide (p) in reading frame with a dimerization domain X, and where each of the DNA sequences encode a unique peptide (p) of between 7 and 25 amino acid residues, and carrying a DNA sequence encoding a dimerization domain Y fused to the pVIII coat protein, where the X and Y dimerization domains are capable of dimerizing to each other;
ii) introducing the collection of phagemids of step (i) into the cells of a growing E. coli culture, by e.g. transformation;
iii) adding Helper Phage, to produce phage particles displaying the peptide (p) on a phage coat protein;
iv) adding a MHC complex, such as an empty MHC2 complex;
thereby producing a collection of pMHC Multiplexers.

23. A process for making a collection of pMHC Multiplexers, comprising the following steps:
i) preparing a collection of encoding molecules, capable of being transcribed and/or translated into peptides or proteins, where each encoding molecule encodes the peptide of the pMHC complex being displayed in multiple copies in the a final MHC Multiplexer, or where each encodes the precursor peptide or precursor protein of the peptide of the pMHC complex being displayed in multiple copies in a final MHC Multiplexer; and where the encoding molecules may be a collection of RNA molecules or a collection of DNA molecules, where the DNA molecules may be made by synthetic chemistry or may be made by enzymatic means such as by reverse transcription of an mRNA, followed by amplification e.g. by PCR, and where the DNA may be e.g. a wild type virus or a genetically modified recombinant virus e.g. belonging to the following group of viruses: adenovirus, retrovirus, herpes simplex virus, vaccinia virus, influenza virus, and alpha virus; and where the RNA may be a collection of mRNA molecules purified from a cell extract such as a cell extract from a cancer patient, e.g. a cell extract of the cells from a biopsy from the tumor of a cancer patient, or alternatively, cDNA may be prepared from a cell extract, amplified by e.g. PCR and then transcribed into mRNA, or mRNA may be made by transcription from (optionally recombinant) viral DNA or other vector DNA such as plasmids, or may be made from transcription of single-stranded or double-stranded oligonucleotides prepared synthetic chemistry;
ii) introducing the collection of encoding molecules into a dendritic cell or a precursor of a dendritic cell, under conditions ensuring that one cell only receives one or more copies of the same encoding molecule, thereby generating a collection of cells, each comprising one or more copies of an encoding molecule, by e.g. electroporation, infection by e.g. virus, phagocytosis of another cell, e.g. a monocyte or bacteria, uptake of lipid nanoparticles or vesicles or other similar entities, or uptake of small lipid-comprising carriers, infection by e.g. bacterium, or transfection, e.g. using liposomes such as DOTAP;
iii) optionally, adding activating or inhibiting molecule(s);
iv) incubating;
v) allowing the transcription and/or translation of said encoding molecules;
vi) allowing the partial degradation and/or modification and complexation of peptide (p) with MHC complex, to form pMHC complex, and allowing the transfer of the pMHC complex onto the surface of the dendritic cell, thereby resulting in a display of the peptide in complex with MHC protein;
where steps ii), iii), iv), v), and vi) may be performed in any order, thereby producing pMHC Multiplexers.

24. A process for making a collection of pMHC Multiplexer, comprising the following steps:
i) preparing a collection of encoding molecules, capable of being transcribed and/or translated into peptides or proteins, where each encoding molecule encodes the peptide of the pMHC complex being displayed in multiple copies in the a final MHC Multiplexer, or where each encodes the precursor peptide or precursor protein of the peptide of the pMHC complex being displayed in multiple copies in a final MHC Multiplexer; and where the encoding molecules may be a collection of RNA molecules or a collection of DNA molecules, where the DNA molecules may be made by synthetic chemistry or may be made by enzymatic means such as by reverse transcription of an mRNA, followed by amplification e.g. by PCR, and where the DNA may be e.g. a wild type virus or a genetically modified recombinant virus e.g. belonging to the following group of viruses: adenovirus, retrovirus, herpes simplex virus, vaccinia virus, influenza virus, and alpha virus; and where the RNA may be a collection of mRNA molecules purified from a cell extract such as a cell extract from a cancer patient, e.g. a cell extract of the cells from a biopsy from the tumor of a cancer patient, or alternatively, cDNA may be prepared from a cell extract, amplified by e.g. PCR and then transcribed into mRNA, or mRNA may be made by transcription from (optionally recombinant) viral DNA or other vector DNA such as plasmids, or may be made from transcription of single-stranded or double-stranded oligonucleotides prepared synthetic chemistry;
ii) introducing the collection of encoding molecules into a dendritic cell or a precursor of a dendritic cell, under conditions ensuring that one cell only receives one or more copies of the same encoding molecule, thereby generating a collection of cells, each comprising one or more copies of an encoding molecule, by e.g. electroporation, infection by e.g. virus, phagocytosis of another cell, e.g. a monocyte or bacteria, uptake of lipid nanoparticles or vesicles or other similar entities, or uptake of small lipid-comprising carriers, infection by e.g. bacterium, or transfection, e.g. using liposomes such as DOTAP;
iii) adding activating or inhibiting molecule(s);
iv) incubating;
v) allowing the transcription and/or translation of said encoding molecules;
vi) allowing the partial degradation and/or modification and complexation of peptide (p) with MHC complex, to form pMHC complex, and allowing the transfer of the pMHC complex onto the surface of the dendritic cell, thereby resulting in a display of the peptide in complex with MHC protein;
where steps ii), iii), iv), v), and vi) may be performed in any order, thereby producing pMHC Multiplexers.

25. A process for making a collection of pMHC Multiplexers, comprising the following steps:
i) preparing a collection of phagemids that all carry a DNA sequence encoding the Acid Peptide in reading frame with and N-terminal to the pIII coat protein of phage M13, and where each of the phagemids carry a unique DNA sequence leading to the expression of a unique peptide of between 7 and 25 amino acid residues;
ii) preparing a DNA vector such as a plasmid that comprises a sequence encoding the beta2M protein and a fusion protein, Base Peptide-HC protein;
iii) introducing the collection of phagemids and the vector of step (i) and (ii) into the cells of a growing E. coli culture, by e.g. transformation;
iv) adding Helper Phage, to produce intracellular phage particles displaying the Acid Peptide on pIII coat protein;
v) allowing the assembly of the Base Peptide-HC with the beta2M and the unique peptide, to form the pMHC complex thereof;
vi) allowing the assembly of the Acid-Base dimer, thereby producing intracellular phage particles displaying a pMHC complex on the pIII coat protein;
vii) adding redox buffer and then change to more oxidizing conditions, to form the Acid Peptide-Base Peptide dimer, thereby covalently attaching the pMHC complex to the phage coat protein,
thereby producing pMHC Multiplexers.

26. A process for making a collection of pMHC Multiplexers, comprising the following steps:
i) preparing a collection of encoding molecules, capable of being transcribed and/or translated into peptides or proteins;
ii) introducing the collection of encoding molecules into a dendritic cell or a precursor of a dendritic cell by e.g. electroporation or infection;
iii) adding activating or inhibiting molecule(s);
iv) incubating;
v) allowing the transcription and/or translation of said encoding molecules;
vi) allowing the partial degradation and/or modification and complexation of peptide (p) with MHC complex, to form pMHC complex, displayed on a dendritic cell;
where steps ii), iii), iv), and v) may be performed in any order, thereby producing pMHC Multiplexers.

27. A pair of pMHC Multiplexers as defined in any one of claims 1-12, where the encoding molecule is a single-stranded oligonucleotide and where the 3′-terminus of the encoding molecule of one pMHC Multiplexer consists of at least 3 nucleotides that is complementary to at least 3 nucleotides of the 3′-terminus of the encoding molecule of the other pMHC Multiplexer.

28. A composition of two or more, such as at least 10 or more, such as at least 100 or more, such as at least 1000 or more, such as at least 10000 or more, such as at least 100000 or more, such as at least 1000000 or more, such as at least 10000000 or more, such as at least 100000000 or more, such as at least 1000000000 or more, such as at least 10000000000 or more, such as at least 100000000000 or more pairs of pMHC Multiplexers according to claim 27.

29. A method for the detection or isolation of an antigen-specific T cell, comprising the following steps:
i) providing one or more T cells,
ii) providing one or more pairs of pMHC Multiplexers according to claim 27,
iii) allowing the one or more pairs of pMHC Multiplexers to bind to the one or more T cells, and allowing any pair of pMHC Multiplexers bound to the same T cell to form a duplex by having their encoding molecules form a duplex,
iv) extending each of the oligonucleotides of the duplex from the 3′-end in a template-dependent manner, optionally incorporating labelled dNTPs into the extended DNA strand,
v) optionally, determining the degree of incorporation of dNTPs by determining the amount of incorporated label,
vi) determining the identity of the pMHC Multiplexers bound to a T cell, by one of the processes (a), (b), or (c):
a. performing flow sorting, to isolate labelled T cells, followed by sequencing of the encoding molecules bound to the isolated cells, thereby determining the identity of the pMHC complexes of the pMHC Multiplexer that was bound to a T cell,
b. adding primers that anneal to the 3′-ends of the oligonucleotides, and performing a PCR reaction, optionally incorporating labelled dNTPs, and measuring the amount of label attached to the pMHC Multiplexers or sequencing the PCR products thereby determining the identity of the pMHC complexes of the pMHC Multiplexer that was bound to a T cell,
c. sequencing the encoding molecules of the pMHC Multiplexers thereby determining the identity of the pMHC complexes of the pMHC Multiplexer that was bound to a T cell,
thereby identifying the pMHC-binding specificity of the T cell that was bound to said pMHC Multiplexer.

30. A process for making a collection of pMHC Multiplexer, comprising the following steps:
i) providing 1000 wells each comprising a dimerization domain Y, each domain Y being capable of binding to domain X, and being attached to a unique pMHC complex;
ii) adding to each of the 1000 wells a unique DNA molecule, attached to a dimerization domain Y, capable of binding to domain X;
iii) adding to each of the 1000 wells a SP1 protein, where each of the 12 subunits of the SP1 protein is attached to a dimerization domain X, said domain X being capable of binding to a dimerization domain Y;
where steps (i) to (iii) can be performed in any order;
iv) allowing the X and Y dimerization domain to form an XY dimer,
thereby producing 1000 pMHC Multiplexers, each of which comprise a unique peptide (p) complexed to MHC protein, and comprising a unique DNA encoding said unique peptide (p).
